(19) United States
(12) Patent Application Publication
(10) Pub. No.: US 2005/0239841 A1
(43) Pub. Date: Oct. 27, 2005

(54) New compounds

(76) Inventors: Andrew Browning, Knivsta (SE); Jonas Nilsson, Stockholm (SE); Martin Scobie, Uppsala (SE); Johan Angbrant, Uppsala (SE); Rune Ringom, Uppsala (SE)

Correspondence Address: FISH & RICHARDSON PC, P.O. BOX 1022, MINNEAPOLIS, MN, 55440-1022, US

(21) Appl. No.: 10/997,675

(22) Filed: Nov. 24, 2004

Related U.S. Application Data
(60) Provisional application No. 60/581,057, filed on Jun. 18, 2004.

(30) Foreign Application Priority Data
Nov. 26, 2003 (SE) 0303182-0

(51) Int. Cl. A61K 31/454; A61K 31/416; A61K 31/4178; A61K 31/405; C07D 43/02

(52) U.S. Cl. 514/323; 514/419; 514/406; 546/200; 548/495; 514/339; 514/414; 514/397; 548/465; 548/364.7; 548/311.1

(57) ABSTRACT

The invention relates to compounds of the general formula (I) 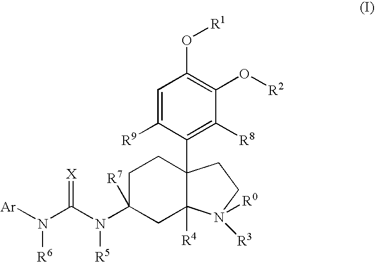
wherein R⁰, R¹, R², R³, R⁴, R⁵, R⁶, R⁷, R⁸, R⁹, Ar, and X are as defined in the description, or a pharmaceutically acceptable salt, hydrates, geometrical isomers, racemates, tautomers, optical isomers, N-oxides and prodrug forms thereof. The compounds may be used for the treatment or prophylaxis of disorders related to the MCH1R receptor and for modulation of appetite. The invention also relates to such use as well as to pharmaceutical formulations comprising a compound of formula (I).

### RELATED APPLICATIONS

This application claims priority to Swedish application number 0303182-0, filed on Nov. 26, 2003, and U.S. provisional application 60/581,057, filed on Jun. 18, 2004, the contents of which is incorporated herein by reference.

### TECHNICAL FIELD

The present invention relates to substituted octahydroindoles that act as antagonists for the melanin concentrating hormone receptor 1 (MCH1R). The invention further relates to pharmaceutical compositions comprising these compounds, and to the use of the compounds for the preparation of a medicament for the prophylaxis and treatment of obesity as well as method of treatment of these disorders.

### BACKGROUND

Melanin Concentrating Hormone (MCH) is a 19 amino acid cyclic peptide, which is expressed in hypothalamus in the mammalian brain (Nahon J L et al., Endocrinology, 1989; 125 (4):2056-65 and Tritos N A, et al., Diabetes, 1998; 47 (11): 1687-92). A substantial body of evidence has shown that this peptide plays a critical role in the moderation of feeding behavior and energy expenditure. Studies have shown that ICV administration of MCH directly into rat brains results in a marked increase in food intake (Ludwig D S et al., Am. J. Physiol., 1998; 274 (4 Pt 1):E627-33). It has also been shown that messenger RNA for the MCH precursor is up-regulated in the hypothalamus of fasted animals and in animals that are genetically obese (Qu D, Ludwig D S et al., Nature, 1996; 380 (6571):243-7). Furthermore, mice lacking MCH are hypophagic and lean, and have increased energy expenditure (20% increase over control animals when expressed on a per kg basis) (Shimada M et al., Nature, 1998; 396 (6712):670-4). Studies of transgenic mice overexpressing MCH in the lateral hypothalamus show that these animals are more prone to diet-induced obesity when fed a high fat diet, and they have higher systemic leptin levels (Ludwig D S et al., J. Clin. Invest., 2001; 107 (3):379-86). Blood glucose levels were increased both preprandially and after intraperitoneal injection of glucose. The animals also had increased insulin levels and insulin tolerance test indicated peripheral insulin resistance. Further support for the role of MCH in metabolic regulation comes from studies showing that, in mice, mRNA for the MCH receptor is upregulated 7-fold by 48 h fasting and in genetic leptin deficiency (ob/ob mice). These effects could be completely blunted by leptin treatment (Kokkotou E G et al., Endocrinology, 2001; 142 (2):680-6.). In addition to its role in regulating feeding behavior, MCH antagonists have been demonstrated to have anxiolytic and antidepressant effects (Borowsky, B D et al., Nature Medicine, 2002. 8 (8): 825-830).

Obesity is linked to a wide range of medical complications, such as diabetes, cardiovascular disease and cancer. In addition, being overweight can exacerbate the development of osteoporosis and asthma. For example, at least 75% of Type II diabetics are overweight and a clear correlation has been demonstrated between weight and the prevalence of Type II diabetes. Obesity is also proven to double the risk of hypertension. It is estimated that between 2% and 8% of total health-care costs in the Western world are related to obesity, i.e. in excess of 10 billion USD.

Initial treatment for obesity is simple diet and exercise. Initial drug therapy tends to be focused around suppression of appetite. Many of the older appetite-suppressant agents act via the noradrenergic (and possibly dopaminergic) receptors to produce a feeling of satiety. Amphetamine was the archetypal agent in this class, but it has substantial potential for stimulating the central nervous system and consequent abuse. More recent developments, such as Xenical® (orlistat), marketed by Roche, have focused on preventing fat absorption in the gut. Xenical® inhibits the action of the enzyme lipases, thereby reducing the digestion of triglycerides and subsequent absorption by the intestinal tract. Unfortunately, this does not address overeating and excess calorie intake. Other pharmacological approaches for the treatment of obesity include serotonin re-uptake inhibitors, such as Reductil® (sibutramine) marketed by Abbot, which acts as an appetite-suppressant. The concept of using MCH1R antagonists for the treatment of obesity has recently been published. A review is presented by Carpenter and Hertzog, Expert Opin. Ther. Patents, 2002, 12 (11): 1639-1646.

WO01/21169 (Takeda Chemical Industries) describes diaryl compounds as MCH-1R antagonists useful for the treatment of obesity. Also JP13226269 (Takeda), describing several piperidine-substituted benzazepines and benzazepinones; WO01/82925 (Takeda), disclosing different amines; and WO01/87834 (Takeda) describing piperidine compound with benzene (1:1), claim compounds for the treatment of obesity. WO01/21577 (Takeda) discloses a series of amines claimed to be anorectic, antidiabetic and antidepressant agents. WO01/57070 (Merck) describes in a series of truncated and modified peptidic MCH analogues as either significant agonist or antagonist activity. In WO02/10146 (GlaxoSmithKmine) the preparation of carboxamide compounds claimed for the treatment of obesity as well as diabetes, depression and anxiety is disclosed. WO02/04433 (The Neurogen Corporation) describes in N-arylpiperazine derivatives and related 4-arylpiperidine derivatives as selective modulators of MCH-1R for the treatment of a variety of metabolic, feeding and sexual disorders. In WO02/06245 (Synaptic Pharmaceutical Corporation) a class of dihydropyrimidinones as MCH-1R antagonists for the treatment of feeding disorders, such as obesity and bulimia is disclosed. In WO02/051809 (Schering Corporation) 4-substituted piperidine derivatives are disclosed as MCH antagonists as well as their use in the treatment of obesity. In WO02/057233 aryl-substituted ureas are disclosed as MCH antagonists as well as their use in the treatment of obesity. The central core in the WO02/057233 is a(n) (hetero)arylene group. Mesembrine, 3a-(3,4-dimethoxyphenyl)-1-methyloctahydro-6H-indol-6-one, is a natural product obtained as an extract of plants of the Mesembryanthemaceae family, including Sceletium tortuosum. In small doses the mesembrine have a meditative or narcotic effect. Hottentots used Sceletium expansum and tortuosum as a psychedelic called “channa”. The use of mesembrine as a serotonin-uptake inhibitor for the treatment of an array of mental disorders is disclosed in WO97/46234. U.S. Pat. No. 6,288,104 discloses mesembrine-like. This document relates to serotonin-uptake inhibitors for the treatment of depression and anxiety.

### SUMMARY OF THE INVENTION

According to the present invention, novel substituted octahydroindoles have been found that are active towards the MCH1R receptor. The compounds are relatively easy to prepare and can be used for the treatment or prevention of obesity, diabetes mellitus, hyperlipidemia, hyperglycemia, modulation of appetite, depression, anxiety or urinary incontinence. The compounds can further be used in conjunction with other compounds acting through other mechanisms, such as MC-4 agonists, 5HT_{2c} agonists, or 5HT₆ antagonists. The compounds can also be used in conjunction with anti-obesity medicaments.

The following definitions shall apply throughout the specification and the appended claims.

Unless otherwise stated or indicated, the term “C₁₋₆-alkyl” denotes a straight or branched alkyl group having from 1 to 6 carbon atoms, inclusive (i.e., 1, 2, 3, 4, 5 or 6 carbon atoms). Examples of said lower alkyl include methyl, ethyl, n-propyl, iso-propyl, n-butyl, iso-butyl, sec-butyl, t-butyl and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. “Halo-C₁₋₆-alkyl” means a C₁₋₆-alkyl group substituted by one or more halogen atoms.

Unless otherwise stated or indicated, the term “C₃₋₈-cycloalkyl” denotes a cyclic alkyl group having a ring size from 3 to 8 carbon atoms, inclusive (i.e., 3, 4, 5, 6, 7 or 8 carbons atoms). Examples of said cycloalkyl include cyclopropyl, cyclobutyl, cyclopentyl, cyclohexyl, methylcyclohexyl, cycloheptyl, cyclooctyl, and bicyclo[2.2.1]hept-2-yl. For parts of the range “C₃₋₈-cycloalkyl” all subgroups thereof are contemplated such as C₃₋₇-cycloalkyl, C₃₋₄-cycloalkyl, C₃₋₅-cycloalkyl, C₃₋₄-cycloalkyl, C₄₋₈-cycloalkyl, C₄₋₇-cycloalkyl, C₄₋₆-cycloalkyl, C₄₋₅-cycloalkyl, C₅₋₇-cycloalkyl, C₆₋₇-cycloalkyl, etc.

Unless otherwise stated or indicated, the term “C₁₋₆ alkoxy” denotes a straight or branched alkoxy group having from 1 to 6 carbon atoms, inclusive (i.e., 1, 2, 3, 4, 5 or 6 carbon atoms). Examples of said lower alkoxy include methoxy, ethoxy, n-propoxy, iso-propoxy, n-butoxy, iso-butoxy, sec-butoxy, t-butoxy and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc. “Halo-C₁₋₆-alkoxy” means a C₁₋₆-alkoxy group substituted by one or more halogen atoms.

Unless otherwise stated or indicated, the term “C₁₋₆-acyl” denotes a straight, branched, cyclic or aromatic hydrocarbon having from 1 to 6 carbon atoms, with a carbonyl group. Exemplary acyl groups include formyl, acetyl, propanoyl, butanoyl, pentanoyl, hexanoyl, benzoyl, etc. For parts of the range “C₁₋₆-acyl” all subgroups thereof are contemplated such as C₁₋₅-acyl, C₁₋₄-acyl, C₁₋₃-acyl, C₁₋₂-acyl, C₂₋₆-acyl, C₂₋₅-acyl, C₂₋₄-acyl, C₂₋₃-acyl, C₃₋₆-acyl, C₄₋₅-acyl, etc.

Unless otherwise stated or indicated, the term “C₁₋₃-alkylene” denotes a straight or branched alkylene group having from 1 to 3 carbon atoms, inclusive. Examples of said alkylene include methylene —(CH₂)—, ethylene —(CH₂CH₂)—, and propylene —(CH₂CH₂CH₂)—.

Unless otherwise stated or indicated, the term “C₂₋₆-alkenyl” denotes a straight or branched alkenyl group having from 2 to 6 carbon atoms, inclusive. Examples of said alkenyl include vinyl, allyl, 1-butenyl, 1-pentenyl, and 1-hexenyl. For parts of the range “C₂₋₆-alkenyl” all subgroups thereof are contemplated such as C₂₋₅-alkenyl, C₂₋₄-alkenyl, C₂₋₃-alkenyl, C₃₋₆-alkenyl, C₃₋₅-alkenyl, C₃₋₄-alkenyl, C₄₋₆-alkenyl, C₄₋₅-alkenyl, etc.

Unless otherwise stated or indicated, the term “halogen” shall mean fluorine, chlorine, bromine or iodine.

Unless otherwise stated or indicated, the term “aryl” refers to a hydrocarbon ring system having at least one aromatic ring. Examples of aryls are phenyl, pentalenyl, indenyl, dihydroindenyl, isoindolinyl, chromanyl, naphthyl, fluorenyl, anthryl, phenanthryl and pyrenyl. The aryl rings may optionally be substituted by C₁₋₆-alkyl. Examples of substituted aryl groups are benzyl and 2-methylphenyl.

The term “heteroaryl” refers to a hydrocarbon ring system having at least one aromatic ring which contains at least one heteroatom such as O, N, or S. Examples of heteroaryl groups include furyl, pyrrolyl, thienyl, oxazolyl, isoxazolyl, imidazolyl, thiazolyl, isothiazolyl, pyridinyl, pyrimidinyl, quinazolinyl, indolyl, pyrazolyl, pyridazinyl, quinolinyl, benzofuranyl, dihydrobenzofuranyl, benzodioxolyl, benzodioxinyl, benzothiazolyl, benzothiadiazolyl, benzimidazolyl, benzothienyl, 1,1-dioxobenzothienyl, and benzotriazolyl groups. The term “heterocyclyl” refers to a hydrocarbon ring system containing 4 to 8 ring members that have at least one heteroatom (e.g., S, N, or O) as part of the ring. It includes saturated, unsaturated, aromatic, and nonaromatic heterocycles.

Suitable heterocyclic groups include the above-mentioned heteroaryl groups, pyrrolidinyl, piperidyl, azepinyl, morpholinyl, thiomorpholinyl, pyranyl, and dioxanyl groups.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “prodrug forms” means a pharmacologically acceptable derivative, such as an ester or an amide, which derivative is biotransformed in the body to form the active drug. Reference is made to Goodman and Gilman's, The Pharmacological basis of Therapeutics, 8^{th} ed., Mc-Graw-Hill, Int. Ed. 1992, “Biotransformation of Drugs”, p. 13-15.

The term “coupling agent” refers to a substance capable of catalyzing a coupling reaction, such as amidation, or esterification. Examples of coupling agents include, but are not limited to, carbonyldiimidazole, dicyclohexylcarbodimide, pyridine, 4-dimethylaminopyridine, and triphenylphosphine.

The term “reducing agent” refers to a substance capable of reducing another substance and it itself is oxidized. Examples of reducing agents include, but are not limited to, hydrogen, sodium, potassium, sodium borohydride, sodium cyanoborohydride, sodium triacetoxyborohydride, lithium aluminiumhydride, and diisobutylaluminium hydride.

When two of the above-mentioned terms are used together, it is intended that the latter group is substituted by the former. For example, aryl-C₁₋₆ alkyl means a C₁₋₆ alkyl group that is substituted by an aryl group. Likewise, halo Clot alkoxy means a C₁₋₆ alkoxy group that is substituted by one or more halogen atoms.

The following abbreviations have been used:
(Boc)₂O means di-tert-butyl carbonate,
DCE means dichloroethane,
DCM means dichloromethane,
DIBAL-H means diisobutylaluminium hydride,
DIPEA means diisopropylethylamine,
DMF means dimethylformamide,
DPPA means diphenylphosphorylazide,
HOBt means hydroxybenzotriazole,
HPLC means high performance liquid chromatography,
LDA means lithium diisopropylamide,
NOE means nuclear Overhauser effect,
PNP means para-nitrophenyl;
R.T. (rt) means room temperature,
TFA means trifluoroacetic acid,
THF means tetrahydrofuran.

In a first aspect, the present invention provides a compound of the general formula (I) 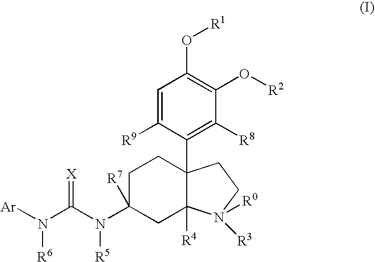
or a pharmaceutically acceptable salt, hydrates, geometrical isomers, racemates, tautomers, optical isomers, N-oxides and prodrug forms thereof, wherein:
R⁰ is C₁₋₆ alkyl or is absent;
each of R¹ and R² is independently hydrogen, C₁₋₆ alkyl, haloC₁₋₆ alkyl, aryl-C₁₋₆ alkyl; or
R¹ and R² are linked to form a C₁₋₃ alkylene;
R³ is H, carbamoyl, thiocarbamoyl, cyano, C₁₋₆ alkyl, C₂₋₆ alkenyl, C₂₋₆ alkenylsulfonyl, C₃₋₈ cycloalkyl optionally substituted by one or more of C₁₋₆ alkyl, C₃₋₈ cycloalkyl-C₁₋₆ alkyl, hydroxyC₁₋₆ alkyl, halo C₁₋₆ alkyl, halo C₁₋₆ alkylsulfonyl, halo C₁₋₆ acyl, mono- or di-C₁₋₆ alkylamino-C₁₋₆ alkyl, C₁₋₆ acyl, carboxyC₁₋₆ acyl, amino C₁₋₆ acyl, mono- or di-C₁₋₆ alkylamino C₁₋₄ acyl, hydroxyC₁₋₆ alkylaminoC₁₋₆ acyl, arylamino C₁₋₆ acyl, arylC₁₋₆ alkylaminoC₁₋₆ acyl, heteroarylC₁₋₆ alkylaminoC₁₋₆ acyl, heterocyclylC₁₋₆ acyl, azido-C₁₋₆ alkyl, C₁₋₆ alkoxycarbonylthiocarbamoyl, 3- to 7-membered heterocyclyl, heterocyclylC₁₋₆ alkyl, mono- or di-C₁₋₆ alkylaminocarbamoylC₁₋₆ alkyl, heterocyclyl, heterocyclylcarbonyl, aryl, aryl-C₁₋₆ alkyl, cyano C₁₋₆ alkyl, heteroaryl or heteroaryl-C₁₋₆ alkyl,
   wherein any heterocyclyl, aryl or heteroaryl may be unsubstituted or independently substituted in one, two or three positions with oxo, C₁₋₆ alkyl, C₁₋₆ alkylamino, C₁₋₆ alkoxy, C₁₋₆ alkoxy-C₁₋₆ alkyl, C₁₋₆ alkylsilyl, halogen, aryl substituted by one or more of C₁₋₆ alkoxy, heteroaryl, arylC₁₋₆ alkyl, arylsulfonyl or a carbamimidoyl;
R⁴ is H, C₁₋₆ alkyl, or aryl;
each of R⁵ and R⁶ is independently H, C₁₋₆ alkyl, C₂₋₆ alkenyl, C₁₋₆ alkoxy-C₁₋₆ alkyl, C₃₋₈ cycloalkyl, 1-methyl-1,2,3,6-tetrahydro-pyridin-4-yl, arylC₁₋₆ alkyl, biphenylyl optionally substituted by cyano, heterocyclylC₁₋₆ alkyl, wherein the C₃₋₈ cycloalkyl, aryl or heterocyclyl may be unsubstituted or substituted, in one or more than one positions, by C₁₋₆ alkyl;
R⁷ is H or C₁₋₆alkyl;
R⁸ is H or halogen;
R⁹ is H or forms CH₂ together with R³;
Ar is a 5- to 7-membered, aromatic carbon ring; a 5- to 7-membered, unsaturated heterocyclic ring containing 1 to 4 heteroatoms selected from oxygen, nitrogen and sulfur; a bicyclic ring system comprising at least one 5- to 7-membered, aromatic carbon ring; a bicyclic ring system comprising at least one 5- to 7-membered, unsaturated heterocyclic ring containing 1 to 4 heteroatoms selected from oxygen, nitrogen and sulfur; or a tricyclic ring system comprising at least two 5- to 7-membered, aromatic carbon rings;
   wherein the group Ar may be unsubstituted or independently substituted in one, two, three, four or five positions with C₁₋₆ alkyl, halo-C₁₋₆ alkyl, C₁₋₆ alkoxy, halo C₁₋₆ alkoxy, C₁₋₆ acyl, C₁₋₆ alkoxycarbonyl, C₁₋₆ alkylthio, halo C₁₋₆ alkylthio, C₁₋₆ alkylsulfonyl, haloC₁₋₆ alkylsulfonyl, halogen, mono- or di-C₁₋₆ alkylamino, nitro, cyano, C₁₋₆ alkylaminosulfonyl, aryl optionally substituted by one or more halogen atoms, aryloxy, aryl-C₁₋₆ alkoxy, cyanoaryl, 3- to 7-membered saturated or partly unsaturated heterocyclyl, heterocyclylsulfonyl, heteroaryl, or a bicyclic ring system comprising at least one 5- to 7-membered, unsaturated heterocyclic ring containing 1 to 4 heteroatoms selected from oxygen, nitrogen and sulfur, which bicyclic ring system may be unsubstituted or substituted in one or two positions with C₁₋₄ alkyl; and
X is O, S, NH, CH—NO₂, or N—CN.

In some embodiments R¹ and R² are not both H.

In some embodiments R⁰ is methyl or is absent.

In some embodiments each of R¹ and R² independently is hydrogen, methyl, ethyl, n-propyl, isopropyl, benzyl, difluoromethyl, trifluoromethyl or together form a group —(CH₂)—, —(CH₂CH₂)— or —(CH₂CH₂CH₂)—.

In some embodiments R³ is selected from hydrogen, methyl, ethyl, n-propyl, isopropyl, n-butyl, isobutyl, 3-methylbutyl, 2-ethylbutyl, 3,3-dimethylbutyl, cyclobutyl, cyclopentyl, cyclohexyl, cyclopropylmethyl, 2-hydroxyethyl, 2-fluoroethyl, 3,3,3-trifluoropropyl, 3,3,3-trifluoro-2-methylpropyl, dimethylaminoethyl, 3-dimethylamino-2,2-dimethyl-propyl, acetyl, dimethylaminoacetyl, propylaminoacetyl, 2-azidoethyl, 1-methylpiperidin-4-yl, benzyl, 4-methylbenzyl, 4-fluorobenzyl, tetrahydro-2H-pyran-4-yl, tetrahydro-3-thienyl, 3-furylmethyl, (5-methyl-2-furyl)methyl, (4,5-dimethyl-2-furyl)methyl, 3-thienylmethyl, (5-ethyl-2-thienyl)methyl, 1-methyl-1H-pyrrol-2-yl-methyl, pyridylmethyl, (3,5-dimethyl-1H-isooxazol-4-yl)methyl, 1,3-thiazol-2-yl-methyl, 1H-imidazol-2-yl-methyl, 1H-imidazol-4-yl-methyl, (1-methyl-1H-imidazol-2-yl)methyl, (2-methyl-1H-imidazol-4-yl)methyl, (2-ethyl-1H-imidazol-4-yl)methyl, (2-ethyl-4-methyl-1H-imidazol-5-yl)methyl, (4-methyl-1H-imidazol-5-yl)methyl, 1H-pyrazol-3-yl-methyl, (1,3-dimethyl-1H-pyrazol-5-yl)methyl, (5-chloro-1,3-dimethyl-1H-pyrazol-4-yl)methyl, 2-[4-(methoxymethyl)-1H-1,2,3-triazol-1-yl]ethyl, [5-(methoxymethyl)-1H-1,2,3-triazol-1-yl]ethyl, [4-(trimethylsilyl)-1H-1,2,3-triazol-1-yl]ethyl, {5-[(4-methylphenyl)sulfonyl]-1H-tetrazol-1-yl}ethyl, N-cyano-N′-ethyl-carbamimidoyl, isopropylaminoacetyl, phenylaminoacetyl, chloroacetyl, aminoacetyl, methylaminoacetyl, 3-(4-methoxyphenyl)isoxazol-5-ylmethyl, 1,2,3-triazol-5-ylmethyl, cyanomethyl, 2-furylmethylaminoacetyl, 4-pyridylmethylaminoacetyl, 4-chlorobenzylaminoacetyl, ethylaminoacetyl, 4-methyl-1-piperidinylacetyl, 1-pyrrolidinylacetyl, 2-hydroxyethylaminoacetyl, 1-benzyl-3-pyrrolidinyl, 3,3,5,5-tetramethyl-1-cyclohexyl, bicyclo[2.2.1]hept-2-yl, 4-methyl-1-cyclohexyl, 2-(5-methoxytetrazol-1-yl)ethyl, 2-(5-oxotetrazol-1-yl)ethyl, 2-(5-n-propylaminotetrazol-1-yl)ethyl, 2-[5-(1-imidazolyl)tetrazol-1-yl]ethyl, allyl, dimethylaminocarbamoylmethyl, tert-butyl, carbethoxythiocarbamoyl, 4-methyl-2-thiazolyl, 4-methyl-1-piperazinylcarbonyl, 2-carboxyethylcarbonyl, cyclopropyl, carbamoyl, thiocarbamoyl, cyano, 2-pyrimidinyl, vinylsulfonyl, 2-(4-methyl-1-piperidinyl)ethyl, 3-(1-piperidinyl)propyl, phenyl, chloromethylsulfonyl, formyl, and 2-(1-piperidinyl)ethyl.

In some embodiments R⁴ is hydrogen.

In some embodiments R⁵ is selected from hydrogen, methyl, ethyl, n-propyl, isopropyl, n-butyl, allyl, methoxyethyl, cyclohexyl, benzyl, 3-cyano-(1,1′-biphenyl)-4-yl, 3-(4-methyl-1-piperazinyl)propyl, and tetrahydropyridyl substituted by methyl.

In some embodiments R⁶ is hydrogen or methyl.

In some embodiments R⁷ is hydrogen or n-propyl.

In some embodiments R⁹ is hydrogen or fluoro.

In some embodiments R³ and R⁹ form together CH₂.

In some embodiments Ar is phenyl, furyl, thienyl, pyridinyl, isoxazolyl, isothiazolyl, thiazolyl, pyrazol, pyridazinyl, indenyl, dihydroindenyl, naphthyl, pyrimidinyl, fluorenyl, indolyl, quinolinyl, benzimidazolyl, benzofuranyl, 1,1-dioxobenzothienyl, dihydrobenzofuranyl, benzodioxolyl, benzodioxinyl, benzothiazolyl, benzothiadiazolyl, or benzotriazolyl, wherein the group Ar may be unsubstituted or independently substituted in one, two, three, four or five positions with C₁₋₆ alkyl, haloC₁₋₆ alkyl, C₁₋₆ alkoxy, haloC₁₋₆ alkoxy, C₁₋₆ acyl, C₁₋₆ alkoxycarbonyl, C₁₋₆ alkylthio, haloC₁₋₆ alkylthio, C₁₋₆ alkylsulfonyl, haloC₁₋₆ alkylsulfonyl, halogen, mono- or di-C₁₋₆ alkylamino, nitro, cyano, C₁₋₆ alkylaminosulfonyl, aryl optionally substituted by one or more halogen atoms, aryloxy, cyanoaryl, aryl-C₁₋₆ alkoxy, 3- to 7-membered saturated or partly unsaturated heterocyclyl, heterocyclylsulfonyl, heteroaryl, or C₁₋₆ alkyl substituted bicyclic ring system comprising at least one 5- to 7-membered, unsaturated heterocyclic ring containing 1 to 4 heteroatoms selected from oxygen, nitrogen and sulfur.

In certain of the embodiments in which X is oxygen, Ar is
phenyl, which may be unsubstituted or independently substituted in one, two, three, four or five positions with C₁₋₆ alkyl, haloC₁₋₄ alkyl, C₁₋₆ alkoxy, haloC₁₋₆ alkoxy, C₁₋₆ alkylthio, halo C₁₋₆ alkylthio, C₁₋₆ alkylsulfonyl, haloC₁₋₆ alkylsulfonyl, C₁₋₆ acyl, C₁₋₆ alkoxycarbonyl, halogen, mono- or di-C₁₋₆ alkylamino, nitro, cyano, C₁₋₆ alkylaminosulfonyl, phenyl, phenoxy, benzyloxy, cyanophenyl, tetrazolyl, oxazolyl, 4-bromo-1-methyl-1H-pyrazol-3-yl, heterocyclyl, C₁₋₆ alkylbenzothiazolyl, or thiadiazolyl;
furyl, which may be unsubstituted or independently substituted in one or two positions with C₁₋₆ alkyl or halo C₁₋₆ alkyl;
thienyl;
pyridinyl, which may be unsubstituted or independently substituted in one or two positions with C₁₋₆ alkyl, halo C₁₋₆ alkyl, C₁₋₆ alkoxy, halogen or nitro;
pyrazolyl, which may be unsubstituted or independently substituted in two or three positions with C₁₋₆ alkyl or halogen;
pyridazinyl;
isoxazolyl, which may be unsubstituted or independently substituted in one or two positions with C₁₋₆ alkyl;
isothiazolyl, which may be unsubstituted or substituted in one position with C₁₋₆ alkyl;
indenyl, which may be unsaturated or partly saturated;
naphthyl;
fluorenyl;
indolyl, which may be substituted in one position with C₁₋₆ alkyl;
quinolinyl; which may be unsubstituted or independently substituted in one position with C₁₋₆ alkyl, halogen, and haloC₁₋₆ alkyl;
benzofuranyl; which may be unsaturated or partly saturated;
benzodioxolyl;
benzodioxinyl, which may be unsubstituted or independently substituted in one or two positions with one or two halogen atoms in each position;
benzothiazolyl, which may be unsubstituted or independently substituted in one position with C₁₋₆ alkyl and halogen;
benzothiadiazolyl, which may be unsubstituted or substituted in one position with C₁₋₆ alkyl;
benzotriazolyl; thiazolyl, which may be unsubstituted or independently substituted in one position with C₁₋₆ alkyl, halogen and phenyl substituted by one or more halogen atoms;
1,1-dioxobenzothienyl;
benzimidazolyl, which may be unsubstituted or independently substituted in one or more positions with haloC₁₋₆ alkyl; or
pyrimidinyl, which may be unsubstituted or independently substituted in one or more positions with halogen.

In certain of the embodiments in which X is oxygen Ar is
phenyl, which may be unsubstituted or independently substituted in one, two, three, four, or five positions with methyl, ethyl, isopropyl, n-butyl, tert-butyl, trifluoromethyl, methoxy, difluoromethoxy, trifluoromethoxy, 1,1,2,2-tetrafluoroethoxy, methylthio, trifluoromethylthio, acetyl, ethoxycarbonyl, methylsulfonyl, trifluoromethylsulfonyl, fluoro, chloro, bromo, iodo, dimethylamino, nitro, cyano, n-butylaminosulfonyl, morpholinyl, phenyl, cyanophenyl, thiadiazolyl, phenoxy, benzyloxy, tetrazolyl, oxazolyl, 4-bromo-1-methyl-1H-pyrazol-3-yl, 6-methyl-1,3-benzothiazol-2-yl;
difluoromethylenedioxyphenyl;
furyl, which may be unsubstituted or independently substituted in one or two positions with methyl or trifluoromethyl;
thienyl;
pyridinyl, which may be unsubstituted or independently substituted in one or two positions with methyl, ethyl, methoxy, ethoxy, trifluoromethyl, chloro, bromo or nitro;
pyrazolyl, which may be unsubstituted or independently substituted in one two or three positions with methyl or bromo;
pyridazinyl;
isoxazolyl, which may be unsubstituted or independently substituted in one or two positions with methyl;
isothiazolyl, substituted in one position with methyl;
dihydroindenyl;
naphthyl;
fluorenyl;
indolyl, substituted in one position with methyl;
quinolinyl, which may be unsubstituted or independently substituted in one position with methyl, halogen and trifluoromethyl;
dihydrobenzofuranyl;
benzodioxolyl;
benzodioxinyl, which may be unsubstituted or independently substituted in one or two positions with one or two fluoro atoms in each position;
benzothiazolyl, which may be unsubstituted or independently substituted in one position with methyl or halogen;
benzothiadiazolyl;
benzotriazolyl;
thiazolyl, which may be unsubstituted or independently substituted in one position with methyl, halogen and 2-chloro-6-fluorophenyl;
1,1-dioxobenzothienyl;
benzimidazolyl, which may be unsubstituted or independently substituted in one position with trifluoromethyl; or pyrimidinyl, which may be unsubstituted or independently substituted in one or more positions with halogen.

In certain of the embodiments in which X is sulfur, R⁰ is absent; R¹ and R² are both methyl; R³ is methyl or benzyl; and R⁴-R⁷ are all hydrogen.

In certain of the embodiments in which X is sulfur, Ar is
phenyl, which may be unsubstituted or independently substituted in one or two positions with methyl, isopropyl, methoxy, methylthio, trifluoromethyl, fluoro, chloro, dimethylamino, nitro, piperidinylsulfonyl, pyrazolyl or oxazolyl;
thienyl, which may be unsubstituted or substituted in one position with methoxycarbonyl;
pyridyl, which may be unsubstituted or substituted in one position with phenoxy or morpholinyl; or
dihydroindenyl.

In certain of the embodiments in which X is NH, R⁰ is absent; R¹ and R² are both methyl; R³ is methyl; R⁴-R⁷ are all hydrogen; and Ar is phenyl which is substituted in two positions with fluoro or which is substituted in two positions with chloro.

In certain of the embodiments in which X is CH—NO₂, it is preferred that R⁰ is absent; R¹, R² and R³ are all methyl; R⁴-R⁷ are all hydrogen; and Ar is phenyl which is substituted in two positions with chloro.

In certain of the embodiments in which X is N—CN, R⁰ is absent; R¹ and R² are both methyl; R³ is methyl; R⁴-R⁷ are all hydrogen; and Ar is phenyl that is independently substituted in one or two positions with trifluoromethyl, chloro or bromo; or Ar is pyridyl that is independently substituted in one or two positions with methoxy or chloro.

Desirable compound include those in Examples 12-328, 336-484, 486-490, 492-495, 497-502, 505-511, and 514-548 below.

All diastereomeric forms possible (pure enantiomers, tautomers, racemic mixtures and unequal mixtures of two enantiomers) are within the scope of the invention. Such compounds can also occur as cis- or trans-, E- or Z-double bond isomer forms. All isomeric forms are contemplated.

Another object of the present invention is a process for the preparation of a compound above comprising at least one of the following reaction sequences:
(a) treatment of an amine with an isocyanate or isothiocyanate,
(b) treatment of a first amine with para-nitrophenyl chloroformate and then with Hunigs base to give a PNP-carbamate, and finally treatment of a second amine with the PNP-carbamate,
(c) treatment of an amine with a carboximidamide hydrochloride in the presence of a base,
(d) treatment of mesembrine with an amine, reduction of the imine formed and then treatment of the resultant amine with an isocyanate,
(e) treatment of an amine with a cyanoimidocarbamate,
(f) treatment of an amine with a nitrovinyl compound,
(g) alkylation of an amine with a ketone via reductive amination and then treatment with an isocyanate,
(h) N-debenzylation of an amine by treatment with hydrogen,
(i) alkylation of an amine with an aldehyde via reductive amination,
(j) treatment of a first amine with triphosgene and then with a second amine,
(k) acylation of an exocyclic amino nitrogen of an amine with a carboxylic acid in the presence of a base and diphenylphosphorylazide,
(l) O-alkylation of an amine with an alkyl halide,
(m) acylation of an endocyclic amino nitrogen of an amine with a carboxylic acid in the presence of a base and a coupling agent,
(n) alkylation of an amine with a halo substituted aldehyde via reductive amination, then nucleophilic displacement of the halogen atom with an azide, and optionally ring closure to give either a triazole or a tetrazole,
(o) alkylation of an amine with a ketone via reductive amination,
(p) alkylation of an amine with a halo substituted aldehyde via reductive amination and then nucleophilic displacement of the halogen atom with an amine,
(q) treatment of a ketone with benzylamine, then a Grignard reagent, debenzylation and finally with an isocyanate,
(r) N-alkylation of an amine with an alkyl halide,
(s) O-dealkylation of an amine by treatment with boron tribromide, and
(t) O-debenzylation of an amine by treatment with hydrogen,
(u) alkylation of a nitrile by treatment with 1-bromo-2-chloroethane,
(v) reduction of a nitrile to an aldehyde,
(w) reaction of an aldehyde with benzylamine to give an imine,
(x) reaction of an imine with but-3-en-2-one to give a ketone,
(y) reaction of a ketone with a reducing agent and an ammonium salt to give an amine,
(z) acylation of an amine with a haloacyl halide to give a haloamide,
(aa) amidation of a haloamide with ammonia or an amine,
(bb) treatment of an amine with an isothiocyanate and the subsequent basic hydrolysis to give a thiourea derivative,
(cc) ring closure of a thiourea derivative with a haloketone to give a thiazole derivative,
(dd) acylation of an amine with an acyl halide to give an amide,
(ee) acylation of an amine with a carboxylic acid anhydride to give a carboxylic acid,
(ff) treatment of an amine with cyanogen bromide,
(gg) treatment of an amine with an aryl halide,
(hh) treatment of an amine with a sulfonyl halide,
(ii) reaction of an aldehyde with an aromatic amine to give an imine,
(jj) acylation of an amine with a carboxylic ester,
(kk) alkylation of an amine with a silane, and
(ll) ring closure of an amine with formaldehyde.

Another object of the present invention is a compound as described above for use in therapy. The compound can be used in the treatment or prophylaxis of obesity, diabetes mellitus, hyperlipidemia, hyperglycemia, depression, anxiety, urinary incontinence, and for modulation of appetite. It may also be used in the treatment or prophylaxis of disorders relating to the MCH1R receptor and for modulation of appetite. Examples of such disorders are obesity, diabetes mellitus, hyperlipidemia, hyperglycemia, depression, anxiety, and urinary incontinence. The compound can further be used in conjunction with other compounds active towards other receptors, such as MC-4 agonists, 5HT_{2c} agonists, or 5HT₆ antagonists. The compound can also be used in conjunction with anti-obesity medicaments.

Another object of the present invention is a pharmaceutical formulation containing a compound as described above as an active ingredient, in combination with a pharmaceutically acceptable diluent or carrier. The pharmaceutical formulation may be used in the treatment or prophylaxis of obesity wherein the active ingredient is a compound as described above.

Another object of the present invention is a method for the treatment or prophylaxis of obesity, diabetes mellitus, hyperlipidemia, hyperglycemia, depression, anxiety, urinary incontinence, and for modulation of appetite, said method comprising administering to a subject (e.g., mammal, human, or animal) in need of such treatment an effective amount of a compound as described above. The compound can further be used in conjunction with other compounds active towards other receptors, such as MC-4 agonists, 5HT_{2c} agonists, or 5HT₆ antagonists. The compound can also be used in conjunction with anti-obesity medicaments.

Another object of the present invention is a method for the treatment or prophylaxis of disorders related to the MCH1R receptor and for modulation of appetite, said method comprising administering to a subject (e.g., mammal, human, or animal) in need of such treatment an effective amount of a compound as described above. The MCH1R receptor related disorder is any disorder or symptom wherein the MCH1R receptor is involved in the process or presentation of the disorder or the symptom. The MCH1R related disorders include, but are not limited to obesity, diabetes mellitus, hyperlipidemia, hyperglycemia, depression, anxiety, and urinary incontinence. The compound can further be used in conjunction with other compounds active towards other receptors, such as MC-4 agonists, 5HT₂, agonists, or 5HT₆ antagonists. The compound can also be used in conjunction with anti-obesity medicaments.

The methods delineated herein can also include the step of identifying that the subject is in need of treatment of the MCH1R receptor-related disorder. Identifying a subject in need of such treatment can be in the judgment of a subject or a health care professional and can be subjective (e.g., opinion) or objective (e.g., measurable by a test or diagnostic method).

Another object of the present invention is a method for modulating MCH1R receptor activity (e.g., antagonizing the human MCH1R receptor), comprising administering to a subject (e.g., mammal, human, or animal) in need thereof an effective amount of a compound as described above or a composition comprising a compound as described above.

Another object of the present invention is the use of a compound as described above in the manufacture of a medicament for use in the treatment or prophylaxis of obesity, diabetes mellitus, hyperlipidemia, hyperglycemia, depression, anxiety, and urinary incontinence, and for modulation of appetite.

Another object of the present invention is the use of a compound as described above in the manufacture of a medicament for use in the treatment or prophylaxis of disorders related to the MCH1R receptor and for modulation of appetite, said method comprising administering to a subject (e.g., mammal, human, or animal) in need of such treatment an effective amount of a compound as described above. The MCH1R receptor related disorder is any disorder or symptom wherein the MCH1R receptor is involved in the process or presentation of the disorder or the symptom. The MCH1R related disorders include, but are not limited to obesity, diabetes mellitus, hyperlipidemia, hyperglycemia, depression, anxiety, and urinary incontinence. The compound can further be used in conjunction with other compounds active towards other receptors, such as MC-4 agonists, 5HT_{2c} agonists, or 5HT₆ antagonists. The compound can also be used in conjunction with anti-obesity medicaments.

The compounds of the formula (I) may be used as such or, where appropriate, as pharmacologically acceptable salts (acid or base addition salts) thereof. The pharmacologically acceptable addition salts mentioned above are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulfuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulfonic acid, toluenesulfonic acid, methanesulfonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

In a further aspect the invention relates to methods of making compounds of any of the formulae herein comprising reacting any one or more of the compounds of the formulae delineated herein, including any processes delineated herein. The compounds of the formula (I) above may be prepared by, or in analogy with, conventional methods.

The processes described above may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g., as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof. The necessary starting materials for preparing the compounds of formula (I) are either known or may be prepared in analogy with the preparation of known compounds. The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The invention will now be further illustrated by the following non-limiting Examples.
General Procedures for the Preparation of Compounds of the Present Invention

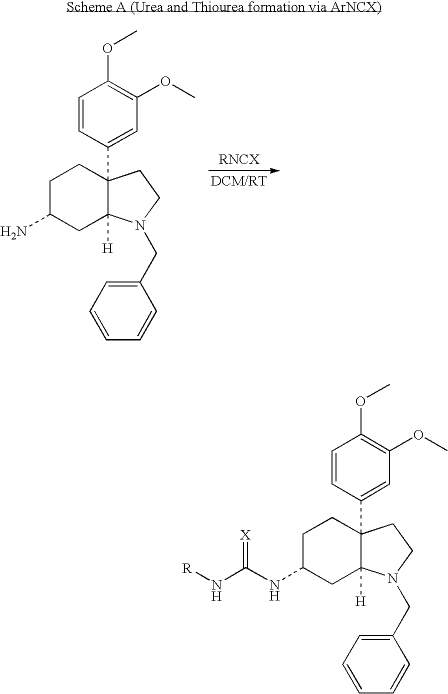

A solution of the amine, (3aS*,6R*,7aS*)-1-benzyl-3a-(3,4-dimethoxyphenyl)octahydro-1H-indol-6-amine, Example 5 (18.3 mg; 0.05 mmol) in methylene chloride (2.0 ml) was treated with an isocyanate or isothiocyanate (1 equiv.; 0.05 mmol) The mixture was shaken at room temperature for 18 h, then the solvent was removed by evaporation

The residues were purified by preparative HPLC using a gradient elution of 20% MeCN—H₂O (containing 0.1% TFA) to 65% MeCN—H₂O (containing 0.1% TFA) over 6 min.

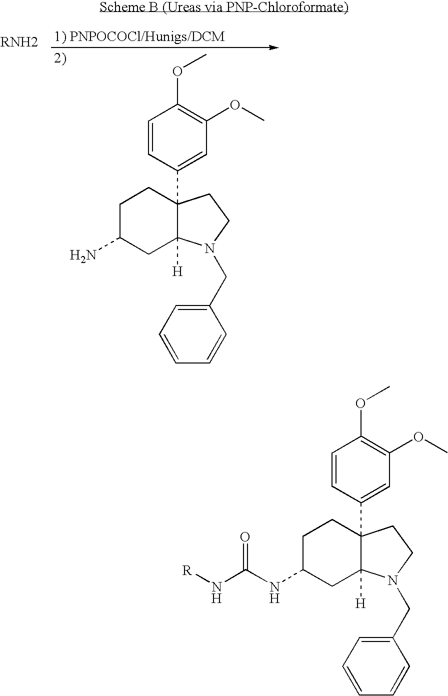

A solution of the appropriate heterocyclic amine (1 mmol) in DCM (5.0 ml) was treated with para-nitrophenyl chloroformate (1 mmol). The resulting solution was then treated dropwise at room temperature with Hunigs base (1 mmol). The mixtures were strirred at room temperature for 5 h.

An aliquot (0.25 ml; 0.05 mmol) of the crude PNP-carbamate from the reaction mixtures described above was then transferred to a solution of the amine, (3aS*,6R*,7aS*)-1-benzyl-3a-(3,4-dimethoxyphenyl)octahydro-1H-indol-6-amine, Example 5 (18 mg; 0.05 mmol) in methylene chloride (3.0 ml) and the resulting solution shaken at R.T overnight.

The solvent was removed by evaporation and the crude reaction mixtures purified by preparative HPLC using a gradient elution of 20% MeCN—H₂O (containing 0.1% TFA) to 65% MeCN—H₂O (containing 0.1% TFA) over 6 min.

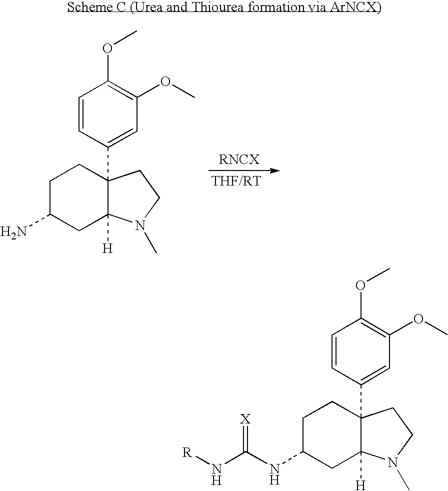

A solution of the amine, (3aS*,6R*,7aS*)-1-methyl-3a-(3,4-dimethoxyphenyl)octahydro-1H-indol-6-amine, Example 7 (7.3 mg; 0.025 mmol) in tetrahydrofuran (1.0 ml) was treated with and isocyanate or isothiocyanate (1 equiv.; 0.025 mmol) The mixture was shaken at room temperature for 18 h, then the solvent was removed by evaporation. The residues were purified by preparative HPLC using a gradient elution of 20% MeCN—H₂O (containing 0.1% TFA) to 65% MeCN—H₂O (containing 0.1% TFA) over 6 min.

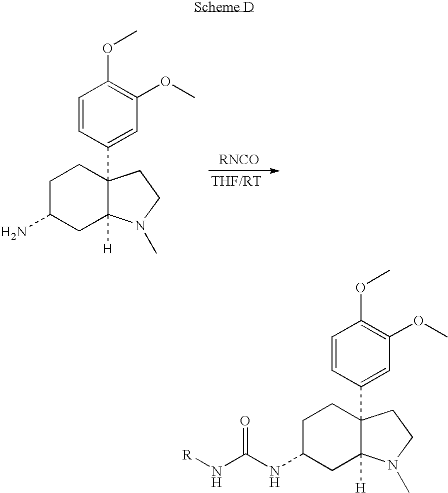

The amine, (3aS*,6R*,7aS*)-1-methyl-3a-(3,4-dimethoxyphenyl)octahydro-1H-indol-6-amine, Example 7 (7 mg, 0.024 mmol) and isocyanate (1.3 eq) were dissolved in dry THF (1.5 ml) Reaction in R.T., under N₂ and overnight. The solvent was evaporated under reduced pressure. Purification on a Gilson Finnigan preparative HPLC.

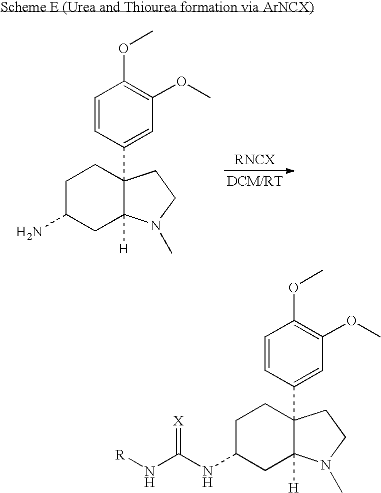

A solution of the amine, (3aS*,6R*,7aS*)-1-methyl-3a-(3,4-dimethoxyphenyl)octahydro-1H-indol-6-amine, Example 7 (10 mg; 0.034 mmol) in methylene chloride (2.0 ml) was treated with the isocyanate or isothiocyanate (1 equiv.; 0.034 mmol) The mixture was shaken at room temperature for 18 h, then the solvent was removed by evaporation The residues were purified by preparative HPLC using a gradient elution of 20% MeCN—H₂O (containing 0.1% TFA) to 65% MeCN—H₂O (containing 0.1% TFA) over 6 min.

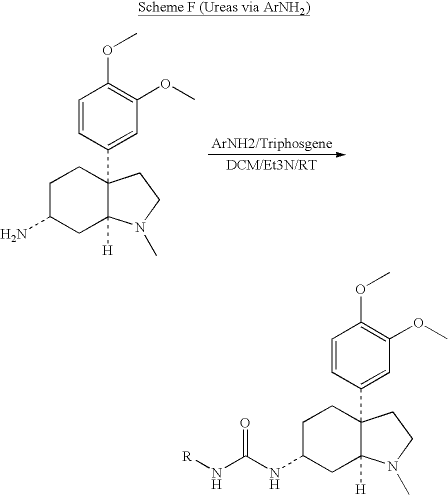

A solution of the appropriate amine/aniline (0.12 mmol) and triethylamine (0.2 mmol) in methylene chloride (2.0 ml) was treated dropwise with a solution of triphosgene (0.4 mmol) in DCM (1.0 ml). The mixtures were shaken at room temperature for 3 h. then treated with a solution of the amine, (3aS*,6R*,7aS*)-1-methyl-3a-(3,4-dimethoxyphenyl)octahydro-1H-indol-6-amine, Example 7 (0.029 g; 0.1 mmol) in dry DCM (1.0 ml) and shaking continued at RT overnight

The residues were purified by preparative HPLC using a gradient elution of 15% MeCN—H₂O (containing 0.1% TFA) to 65% MeCN—H₂O (containing 0.1% TFA) over 6 min.

Synthesis of Starting Materials

### COMPARATIVE EXAMPLE 1

### 1-(3,4-dimethoxyphenyl)cyclolpropanecarbonitrile

Dimethoxyphenyl acetonitrile (4.43 g, 2.5 mmol) was dissolved in DMF (20 mL). Sodium hydride (4 g of a 60% dispersion, 2.4 g, 100 mmol) was added in portions and the mixture was stirred at room temperature for 10 minutes. Bromochloroethane (2.1 mL, 3.62 g, 25.2 mmol) was added, and the mixture stirred at room temperature overnight. The reaction was cautiously quenched by addition of a methanol/water mixture (1:1, 300 mL) and the reaction products were extracted into ethyl acetate (3×200 mL). The combined extracts were washed with water (4×200 mL), brine (1×200 mL) and then dried (Na₂SO₄). The solvent was then removed under reduced pressure and the crude product chromatographed (SiO₂, EtOAc/petroleum ether 1:3 as eluent) to give the title compound as an off-white sold (2.4 g, 47%).

¹H NMR (270 MHz, CDCl₃) δ ppm 1.32 (m, 2H) 1.64 (m, 2H) 3.84 (s, 3H) 3.88 (s 3H) 6.79 (d, J=1.0 Hz, 2H) 6.84 (s 1H)

MS (ESI+) for C₁₂H₁₃NO₂: m/z 204.1 (M+1).

### COMPARATIVE EXAMPLE 2

### 1-(3,4-dimethoxyphenyl)cyclopropanecarbaldehyde

1-(3,4-dimethoxyphenyl)cyclopropanecarbonitrile (Comparative Example 1; 2.0 g, 9.84 mmol) was dissolved in THF (30 mL). DIBAL-H (15 mL of a 1.0 M solution in toluene, 15 mmol) was added and the mixture was stirred at room temperature for 3 hours. The reaction was cautiously quenched by addition of 2 M HCl and organic components were extracted into dichloromethane (3×125 mL). The combined extracts were washed with water (2×100 mL), brine (2×100 mL) and then dried (Na₂SO₄), giving the title compound as an off-white sold (1.95 g, 98%).

¹H NMR (270 MHz, CDCl₃) δ ppm 1.38 (m, 2H) 1.53 (m, 2H) 3.87 (s, 6H) 6.81 (s, 1H) 6.84 (d, J=1.0 Hz, 2H) 9.23 (s, 1H)

MS (ESI+) for C₁₂H₁₄O₃: no ion detected

### COMPARATIVE EXAMPLE 3

### (3aS*,7aS*)-1-benzyl-3a-(3,4-dimethoxyphenyl)octahydro-6H-indol-6-one

1-(3,4-dimethoxyphenyl)cyclopropanecarbaldehyde (Comparative Example 2; 3.25 g, 16.0 mmol) was dissolved in dichloromethane (35 mL). Benzylamine (1.77 mL, 1.74 g, 16.2 mmol) was added, followed by sodium sulfate (15 g, 105.6 mmol). The mixture was stirred at room temperature overnight before being filtered and evaporated to yield the crude imine as a clear oil. This material was then dissolved in DMF (15 m-L), and sodium iodide (246 mg, 1.64 mmol) and trimethylsilyl chloride (202 μL, 172 mg, 1.58 mmol) were added. The resulting mixture was heated to 70° C. for 3 hours and then partitioned between water (150 mL) and ethyl acetate (200 mL). The aqueous phase was extracted with a further portion of ethyl acetate (1×200 mL) and the combined extracts were washed with brine (1×200 mL) and dried (Na₂SO₄). The solvent was removed under reduced pressure, and the crude product dissolved in dichloromethane (30 mL). To this was added HCl in ether (70 mL of a 1.0 M solution, 70 mmol) and the crude HCl salt was evaporated to dryness. This material was then dissolved in acetonitrile (70 mL), methyl vinyl ketone (1.42 mL, 1.19 g, 17 mmol) was added and the mixture heated to reflux for 16 hours. On cooling the solvent was removed under reduced pressure and the resulting dark oil partitioned between 3M HCl solution (200 mL) and ether (150 mL). The aqueous fraction was washed with further ether (3×150 mL), and then brought to basic pH using 3 M NaOH solution. The organic components were then extracted into diethyl ether (3×150 mL) and the combined extracts washed with brine (1×200 mL) and dried (Na₂SO₄). On removal of the solvent under reduced pressure, the crude product was purified by chromatography (SiO₂, ethyl acetate/petroleum ether 2:3 as eluent) to give the title compound as a clear oil (3.10 g, 53%).

¹H NMR (270 MHz, CDCl₃) δ ppm: 1.87-2.38 (m, 6H); (2.38-2.82 (m, 3H); 2.82-3.05 (m, 1H); 3.05-3.20 (m, J=12.6 Hz, 1H); 3.20-3.35 (m, 1H); 3.92 (s, 6H); 3.96-4.19 (m, J=12.6 Hz, 1H); 6.73-7.03 (m, 3H) 7.09-7.42

¹³C NMR (68 MHz, CDCl₃) δ ppm: 34.80, 36.21, 38.61, 40.63, 47.18, 51.67, 53.38, 57.38, 60.32, 68.15, 109.90, 110.95, 117.76, 126.89, 128.15, 128.76, 138.79, 140.32, 147.47, 148.98, 211.36.

### COMPARATIVE EXAMPLE 4

### (3aS*,7aS*)-3a-(3,4-dimethoxyphenyl) 1-methyloctahydro-6H-indol-6-one

1-(3,4-dimethoxyphenyl)cyclopropanecarbaldehyde (Comparative Example 2; 8.0 g, 38.8 mmol) was dissolved in dichloroethane (100 mL). Sodium sulfate (25 g, 176 mmol) was added and methylamine gas was bubbled through the solution for 10 minutes. The reaction vessel was then sealed and the mixture stirred at room temperature overnight before being filtered and evaporated to yield the crude imine as a yellow oil. This material was then dissolved in DMF (30 mL), and sodium iodide (585 mg, 3.90 mmol) and trimethylsilyl chloride (500 μL, 426 mg, 3.92 mmol) were added. The resulting mixture was heated to 90 C for 3 hours and then partitioned between water (200 mL) and ethyl acetate (200 mL). The aqueous phase was extracted with a further ethyl acetate (2×100 mL) and the combined extracts were dried (Na₂SO₄). The solvent was removed under reduced pressure, and the crude product dissolved in dichloromethane (100 mL). To this was added HCl in ether (100 mL of a 1.0 M solution, 100 mmol) and the crude HCl salt was evaporated to dryness. This material was then dissolved in acetonitrile (100 mL), methyl vinyl ketone (3.5 mL, 2.95 g, 42.1 mmol) was added and the mixture heated to reflux for 16 hours. On cooling the solvent was removed under reduced pressure and the resulting dark oil partitioned between 3M HCl solution (200 mL) and ether (200 mL). The aqueous fraction was washed with further ether (2×100 mL), and then brought to basic pH using 3 M NaOH solution. The organic components were then extracted into ethyl acetate (4×150 mL) and the combined extracts washed with brine (1×200 mL) and dried (Na₂SO₄). On removal of the solvent under reduced pressure, the crude product was purified by chromatography (SiO₂, ethyl acetate as eluent) to give the title compound as a yellow oil (4.5 g, 40%).

¹H NMR (270 MHz, CDCl₃) δ ppm: 1.99-2.12 (m, 2H); 2.12-2.26 (m, 3H); 2.28 (s, 3H); 2.30-2.47 (m, 2H); 2.52-2.62 (m, 2H); 2.88-2.95 (m, 1H) 3.06-3.15 (m, 1H) 3.85 (s, 3H); 3.87 (s, 3H); 6.76-6.93 (m, 3H).

¹³C NMR (68 MHz, CDCl₃) δ ppm: 35.20, 36.16, 38.76, 40.01, 40.48, 47.42, 54.78, 55.82, 55.92, 70.31, 109.84, 110.87, 117.83, 140.12, 147.39, 148.90, 211.40.

MS (ESI+) for C₁₇H₂₃NO₃ m/z 290.2 (M+H)⁺. HRMS (EI) calcd for C₁₇H₂₃NO₃: 289.1678, found 289.1684

### COMPARATIVE EXAMPLE 5

### (3aS*,6R*,7aS*)-1-benzyl-3a-(3,4-dimethoxyphenyl)octahydro-1H-indol-6-amine and

### COMPARATIVE EXAMPLE 6

### (3aS*,6S*,7aS*)-1-benzyl-3a-(3,4-dimethoxyphenyl)octahydro-1H-indol-6-amine

(3aS*,7aS*)-1-benzyl-3a-(3,4-dimethoxyphenyl)octahydro-6H-indol-6-one (Comparative Example 3) (750 mg, 2.05 mmol) was dissolved in methanol (60 mL). Ammonium acetate (1.6 g, 20.8 mmol) was added and the solution allowed to stir at room temperature for 2 hours before sodium cyanoborohydride (100 mg, 1.59 mmol) was added. The mixture was stirred at room temperature for 16 hours, diluted with 3 M NaOH solution (100 mL) and extracted into dichloromethane (2×150 mL). The combined extracts were dried (Na₂SO₄) and the solvent removed to give the crude mixture of amines (410 mg, 55%). This crude material was used as a mixture without further purification, or the cis (6R*)- and trans-isomer (6S*) separated by flash chromatography using chloroform saturated with NH₃ (g).

### COMPARATIVE EXAMPLE 7

### (3aS*,6R*,7aS*)-1-methyl-3a-(3,4-dimethoxyphenyl)octahydro-1H-indol-6-amine and

### COMPARATIVE EXAMPLE 8

### (3aS*,6S*,7aS*)-1-methyl-3a-(3,4-dimethoxyphenyl)octahydro-1H-indol-6-amine

Same procedure as for Comparative Example 5 and Comparative Example 6 starting from (3aS*,7aS*)-3a-(3,4-dimethoxyphenyl)-1-methyloctahydro-6H-indol-6-one (Comparative Example 4). This crude material was used as a mixture without further purification, or the cis (6R*)- and trans-isomer (6S*) separated by flash chromatography using chloroform saturated with NH₃ (g).

### COMPARATIVE EXAMPLE 9

### tert-butyl (3aS*,7aS*)-3a-(3,4-dimethoxyphenyl)-6-oxooctahydro-1H-indole-1-carboxylate

Into a solution of Comparative Example 3 (3.0 g, 8.2 mmol) and (Boc)₂O (3.0 g, 13.7 mmol) in i-PrOH (200 mL) was suspended 10% Pd on charcoal (0.8 g), and the resulting mixture was vigorously agitated under H₂ (1.4 atm) during 4 h at rt. The catalyst was filtered off and the filtrate was shaken with PS-trisamine (polystyrene supported trisamine) (3.0 g, 4 mmol/g) at rt overnight. The resin was filtered off and the solvent evaporated, leaving the title compound (2.4 g, 80%) as a thick oil, which was used in the next step without further purification.

¹H NMR (270 MHz, CDCl₃): δ ppm 1.28-1.51 (m, 9H), 1.96-2.38 (m, 6H), 2.43-2.72 (m, 1H), 2.72-2.89 (m, 1H), 3.14-3.45 (m, 1H), 3.68-3.84 (m, 6H), 4.27-4.58 (m, 1H), 6.60-6.84 (m, 3H).

¹³C NMR (270 MHz, CDCl₃): δ ppm 14.22, 21.04, 28.50, 33.22, 36.59, 44.76, 55.90, 55.96, 60.29, 79.87, 100.00, 109.49, 111.20, 117.99, 137.66, 147.86, 149.08, 210.41.

Synthesis of Enantiopure Starting Materials

### COMPARATIVE EXAMPLE 10

### (3aS,6R,7aS)-3a-(3,4-dimethoxyphenyl)-1-methyloctahydro-1H-indol-6-amine

a)

A solution of (3aS*,6R*,7aS*)-1-benzyl-3a-(3,4-dimethoxyphenyl)octahydro-1H-indol-6-amine (prepared in Comparative Example 5) (12.0 g; 0.033 mol) in MeOH (800 ml) was treated with BOC-anhydride (0.034 mol) followed by a 10% w/v solution of sodium bicarbonate (100 ml). The mixture was stirred at RT for 3 h, then concentrated by evaporation to remove most of the methanol and extracted with ethyl acetate to give the product tert-butyl [(3aS*,6R*,7aS*)-1-benzyl-3a-(3,4-dimethoxyphenyl)octahydro-1H-indol-6-yl]carbamate (13.25 g; 87%) which was dissolved in MeOH (280 ml) and treated with glacial acetic acid (20.0 ml) and 10% palladium-on-charcoal (1.32 g). The resulting suspension was hydrogenated under hydrogen overnight.

The mixture was filtered through celite and evaporated under reduced pressure to afford the product as its acetate salt, tert-butyl [(3aS*,6R*,7aS*)-3a-(3,4-dimethoxyphenyl)octahydro-1H-indol-6-yl]carbamate acetate.

Yield=15.2 g (Quant.—contains some excess acetic acid)

b) Resolution of Enantiomers

tert-Butyl [(3aS*,6R*,7aS*)-3a-(3,4-dimethoxyphenyl)octahydro-1H-indol-6-yl]carbamate acetate (7.0 g) was resolved into separate enantiomers by HPLC over a chiral column according to the following procedure.
Column—Chirobiotic V (modified), 250×22.1 mm, 5 micron Silica
Mobile phase 100/0.5/0.5 MeOH/AcOH/Triethylamine
Flow Rate 15 ml/min
Detection 254 nM
Run Time 18 min
Loading 140 mg in 2 ml MeOH

The pooled eluents were concentrated by evaporation and each product further purified by flash-chromatography over silica to afford 2.2 g of the first eluted enantiomer tert-butyl [(3aS,6R,7aS)-3a-(3,4-dimethoxyphenyl)octahydro-1H-indol-6-yl]carbamate (99.7% ee). Similar work-up of the 2nd eluted enantiomer gave 2.2 g tert-butyl [(3aR,6S,7aR)-3a-(3,4-dimethoxyphenyl)octahydro-1H-indol-6-yl]carbamate (97.7% ee)

c)

A solution of the first eluted enantiomer tert-butyl [(3aS,6R,7aS)-3a-(3,4-dimethoxyphenyl)octahydro-1H-indol-6-yl]carbamate (300 mg; 0.8 mmol) in methanol (15.0 ml) was treated with 38% aq formaldehyde (2.5 ml) then sodium cyanoborohydride (0.75 g). The mixture was stirred at RT overnight.

The resulting solution was evaporated then treated with 2M NaOH (5.0 ml) and extracted with DCM to afford the crude product (0.22 g; 56%). An aliquot of this material (100 mg; 0.256 mmol) was treated with a solution of 50% v/v TFA-DCM (5.0 ml) and the resulting solution stirred at RT for 45 min. The mixture was evaporated and the residue treated with 2M NaOH then extracted with DCM to afford the product (3aS,6R,7aS)-3a-(3,4-dimethoxyphenyl)-1-methyloctahydro-1H-indol-6-amine (57 mg, 77%)

### COMPARATIVE EXAMPLE 11

### (3aR,6S,7aR)-3a-(3,4-dimethoxyphenyl)-1-methyloctahydro-1H-indol-6-amine

tert-butyl [(3aR,6S,7aR)-3a-(3,4-dimethoxyphenyl)octahydro-1H-indol-6-yl]carbamate (300 mg; 0.8 mmol), prepared as described in Comparative Example 10 above, was dissolved in methanol (15.0 ml) and treated with a solution of 38% aq formaldehyde (2.5 ml) then sodium cyanoborohydride (0.75 g). The mixture was stirred at RT overnight. The resulting solution was evaporated then treated with 2M NaOH (5.0 ml) and extracted with DCM to afford the product (0.26 g; 83%). An aliquot of this material (200 mg; 0.51 mmol) was treated with a solution of 50% v/v TFA-DCM (5.0 ml) and the resulting solution stirred at RT for 45 min. The mixture was evaporated and the residue treated with 2M NaOH then extracted with DCM to afford the product (3aR,6S,7aR)-3a-(3,4-dimethoxyphenyl)-1-methyloctahydro-1H-indol-6-amine (25 mg, 16%)

Confirmation of Absolute Stereochemistry: Conversion of (3aR,6S,7aR)-3a-(3,4-dimethoxyphenyl)-1-methyloctahydro-1H-indol-6-amine to (+)mesembrine

The amine from Comparative Example 11 (145 mg, 0.5 mmol) was dissolved in THF:methanol 1:1 (6 mL) under nitrogen. 3,5-ditertbutyl-1,2-benzoquinone (110 mg, 0.5 mmol) was added and the mixture was stirred for one hour. A solution of oxalic acid (90 mg, 1 mmol) in water (3 mL) was added and the mixture was stirred overnight. Water (10 mL) was added and mixture was basified with 3 potassium hydroxide pellets. The product was extracted into CH₂Cl₂ (3×50 mL), evaporated and purified by preparative hplcms (20-50% basic). This product was dissolved in chloroform and passed through a small pad of silica using CHCl₃:acetone 8:1. Evaporation gave an oil (4.6 mg). Optical rotation (methanol) [α]_{D}=+48. Natural mesembrine gives a rotation of between −53 and −62, indicating that the isomer formed above was the opposite enantiomer to the natural product.

### EXAMPLE 12

### N-(3,4-difluorophenyl)-N′-[(3aS,6R,7aS)-3a-(3,4-dimethoxyphenyl)-1-methyloctahydro-1H-indol-6-yl]urea hydrochloride

A solution of the amine, (3aS,6R,7aS)-3a-(3,4-dimethoxyphenyl)-1-methyloctahydro-1H-indol-6-amine, Comparative Example 10 (0.14 g; 0.48 mmol) in dry THF (5.0 ml) was treated with 3,4-difluorophenyl isocyanate (77 mg; 0.5 mmol) and the resulting solution stirred at RT overnight.

The mixture was evaporated to afford a gum, which was flash-chromatographed over silica. Elution with ethyl acetate gave the product, which was treated with a 4M solution of hydrogen chloride in dioxane to give the corresponding HCl salt. Yield: 128.6 mg (56%) M+H=446

### EXAMPLE 13

### N-[(3aS*,6R*,7aS*)-1-benzyl-3a-(3,4-dimethoxyphenyl)octahydro-1H-indol-6-yl]-N′-pyridin-3-ylthiourea trifluoroacetate

Reagent: 3-pyridyl isothiocyanate

Synthetic procedure: Scheme A

Measured mass: 502.2400

Calc. Mass: 502.2402

### EXAMPLE 14

### Methyl 3-[({[(3aS*,6R*,7aS*)-1-benzyl-3a-(3,4-dimethoxyphenyl)octahydro-1H-indol-6-yl]amino}carbonothioyl)amino]thiophene-2-carboxylate trifluoroacetate

Reagent: methyl 3-isothiocyanatothiophene-2-carboxylate

Synthetic procedure: Scheme A

Measured mass: 565.2080

Calc. mass: 565.2069

### EXAMPLE 15

### N-[(3aS*,6R*,7aS*)-1-benzyl-3a-(3,4-dimethoxyphenyl)octahydro-1H-indol-6-yl]-N′-[4-(dimethylamino)phenyl]thiourea trifluoroacetate

Reagent: 4-(dimethylamino)phenyl isothiocyanate

Synthetic procedure: Scheme A

Measured mass: 544.2890

Calc. mass: 544.2872

### EXAMPLE 16

### N-[(3aS*,6R*,7aS*)-1-benzyl-3a-(3,4-dimethoxyphenyl)octahydro-1H-indol-6-yl]-N′-[4-(dimethylamino)phenyl]urea trifluoroacetate

Reagent: 4-(dimethylamino)phenyl isocyanate

Synthetic procedure: Scheme A

Measured mass: 528.3110

Calc. mass: 528.3100

### EXAMPLE 17

### N-[(3aS*,6R*,7aS*)-1-benzyl-3a-(3,4-dimethoxyphenyl)octahydro-1H-indol-6-yl]-N′-(3,5-dimethylisoxazol-4-yl)urea trifluoroacetate

Reagent: 4-isocyanato-3,5-dimethylisoxazole

Synthetic procedure: Scheme A

Measured mass: 504.2749

Calc. mass: 504.2737

### EXAMPLE 18

### N-[(3aS*,6R*,7aS*)-1-benzyl-3a-(3,4-dimethoxyphenyl)octahydro-1H-indol-6-yl]-N′-(3,5-dimethoxyphenyl)urea trifluoroacetate

Reagent: 3,5-dimethoxyphenyl isocyanate

Synthetic procedure: Scheme A

Measured mass: 545.2890

Calc. mass: 545.2890

### EXAMPLE 19

### N-[(3aS*,6R*,7aS*)-1-benzyl-3a-(3,4-dimethoxyphenyl)octahydro-1H-indol-6-yl]-N′-(2,6-dichloropyridin-4-yl)urea trifluoroacetate

Reagent: 2,6-dichloropyridin-4-isocyanate

Synthetic procedure: Scheme A

### EXAMPLE 20

### N-[(3aS*,6R*,7aS*)-1-benzyl-3a-(3,4-dimethoxyphenyl)octahydro-1H-indol-6-yl]-N′-pyridin-4-ylurea trifluoroacetate

Reagent: 4-aminopyridine

Synthetic procedure: Scheme B

Measured mass: 486.2642

Calc. mass: 486.2631

### EXAMPLE 21

### N-[(3aS*,6R*,7aS*)-1-benzyl-3a-(3,4-dimethoxyphenyl)octahydro-1H-indol-6-yl]-N′-(3,5-dichloropyridin-4-yl)urea trifluoroacetate

Reagent: 4-amino-3,5-dichloropyridine

Synthetic procedure: Scheme B

Measured mass: 554.1851

Calc. mass: 554.1851

### EXAMPLE 22

### N-[(3aS*,6R*,7aS*)-1-benzyl-3a-(3,4-dimethoxyphenyl)octahydro-1H-indol-6-yl]-N′-(1,3,5-trimethyl-1H-pyrazol-4-yl)urea trifluoroacetate

Reagent: 4-amino-1,3,5-trimethylpyrazole

Synthetic procedure: Scheme B

Measured mass: 517.3062

Calc. mass: 517.3053

### EXAMPLE 23

### N-(2,3-dichlorophenyl)-N′-[(3aS*,7aS*)-3a-(3,4-dimethoxyphenyl) 1-methyloctahydro-1H-indol-6-yl]urea

A solution of the the amine (diastereomeric mixture, Comparative Example 7/8) (29 mg; 0.1 mmol) in DCM (4.0 ml) was treated with 2,3-dichlorophenyl isocyanate (0.1 mmol) and stirred at RT overnight the mixture was evaporated and the residue purified by preparative HPLC to give the product as a mixture of diastereomers (46 mg)

Measured mass: 477.1586

Calc. mass: 477.1586

### EXAMPLE 24

### N-(2,6-dichloropyridin-4-yl)-N′-[(3aS*,6R*,7aS*)-3a-(3,4-dimethoxyphenyl)-1-methyloctahydro-1H-indol-6-yl]urea trifluoroacetate

Reagent: 2,6-dichloropyridin-4-isocyanate

Synthetic procedure: Scheme C

Measured mass: 478.1548

Calc. mass: 478.1538

### EXAMPLE 25

### N-(3-chlorophenyl)-N′-[(3aS*,6R*,7aS*)-3a-(3,4-dimethoxyphenyl)-1-methyloctahydro-1H-indol-6-yl]urea trifluoroacetate

Reagent: 1-chloro-3-isocyanatobenzene

Synthetic procedure: Scheme D

Measured mass: 443.1964

Calc. mass: 443.1976

### EXAMPLE 26

### N-(4-cyanophenyl)-N′-[(3aS*,6R*,7aS*)-3a-(3,4-dimethoxyphenyl)-1-methyloctahydro-1H-indol-6-yl]urea trifluoroacetate

Reagent: 4-cyanophenylisocyanate

Synthetic procedure: Scheme C

Measured mass: 434.2311

Calc. mass: 434.2318

### EXAMPLE 27

### N-(2,4-difluorophenyl)-N′-[(3aS*,6R*,7aS*)-3a-(3,4-dimethoxyphenyl)-1-methyloctahydro-1H-indol-6-yl]urea trifluoroacetate

Reagent: 2,4-difluorophenyl isocyanate

Synthetic procedure: Scheme C

### EXAMPLE 28

### N-(5-chloro-2-methoxyphenyl)-N′-[(3aS*,6R*,7aS*)-3a-(3,4-dimethoxyphenyl)-1-methyloctahydro-1H-indol-6-yl]urea trifluoroacetate

Reagent: 5-chloro-2-methoxyphenyl isocyanate

Synthetic procedure: Scheme C

Measured mass: 473.2062

Calc. mass: 473.2081

### EXAMPLE 29

### N-[4-chloro-3-(trifluoromethyl)phenyl]-N′-[(3aS*,6R*,7aS*)-3a-(3,4-dimethoxyphenyl)-1-methyloctahydro-1H-indol-6-yl]urea trifluoroacetate

Reagent: 4-chloro-3-(trifluoromethyl)phenyl isocyanate

Synthetic procedure: Scheme C

Measured mass: 511.1862

Calc. mass: 511.1850

### EXAMPLE 30

### N-(3-chloro-2-methylphenyl)-N′-[(3aS*,6R*,7aS*)-3a-(3,4-dimethoxyphenyl)-1-methyloctahydro-1H-indol-6-yl]urea trifluoroacetate

Reagent: 3-chloro-2-methylphenyl isocyanate

Synthetic procedure: Scheme C

Measured mass: 457.2130

Calc. mass: 457.2132

### EXAMPLE 31

### N-1,3-benzodioxol-5-yl-N′-[(3aS*,6R*,7aS*)-3a-(3,4-dimethoxyphenyl)-1-methyloctahydro-1H-indol-6-yl]urea trifluoroacetate

Reagent: 3,4-(methylenedioxy)phenyl isocyanate

Synthetic procedure: Scheme C

Measured mass: 453.2264

Calc. mass: 453.2264

### EXAMPLE 32

### N-(2,3-dichlorophenyl)-N′-[(3aS*,6R*,7aS*)-3a-(3,4-dimethoxyphenyl)-1-methyloctahydro-1H-indol-6-yl]thiourea trifluoroacetate

Reagent: 2,3-dichlorophenyl isocyanate

Synthetic procedure: Scheme C

Measured mass: 493.1358

Calc. mass: 493.1358

### EXAMPLE 33

### N-(2,4-difluorophenyl)-N′-[(3aS*,6R*,7aS*)-3a-(3,4-dimethoxyphenyl)-1-methyloctahydro-1H-indol-6-yl]thiourea trifluoroacetate

Reagent: 2,4-difluorophenyl isocyanate

Synthetic procedure: Scheme C

Measured mass: 461.1950

Calc. mass: 461.1949

### EXAMPLE 34

### N-[(3aS*,6R*,7aS*)-3a-(3,4-dimethoxy henyl)-1-methyloctahydro-1H-indol-6-yl]-N′-[4-(methylthio)phenyl]thiourea trifluoroacetate

Reagent: 4-(methylthio)phenyl isothiocyanate

Synthetic procedure: Scheme C

Measured mass: 471.2012

Calc. mass: 471.2014

### EXAMPLE 35

### N-(2,3-dihydro-1H-inden-5-yl)-N′-[(3aS*,6R*,7aS*)-3a-(3,4-dimethoxyphenyl)-1-methyloctahydro-1H-indol-6-yl]thiourea trifluoroacetate

Reagent: 5-isothiocyanatoindane

Synthetic procedure: Scheme C

Measured mass: 465:2452

Calc. mass: 465.2450

### EXAMPLE 36

### N-(3,5-dichlorophenyl)-N′-[(3aS*,6R*,7aS*)-3a-(3,4-dimethoxyphenyl)-1-methyloctahydro-1H-indol-6-yl]urea trifluoroacetate

Reagent: 1,3-dichloro-5-isocyanatobenzene

Synthetic procedure: Scheme D

Measured mass: 477.1604

Calc. mass: 477.1586

### EXAMPLE 37

### N-(4-bromophenyl)-N′-[(3aS*,6R*,7aS*)-3a-(3,4-dimethoxyphenyl)-1-methyloctahydro-1H-indol-6-yl]urea trifluoroacetate

Reagent: 1-bromo-4-isocyanatobenzene

Synthetic procedure: Scheme D

Measured mass: 487.1495

Calc. mass: 487.1471

¹H NMR (270 MHz, CDCl₃) δ 1.92 (m, 1H) 2.25 (m, 3H) 3.00 (m, 3H) 3.16 (m, 5H) 3.62 (m, 1H) 3.88 (s, 3H) 3.89 (s, 3H) 4.07 (m, 2H) 4.57 (m, 1H) 6.55 (m, 1H) 6.67 (m, 1H) 6.86 (m, 1H) 7.36 (d, J=8.18 Hz, 2H) 7.50 (m, 2H) 8.83 (br. s, 1H)

### EXAMPLE 38

### N-[(3aS*,6R*,7aS*)-3a-(3,4-dimethoxyphenyl)-1-methyloctahydro-1H-indol-6-yl]-N′-phenylurea trifluoroacetate

Reagent: phenyl isocyanate

Synthetic procedure: Scheme D

Measured mass: 409.2353

Calc. mass: 409.2365

### EXAMPLE 39

### N-(3,5-difluorophenyl)-N′-[(3aS*,6R*,7aR*)-3a-(3,4-dimethoxyphenyl)-1-methyloctahydro-1H-indol-6-yl]urea trifluoroacetate

Reagent: 3,5-difluorophenyl isocyanate

Synthetic procedure: Scheme D

Measured mass: 445.2196

Calc. mass: 445.2177

### EXAMPLE 40

### N-(3-chloro-4-fluorophenyl)-N′-[(3aS*,6R*,7aS*)-3a-(3,4-dimethoxyphenyl)-1-methyloctahydro-1H-indol-6-yl]urea trifluoroacetate

Reagent: 3-chloro-4-fluorophenyl isocyanate

Synthetic procedure: Scheme D

Measured mass: 461.1871

Calc. mass: 461.1881

### EXAMPLE 41

### N-(2,3-dihydro-1H-inden-5-yl)-N′-[(3aS*,6R*,7aS*)-3a-(3,4-dimethoxyphenyl)-1-methyloctahydro-1H-indol-6-yl]urea trifluoroacetate

Reagent: 5-indanyl isocyanate

Synthetic procedure: Scheme D

Measured mass: 449.2673

Calc. mass: 449.2678

### EXAMPLE 42

### N-[(3aS*,6R*,7aS*)-3a-(3,4-dimethoxyphenyl)-1-methyloctahydro-1H-indol-6-yl]-N′-2-naphthylurea trifluoroacetate

Reagent: 2-naphthyl isocyanate

Synthetic procedure: Scheme D

Measured mass: 459.2511

Calc. mass: 459.2522

### EXAMPLE 43

### N-[(3aS*,6R*,7aS*)-3a-(3,4-dimethoxyphenyl)-1-methyloctahydro-1H-indol-6-yl]-N′-{3-[(trifluoromethyl)thio]phenyl}urea trifluoroacetate

Reagent: 1-isocyanato-3-[(trifluoromethyl)thio]benzene

Synthetic procedure: Scheme C

### EXAMPLE 44

### N-[(3aS*,6R*,7aS*)-3a-(3,4-dimethoxyphenyl)-1-methyloctahydro-1H-indol-6-yl]-N′-(3-methoxyphenyl)urea trifluoroacetate

Reagent: 3-methoxyphenyl isocyanate

Synthetic procedure: Scheme D

Measured mass: 439.2453

Calc. mass: 439.2471

### EXAMPLE 45

### N-[(3aS*,6R*,7aS*)-3a-(3,4-dimethoxyphenyl)-1-methyloctahydro-1H-indol-6-yl]-N′-(2,6-dimethylphenyl)urea trifluoroacetate

Reagent: 2,6-dimethylphenyl isocyanate

Synthetic procedure: Scheme D

Measured mass: 437.2673

Calc. mass: 437.2678

### EXAMPLE 46

### N-(2,6-dichlorophenyl)-N′-[(3aS*,6R*,7aS*)-3a-(3,4-dimethoxyphenyl)-1-methyloctahydro-1H-indol-6-yl]urea trifluoroacetate

Reagent: 2,6-dichlorophenyl isocyanate

Synthetic procedure: Scheme D

Measured mass: 477.1610

Calc. mass: 477.1586

### EXAMPLE 47

### N-[(3aS*,6R*,7aS*)-3a-(3,4-dimethoxyphenyl)-1-methyloctahydro-1H-indol-6-yl]-N′-[3-(trifluoromethyl)phenyl]urea trifluoroacetate

Reagent: 3-(trifluoromethyl)phenyl isocyanate

Synthetic procedure: Scheme D

Measured mass: 477.2250

Calc. mass: 477.2239

### EXAMPLE 48

### N-[(3aS*,6R*,7aS*)-3a-(3,4-dimethoxyphenyl)-1-methyloctahydro-1H-indol-6-yl]-N′-[4-fluoro-3-(trifluoromethyl)phenyl]urea trifluoroacetate

Reagent: 4-fluoro-3-(trifluoromethyl)phenyl isocyanate

Synthetic procedure: Scheme D

Measured mass: 495.2154

Calc. mass: 495.2145

### EXAMPLE 49

### N-[(3aS*,6R*,7aS*)-3a-(3,4-dimethoxyphenyl)-1-methyloctahydro-1H-indol-6-yl]-N′-(pentafluorophenyl)urea trifluoroacetate

Reagent: pentaflurophenyl isocyanate

Synthetic procedure: Scheme D

Measured mass: 499.1908

Calc. mass: 499.1894

### EXAMPLE 50

### N-[(3aS*,6R*,7aS*)-3a-(3,4-dimethoxyphenyl)-1-methyloctahydro-1H-indol-6-yl]-N′-(2,3,5,6-tetrachlorophenyl)urea trifluoroacetate

Reagent: 2,3,5,6-tetrachlorophenyl isocyanate

Synthetic procedure: Scheme D

Measured mass: 545.0813

Calc. mass: 545.0806

### EXAMPLE 51

### N-(3-bromophenyl)-N′-[(3aS*,6R*,7aS*)-3a-(3,4-dimethoxyphenyl)-1-methyloctahydro-1H-indol-6-yl]urea trifluoroacetate

Reagent: 3-bromophenyl isocyanate

Synthetic procedure: Scheme D

Measured mass: 487.1480

Calc. mass: 487.1471

### EXAMPLE 52

### N-(3-chloro-4-methoxyphenyl)-N′-[(3aS*,6R*,7aS*)-3a-(3,4-dimethoxyphenyl)-1-methyloctahydro-1H-indol-6-yl]urea trifluoroacetate

Reagent: 3-chloro-4-methoxyphenyl isocyanate

Synthetic procedure: Scheme D

Measured mass: 473.2063

Calc. mass: 473.2081

### EXAMPLE 53

### N-(3,5-dichlorophenyl)-N′-[(3aS*,6R*,7aS*)-3a-(3,4-dimethoxyphenyl)-1-methyloctahydro-1H-indol-6-yl]guanidine trifluoroacetate

N-(3,5-dichlorophenyl)-1H-pyrazole-1-carboximidamide hydrochloride (20 mg, 0.07 mmol), (3aS*,6R*,7aS*)-3a-(3,4-dimethoxyphenyl)-1-methyloctahydro-1H-indol-6-amine (Comparative Example 7; 23 mg, 0.08 mmol) and diisopropylethyl amine (0.02 ml, 0.11 mmol) were mixed in anhydrous DMF (0.5 ml) and heated in microwave at 180° C. for 300 s. The crude mixture was purified by preparative HPLC to give the title compound. HRMS (EI) calc: 476.1766 found: 476.1755

### EXAMPLE 54

### N-(2,4-difluorophenyl)-N′-[(3aS*,6R*,7aS*)-3a-(3,4-dimethoxyphenyl)-1-methyloctahydro-1H-indol-6-yl]guanidine trifluoroacetate

N-(2,4-difluorophenyl)-1H-pyrazole-1-carboximidamide hydrochloride (20 mg, 0.08 mmol), (3aS*,6R*,7aS*)-3a-(3,4-dimethoxyphenyl)-1-methyloctahydro-1H-indol-6-amine (Comparative Example 7; 25 mg, 0.09 mmol) and diisopropylethyl amine (0.02 ml, 0.11 mmol) were mixed in anhydrous DMF (0.5 ml) and heated in microwave at 180° C. for 300 s. The crude mixture was purified by preparative HPLC to give the title compound, 5 mg (11%). HRMS (EI) calc: 444.2337 found: 444.2344

### EXAMPLE 55

### N-(3,5-difluorophenyl)-N′-[(3aS*,6R*,7aS*)-3a-(3,4-dimethoxyphenyl)-1-methyloctahydro-1H-indol-6-yl]guanidine trifluoroacetate

N-(3,5-difluorophenyl)-1H-pyrazole-1-carboximidamide hydrochloride (20 mg, 0.08 mmol) and diisopropylethyl amine (0.02 ml, 0.11 mmol) and (3aS*,6R*,7aS*)-3a-(3,4-dimethoxyphenyl)-1-methyloctahydro-1H-indol-6-amine (Comparative Example 7; 25 mg, 0.09 mmol) were mixed in anhydrous DMF (0.5 ml) and heated in microwave at 180° C. for 300 s. The crude mixture was purified by preparative HPLC to give the title compound, 5 mg (14%). HRMS (EI) calc: 444.2337 found: 444.2337

### EXAMPLE 56

### N-[(3aS*,6R*,7aS*)-3a-(3,4-dimethoxyphenyl)-1-methyloctahydro-1H-indol-6-yl]-N′-pyridazin-4-ylurea trifluoroacetate

4-Amino-pyridazine, p-nitrophenyl chloroformate and Hünig's base (1:1:1) were dissolved in 2 ml dry DCM. Reaction in r.t., under N₂ and for 5 h.

The amine (3aS*,6R*,7aS*)-3a-(3,4-dimethoxyphenyl)-1-methyloctahydro-1H-indol-6-amine (Comparative Example 7; 6 mg, 0,021 mmol) and another eq. base were added.

Reaction in r.t., under N₂ and overnight.

MS (electrospray; [M+H]⁺) m/z 413.

Measured mass: 412.2147

Calc. mass: 411.2270

¹H NMR (500 MHz, CDCl₃) δ 3.68 (m, 1H) 3.90 (s, 3H) 3.92 (s, 3H) 4.34 (dt, J=12.02, 8.39 Hz, 1H) 4.50 (m, 1H) 6.48 (dd, J=8.30, 2.93 Hz, 1H) 6.78 (d, J=1.71 Hz, 1H) 6.81 (dd, J=8.55, 1.71 Hz, 1H) 6.88 (d, J=8.30 Hz, 1H) 7.17 (d, J=6.84 Hz, 1H) 7.71 (d, J=2.69 Hz, 1H) 8.74 (d, J=8.30 Hz, 1H)

### EXAMPLE 57

### N-[(3aS*,6R*,7aS*)-3a-(3,4-dimethoxy henyl)-1-methyloctahydro-1H-indol-6-yl]-N′-(3,5-dimethylisoxazol-4-yl)urea trifluoroacetate

Reagent: 4-isocyanato-3,5-dimethylisoxazole

Synthetic procedure: Scheme D

Measured mass: 428.2409

Calc. mass: 428.2424

### EXAMPLE 58

### N′-[4-chloro-3-(trifluoromethyl)phenyl]-N-[(3aS*,6S*,7aS*)-3a-(3,4-dimethoxyphenyl)-1-methyloctahydro-1H-indol-6-yl]-N-methylurea

Mesembrine (250 mg, 870 μmol) was dissolved in DCM (4 mL). An aqueous solution of methylamine (12 mL of a 50% solution) was added, followed by sodium cyanoborohydride (250 mg, 3.98 mmol). The mixture was stirred overnight at room temperature and the solvent removed under reduced pressure. The crude product was partitioned between NaOH solution (25 mL, 3M) and DCM (25 mL). The aqueous portion was extracted with further DCM (2×20 mL), the combined extracts dried (Na₂SO₄), and the solvent was removed under reduced pressure. The oily residue was dissolved in DCM (5 mL), and treated with 4-chloro-3-(trifluoromethyl)phenyl isocyanate 960 μmol). After stirring at room temperature for 16 hours, the solvent was removed and the crude products purified by preparative HPLC.

Yield: 59.8 mg (13%):

MS (ESI+) for C₂₆H₃₁ClF₃N₃O₃: m/z 526.0 (M+1).

HRMS (EI) calcd C₂₆H₃₁ClF₃N₃O₃: 525.2006, found 525.2002

### EXAMPLE 59

### N′-(3-bromophenyl)-N-[(3aS*,6R*,7aS*)-3a-(3,4-dimethoxyphenyl)-1-methyloctahydro-1H-indol-6-yl]-N-methylurea trifluoroacetate and

### EXAMPLE 60

### N′-(3-bromophenyl)-N-[(3aS*,6S*,7aS*)-3a-(3,4-dimethoxyphenyl)-1-methyloctahydro-1H-indol-6-yl]-N-methylurea trifluoroacetate

Compounds were prepared and purified in an analogous method to that above (Example 58)

N′-(3-bromophenyl)-N-[(3aS*,6R*,7aS*)-3a-(3,4-dimethoxyphenyl)-1-methyloctahydro-1H-indol-6-yl]-N-methylurea (58.8 mg, 13%):

MS (ESI+) for C₂₅H₃₂BrN₃O₃: m/z 502.2 (M+1).

HRMS (EI) calcd C₂₅H₃₂BrN₃O₃: 501.1627, found 501.1646

N′-(3-bromophenyl)-N-[(3aS*,6S*,7aS*)-3a-(3,4-dimethoxyphenyl)-1-methyloctahydro-1H-indol-6-yl]-N-methylurea (108.2 mg, 25%):

MS (ESI+) for C₂₅H₃₂BrN₃O₃: m/z 502.2 (M+1).

HRMS (EI) calcd C₂₅H₃₂BrN₃O₃: 501.1627, found 501.1645

### EXAMPLE 61

### ethyl 4-[({[(3aS*,6R*,7aS*)-3a-(3,4-dimethoxyphenyl)-1-methyloctahydro-1H-indol-6-yl]amino}carbonyl)amino]benzoate trifluoroacetate

Reagent: ethyl 4-isocyanatobenzoate

Synthetic procedure: Scheme E

Yield: 7.9 mg (39%)

Measured mass: 481.2572

Calc. mass: 481.2577

### EXAMPLE 62

### ethyl 3-[({[(3aS*,6R*,7aS*)-3a-(3,4-dimethoxyphenyl)-1-methyloctahydro-1H-indol-6-yl]amino}carbonyl)amino]benzoate trifluoroacetate

Reagent: ethyl 3-isocyanatobenzoate

Synthetic procedure: Scheme E

Yield: 7.4 mg (36%)

Measured mass: 481.2572

Calc. mass: 481.2577

### EXAMPLE 63

### N-(3-cyanophenyl)-N′-[(3aS*,6R*,7aS*)-3a-(3,4-dimethoxyphenyl)-1-methyloctahydro-1H-indol-6-yl]urea trifluoroacetate

Reagent: 3-cyanophenyl isocyanate

Synthetic procedure: Scheme E

Yield: 6.3 mg (33%)

Measured mass: 434.2308

Calc. mass: 434.2318

### EXAMPLE 64

### N-[(3aS*,6R*,7aS*)-3a-(3,4-dimethoxyphenyl)-1-methyloctahydro-1H-indol-6-yl]-N′-(2-methoxy-5-nitrophenyl)urea trifluoroacetate

Reagent: 2-methoxy-5-nitrophenyl isocyanate

Synthetic procedure: Scheme E

Yield: 5.9 mg (29%)

Measured mass: 484.2307

Calc. mass: 484.2322

### EXAMPLE 65

### N-(2-chlorophenyl)-N′-[(3aS*,6R*,7aS*)-3a-(3,4-dimethoxyphenyl)-1-methyloctahydro-1H-indol-6-yl]urea trifluoroacetate

Reagent: 2-chlorophenyl isocyanate

Synthetic procedure: Scheme E

Yield: 8.1 mg (42%)

Measured mass: 443.1976

Calc. mass: 443.1977

### EXAMPLE 66

### N-[(3aS*,6R*,7aS*)-3a-(3,4-dimethoxyphenyl)-1-methyloctahydro-1H-indol-6-yl]-N′-(4-methoxy-2-nitrophenyl)urea trifluoroacetate

Reagent: 4-methoxy-2-nitrophenyl isocyanate

Synthetic procedure: Scheme E

Yield: 2.6 mg (13%)

Measured mass: 484.2314

Calc. mass: 484.2322

### EXAMPLE 67

### N-[(3aS*,6R*,7aS*)-3a-(3,4-dimethoxyphenyl)-1-methyloctahydro-1H-indol-6-yl]-N′-[4-(trifluoromethyl)phenyl]urea trifluoroacetate

Reagent: 4-(trifluoromethyl)phenyl isocyanate

Synthetic procedure: Scheme E

Yield: 8.7 mg (43%)

Measured mass: 477.2235

Calc. mass: 477.2239

### EXAMPLE 68

### N-[(3aS*,6R*,7aS*)-3a-(3,4-dimethoxyphenyl)-1-methyloctahydro-1H-indol-6-yl]-N′-(2-methoxy-4-nitrophenyl)thiourea trifluoroacetate

Reagent: 2-methoxy-4-nitrophenyl isothiocyanate

Synthetic procedure: Scheme E

Yield: 3.2 mg (15%)

Measured mass: 500.2077

Calc. mass: 500.2093

### EXAMPLE 69

### N-[(3aS*,6R*,7aS*)-3a-(3,4-dimethoxyphenyl)-1-methyloctahydro-1H-indol-6-yl]-N′-[4-(dimethylamino)phenyl]urea trifluoroacetate

Reagent: 4-(dimethylamino)phenyl isocyanate

Synthetic procedure: Scheme E

Yield: 4.3 mg (22%)

Measured mass: 452.2783

Calc. mass: 452.2787

### EXAMPLE 70

### N-[(3aS*,6R*,7aS*)-3a-(3,4-dimethoxyphenyl)-1-methyloctahydro-1H-indol-6-yl]-N′-[4-(trifluoromethoxy)phenyl]urea trifluoroacetate

Reagent: 4-(trifluoromethoxy)phenyl isocyanate

Synthetic procedure: Scheme E

Yield: 9.2 mg (44%)

Measured mass: 493.2188

Calc. mass: 493.2188

### EXAMPLE 71

### N-[4-bromo-2-(trifluoromethyl)phenyl]-N′-[(3aS*,6R*,7aS*)-3a-(3,4-dimethoxyphenyl)-1-methyloctahydro-1H-indol-6-yl]urea trifluoroacetate

Reagent: 4-bromo-2-(trifluoromethyl)phenyl isocyanate

Synthetic procedure: Scheme E

Yield: 10.3 mg (45%)

Measured mass: 555.1334

Calc. mass: 555.1344

### EXAMPLE 72

### N-[(3aS*,6R*,7aS*)-3a-(3,4-dimethoxyphenyl)-1-methyloctahydro-1H-indol-6-yl]-N′-(4-fluoro-3-nitrophenyl)urea trifluoroacetate

Reagent: 4-fluoro-3-nitrophenyl isocyanate

Synthetic procedure: Scheme E

Yield: 7.5 mg (37%)

Measured mass: 472.2142

Calc. mass: 472.2122

### EXAMPLE 73

### N-[(3aS*,6R*,7aS*)-3a-(3,4-dimethoxyphenyl)-1-methyloctahydro-1H-indol-6-yl]-N′-(4-fluorophenyl)urea trifluoroacetate

Reagent: 4-fluorophenyl isocyanate

Synthetic procedure: Scheme E

Yield: 5.4 mg (29%)

Measured mass: 427.2289

Calc. mass: 427.2271

### EXAMPLE 74

### N-[(3aS*,6R*,7aS*)-3a-(3,4-dimethoxyphenyl)-1-methyloctahydro-1H-indol-6-yl]-N′-[3-(methylthio)phenyl]urea trifluoroacetate

Reagent: 3-(methylthio)phenyl isocyanate

Synthetic procedure: Scheme E

Yield: 7.2 mg (37%)

Measured mass: 455.2230

Calc. mass: 455.2243

### EXAMPLE 75

### N-[(3aS*,6R*,7aS*)-3a-(3,4-dimethoxyphenyl)-1-methyloctahydro-1H-indol-6-yl]-N′-(4-methoxy-2-methylphenyl)urea trifluoroacetate

Reagent: 4-methoxy-2-methylphenyl isocyanate

Synthetic procedure: Scheme E

Yield: 4.4 mg (23%)

Measured mass: 453.2629

Calc. mass: 453.2628

### EXAMPLE 76

### methyl 3-[({[3aS*,6R*,7aS*)-3a-(3,4-dimethoxyphenyl)-1-methyloctahydro-1H-indol-6-yl]amino}carbonothioyl)amino]thiophene-2-carboxylate trifluoroacetate

Reagent: methyl 3-isothiocyanatothiophene-2-carboxylate

Synthetic procedure: Scheme E

Yield: 4.9 mg (24%)

Measured mass: 489.1752

Calc. mass: 489.1756

### EXAMPLE 77

### N-(2-chloro-5-methylphenyl)-N′-[(3aS*,6R*,7aS*)-3a-(3,4-dimethoxyphenyl)-1-methyloctahydro-1H-indol-6-yl]urea trifluoroacetate

Reagent: 2-chloro-5-methylphenyl isocyanate

Synthetic procedure: Scheme E

Yield: 8.0 mg (41%)

Measured mass: 457.2130

Calc. mass: 457.2132

### EXAMPLE 78

### N-[(3aS*,6R*,7aS*)-3a-(3,4-dimethoxyphenyl)-1-methyloctahydro-1H-indol-6-yl]-N′-(2-methylphenyl)urea trifluoroacetate

Reagent: 2-methylphenyl isocyanate

Synthetic procedure: Scheme E

Yield: 5.6 mg (30%)

Measured mass: 423.2516

Calc. mass: 423.2522

### EXAMPLE 79

### N-[(3aS*,6R*,7aS*)-3a-(3,4-dimethoxyphenyl)-1-methyloctahydro-1H-indol-6-yl]-N′-(2-fluorophenyl)urea trifluoroacetate

Reagent: 2-flurophenyl isocyanate

Synthetic procedure: Scheme E

Yield: 6.9 mg (37%)

Measured mass: 427.2281

Calc. mass: 427.2271

### EXAMPLE 80

### N-(2,4-dichlorophenyl)-N′-[(3aS*,6R*,7aS*)-3a-(3,4-dimethoxyphenyl)-1-methyloctahydro-1H-indol-6-yl]urea trifluoroacetate

Reagent: 2,4-dichlorophenyl isocyanate

Synthetic procedure: Scheme E

Yield: 8.3 mg (41%)

Measured mass: 477.1589

Calc. mass: 477.1586

### EXAMPLE 81

### N-[(3aS*,6R*,7aS*)-3a-(3,4-dimethoxyphenyl)-1-methyloctahydro-1H-indol-6-yl]-N′-[2-(trifluoromethyl)phenyl]urea trifluoroacetate

Reagent: 2-(trifluoromethyl)phenyl isocyanate

Synthetic procedure: Scheme E

Yield: 9.1 mg (45%)

Measured mass: 477.2134

Calc. mass: 477.2239

### EXAMPLE 82

### N-[(3aS*,6R*,7aS*)-3a-(3,4-dimethoxyphenyl)-1-methyloctahydro-1H-indol-6-yl]-N′-(4-methyl-3-nitrophenyl)urea trifluoroacetate

Reagent: 4-methyl-3-nitrophenyl isocyanate

Synthetic procedure: Scheme E

Yield: 8.5 mg (42%)

Measured mass: 468.2375

Calc. mass: 468.2373

### EXAMPLE 83

### N-[(3aS*,6R*,7aS*)-3a-(3,4-dimethoxyphenyl)-1-methyloctahydro-1H-indol-6-yl]-N′-(2,4-dimethylphenyl)urea trifluoroacetate

Reagent: 2,4-dimethylphenyl isocyanate

Synthetic procedure: Scheme E

Yield: 10.6 mg (56%)

Measured mass: 437.2688

Calc. mass: 437.2678

### EXAMPLE 84

### N-(4-tert-butylphenyl)-N′-[(3aS*,6R*,7aS*)-3a-(3,4-dimethoxyphenyl)-1-methyloctahydro-1H-indol-6-yl]urea trifluoroacetate

Reagent: 4-tert-butylphenyl isocyanate

Synthetic procedure: Scheme E

Yield: 10.0 mg (50%)

Measured mass: 465.2969

Calc. mass: 465.2991

### EXAMPLE 85

### N-[(3aS*,6R*,7aS*)-3a-(3,4-dimethoxyphenyl)-1-methyloctahydro-1H-indol-6-yl]-N′-{4-[(trifluoromethyl)thio]phenyl}urea trifluoroacetate

Reagent: 4-[(trifluoromethyl)thio]phenyl isocyanate

Synthetic procedure: Scheme E

Yield: 8.8 mg (41%)

Measured mass: 509.1983

Calc. mass: 509.1960

### EXAMPLE 86

### N-[(3aS*,6R*,7aS*)-3a-(3,4-dimethoxyphenyl)-1-methyloctahydro-1H-indol-6-yl]-N′-(4-methoxyphenyl)urea trifluoroacetate

Reagent: 4-methoxyphenyl isocyanate

Synthetic procedure: Scheme E

Yield: 7.9 mg (42%)

Measured mass: 439.2457

Calc. mass: 439.2471

### EXAMPLE 87

### N-(2-bromophenyl)-N′-[(3aS*,6R*,7aS*)-3a-(3,4-dimethoxyphenyl) methyloctahydro-1H-indol-6-yl]urea trifluoroacetate

Reagent: 2-bromophenyl isocyanate

Synthetic procedure: Scheme E

Yield: 3.9 mg (19%)

Measured mass: 487.1455

Calc. mass: 487.1471

### EXAMPLE 88

### N-[(3aS*,6R*,7aS*)-3a-(3,4-dimethoxyphenyl)-1-methyloctahydro-1H-indol-6-yl]-N′-[3-(trifluoromethyl)phenyl]thiourea trifluoroacetate

Reagent: 3-(trifluoromethyl)phenyl isothiocyanate

Synthetic procedure: Scheme E

Yield: 8.4 mg (40%)

Measured mass: 493.2009

Calc. mass: 493.2011

### EXAMPLE 89

### N-(4-chloro-2-nitrophenyl)-N′-[(3aS*,6R*,7aS*)-3a-(3,4-dimethoxyphenyl)-1-methyloctahydro-1H-indol-6-yl]urea trifluoroacetate

Reagent: 4-chloro-2-nitrophenyl isocyanate

Synthetic procedure: Scheme E

Yield: 8.4 mg (40%)

Measured mass: 488.1809

Calc. mass: 488.1826

### EXAMPLE 90

### N-(3-acetylphenyl)-N′-[(3aS*,6R*,7aS*)-3a-(3,4-dimethoxyphenyl)-1-methyloctahydro-1H-indol-6-yl]urea trifluoroacetate

Reagent: 3-acetylphenyl isocyanate

Synthetic procedure: Scheme E

Yield: 6.4 mg (33%)

Measured mass: 451.2487

Calc. mass: 451.2471

### EXAMPLE 91

### N-[(3aS*,6R*,7aS*)-3a-(3,4-dimethoxyphenyl)-1-methyloctahydro-1H-indol-6-yl]-N′-(3-fluoro-4-methylphenyl)urea trifluoroacetate

Reagent: 3-fluoro-4-methylphenyl isocyanate

Synthetic procedure: Scheme E

Yield: 7.1 mg (37%)

Measured mass: 441.2406

Calc. mass: 441.2408

### EXAMPLE 92

### N-[(3aS*,6R*,7aS*)-3a-(3,4-dimethoxyphenyl)-1-methyloctahydro-1H-indol-6-yl]-N′-(4-methylphenyl)urea trifluoroacetate

Reagent: 4-methylphenyl isocyanate

Synthetic procedure: Scheme E

Yield: 5.3 mg (29%)

Measured mass: 423.2503

Calc. mass: 423.2522

### EXAMPLE 93

### N-[(3aS*,6R*,7aS*)-3a-(3,4-dimethoxyphenyl)-1-methyloctahydro-1H-indol-6-yl]-N′-(4,5-dimethyl-2-nitrophenyl)urea trifluoroacetate

Reagent: 4,5-dimethyl-2-nitrophenyl isocyanate

Synthetic procedure: Scheme E

Yield: 9.3 mg (45%)

Measured mass: 482.2528

Calc. mass: 482.2529

### EXAMPLE 94

### N-(5-chloro-2,4-dimethoxyphenyl)-N′-[(3aS*,6R*,7aS*)-3a-(3,4-dimethoxyphenyl)-1-methyloctahydro-1H-indol-6-yl]urea trifluoroacetate

Reagent: 5-chloro-2,4-dimethoxyphenyl isocyanate

Synthetic procedure: Scheme E

Yield: 5.5 mg (26%)

Measured mass: 503.2188

Calc. mass: 503.2187

### EXAMPLE 95

### N-[(3aS*,6R*,7aS*)-3a-(3,4-dimethoxyphenyl)-1-methyloctahydro-1H-indol-6-yl]-N′-(2-methoxy-5-methylphenyl)urea trifluoroacetate

Reagent: 2-methoxy-5-methylphenyl isocyanate

Synthetic procedure: Scheme E

Yield: 6.4 mg (33%)

Measured mass: 453.2615

Calc. mass: 453.2628

### EXAMPLE 96

### N-(4-chloro-3-nitrophenyl)-N′-[(3aS*,6R*,7aS*)-3a-(3,4-dimethoxyphenyl)-1-methyloctahydro-1H-indol-6-yl]urea trifluoroacetate

Reagent: 4-chloro-3-nitrophenyl isocyanate

Synthetic procedure: Scheme E

Yield: 5.6 mg (27%)

Measured mass: 488.1837

Calc. mass: 488.1826

### EXAMPLE 97

### N-[(3aS*,6R*,7aS*)-3a-(3,4-dimethoxyphenyl)-1-methyloctahydro-1H-indol-6-yl]-N′-(3-nitrophenyl)urea trifluoroacetate

Reagent: 3-nitrophenyl isocyanate

Synthetic procedure: Scheme E

Yield: 6.0 (31%)

Measured mass: 454.2217

Calc. mass: 454.2216

### EXAMPLE 98

### N-[(3aS*,6R*,7aS*)-3a-(3,4-dimethoxyphenyl)-1-methyloctahydro-1H-indol-6-yl]-N′-(4-ethylphenyl)urea trifluoroacetate

Reagent: 4-ethylphenyl isocyanate

Synthetic procedure: Scheme E

Yield: 8.6 mg (45%)

Measured mass: 437.2674

Calc. mass: 437.2678

### EXAMPLE 99

### N-[2-chloro-5-(trifluoromethyl)phenyl]-N′-[(3aS*,6R*,7aS*)-3a-(3,4-dimethoxyphenyl)-1-methyloctahydro-1H-indol-6-yl]urea trifluoroacetate

Reagent: 2-chloro-5-(trifluoromethyl)phenyl isocyanate

Synthetic procedure: Scheme E

Yield: 11.9 mg (55%)

Measured mass: 511.1862

Calc. mass: 511.1850

### EXAMPLE 100

### N-[(3aS*,6R*,7aS*)-3a-(3,4-dimethoxyphenyl)-1-methyloctahydro-1H-indol-6-yl]-N′-[3-fluoro-5-(trifluoromethyl)phenyl]urea trifluoroacetate

Reagent: 3-fluoro-5-(trifluoromethyl)phenyl isocyanate

Synthetic procedure: Scheme E

Yield: 8.1 mg (39%)

Measured mass: 495.2155

Calc. mass: 495.2145

### EXAMPLE 101

### N-[(3aS*,6R*,7aS*)-3a-(3,4-dimethoxyphenyl)-1-methyloctahydro-1H-indol-6-yl]-N′-[2-fluoro-3-(trifluoromethyl)phenyl]urea trifluoroacetate

Reagent: 2-fluoro-3-(trifluoromethyl)phenyl isocyanate

Synthetic procedure: Scheme E

Yield: 8.8 mg (42%)

Measured mass: 495.2146

Calc. mass: 495.2145

### EXAMPLE 102

### N-[(3aS*,6R*,7aS*)-3a-(3,4-dimethoxyphenyl)-1-methyloctahydro-1H-indol-6-yl]-N′-(2-fluoro-5-methylphenyl)urea trifluoroacetate

Reagent: 2-fluoro-5-methylphenyl isocyanate

Synthetic procedure: Scheme E

Yield: 9.1 mg (48%)

Measured mass: 441.2423

Calc. mass: 441.2428

### EXAMPLE 103

### N-[(3aS*,6R*,7aS*)-3a-(3,4-dimethoxyphenyl)-1-methyloctahydro-1H-indol-6-yl]-N′-(3,5-dinitrophenyl)urea trifluoroacetate

Reagent: 3,5-dinitrophenyl isocyanate

Synthetic procedure: Scheme E

Yield: 8.7 mg (41%)

Measured mass: 499.2050

Calc. mass: 499.2067

### EXAMPLE 104

### N-(2,5-difluorophenyl)-N′-[(3aS*,6R*,7aS*)-3a-(3,4-dimethoxyphenyl)-1-methyloctahydro-1H-indol-6-yl]urea trifluoroacetate

Reagent: 2,5-difluorophenyl isocyanate

Synthetic procedure: Scheme E

Yield: 5.9 mg (31%)

Measured mass: 445.2191

Calc. mass: 445.2177

### EXAMPLE 105

### N-(3-chloro-4-methylphenyl)-N′-[(3aS*,6R*,7aS*)-3a-(3,4-dimethoxyphenyl)-1-methyloctahydro-1H-indol-6-yl]urea trifluoroacetate

Reagent: 3-chloro-4-methylphenyl isocyanate

Synthetic procedure: Scheme E

Yield: 7.9 mg (40%)

Measured mass: 457.2110

Calc. mass: 457.2132

### EXAMPLE 106

### N-[(3aS*,6R*,7aS*)-3a-(3,4-dimethoxyphenyl)-1-methyloctahydro-1H-indol-6-yl]-N′-(2-methyl-5-nitrophenyl)urea trifluoroacetate

Reagent: 2-methyl-5-nitrophenyl isocyanate

Synthetic procedure: Scheme E

Yield: 5.5 mg (27%)

Measured mass: 468.2376

Calc. mass: 468.2373

### EXAMPLE 107

### N-[4-(difluoromethoxy)phenyl]-N′-[(3aS*,6R*,7aS*)-3a-dimethoxyphenyl)-1-methyloctahydro-1H-indol-6-yl]urea trifluoroacetate

Reagent: 4-(difluoromethoxy)phenyl isocyanate

Synthetic procedure: Scheme E

Yield: 6.5 mg (32%)

Measured mass: 475.2285

Calc. mass: 475.2283

### EXAMPLE 108

### N-(4-chloro-2-methylphenyl)-N′-[(3aS*,6R*,7aS*)-3a-(3,4-dimethoxyphenyl)-1-methyloctahydro-1H-indol-6-yl]urea trifluoroacetate

Reagent: 4-chloro-2-methylphenyl isocyanate

Synthetic procedure: Scheme E

Yield: 4.0 mg (20%)

Measured mass: 457.2132

Calc. mass: 457.2132

### EXAMPLE 109

### N-(4-bromo-3-methylphenyl)-N′-[(3aS*,6R*,7aS*)-3a-(3,4-dimethoxyphenyl)-1-methyloctahydro-1H-indol-6-yl]urea trifluoroacetate

Reagent: 4-bromo-3-methylphenyl isocyanate

Synthetic procedure: Scheme E

Yield: 7.7 mg (36%)

Measured mass: 501.1622

Calc. mass: 501.1627

### EXAMPLE 110

### N-(4-bromo-2-fluorophenyl)-N′-[(3aS*,6R*,7aS*)-3a-(3,4-dimethoxyphenyl)-1-methyloctahydro-1H-indol-6-yl]urea trifluoroacetate

Reagent: 4-bromo-2-fluorophenyl isocyanate

Synthetic procedure: Scheme E

Yield: 7.4 mg (35%)

Measured mass: 505.1315

Calc. mass: 505.1376

### EXAMPLE 111

### N-[3,5-bis(trifluoromethyl)phenyl]-N′-[(3aS*,6R*,7aS*)-3a-(3,4-dimethoxyphenyl)-1-methyloctahydro-1H-indol-6-yl]urea trifluoroacetate

Reagent: 3,5-bis(trifluoromethyl)phenyl isocyanate

Synthetic procedure: Scheme E

Yield: 5.1 mg (22%)

Measured mass: 545.2125

Calc. mass: 545.2113

### EXAMPLE 112

### N-[(3aS*,6R*,7aS*)-3a-(3,4-dimethoxyphenyl)-1-methyloctahydro-1H-indol-6-yl]-N′-(4-fluoro-2-methylphenyl)urea trifluoroacetate

Reagent: 4-fluoro-2-methylphenyl isocyanate

Synthetic procedure: Scheme E

Yield: 5.3 mg (28%)

Measured mass: 441.2412

Calc. mass: 41.2428

### EXAMPLE 113

### N-(4-bromo-2-chlorophenyl)-N′-[(3aS*,6R*,7aS*)-3a-(3,4-dimethoxyphenyl)-1-methyloctahydro-1H-indol-6-yl]urea trifluoroacetate

Reagent: 4-bromo-2-chlorophenyl isocyanate

Synthetic procedure: Scheme E

Yield: 3.8 mg (17%)

Measured mass: 521.1073

Calc. mass: 521.1081

### EXAMPLE 114

### N-[(3aS*,6R*,7aS*)-3a-(3,4-dimethoxyphenyl)-1-methyloctahydro-1H-indol-6-yl]-N′-(3,5-dimethylphenyl)urea trifluoroacetate

Reagent: 3,5-dimethylphenyl isocyanate

Synthetic procedure: Scheme E

Yield: 8.0 mg (42%)

Measured mass: 437.2677

Calc. mass: 437.2678

### EXAMPLE 115

### N-[(3aS*,6R*,7aS*)-3a-(3,4-dimethoxyphenyl)-1-methyloctahydro-1H-indol-6-yl]-N′-(2,5-dimethylphenyl)urea trifluoroacetate

Reagent: 2,5-dimethylphenyl isocyanate

Synthetic procedure: Scheme E

Yield: 6.0 mg (31%)

Measured mass: 437.2680

Calc. mass: 437.2678

### EXAMPLE 116

### N-[(3aS*,6R*,7aS*)-3a-(3,4-dimethoxyphenyl)-1-methyloctahydro-1H-indol-6-yl]-N′-(3,4-dimethoxyphenyl)urea trifluoroacetate

Reagent: 3,4-dimethylphenyl isocyanate

Synthetic procedure: Scheme E

Yield: 7.5 mg (40%)

Measured mass: 437.2667

Calc. mass: 437.2678

### EXAMPLE 117

### N-(3,4-difluorophenyl)-N′-[(3aS*,6R*,7aS*)-3a-(3,4-dimethoxyphenyl)-1-methyloctahydro-1H-indol-6-yl]urea trifluoroacetate

Reagent: 3,4-difluorophenyl isocyanate

Synthetic procedure: Scheme E

Yield: 6.1 mg (32%)

Measured mass: 445.2171

Calc. mass: 445.2177

### EXAMPLE 118

### N-[2-chloro-4-(trifluoromethyl)phenyl]-N′-[(3aS*,6R*,7aS*)-3a-(3,4-dimethoxyphenyl)-1-methyloctahydro-1H-indol-6-yl]urea trifluoroacetate

Reagent: 2-chloro-4-(trifluoromethyl)phenyl isocyanate

Synthetic procedure: Scheme E

Yield: 8.4 mg (39%)

Measured mass: 511.1842

Calc. mass: 511.1850

### EXAMPLE 119

### N-[(3aS*,6R*,7aS*)-3a-(3,4-dimethoxyphenyl)-1-methyloctahydro-1H-indol-6-yl]-N′-(4-nitrophenyl)urea trifluoroacetate

Reagent: 4-nitrophenyl isocyanate

Synthetic procedure: Scheme E

Yield: 3.2 mg (16%)

Measured mass: 454.2223

Calc. mass: 454.2216

### EXAMPLE 120

### N-[(3aS*,6R*,7aS*)-3a-(3,4-dimethoxyphenyl)-1-methyloctahydro-1H-indol-6-yl]-N′-(2-fluoro-5-nitrophenyl)urea trifluoroacetate

Reagent: 2-fluoro-5-nitrophenyl isocyanate

Synthetic procedure: Scheme E

Yield: 5.5 mg (27%)

Measured mass: 472.2110

Calc. mass: 472.2122

### EXAMPLE 121

### N-[(3aS*,6R*,7aS*)-3a-(3,4-dimethoxyphenyl)-1-methyloctahydro-1H-indol-6-yl]-N′-(3-ethylphenyl)urea trifluoroacetate

Reagent: 3-ethylphenyl isocyanate

Synthetic procedure: Scheme E

Yield: 7.7 mg (41%)

Measured mass: 437.2666

Calc. mass: 437.2678

### EXAMPLE 122

### N-(4-chlorophenyl)-N′-[(3aS*,6S*,7aS*)-3a-(3,4-dimethoxyphenyl)-1-methyloctahydro-1H-indol-6-yl]-N-methylurea trifluoroacetate

A solution of the trans-amine, (3aS*,6S*,7aS*)-3a-(3,4-dimethoxyphenyl)-1-methyloctahydro-1H-indol-6-amine (Comparative Example 8; 145 mg; 0.5 mol) in methylene chloride (20.0 ml) was treated with Hunigs base (0.5 mol) and para-nitrophenyl chloroformate (0.5 mol). The mixture was shaken at room temperature for 18 h. Then, the solvent was removed by evaporation to afford a yellow gum which was partitioned between saturated aqueous sodium bicarbonate and methylene chloride. Evaporation of the organic extract gave the crude para-nitrophenyl carbamate which was used directly without further purification.

A solution of N-methyl-4-chloroaniline (0.25 mmol) in dry tetrahydrofuran (2.0 ml) was treated at room temperature with a solution of 1M LDA in THF (0.27 ml; 0.27 mmol) and the mixture shaken at room temperature for 30 min. The mixture was then treated with an aliquot of the crude carbamate formed above (0.27 mmol) in THF (1.0 ml) and shaking continued for 18 h. The mixture was quenched with 3-4 drops of water and the solvent removed by evaporation. The residue was purified by preparative HPLC.

Yield=6.5 mg. Measured mass: 457.2139. Calc. mass: 457.2132

### EXAMPLE 123

### N-biphenyl-4-yl-N′-[(3aS*,6R*,7aS*)-3a-(3,4-dimethoxyphenyl)-1-methyloctahydro-1H-indol-6-yl]urea trifluoroacetate

Reagent: 4-biphenylyl isocyanate

Synthetic procedure: Scheme E

Yield: 7.6 mg (37%)

Measured mass: 485.2678

Calc. mass: 485.2678

### EXAMPLE 124

### N-(2,5-dichlorophenyl)-N′-[(3aS*,6R*,7aS*)-3a-(3,4-dimethoxyphenyl)-1-methyloctahydro-1H-indol-6-yl]urea trifluoroacetate

Reagent: 2,5-dichlorophenyl isocyanate

Synthetic procedure: Scheme E

Yield: 4.9 mg (24%)

Measured mass: 477.1582

Calc. mass: 477.1586

### EXAMPLE 125

### N-(3,4-dichlorophenyl)-N′-[(3aS*,6R*,7aS*)-3a-(3,4-dimethoxyphenyl)-1-methyloctahydro-1H-indol-6-yl]urea trifluoroacetate

Reagent: 3,4-dichlorophenyl isocyanate

Synthetic procedure: Scheme E

Yield: 2.2 mg (11%)

Measured mass: 477.1585

Calc. mass: 477.1586

### EXAMPLE 126

### N-[(3aS*,6R*,7aS*)-3a-(3,4-dimethoxyphenyl)-1-methyloctahydro-1H-indol-6-yl]-N′-(2,2,4,4-tetrafluoro-4H-1,3-benzodioxin-6-yl)urea trifluoroacetate

Reagent: 2,2,4,4-tetrafluoro-6-isocyanato-1,3-benzodioxene

Synthetic procedure: Scheme E

Yield: 9.8 mg (44%)

Measured mass: 539.2022

Calc. mass: 539.2043

### EXAMPLE 127

### N-(3,4-dichlorophenyl)-N′-[(3aS*,6S*,7aS*)-3a-(3,4-dimethoxyphenyl)-1-methyloctahydro-1H-indol-6-yl]-N-methylurea trifluoroacetate

A solution of the trans-amine, Comparative Example 8, (145 mg; 0.5 mol) in methylene chloride (20.0 ml) was treated with Hunigs base (0.5 mol) and para-nitrophenyl chloroformate (0.5 mol). The mixture was shaken at room temperature for 18 h. Then, the solvent was removed by evaporation to afford a yellow gum, which was partitioned between saturated aqueous sodium bicarbonate and methylene chloride. Evaporation of the organic extract gave the crude para-nitrophenyl carbamate, which was used directly without further purification.

A solution of N-methyl-3,4-chloroaniline (0.25 mmol) in dry dimethylformamide (2.0 ml) was treated at room temperature with NaH (0.27 mmol) and the mixture shaken at room temperature for 30 min. The mixture was then treated with an aliquot of the crude carbamate formed above (0.27 mmol) in THF (1.0 ml) and shaking continued for 18 h.

The mixture was quenched with 3-4 drops of water and the solvent removed by evaporation.

The residue was purified by preparative HPLC.

Measured mass: 491.1745

Calc. mass: 491.1742

### EXAMPLE 128

### N-(5-chloro-2-methylphenyl)-N′-[(3aS*,6R*,7aS*)-3a-(3,4-dimethoxyphenyl)-1-methyloctahydro-1H-indol-6-yl]urea trifluoroacetate

Reagent: 5-chloro-2-methylphenyl isocyanate

Synthetic procedure: Scheme E

Yield: 4.9 mg (25%)

Measured mass: 457.2117

Calc. mass: 457.2132

### EXAMPLE 129

### N-(3-bromophenyl)-N′-cyano-N″-[(3aS*,6R*,7aS*)-3a-(3,4-dimethoxyphenyl)-1-methyloctahydro-1H-indol-6-yl]guanidine trifluoroacetate

Phenyl N-(3-bromophenyl)-N′-cyanoimidocarbamate (10 mg, 0.03 mmol) and (3aS*,6R*,7aS*)-3a-(3,4-dimethoxyphenyl)-1-methyloctahydro-1H-indol-6-amine (Comparative Example 7; 19 mg, 0.06 mmol) were mixed in isopropanol (2 ml) and heated in microwave at 200° C. for 300 s. The crude mixture was purified by preparative HPLC to give the title compound, 1 mg. HRMS: measured: 511.1580; calc.: 511.1583.

### EXAMPLE 130

### N-[(3aS*,6R*,7aS*)-3a-(3,4-dimethoxyphenyl)-1-methyloctahydro-1H-indol-6-yl]-N′-(3-nitrophenyl)thiourea trifluoroacetate

Reagent: 3-nitrophenyl isothiocyanate

Synthetic procedure: Scheme E

Yield: 2.7 mg (17%)

Measured mass: 470.1997

Calc. mass: 470.1988

### EXAMPLE 131

### N-[(3aS*,6R*,7aS*)-3a-(3,4-dimethoxyphenyl)-1-methyloctahydro-1H-indol-6-yl]-N′-(4-fluorophenyl)thiourea

Reagent: 4-fluorophenyl isothiocyanate

Synthetic procedure: Scheme E

Yield: 0.4 mg (3%)

Measured mass: 443.2023

Calc. mass: 443.2043

### EXAMPLE 132

### N-(3-chlorophenyl)-N′-[(3aR,6S,7aR)-3a-(3,4-dimethoxyphenyl)-1-methyloctahydro-1H-indol-6-yl]urea

A solution of the amine, (3aR,6S,7aR)-3a-(3,4-dimethoxyphenyl)-1-methyloctahydro-1H-indol-6-amine, Comparative Example 11 (12 mg; 0.04 mmol) in dry THF (1.0 ml) was treated with 3-chlorophenylisocyanate (1 eq; 0.04 mmol) and the resulting solution stirred at RT overnight.

The mixture was evaporated and the residue was purified by preparative HPLC.

Yield: 6.7 mg (37%)

Measured mass: 443.1987

Calc. mass: 443.1976

### EXAMPLE 133

### N-[4-chloro-3-(trifluoromethyl)phenyl]-N′-[(3aR,6S,7aR)-3a-(3,4-dimethoxyphenyl)-1-methyloctahydro-1H-indol-6-yl]urea

A solution of the amine, (3aR,6S,7aR)-3a-(3,4-dimethoxyphenyl)-1-methyloctahydro-1H-indol-6-amine, Comparative Example 11 (12 mg; 0.04 mmol) in dry THF (1.0 ml) was treated with 4-chloro-3-trifluoromethyphenylisocyanate (1 eq; 0.04 mmol) and the resulting solution stirred at RT overnight.

The mixture was evaporated and the residue was purified by preparative HPLC.

Yield: 7.8 mg (37%)

Measured mass: 511.1846

Calc. mass: 511.1850

### EXAMPLE 134

### N-(3-chlorophenyl)-N′-[(3aS,6R,7aS)-3a-(3,4-dimethoxyphenyl)-1-methyloctahydro-1H-indol-6-yl]urea trifluoroacetate

A solution of the amine, (3aS,6R,7aS)-3a-(3,4-dimethoxyphenyl)-1-methyloctahydro-1H-indol-6-amine, Comparative Example 10 (25 mg; 0.08 mmol) in dry THF (1.0 ml) was treated with 3-chlorophenylisocyanate (1 eq; 0.08 mmol) and the resulting solution stirred at RT overnight.

The mixture was evaporated and the residue was purified by preparative HPLC.

Yield: 15.4 mg (32%)

Measured mass: 443.1958

Calc. mass: 443.1976

### EXAMPLE 135

### N-[4-chloro-3-(trifluoromethyl)phenyl]-N′-[(3aS,6R,7aS)-3a-(3,4-dimethoxyphenyl)-1-methyloctahydro-1H-indol-6-yl]urea trifluoroacetate

A solution of the amine (3aS,6R,7aS)-3a-(3,4-dimethoxyphenyl)-1-methyloctahydro-1H-indol-6-amine, Comparative Example 10 (25 mg; 0.08 mmol) in dry THF (1.0 ml) was treated with 4-chloro-3-trifluoromethylphenylisocyanate (1 eq; 0.08 mmol) and the resulting solution stirred at RT overnight.

The mixture was evaporated and the residue was purified by preparative HPLC.

Yield: 28.5 mg (52%)

Measured mass: 511.1840

Calc. mass: 511.1850

### EXAMPLE 136

### N-(3-bromophenyl)-N′-[(3aS,6R,7aS)-3a-(3,4-dimethoxyphenyl)-1-methyloctahydro-1H-indol-6-yl]urea trifluoroacetate

A solution of the amine, amine (3aS,6R,7aS)-3a-(3,4-dimethoxyphenyl)-1-methyloctahydro-1H-indol-6-amine, Comparative Example 10 (25 mg; 0.08 mmol) in dry THF (1.0 ml) was treated with 3-bromophenyl isocyanate (1 eq; 0.08 mmol) and the resulting solution stirred at RT overnight.

The mixture was evaporated and the residue was purified by preparative HPLC.

Yield: 27.4 mg (53%)

Measured mass: 487.1474

Calc. mass: 487.1471

### EXAMPLE 137

### N-cyano-N′-(3,5-dichlorophenyl)-N″-[(3aS*,6R*,7aS*)-3a-(3,4-dimethoxyphenyl)-1-methyloctahydro-1H-indol-6-yl]guanidine

Phenyl N-cyano-N′-(3,5-dichlorophenyl)imidocarbamate (25 mg, 0.08 mmol) and (3aS*,6R*,7aS*)-3a-(3,4-dimethoxyphenyl)-1-methyloctahydro-1H-indol-6-amine (Comparative Example 7; 18 mg, 0.12 mmol) were mixed in anhydrous DMF (1.5 ml) and heated in microwave at 200° C. for 300 s. CH₂Cl₂ was added and the mixture was extracted repeatedly with H₂O, dried over MgSO₄ and concentrated. The crude product was purified by column chromatography on silica (CH₂Cl₂/MeOH 30:1) to give the title compound.

HRMS (EI) calc: 501.1698 found: 501.1692.

### EXAMPLE 138

### N-[(3aS*,6R*,7aS*)-3a-(3,4-dimethoxyphenyl)-1-methyloctahydro-1H-indol-6-yl]-N′-[5-methyl-2-(trifluoromethyl)-3-furyl]urea trifluoroacetate

Reagent: 5-methyl-2-(trifluoromethyl)-3-furyl isocyanate

Synthetic procedure: Scheme E

Yield: 3.9 mg (19%)

Measured mass: 481.2189

Calc. mass: 481.2188

### EXAMPLE 139

### N-(2,3-dihydro-1-benzofuran-5-yl)-N′-[(3aS*,6R*,7aS*)-3a-(3,4-dimethoxyphenyl)-1-methyloctahydro-1H-indol-6-yl]urea trifluoroacetate

Reagent: 2,3-dihydro-1-benzofuran-5-yl isocyanate

Synthetic procedure: Scheme E

Yield: 1.5 mg (8%)

Measured mass: 451.2479

Calc. mass: 451.2471

### EXAMPLE 140

### N-[(3aS*,6R*,7aS*)-3a-(3,4-diethoxyphenyl)-1-methyloctahydro-1H-indol-6-yl]-N′-(6-fluoro-4H-1,3-benzodioxin-7-yl)urea trifluoroacetate

Reagent: 6-fluoro-7-isocyanato-4H-1,3-benzodioxine

Synthetic procedure: Scheme E

Yield: 4.2 mg (20%)

Measured mass: 485.2343

Calc. mass: 485.2326

### EXAMPLE 141

### N-[(3aS*,6R*,7aS*)-3a-(3,4-dimethoxyphenyl)-1-methyloctahydro-1H-indol-6-yl]-N′-3-thienylurea trifluoroacetate

Reagent: 3-thienyl isocyanate

Synthetic procedure: Scheme E

Yield: 2.8 mg (15%)

Measured mass: 415.1940

Calc. mass: 415.1930

### EXAMPLE 142

### N-[(3aS*,6R*,7aS*)-3a-(3,4-dimethoxyphenyl)-1-methyloctahydro-1H-indol-6-yl]-N′-(6-morpholin-4-ylpyridin-3-yl)thiourea trifluoroacetate

Reagent: 6-morpholino-3-pyridinyl isothiocyanate

Synthetic procedure: Scheme E

Yield: 6.6 mg (31%)

Measured mass: 511.2617

Calc. mass: 511.2617

### EXAMPLE 143

### N-[(3aS*,6R*,7aS*)-3a-(3,4-dimethoxyphenyl)-1-methyloctahydro-1H-indol-6-yl]-N′-[4-(1H-pyrazol-1-yl)phenyl]thiourea trifluoroacetate

Reagent: 4-(1H-pyrazol-1-yl)phenyl isothiocyanate

Synthetic procedure: Scheme E

Yield: 11.4 mg (55%)

Measured mass: 491.2349

Calc. mass: 491.2355

### EXAMPLE 144

### (E)-N˜1˜-(3,5-dichlorophenyl)-N˜1˜-[(3aS*,6R*,7aS*)-3a-(3,4-dimethoxyphenyl)-1-methyloctahydro-1H-indol-6-yl]-2-nitroethylene-1,1-diamine trifluoroacetate

3,5-dichloro-N-[(Z)-1-(methylthio)-2-nitrovinyl]aniline (20 mg, 0.07 mmol) and (3aS*,6R*,7aS*)-3a-(3,4-dimethoxyphenyl)-1-methyloctahydro-1H-indol-6-amine (Comparative Example 7; 30 mg, 0.10 mmol) were mixed in anhydrous DMF (1.5 ml) and heated in microwave at 200° C. for 300 s. The crude mixture was purified by preparative HPLC to give the title compound, 2 mg. HRMS (EI) calc: 520.1644 found: 520.1647

### EXAMPLE 145

### N-[4-chloro-3-(trifluoromethyl)phenyl]-N′-cano-N″-[(3aS*,6R*,7aS*)-3a-(3,4-dimethoxyphenyl)-1-methyloctahydro-1H-indol-6-yl]guanidine trifluoroacetate

Phenyl N-[4-chloro-3-(trifluoromethyl)phenyl]-N′-cyanoimidocarbamate (10 mg, 0.03 mmol) and (3aS*,6R*,7aS*)-3a-(3,4-dimethoxyphenyl)-1-methyloctahydro-1H-indol-6-amine (Comparative Example 7; 30 mg, 0.10 mmol) were mixed in anhydrous DMF (1.5 ml) and heated in microwave at 200° C. for 300 s. The crude mixture was purified by preparative HPLC to give the title compound, 3 mg. HRMS (EI) calc: 535.1962 found: 535.1966

### EXAMPLE 146

### N′-[4-chloro-3-(trifluoromethyl)phenyl]-N-cyclohexyl-N-[(3aS*,6R*,7aS*)-3a-(3,4-dimethoxyphenyl)-1-methyloctahydro-1H-indol-6-yl]urea hydrochloride

(3aS*,6R*,7aS*)-3a-(3,4-dimethoxyphenyl)-1-methyloctahydro-1H-indol-6-amine (Comparative Example 7; 39.5 mg, 136 μmol) was dissolved in dichloromethane (2μL). Cyclohexanone (16 mL, 15.2 mg, 154 μmol) was added, and the reaction stirred at room temperature for 20 minutes before sodium acetoxyborohydride (100 mg, 472 μmol) was added. After stirring for 16 hours at room temperature, the mixture was partitioned between 2M sodium hydroxide solution (50 mL) and dichloromethane (50 mL). The organic layer was separated, dried (MgSO₄) and the solvent removed under reduced pressure. The crude oily product was dissolved in dichloromethane (3 mL), and 3-trifluoromethyl-4-chlorophenylisocyanate (60 mg, 271 μmol) was added. The mixture was then stirred for 24 hours before the solvent was removed under reduced pressure. The crude product was purified by column chromatography (SiO₂, ethyl acetate as eluent), and the HCl salt formed (HCl in ether solution) to give the title compound as a white solid (51 mg, 60%):

MS (ESI+) for C₃₁H₃₉ClF₃N₃O₃: m/z 594.2 (M+1).

HRMS (EI) calcd C₂₆H₃₁ClF₃N₃O₃: 593.2632, found 593.2648

### EXAMPLE 147

### N′-[4-chloro-3-(trifluoromethyl)phenyl]-N-[(3aS*,6R*,7aS*)-3a-(3,4-dimethoxyphenyl)-1-methyloctahydro-1H-indol-6-yl]-N-(1-methylpiperidin-4-yl)urea and

### EXAMPLE 148

### N′-[4-chloro-3-(trifluoromethyl)phenyl]-N-[(3aS*,6R*,7aS*)-3a-(3,4-dimethoxyphenyl)-1-methyloctahydro-1H-indol-6-yl]-N-(1-methyl-1,2,3,6-tetrahydropyridin-4-yl)urea

Compounds were prepared in an analogous method to that described in Example 146, starting with cis/trans amine, Comparative Example 7 and 8 (37 mg). The compounds were purified by column chromatography (SiO₂, 5% methanol, 1% triethylamine in ethyl acetate as eluent), to give:

N′-[4-chloro-3-(trifluoromethyl)phenyl]-N-[(3aS*,6R*,7aS*)-3a-(3,4-dimethoxyphenyl) 1-methyloctahydro-1H-indol-6-yl]-N-(1-methyl-1,2,3,6-tetrahydropyridin-4-yl)urea (7.1 mg, 9%):

MS (ESI+) for C₃₁H₃₈ClF₃N₄O₃: m/z 607.2 (M+1).

HRMS (EI) calcd C₃₁H₃₈ClF₃N₄O₃: 606.2585, found 606.2588

N′-[4-chloro-3-(trifluoromethyl)phenyl]-N-[(3aS*,6R*,7aS*)-3a-(3,4-dimethoxyphenyl)-1-methyloctahydro-1H-indol-6-yl]-N-(1-methylpiperidin-4-yl)urea (33.3 mg, 43%):

MS (ESI+) for C₃₁H₃₈ClF₃N₄O₃: m/z 609.3 (M+1).

HRMS (EI) calcd C₃₁H₄₀ClF₃N₄O₃: 608.2741, found 608.2731

### EXAMPLE 149

### N-benzyl-N′-[4-chloro-3-(trifluoromethyl)phenyl]-N-[(3aS*,6R*,7aS*)-3a-(3, 4-dimethoxyphenyl)-1-methyloctahydro-1H-indol-6-yl]urea and

### EXAMPLE 150

### N-benzyl-N′-[4-chloro-3-(trifluoromethyl)phenyl]-N-[(3aS*,6S*,7aS*)-3a-(3, 4-dimethoxyphenyl)-1-methyloctahydro-1H-indol-6-yl]urea

Mesembrine (100 mg, 350 μmol) was dissolved in methanol (7 mL). Benzylamine (46 μL, 45 mg, 420 μmol) was added, followed by sodium cyanoborohydride (50 mg, 796 μmol). The mixture was stirred overnight at room temperature and the solvent removed under reduced pressure. The crude product was partitioned between NaOH solution (25 mL, 3M) and DCM (25 mL). The aqueous portion was extracted with further DCM (2×20 mL), the combined extracts dried (Na₂SO₄), and the solvent was removed under reduced pressure. The oily residue was dissolved in DCM (10 mL), and treated with 3-trifluoromethyl-4-chlorophenylisocyanate (100 mg, 451 μmol). After stirring at room temperature for 16 hours, the solvent was removed and the crude products purified by column chromatography (SiO₂, ethyl acetate as eluent).

N-benzyl-N′-[4-chloro-3-(trifluoromethyl)phenyl]-N-[(3aS*,6R*,7aS*)-3a-(3,4-dimethoxyphenyl)-1-methyloctahydro-1H-indol-6-yl]urea (7.0 mg, 3%):

MS (ESI+) for C₃₂H₃₅ClF₃N₃O₃: m/z 602.2 (M+1).

HRMS (EI) calcd C₃₂H₃₅ClF₃N₃O₃: 601.2319, found 601.2348

N-benzyl-N′-[4-chloro-3-(trifluoromethyl)phenyl]-N-[(3aS*,6S*,7aS*)-3a-(3,4-dimethoxyphenyl)-1-methyloctahydro-1H-indol-6-yl]urea (56.3 mg, 27%):

MS (ESI+) for C₃₂H₃₅ClF₃N₃O₃: m/z 602.2 (M+1).

HRMS (EI) calcd C₃₂H₃₅ClF₃N₃O₃: 601.2319, found 601.2329

### EXAMPLE 151

### N-butyl-N′-[4-chloro-3-(trifluoromethyl)phenyl]-N-[(3aS*,6R*,7aS*)-3a-(3,4-dimethoxyphenyl)-1-methyloctahydro-1H-indol-6-yl]urea and

### EXAMPLE 152

### N-butyl-N′-[4-chloro-3-(trifluoromethyl)phenyl]-N-[(3aS*,6S*,7aS*)-3a-(3,4-dimethoxyphenyl)-1-methyloctahydro-1H-indol-6-yl]urea

The compounds were prepared and purified in an analogous method to that described in Example 149 and 150.

N-butyl-N′-[4-chloro-3-(trifluoromethyl)phenyl]-N-[(3aS*,6R*,7aS*)-3a-(3,4-dimethoxyphenyl)-1-methyloctahydro-1H-indol-6-yl]urea (6.6 mg, 3%):

MS (ESI+) for C₂₉H₃₇ClF₃N₃O₃: m/z 568.3 (M+1).

HRMS (EI) calcd C₂₉H₃₇ClF₃N₃O₃: 567.2476, found 567.2454

N-butyl-N′-[4-chloro-3-(trifluoromethyl)phenyl]-N-[(3aS*,6S*,7aS*)-3a-(3,4-dimethoxyphenyl)-1-methyloctahydro-1H-indol-6-yl]urea (29.1 mg, 15%):

MS (ESI+) for C₂₉H₃₇ClF₃N₃O₃: m/z 568.3 (M+1).

HRMS (EI) calcd C₂₉H₃₇ClF₃N₃O₃: 567.2476, found 567.2457

### EXAMPLE 153

### N′-[4-chloro-3-(trifluoromethyl)phenyl]-N-[(3aS*,6S*,7aS*)-3a-(3,4-dimethoxyphenyl)-1-methyloctahydro-1H-indol-6-yl]-N-isopropylurea

The compound was prepared and purified in an analogous method to that described in Example 149 and 150: (33.9 mg, 17%):

MS (ESI+) for C₂₈H₃₅ClF₃N₃O₃: m/z 554.3 (M+1).

HRMS (EI) calcd C₂₈H₃₅ClF₃N₃O₃: 553.2319, found 553.2337

### EXAMPLE 154

### N′-[4-chloro-3-(trifluoromethyl)phenyl]-N-[(3aS*,6R*,7aS*)-3a-(3,4-dimethoxyphenyl)-1-methyloctahydro-1H-indol-6-yl]-N-(2-methoxyethyl)urea

The compound was prepared and purified in an analogous method to that described Example 149 and 150 (9.3 mg, 5%):

MS (ESI+) for C₂₈H₃₅ClF₃N₃O₄: m/z 570.2 (M+1).

HRMS (EI) calcd C₂₈H₃₅ClF₃N₃O₃: 569.2268, found 569.2261

### EXAMPLE 155

### N′-[4-chloro-3-(trifluoromethyl)phenyl]-N-[(3aS*,6S*,7aS*)-6-cyano-3a-(3,4-dimethoxyphenyl)-1-methyloctahydro-1H-indol-6-yl]-N-ethylurea and

### EXAMPLE 156

### N′-[4-chloro-3-(trifluoromethyl)phenyl]-N-[(3aS*,6R*,7aS*)-3a-(3,4-dimethoxyphenyl)-1-methyloctahydro-1H-indol-6-yl]-N-ethylurea

The compounds were prepared analogous to the procedure in Example 149 and 150 using ethylamine and separated by flash chromatography using chloroform sat. with NH₃ (g) as eluent.

The faster eluting isomer showed spectral data in accordance with the cyanohydrin analogue, which was presumably formed in the reductive amination step.

N′-[4-chloro-3-(trifluoromethyl)phenyl]-N-[(3aS*,6S*,7aS*)-6-cyano-3a-(3,4-dimethoxyphenyl)-1-methyloctahydro-1H-indol-6-yl]-N-ethylurea:

¹H NMR (500 MHz, CDCl₃) δ 1.21 (t, J=7.08 Hz, 2H) 1.52 (m, J=14.65 Hz, 2H) 1.65 (td, J=13.67, 3.17 Hz, 1H) 1.94 (td, J=11.96, 5.13 Hz, 1H) 2.03 (m, 2H) 2.08 (dd, J=15.63, 4.39 Hz, 1H) 2.13 (d, J=15.38 Hz, 1H) 2.31 (td, J=14.10, 3.05 Hz, 1H) 2.38 (s, 3H) 2.45 (td, J=10.44, 6.23 Hz, 1H) 2.88 (m, J=7.08, 7.08, 7.08, 7.08, 7.08 Hz, 1H) 2.97 (s, 1H) 3.32 (m, J=7.08, 7.08, 7.08, 7.08, 7.08 Hz, 1H) 3.37 (td, J=9.34, 5.25 Hz, 1H) 6.88 (d, J=1.95 Hz, 1H) 6.89 (d, J=8.30 Hz, 1H) 6.93 (dd, J=8.55, 1.95 Hz, 1H) 7.69 (dd, J=8.67, 2.32 Hz, 1H) 7.92 (d, J=2.20 Hz, 1H) 10.50 (s, 1H).

MS (ESI+) for C₂₉H₃₄ClF₃N₄O₃: m/z 565 (M+1).

HRMS (EI): Calcd for C₂₈H₃₂ClF₃N₄O₃: 564.2115. Found: 564.2078.

The slower eluting isomer, (silica/CHCl₃ sat. with NH₃), N′-[4-chloro-3-(trifluoromethyl)phenyl]-N-[(3aS*,6R*,7aS*)-3a-(3,4-dimethoxyphenyl)-1-methyloctahydro-1H-indol-6-yl]-N-ethylurea:

¹H NMR (500 MHz, CDCl₃) δ 1.16 (t, J=7.1 Hz, 3H); 1.45-1.68 (m, 2H); 1.80-2.03 (m, 4H); 2.08-2.26 (m, 2H); 2.34-2.47 (m, 4H); 2.87 (s, 1H); 3.12-3.37 (m, 3H); 3.91 (s, 6H); 4.22-4.35 (m, 1H); 6.72 (s, 1H); 6.83-6.98 (m, 3H); 7.38 (d, J=8.7 Hz, 1H); 7.63 (dd, J=8.7, 2.7 Hz, 1H); 7.76 (d, J=2.6 Hz, 1H); MS (ESI+): m/z 540 (M+1).

HRMS (EI): Calcd for C₂₇H₃₃ClF₃N₃O₃: 539.2163. Found: 539.2147

### EXAMPLE 157

### N′-[4-chloro-3-(trifluoromethyl)phenyl]-N-[(3aS*,6S*,7aS*)-6-cyano-3a-(3,4-dimethoxyphenyl)-1-methyloctahydro-1H-indol-6-yl]-N-propylurea and

### EXAMPLE 158

### N′-[4-chloro-3-(trifluoromethyl)phenyl]-N-[(3aS*,6S*,7aS*)-3a-(3,4-dimethoxyphenyl)-1-methyloctahydro-1H-indol-6-yl]-N-propylurea

The compounds were prepared analogous to the procedure in Example 149 and 150 using n-propylamine and separated by flash chromatography using chloroform sat. with NH₃ (g) as eluent.

The faster eluting isomer showed spectral data in accordance with the cyanohydrin analogue, which was presumably formed in the reductive amination step, N′-[4-chloro-3-(trifluoromethyl)phenyl]-N-[(3aS*,6S*,7aS*)-6-cyano-3a-(3,4-dimethoxyphenyl)-1-methyloctahydro-1H-indol-6-yl]-N-propylurea:

¹H NMR (500 MHz, CDCl₃) δ ppm 0.89 (t, J=7.57 Hz, 3H) 1.94 (td, J=11.72, 4.88 Hz, 1H) 2.31 (td, J=13.92, 2.93 Hz, 1H) 2.39 (s, 3H) 2.44 (m, 1H) 2.69 (ddd, J=14.65, 9.77, 5.86 Hz, 1H) 2.96 (s, 1H) 3.23 (ddd, J=14.41, 9.52, 5.37 Hz, 1H) 3.37 (m, 1H) 3.92 (s, 2H) 3.93 (s, 3H) 6.88 (m, 1H) 6.89 (d, J=8.30 Hz, 1H) 6.92 (dd, J=8.30, 1.95 Hz, 1H) 7.55 (d, J=8.79 Hz, 1H) 7.69 (dd, J=8.79, 2.44 Hz, 1H) 7.92 (d, J=1.95 Hz, 1H) 10.50 (s, 1H).

MS (ESI+) for C₂₉H₃₄ClF₃N₄O₃: m/z 579 (M+1).

The slower eluting isomer (silica/CHCl₃ sat. with NH₃), N′-[4-chloro-3-(trifluoromethyl)phenyl]-N-[(3aS*,6R*,7aS*)-3a-(3,4-dimethoxyphenyl)-1-methyloctahydro-1H-indol-6-yl]-N-propylurea:

¹H NMR (500 MHz, CDCl₃) δ 0.89 (t, 3H, J=7.5 Hz); 1.48-1.68 (m, 3H); 1.79-2.02 (m, 4H); 2.08-2.23 (m, 2H); 2.31-2.38 (m, 1H); 2.40 (s, 3H); 2.85 (s, 1H); 2.96-3.15 (m, 2H); 3.26-3.35 (m, 1H); 3.91 (s, 6H); 4.16-4.28 (m, 1H); 6.70 (s, 1H); 6.84-6.97 (m, 3H); 7.38 (d, 1H, J=8.9 Hz); 7.68 (d, 1H, J=8.9 Hz); 7.74 (s, 1H).

MS (ESI+) for: m/z 554 (M+1).

HRMS (EI): Calcd for C₂₈H₃₅ClF₃N₃O₃: 553.2319. Found: 553.2321.

### EXAMPLE 159

### N-ally-N′-[4-chloro-3-(trifluoromethyl)phenyl]-N-[(3aS*,6R*,7aS*)-3a-(3,4-dimethoxyphenyl)-1-methyloctahydro-1H-indol-6-yl]urea and

### EXAMPLE 160

### N-allyl-N′-[4-chloro-3-(trifluoromethyl)phenyl]-N-[(3aS*,6S*,7aS*)-3a-(3,4-dimethoxyphenyl)-1-methyloctahydro-1H-indol-6-yl]urea

The compounds were prepared analogous to the procedure in Example 149 and 150 using allylamine and separated by flash chromatography using chloroform sat. with NH₃ (g) as eluent.

Faster eluting isomer (silica gel/CHCl₃ sat with NH₃), N-allyl-N′-[4-chloro-3-(trifluoromethyl)phenyl]-N-[(3aS*,6R*,7aS*)-3a-(3,4-dimethoxyphenyl)-1-methyloctahydro-1H-indol-6-yl]urea:

¹H NMR (500 MHz, CDCl₃) δ 1.34-1.45 (m, 1H); 1.52-2.36 (m, 8H); 2.37 (s, 3H); 2.77-2.82 (m, 1H); 3.22-3.31 (m, 1H); 3.72-3.77 (m, 2H); 3.85-3.92 (m, 6H); 4.53-4.67 (m, 1H); 5.25-5.40 (m, 2H); 5.82-5.92 (m, 1H); 6.61-6.67 (m, 1H); 6.81-6.96 (m, 3H); 7.36 (d, 1H, J=9.1 Hz); 7.53 (dd, 1H, J=9.1, 3.1 Hz); 7.65 (d, 1H, J=3.1 Hz).

MS (ESI+) for C₂₈H₃₃ClF₃N₃O₃ m/z 552 (M+1).

HRMS (EI) calcd for C₂₈H₃₃ClF₃N₃O₃: 551.2163, found 551.2160.

Slower eluting isomer (silica gel/CHCl₃ sat with NH₃), —N-allyl-N′-[4-chloro-3-(trifluoromethyl)phenyl]-N-[(3aS*,6S*,7aS*)-3a-(3,4-dimethoxyphenyl)-1-methyloctahydro-1H-indol-6-yl]urea:

¹H NMR (500 MHz, CDCl₃) δ ppm 1.69 (m, 1H) 1.78 (m, 2H) 1.93 (m, 2H) 2.15 (m, 1H) 2.20 (m, 1H) 2.26 (m, 1H) 2.30 (s, 3H) 2.56 (m, 1H) 3.04 (m, J=20.26 Hz, 1H) 3.04 (m, J=20.26 Hz, 1H) 3.88 (s, 3H) 3.91 (s, 3H) 3.98 (dd, J=17.21, 5.49 Hz, 1H) 4.10 (dd, J=17.33, 4.88 Hz, 1H) 4.17 (m, 1H) 5.34 (d, J=10.99 Hz, 1H) 5.38 (d, J=17.82 Hz, 1H) 5.96 (m, J=15.38, 10.25, 5.13, 5.13 Hz, 1H) 6.83 (d, J=8.30 Hz, 1H) 6.91 (d, J=2.20 Hz, 1H) 6.94 (dd, J=8.30, 2.20 Hz, 1H) 7.38 (d, J=8.55 Hz, 1H) 7.63 (d, J=8.79 Hz, 1H) 7.65 (d, J=2.44 Hz, 1H) 7.99 (s, 1H).

MS (ESI+) for C₂₈H₃₃ClF₃N₃O₃: m/z 552 (M+1).

HRMS (EI) calcd for C₂₈H₃₃ClF₃N₃O₃: 551.2163, found 551.2179.

### EXAMPLE 161

### N-(3′-cyanobiphenyl-4-yl)-N′-[(3aS*,6R*,7aS*)-3a-(3,4-dimethoxyphenyl)-1-methyloctahydro-1H-indol-6-yl]urea trifluoroacetate

4-Bromoaniline (5 g, 29 mmol), 3-cyanophenylboronic acid (5 g, 34 mmol), Pd(PPh₃)₄ (3.3 g, 2.9 mmol) and Na₂CO₃ (18.4 g, 174 mmol) were mixed in a degassed solution of 50 mL toluene:ethanol:water (3:1:1) and heated at 100° C. for 3 d. The mixture was concentrated by evaporation, diluted with ethyl acetate, filtered and then washed with water. Dried with MgSO₄, filtrated and then concentrated. Purification using flash chromatography (system Heptane:Ethyl acetate 4:1) gave 4′-amino-biphenyl-3-carbonitrile as a brown solid (1.6 g 28%).

MS (ESI+) for C₁₃H₁₀N₂ m/z 195 (M+H⁺), HRMS calculated: 194,0844. ¹HNMR (270 MHz, DMSO-d₆) ppm 5.38 (s, 2H); 6.65 (d, J=8.71, 2H); 7.44 (d, J=8.71, 2H); 7.53-7.64 (m, 1H); 7.87 (d, J=7.92, 1H); 7.98 (s, 1H)

4′-Amino-biphenyl-3-carbonitrile (50 mg, 0.26 mmol) was stirred with 4-nitrophenylchloroformate (52 mg, 0.26 mmol) and diisopropylamine (34 mg, 0.26 mmol) in CH₂Cl₂ at room temperature overnight. The reaction mixture was washed with saturated NaHCO₃, dried with MgSO₄, filtrated and excess solvent is evaporated. The crude product, (3-Cyano-biphenyl)-carbamic acid (4-nitro)-phenylester (12.5 mg, 0.03 mmol), were mixed and stirred at room temperature overnight with (3aS*,6R*,7aS*)-3a-(3,4-dimethoxyphenyl)-1-methyloctahydro-1H-indol-6-amine (Comparative Example 7; 10 mg, 0.03 mmol) and DIPEA (4 mg, 0.03 mmol) in CH₂Cl₂. The solvents were evaporated and the residue was purified using preparative HPLC.

MS (ESI+) for C₃₁H₃₄N₄O₃ m/z 511 (M+H⁺), HRMS found: 510,2637 calculated: 510,2631

¹H NMR (270 MHz, Chloroform-d) ppm 0.86-0.95 (m, 1H); 1.21-1.42 (m, 3H); 1.68-1.71 (m, 1H); 1.87-2.08 (m, 3H); 2.15-2.34 (m, 3H); 3.01 (s, 3H); 3.66 (d, 1H); 3.87 (d, J=4.49, 6H); 4.08-4.28 (m, 1H); 6.71 (s, 1H); 6.84 (t, J=13.72, 1H); 7.46-7.59 (m, 4H); 7.67 (d, J=8.45, 2H); 7.75-7.84 (m, 3H); 8.59 (b, 1H)

### EXAMPLE 162

### N-[(3aS*,6R*,7aS*)-1-benzyl-3a-(3,4-dimethoxyphenyl)octahydro-1H-indol-6-yl]-N′-(3,4-difluorophenyl)urea

3,4-Difluorophenyl isocyanate (0.67 ml, 5.7 mmol) was added to a solution of (3aS*,7aS*)-1-benzyl-3a-(3,4-dimethoxyphenyl)octahydro-1H-indol-6-amine (cis,trans-mixture) (Comparative Example 5 and 6; 2.1 g, 5.7 mmol) in CH₂Cl₂ (50 ml) and stirred at room temperature for 2 hours. The mixture was filtered. The white solid precipitate was washed with cold CH₂Cl₂ (2×) and dried to give only the cis isomer.

Yield: 2.15 g (72%).

¹H NMR (400 MHz, CHLOROFORM-D) δ ppm 1.21-1.35 (m, 1H) 1.72-2.11 (m, 4H) 2.15-2.32 (m, 3H) 3.34 (t, J=9.79 Hz, 1H) 3.78-4.05 (m, 8H) 4.10-4.22 (m, 1H) 4.29 (d, J=13.30 Hz, 1H) 4.52 (d, J=12.55 Hz, 1H) 6.45-6.70 (m, 5H) 6.83 (d, J=8.53 Hz, 1H) 7.03-7.20 (m, 2H) 7.37-7.52 (m, 5H) 8.43 (br.s, 1H)

MS (ionspray; [M+H]⁺) m/z: 522. HRMS for C₃₀H₃₃F₂N₃O₃: Calcd, 521.2490; found, 521.2491.

### EXAMPLE 163

### N-[(3aS*,6R*,7aS*)-3a-(3,4-dimethoxyphenyl)-1-methyloctahydro-1H-indol-6-yl]-N′-(4-isopropylphenyl)thiourea trifluoroacetate

Reagent: 4-isopropylphenyl isothiocyanate

Synthetic procedure: Scheme E

Yield: 2.3 mg (11%)

Measured mass: 467.2602

Calc. mass: 467.2606

### EXAMPLE 164

### N-[3,5-bis(trifluoromethyl)phenyl]-N′-[(3aS*,6R*,7aS*)-3a-(3,4-dimethoxyphenyl)-1-methyloctahydro-1H-indol-6-yl]thiourea trifluoroacetate

Reagent: 3,5-bis(trifluoromethyl)phenyl isothiocyanate

Synthetic procedure: Scheme E

Yield: 7.0 mg (30%)

Measured mass: 561.1879

Calc. mass: 561.1885

### EXAMPLE 165

### N-(3,5-dichlorophenyl)-N′-[(3aS*,6R*,7aS*)-3a-(3,4-dimethoxyphenyl)-1-methyloctahydro-1H-indol-6-yl]thiourea trifluoroacetate

Reagent: 3,5-dichlorophenyl isothiocyanate

Synthetic procedure: Scheme E

Yield: 6.1 mg (29%)

Measured mass: 493.1348

Calc. mass: 493.1358

### EXAMPLE 166

### N-[(3aS*,6R*,7aS*)-3a-(3,4-dimethoxyphenyl)-1-methyloctahydro-1H-indol-6-yl]-N′-(5-fluoro-2-methylphenyl)thiourea trifluoroacetate

Reagent: 5-fluoro-2-methylphenyl isothiocyanate

Synthetic procedure: Scheme E

Yield: 1.9 mg (10%)

Measured mass: 457.2206

Calc. mass: 457.2199

### EXAMPLE 167

### N-(3-chlorophenyl)-N′-[(3aS*,6R*,7aS*)-3a-(3,4-dimethoxyphenyl) 1-methyloctahydro-1H-indol-6-yl]thiourea trifluoroacetate

Reagent: 3-chlorophenyl isothiocyanate

Synthetic procedure: Scheme E

Yield: 2.8 mg (14%)

Measured mass: 459.1739

Calc. mass: 459.1747

### EXAMPLE 168

### N-(2,5-difluorophenyl)-N′-[(3aS*,6R*,7aS*)-3a-(3,4-dimethoxyphenyl)-1-methyloctahydro-1H-indol-6-yl]thiourea trifluoroacetate

Reagent: 2,5-difluorophenyl isothiocyanate

Synthetic procedure: Scheme E

Yield: 2.2 mg (11%)

Measured mass: 461.1938

Calc. mass: 461.1949

### EXAMPLE 169

### N-[(3aS*,6R*,7aS*)-3a-(3,4-dimethoxyphenyl)-1-methyloctahydro-1H-indol-6-yl]-N′-(3,5-dimethylphenyl)thiourea trifluoroacetate

Reagent: 3,5-methylphenyl isothiocyanate

Synthetic procedure: Scheme E

Yield: 2.7 mg (14%)

Measured mass: 453.2438

Calc. mass: 453.2450

### EXAMPLE 170

### N-(3-chloro-4-fluorophenyl)-N′-[(3aS*,6R*,7aS*)-3a-(3,4-dimethoxyphenyl)-1-methyloctahydro-1H-indol-6-yl]thiourea trifluoroacetate

Reagent: 3-chloro-4-fluorophenyl isothiocyanate

Synthetic procedure: Scheme E

Yield: 4.0 mg (20%)

Measured mass: 477.1667

Calc. mass: 477.1653

### EXAMPLE 171

### N-(3,4-dichlorophenyl)-N′-[(3aS*,6R*,7aS*)-3a-(3,4-dimethoxyphenyl)-1-methyloctahydro-1H-indol-6-yl]thiourea trifluoroacetate

Reagent: 3,4-dichlorophenyl isothiocyanate

Synthetic procedure: Scheme E

Yield: 4.7 mg (22%)

Measured mass: 493.1359

Calc. mass: 493.1358

### EXAMPLE 172

### N-[(3aS*,6R*,7aS*)-3a-(3,4-dimethoxyphenyl)-1-methyloctahydro-1H-indol-6-yl]-N′-(6-phenoxypyridin-3-yl)thiourea trifluoroacetate

Reagent: 6-phenoxy-3-pyridinyl isothiocyanate

Synthetic procedure: Scheme E

Yield: 3.4 mg (16%)

Measured mass: 518.2354

Calc. mass: 518.2352

### EXAMPLE 173

### N-[(3aS*,6R*,7aS*)-3a-(3,4-dimethoxyphenyl)-1-methyloctahydro-1H-indol-6-yl]-N′-[4-(piperidin-1-ylsulfonyl)phenyl]thiourea trifluoroacetate

Reagent: 1-[(4-isothiocyanatophenyl)sulfonyl]piperidine

Synthetic procedure: Scheme E

Yield: 3.1 mg (13%)

Measured mass: 572.2470

Calc. mass: 572.2491

### EXAMPLE 174

### N-[(3aS*,6R*,7aS*)-3a-(3,4-dimethoxyphenyl)-1-methyloctahydro-1H-indol-6-yl]-N′-[4-(1,3-oxazol-5-yl)phenyl]thiourea trifluoroacetate

Reagent: 4-(1,3-oxazol-5-yl)phenyl isothiocyanate

Synthetic procedure: Scheme E

Yield: 2.1 mg (10%)

Measured mass: 492.2190

Calc. mass: 492.2195

### EXAMPLE 175

### N-(3,4-difluorophenyl)-N′-[(3aS*,6R*,7aS*)-3a-(3,4-dimethoxyphenyl)octahydro-1H-indol-6-yl]urea

Pd/C (10%, 0.057 g) was added to a solution of N-[(3aS*,6R*,7aS*)-1-benzyl-3a-(3,4-dimethoxyphenyl)octahydro-1H-indol-6-yl]-N′-(3,4-difluorophenyl)urea (Example 162; 0.57 g, 1.1 mmol) in MeOH/THF 1:1 (20 ml) and stirred under 1 atmosphere of H₂ (g). The mixture was stirred for 28 h and evaporated. The crude product was purified by column chromatography on silica gel with chloroform saturated with NH₃(g) as the eluent.

Yield: 0.37 g, (79%). White solid.

¹H NMR (400 MHz, CHLOROFORM-D) δ ppm 0.99-1.12 (m, 1H) 1.39-1.51 (m, 1H) 1.73-2.19 (m, 8H) 2.97-3.18 (m, 2H) 3.62-3.73 (m, 1H) 3.76-3.92 (m, 6H) 3.92-4.05 (m, 1H) 4.71 (br.s, 1H) 6.74-6.94 (m, 4H) 6.95-7.05 (m, 1H) 7.28-7.38 (m, 1H).

MS (ionspray; [M+H]⁺) m/z: 432. HRMS for C₂₃H₂₇F₂N₃O₃: Calcd, 431.2020; found, 431.2025

### EXAMPLE 176

### N-(3,4-difluorophenyl)-N′-[(3aS*,6R*,7aS*)-3a-(3,4-dimethoxyphenyl)-1-propyloctahydro-1H-indol-6-yl]urea trifluoroacetate

N-(3,4-difluorophenyl)-N′-[(3aS*,6R*,7aS*)-3a-(3,4-dimethoxyphenyl)octahydro-1H-indol-6-yl]urea (Example 175) (0.020 g, 0.046 mmol) and propionaldehyde (0.017 ml, 0.23 mmol) were dissolved in MeOH and NaCNBH₃ (0.029 g, 0.46 mmol) was added. The mixture was stirred for 2 hours and evaporated. The crude product was purified by reversed phase HPLC. Isolated as the TFA salt. Yield 14 mg (52%). White solid.

HRMS for C₂₆H₃₃F₂N₃O₃: Calcd, 473.2490; found, 473.2498

### EXAMPLE 177

### N-(3,4-difluorophenyl)-N′-[(3aS*,6R*,7aS*)-3a-(3,4-dimethoxyphenyl)-1-(2-hydroxyethyl)octahydro-1H-indol-6-yl]urea trifluoroacetate (salt)

N-(3,4-difluorophenyl)-N′-[(3aS*,6R*,7aS*)-3a-(3,4-dimethoxyphenyl)octahydro-1H-indol-6-yl]urea (Example 175) (0.020 g, 0.046 mmol) and hydroxyacetaldehyde (0.014 g, 0.23 mmol) were dissolved in MeOH (1 ml) and NaCNBH₃ (0.029 g, 0.46 mmol) was added. The mixture was stirred for 3 h and evaporated. The crude product was purified by reversed phase HPLC. Isolated as the TFA salt. Yield 10 mg (37%). Colourless liquid.

HRMS for C₂₅H₃₂F₂N₃O₄: Calcd, 475.2283; found, 473.2288.

### EXAMPLE 178

### N-(3,4-difluorophenyl)-N′-{(3aS*,6R*,7aS*)-3a-(3,4-dimethoxyphenyl)-1-[(1-methyl-1H-pyrrol-2-yl)methyl]octahydro-1H-indol-6-yl}urea trifluoroacetate

N-(3,4-difluorophenyl)-N′-[(3aS*,6R*,7aS*)-3a-(3,4-dimethoxyphenyl)octahydro-1H-indol-6-yl]urea (Example 175) (0.020 g, 0.046 mmol) and 1-methyl-1H-pyrrole 2-carbaldehyde (0.025 g, 0.23 mmol) were dissolved in MeOH (1 ml) and NaCNBH₃ (0.029 g, 0.46 mmol) was added. The mixture was stirred for 3 h and evaporated. The crude product was purified by reversed phase HPLC. Isolated as the TFA salt. Yield 4.0 mg (14%). White solid.

MS (ionspray; [M+H]⁺) m/z: 525.2. HRMS for C₂₉H₃₄F₂N₄O₄: Calcd, 524.2599; found, 524.2608.

### EXAMPLE 179

### N′-[4-chloro-3-(trifluoromethyl)phenyl]-N-[(3aS*,6R*,7aS*)-3a-(3,4-dimethoxyphenyl)-1-methyloctahydro-1H-indol-6-yl]-N-isopropylurea hydrochloride

(3aS*,6R*,7aS*)-3a-(3,4-dimethoxyphenyl)-1-methyloctahydro-1H-indol-6-amine (Comparative Example 7; 41 mg, 141 μmol) was dissolved in dichloromethane (2 mL). Acetone (12 μL, 9.5 mg, 163 μmol) was added, and the reaction stirred at room temperature for 20 minutes before sodium acetoxyborohydride (100 mg, 472 μmol) was added. After stirring for 16 hours at room temperature, the mixture was partitioned between 2M sodium hydroxide solution (50 mL) and dichloromethane (50 mL). The organic layer was separated, dried (MgSO₄) and the solvent removed under reduced pressure. The crude oily product was dissolved in dichloromethane (3 mL), and 3-trifluoromethyl-4-chlorophenylisocyanate (60 mg, 271 μmol) was added. The mixture was then stirred for 24 hours before the solvent was removed under reduced pressure. The crude product was purified by column chromatography (SiO₂, ethyl acetate as eluent), and the HCl salt formed (HCl in ether solution) to give the title compound as a white solid (20.4 mg, 26%):

MS (ESI+) for C₂₈H₃₅ClF₃N₃O₃: m/z 554.3 (M+1).

HRMS (EI) calcd C₂₈H₃₅ClF₃N₃O₃: 553.2319, found 553.2318

### EXAMPLE 180

### N-(3-chlorophenyl)-N′-[(3aS*,6R*,7aS*)-3a-(3,4-dimethoxyphenyl)-1-methyloctahydro-1H-indol-6-yl]-N-methylurea trifluoroacetate

Triethylamine (33 μL, 0.24 mmol) was added to a solution of (3aS*,6R*,7aS*)-3a-(3,4-dimethoxyphenyl)-1-methyloctahydro-1H-indol-6-amine (Comparative Example 7; 35 mg, 0.120 mmol) dissolved in dry CH₂Cl₂ (1 mL). Triphosgene (14 mg, 0.048 mmol) was dissolved in dry CH₂Cl₂ (0.5 mL) and added drop-wise. The solution was stirred under N₂ in room temperature for 3 h. 3-Chloro-N-methylaniline (17 mg, 0.120 mmol) was added and the reaction mixture was stirred at room temperature over night. Volatiles was evaporated and the crude product was purified by preparative HPLC which gave 20 mg (36%) of the title compound. ¹H NMR (400 MHz, MeOH-D4) δ ppm 1.13-1.22 (m, 1H), 1.63-1.79 (m, 3H), 2.12-2.31 (m, 4H), 2.99 (s, 3H), 3.09 (s, 3H), 3.21 (s, 3H), 3.27 (m, 1H), 3.70 and 3.74 (two s, 6H), 3.76 (m, 2H), 3.96 (m, 1H), 6.85 (m, 3H), 7.06-7.27 (m, 4H).

MS (ESI+) m/z 458 (M+H)⁺. HRMS (EI) calc for C₂₅H₃₂ClN₃O₃: 457.2132 found 457.2134.

### EXAMPLE 181

### N-[(3aS*,6R*,7aS*)-3a-(3,4-dimethoxyphenyl)-1-methyloctahydro-1H-indol-6-yl]-N′-pyridin-2-ylurea

Picolinic acid (32 mg, 0.26 mmol) was dissolved in anhydrous toluene (1 ml) and cooled on ice, under N₂. Et₃N (0.03 ml, 0.26 mmol) and diphenylphosphoryl azide (0.06 ml, 0.26 mmol) were added. Stir at ambient temperature for 2 hrs, then heated at 80° C. for 1.5 hrs. The reaction mixture was cooled to ambient temperature and (3aS*,6R*,7aS*)-3a-(3,4-dimethoxyphenyl)-1-methyloctahydro-1H-indol-6-amine (Comparative Example 7; 50 mg, 0.17 mmol), in anhydrous CH₂Cl₂ (1 ml) was added. The reaction mixture was heated at 80° C. for 3 hrs. CH₂Cl₂ was added and the crude mixture was extracted with 1M aq. HCl, washed with H₂O/sat. aq. NaCl, dried over MgSO₄, and concentrated to give 80 mg of a yellow oil. The crude product was purified by preparative HPLC to give 9 mg of the product as the TFA-salt. The salt was washed through a pad of silica (CH₂Cl₂/MeOH 15:1) to give the title compound, 6.3 mg (9%). HRMS (EI) calc.: 410.2318 found: 410.2308

### EXAMPLE 182

### N-[(3aS*,6R*,7aS*)-3a-(3,4-dimethoxyphenyl)-1-methyloctahydro-1H-indol-6-yl]-N′-pyridin-3-ylurea

Nicotinic acid (32 mg, 0.26 mmol) was dissolved in toluene (1 ml) and cooled on ice, under N₂. Et₃N (0.03 ml, 0.26 mmol) and diphenylphosphoryl azide (0.06 ml, 0.26 mmol) was added and the reaction mixture was stirred at r.t. for 2 hrs, heated at 80° C. for 1.5 hrs, and allowed to cool to ambient temperature. (3aS*,6R*,7aS*)-3a-(3,4-dimethoxyphenyl)-1-methyloctahydro-1H-indol-6-amine (Comparative Example 7; 50 mg, 0.17 mmol), in anhydrous CH₂Cl₂ (1 ml), was added and the reaction mixture was stirred at r.t for 18 hrs. CH₂Cl₂ was added and the crude mixture was extracted with 1M aqueous HCl, washed with H₂O/sat. aq. NaCl, dried over MgSO₄, and concentrated to give the crude product as a yellow oil 84 mg. Purification by preparative HPLC gave 18 mg of the product as the TFA-salt. The salt was washed through a pad of silica (CH₂Cl₂/MeOH 15:1) to give 0.3 mg (0.4%) of pure title compound. HRMS (EI) calc: 410.2318 found: 410.2301

### EXAMPLE 183

### N-[(3aS*,6R*,7aS*)-3a-(3,4-dimethoxyphenyl)-1-methyloctahydro-1H-indol-6-yl]-N′-pyridin-4-ylurea

Isonicotinic acid (32 mg, 0.26 mmol) was dissolved in toluene (1 ml) and cooled on ice, under N₂. Et₃N (0.03 ml, 0.26 mmol) and diphenylphosphoryl azide (0.06 ml, 0.26 mmol) was added and the reaction mixture was stirred at r.t. for 2 hrs, heated at 80° C. for 1.5 hrs, and allowed to cool to ambient temperature. (3aS*,6R*,7aS*)-3a-(3,4-dimethoxyphenyl)-1-methyloctahydro-1H-indol-6-amine (Comparative Example 7; 50 mg, 0.17 mmol), in anhydrous CH₂Cl₂ (1 ml), was added and the reaction mixture was stirred at r.t for 18 hrs. CH₂Cl₂ was added and the crude mixture was extracted with 1M aqueous HCl, washed with H₂O/sat. aq. NaCl, dried over MgSO₄, and concentrated to give the crude product as a yellow oil, 96 mg. Purification by preparative HPLC 10 mg of the product as the TFA-salt. The salt was washed through a pad of silica (CH₂Cl₂/MeOH 15:1) to give 0.7 mg (0.4%) of clean title compound. HRMS (EI) calc: 410.2318 found: 410.2306

### EXAMPLE 184

### N-(3,4-difluorophenyl)-N′-[(3aS*,6R*,7aS*)-3a-(3,4-dimethoxyphenyl)-1-(1H-pyrazol-3-ylmethyl)octahydro-1H-indol-6-yl urea trifluoroacetate

N-(3,4-difluorophenyl)-N′-[(3aS*,6R*,7aS*)-3a-(3,4-dimethoxyphenyl)octahydro-1H-indol-6-yl]urea (Example 175) (0.020 g, 0.046 mmol) and 1H-pyrazole-5-carbaldehyde (0.022 g, 0.23 mmol) were dissolved in MeOH (1 ml) and NaCNBH₃ (0.029 g, 0.46 mmol) was added. The mixture was stirred for 3 h and evaporated. The crude product was purified by reversed phase HPLC. Isolated as the TFA salt. Yield 5.9 mg (25%). Yellow oil. MS (ionspray; [M+H]⁺) m/z: 512.4. HRMS for C₂₇H₃₁F₂N₅O₃: Calcd, 511.2386; found, 511.2395.

### EXAMPLE 185

### N-(3,4-difluorophenyl)-N′-[(3aS*,6R*,7aS*)-3a-(3,4-dimethoxyphenyl)-1-(3,3,3-trifluoro-2-methylpropyl)octahydro-1H-indol-6-yl]urea trifluoroacetate

N-(3,4-difluorophenyl)-N′-[(3aS*,6R*,7aS*)-3a-(3,4-dimethoxyphenyl)octahydro-1H-indol-6-yl]urea (Example 175) (0.020 g, 0.046 mmol) and 3,3,3-trifluoro-2-methylpropanal (0.029 g, 0.23 mmol) were dissolved in MeOH (1 ml) and NaCNBH₃ (0.029 g, 0.46 mmol) was added. The mixture was stirred for 18 h and evaporated. The crude product was purified by reversed phase HPLC. Isolated as the TFA salt. Yield: 9.3 mg (31%). Colourless oil. MS (ionspray; [M+H]⁺) m/z: 542.3. HRMS for C₂₇H₃₂F₅N₃O₃: Calcd, 541.2364; found, 541.2362.

### EXAMPLE 186

### N-[(3aS*,6R*,7aS*)-1-butyl-3a-(3,4-dimethoxyphenyl)octahydro-1H-indol-6-yl]-N′-(3,4-difluorophenyl)urea trifluoroacetate

N-(3,4-difluorophenyl)-N′-[(3aS*,6R*,7aS*)-3a-(3,4-dimethoxyphenyl)octahydro-1H-indol-6-yl]urea (Example 175) (0.020 g, 0.046 mmol) and butyraldehyde (0.017 g, 0.23 mmol) were dissolved in MeOH (1 ml) and NaCNBH₃ (0.029 g, 0.46 mmol) was added. The mixture was stirred for 20 h and evaporated. The crude product was purified by reversed phase HPLC. Isolated as the TFA salt. Yield: 6.7 mg (25%). White solid. MS (ionspray; [M+H]⁺) m/z: 488.3. HRMS for C₂₇H₃₅F₂N₃O₃: Calcd, 487.2646; found, 487.2639.

### EXAMPLE 187

### N-(3,4-difluorophenyl)-N′-[(3aS*,6R*,7aS*)-3a-(3,4-dimethoxyphenyl)-1-(H-imidazol-4-ylmethyl)octahydro-1H-indol-6-yl]urea trifluoroacetate

N-(3,4-difluorophenyl)-N′-[(3aS*,6R*,7aS*)-3a-(3,4-dimethoxyphenyl)octahydro-1H-indol-6-yl]urea (Example 175) (0.020 g, 0.046 mmol) and 4(5)-imidazolecarboxaldehyde (0.022 g, 0.23 mmol) were dissolved in MeOH (1 ml) and NaCNBH₃ (0.029 g, 0.46 mmol) was added. The mixture was stirred for 3 h and evaporated. The crude product was purified by reversed phase HPLC. Isolated as the TFA salt. Yield: 22 mg (75%). White solid. (ionspray; [M+H]⁺) m/z: 512.4. HRMS for C₂₇H₃₁F₂N₅O₃: Calcd, 511.2395; found, 511.2395.

### EXAMPLE 188

### N-(3,4-difluorophenyl)-N′-[(3aS*,6R*,7aS*)-3a-(3,4-dimethoxyphenyl)-1-(1H-imidazol-2-ylmethyl)octahydro-1H-indol-6-yl]urea trifluoroacetate

N-(3,4-difluorophenyl)-N′-[(3aS*,6R*,7aS*)-3a-(3,4-dimethoxyphenyl)octahydro-1H-indol-6-yl]urea (Example 175) (0.020 g, 0.046 mmol) and 2-imidazolecarboxaldehyde (0.022 g, 0.23 mmol) were dissolved in MeOH (1 ml) and NaCNBH₃ (0.029 g, 0.46 mmol) was added. The mixture was stirred for 20 h and evaporated. The crude product was purified by reversed phase HPLC. Isolated as the TFA salt. Yield: 1.2 mg (4%). White solid. MS (ionspray; [M+H]⁺) m/z: 512.4. HRMS for C₂₇H₃₁F₂N₅O₃: Calcd, 511.2395; found, 511.2378.

### EXAMPLE 189

### N-(3,4-difluorophenyl)-N′-[(3aS*,6R*,7aS*)-3a-(3,4-dimethoxyphenyl)-1-ethyloctahydro-1H-indol-6-yl]urea trifluoroacetate

N-(3,4-difluorophenyl)-N′-[(3aS*,6R*,7aS*)-3a-(3,4-dimethoxyphenyl)octahydro-1H-indol-6-yl]urea (Example 175) (0.020 g, 0.046 mmol) and acetaldehyde (0.021 g, 0.23 mmol) were dissolved in MeOH (1 ml) and NaCNBH₃ (0.029 g, 0.46 mmol) was added. The mixture was stirred for 18 h and evaporated. The crude product was purified by reversed phase HPLC. Isolated as the TFA salt. Yield: 0.8 mg (3%). White solid. MS (ionspray; [M+H]⁺) m/z: 460.3. HRMS for C₂₅H₃₁F₂N₃O₃: Calcd, 459.2333; found, 459.2333.

### EXAMPLE 190

### N-[(3aS*,6R*,7aS*)-1-acetyl-3a-(3,4-dimethoxyphenyl)octahydro-1H-indol-6-yl]-N′-(3,4-difluorophenyl)urea trifluoroacetate

DCC (0.062 g, 0.3 mmol) was added to a solution of tert-butyl [(3aS*,6R*,7aS*)-3a-(3,4-dimethoxyphenyl)octahydro-1H-indol-6-yl]carbamate, intermediate from Comparative Example 10 and 11 (0.100 g, 0.27 mmol) and N,N-dimethylglycine (0.031 g, 0.30 mmol) in CH₂Cl₂ (2 ml) and stirred for 16 hours. the mixture was filtered and the filtrate was evaporated.

Yield: 0.086 g, (71%). White solid.

The crude material from above (0.086 g, 019 mmol) was dissolved in TFA/H₂O 3:1 and stirred for 2 hours. The mixture was evaporated and partitioned between 1N NaOH and CH₂Cl₂. The organic phase was dried (MgSO₄) and evaporated. The crude product was dissolved in CH₂Cl₂ (2 ml) and 3,4-difluoroisocyanate (0.029 ml, 0.19 mmol) was added. The mixture was stirred for 45 minutes and evaporated. The crude product was purified by reversed phase HPLC. The title compound was isolated as the TFA-salt. Yield: 5.0 mg (6%). Colourless oil. MS (ionspray; [M+H]⁺) m/z: 474.3. HRMS for C₂₅H₂₉F₂N₃O₄: Calcd, 473.2126; found, 473.2123.

### EXAMPLE 191

### N-[(3aS*,6R*,7aS*)-3a-(3,4-dimethoxyphenyl)-1-methyloctahydro-1H-indol-6-yl]-N′-guinolin-3-ylurea

Quinoline-3-carboxylic acid (45 mg, 0.26 mmol) was suspended in toluene (1 ml) and cooled on ice, under N₂. Et₃N (0.03 ml, 0.26 mmol) and diphenylphosphoryl azide (0.06 ml, 0.26 mmol) was added, the reaction mixture was stirred at r.t. for 1.5 h, heated at 80° C. for 1.5 hrs, and then allowed to cool to ambient temperature. (3aS*,6R*,7aS*)-3a-(3,4-dimethoxyphenyl)-1-methyloctahydro-1H-indol-6-amine (Comparative Example 7; 50 mg, 0.17 mmol), in anhydrous CH₂Cl₂ (1 ml), was added and the reaction mixture was stirred at r.t. for 18 hrs. CH₂Cl₂ was added and the crude mixture was extracted with 1M aq. HCl, washed with H₂O/sat. aq. NaCl, dried over MgSO₄, and concentrated. The crude product was purified by preparative HPLC, followed by filtration through a pad of silica (CH₂Cl₂/MeOH 15:1) to give clean title compound, 1.5 mg (1.9%). HRMS (EI) calc: 460.2474 found: 460.2479

### EXAMPLE 192

### N′-[(3aS*,6R*,7aS*)-3a-(3,4-dimethoxyphenyl)-1-methyloctahydro-1H-indol-6-yl]-N-(4-fluorophenyl)-N-methylurea trifluoroacetate

Triethylamine (305 μL, 2.19 mmol) was added to a solution of (3aS*,6R*,7aS*)-3a-(3,4-dimethoxyphenyl)-1-methyloctahydro-1H-indol-6-amine, Comparative Example 7 (318 mg, 1.097 mmol) dissolved in dry CH₂Cl₂ (5 mL). Triphosgene (130 mg, 0.44 mmol) was dissolved in dry CH₂Cl₂ (1 mL) and added drop-wise. The solution was stirred under N₂ in room temperature for 3 h. MS (ESI+) m/z 393 (M+H)⁺. isocyanate was partitioned into 10 reaction vials, to which the appropriate amine (see below) was added).

4-Fluoro-N-methylaniline (14 mg, 0.1097 mmol) was added to the isocyanate (0.1097 mmol) solution. The mixture was stirred at room temperature under N₂ atmosphere over night. Volatiles were evaporated and the crude product was purified by preparative HPLC which gave 31 mg (64%) of the title compound.

MS (ESI+) m/z 442 (M+H)⁺. HRMS (EI) calc for C₂₅H₃₂FN₃O₃: 441.2428 found 441.2420.

### EXAMPLE 193

### N′-[(3aS*,6R*,7aS*)-3a-(3,4-dimethoxyphenyl)-1-methyloctahydro-1H-indol-6-yl]-N-(4-methoxyphenyl)-N-methylurea trifluoroacetate

4-Methoxy-N-methylaniline (15 mg, 0.1097 mmol) was added to the isocyanate (Example 192) (0.1097 mmol) solution. The mixture was stirred at room temperature under N₂ atmosphere over night. Volatiles were evaporated and the crude product was purified by preparative HPLC which gave 23 mg (46%) of the title compound. MS (ESI+) m/z 454 (M+H)⁺. HRMS (EI) calc for C₂₆H₃₅N₃O₄: 453.2628 found 453.2634.

### EXAMPLE 194

### N-(4-bromophenyl)-N′-[(3aS*,6R*,7aS*)-3a-(3,4-dimethoxyphenyl)-1-methyloctahydro-1H-indol-6-yl]-N-methylurea trifluoroacetate

4-Bromo-N-methylaniline (20 mg, 0.1097 mmol) was added to the isocyanate (Example 192) (0.1097 mmol) solution. The mixture was stirred at room temperature under N₂ atmosphere over night. Volatiles were evaporated and the crude product was purified by preparative HPLC which gave 31 mg (56%) of the title compound. MS (ESI+) m/z 502 (splitt 1:1) (M+H)⁺. HRMS (EI) calc for C₂₅H₃₂BrN₃O₃: 501.1627 found 501.1616.

### EXAMPLE 195

### N-[(3aS*,6R*,7aS*)-3a-(3,4-dimethoxyphenyl)-1-methyloctahydro-1H-indol-6-yl]-N′-(5-ethylpyridin-2-yl)urea

5-Ethyl-picolinic acid (20 mg, 0.13 mmol) was suspended in toluene (1 ml) and cooled on ice, under N₂. Et₃N (0.02 ml, 0.13 mmol) and diphenylphosphoryl azide (0.03 ml, 0.13 mmol) were added and the reaction mixture was stirred at r.t. for 2 hrs, heated at 80° C. for 1.5 hrs, and cooled to ambient temperature. (3aS*,6R*,7aS*)-3a-(3,4-dimethoxyphenyl)-1-methyloctahydro-1H-indol-6-amine (Comparative Example 7; 25 mg, 0.09 mmol), in anhydrous CH₂Cl₂ (1 ml), was added and the reaction mixture was heated at 80° C. for 4 hrs. CH₂Cl₂ was added and the crude mixture was extracted with 1M aq. HCl, washed with H₂O/sat. aq. NaCl, dried over MgSO₄, and concentrated. The crude product was purified by preparative HPLC, followed by filtration through silica (CH₂Cl₂/MeOH 4:1) to give clean title compound, 2.5 mg (6.3%). HRMS (EI) calc: 438.2631 found: 438.2628

### EXAMPLE 196

### N-(5-bromopyridin-3-yl)-N′-[(3aS*,6R*,7aS*)-3a-(3,4-dimethoxyphenyl)-1-methyloctahydro-1H-indol-6-yl]urea

5-Br-Nicotinic acid (26 mg, 0.13 mmol) was suspended in toluene (1 ml) and cooled on ice, under N₂. Et₃N (0.02 ml, 0.13 mmol) and diphenylphosphoryl azide (0.03 ml, 0.13 mmol) were added and the reaction mixture was stirred at r.t. for 2 hrs, heated at 80 deg for 1.5 hrs, and cooled to ambient temperature. (3aS*,6R*,7aS*)-3a-(3,4-dimethoxyphenyl)-1-methyloctahydro-1H-indol-6-amine (Comparative Example 7; 25 mg, 0.09 mmol), in anhydrous CH₂Cl₂, was added and the reaction mixture was heated at 80° C. for 4 hrs. CH₂Cl₂ was added and the crude mixture was extracted with 1M aq. HCl, washed with H₂O/sat. aq. NaCl, dried over MgSO₄, and concentrated. The crude product was purified by preparative HPLC, followed by filtration through silica (CH₂Cl₂/MeOH 4:1) to give clean title compound, 4.7 mg (10.7%). HRMS (EI) calc: 488.1423 found: 488.1405

### EXAMPLE 197

### N′-[(3aS*,6R*,7aS*)-3a-(3,4-dimethoxyphenyl)-1-methyloctahydro-1H-indol-6-yl]-N-methyl-N-(4-methylphenyl)urea trifluoroacetate

4-Methyl-N-methylaniline (14 μL, 0.1097 mmol) was added to the isocyanate (Example 192) (0.1097 mmol) solution. The mixture was stirred at room temperature under N₂ atmosphere over night. Volatiles were evaporated and the crude product was purified by preparative HPLC, which gave 20 mg (42%) of the title compound. MS (ESI+) m/z 438 (M+H)⁺. HRMS (EI) calc for C₂₆H₃₅N₃O₃: 437.2678 found 437.2659.

### EXAMPLE 198

### N-(4-chlorophenyl)-N′-[(3aS*,6R*,7aS*)-3a-(3,4-dimethoxyphenyl)-1-methyloctahydro-1H-indol-6-yl]-N-methylurea trifluoroacetate

4-Chloro-N-methylaniline (13 μL, 0.1097 mmol) was added to the isocyanate (Example 192) (0.1097 mmol) solution. The mixture was stirred at room temperature under N₂ atmosphere over night. Volatiles were evaporated and the crude product was purified by preparative HPLC, which gave 27 mg (54%) of the title compound. MS (ESI+) m/z 458 (M+H)⁺. HRMS (EI) calc for C₂₅H₃₂ClN₃O₃: 457.2132 found 457.2140.

### EXAMPLE 199

### N-(2-chloro-6-methoxypyridin-4-yl)-N′-[(3aS*,6R*,7aS*)-3a-(3,4-dimethoxyphenyl)-1-methyloctahydro-1H-indol-6-yl]urea trifluoroacetate

2-Chloro-6-methoxyisonicotinic acid (49 mg, 0.26 mmol) was dissolved in 1 mL toluene and stirred at −0° C. under N₂. Triethylamine (26 mg, 0.26 mmol) were added and then drop-wise addition of DPPA (71 mg, 0.26 mmol). The mixture was stirred at RT for 1,5 hrs and then heated at 80° C. for 2 hrs. After cooling to room temperature (3aS*,6R*,7aS*)-3a-(3,4-dimethoxyphenyl)-1-methyloctahydro-1H-indol-6-amine (Comparative Example 7; 50 mg, 0.17 mmol) in 1 mL dry CH₂Cl₂ were added. The reaction was stirred at room temperature overnight. 6 mL of CH₂Cl₂ was added to the mixture. Extracted with 1M HCl (aq), washed with H₂O and then Brine. Dried with MgSO₄, filtrated and then concentrated. Purification using preparative HPLC gave the product as dark yellow oil (30.8 mg, 25%).

MS (ESI⁺) for C₂₄H₃₁ClN₄O₄ m/z 475 (M+H⁺), HRMS found: 474,2026 calculated: 474,2034

¹HNMR (270 MHz, Chloroform-d) ppm 1.22-1.33 (m, 1H); 1.82-1.96 (m, 3H); 2.18-2.37 (m, 4H); 2.48-2.61 (m, 2H); 3.03 (s, 3H); 3.70 (d, J=16.62, 1H); 3.84-3.95 (m, 9H); 4.14-4.28 (m, 1H); 6.44 (s, 1H); 6.60-6.69 (m, 1H); 6.76-6.88 (m, 3H); 7.11 (s, 1H); 8.98 (b, 1H)

### EXAMPLE 200

### N-[(3aS*,6R*,7aS*)-3a-(3,4-dimethoxyphenyl)-1-methyloctahydro-1H-indol-6-yl]-N′-(2,6-dimethoxypyridin-3-yl)urea trifluoroacetate

2,6-Dimethoxynicotinic acid (48 mg, 0.26 mmol) was dissolved in 1 mL toluene and stirred at ˜0° C. under N₂. Triethylamine (26 mg, 0.26 mmol) and DPPA (71 mg, 0.26 mmol) were added. The mixture was stirred at RT for 1,5 hrs and then heated at 80° C. for 2 hrs. After cooling to room temperature (3aS*,6R*,7aS*)-3a-(3,4-dimethoxyphenyl)-1-methyloctahydro-1H-indol-6-amine (Comparative Example 7; 50 mg, 0,17 mmol) in 1 mL dry CH₂Cl₂ was added. The reaction was stirred at room temperature overnight. 6 mL of CH₂Cl₂ were added to the mixture. Extracted with 1M HCl (aq), washed with H₂O and then Brine. Dried with MgSO₄, filtrated and then concentrated. Purification with preparative HPLC gave the product as dark yellow oil (40.1 mg, 33%).

MS (ESI⁺) for C₂₅H₃₄N₄O₅ m/z 471 (M+H⁺), HRMS found: 470,2526 calculated 470,2529

¹HNMR (270 MHz, Chloroform-d) ppm 1.24-1.31 (m, 3H); 1.80-1.94 (m, 2H); 2.12-2.24 (m, 4H); 2.55 (d, J=12.67, 1H); 2.93 (s, 3H); 3.64 (d, J=12.93, 4H); 3.84-3.91 (m, 12H); 4.00-4.12 (m, 4H); 6.22 (d, J=8.44, 1H); 6.70-6.75 (m, 1H); 6.81 (d, J=8.71, 1H); 7.04-7.10 (m, 1H); 7.17-7.23 (m, 2H); 7.73 (d, J=8.45, 1H)

### EXAMPLE 201

### N-(6-chloropyridin-3-yl)-N′-[(3aS*,6R*,7aS*)-3a-(3,4-dimethoxyphenyl)-1-methyloctahydro-1H-indol-6-yl]urea trifluoroacetate

6-Chloronicotinic acid (41 mg, 0.26 mmol) was dissolved in 1 mL toluene and stirred at −0° C. under N₂. Triethylamine (26 mg, 0.26 mmol) and DPPA (71 mg, 0.26 mmol) were added. The mixture was stirred at room temperature for 1,5 hrs and then heated at 80° C. for 2 hrs. After cooling to room temperature (3aS*,6R*,7aS*)-3a-(3,4-dimethoxyphenyl)-1-methyloctahydro-1H-indol-6-amine (Comparative Example 7; 50 mg, 0.17 mmol) in 1 mL dry CH₂Cl₂ was added. The reaction was stirred at RT overnight. 6 mL of CH₂Cl₂ were added to the mixture. Extracted with 1M HCl(aq), washed with H₂O and then Brine. Dried with MgSO₄, filtrated and then concentrated. Purification using preparative HPLC gave the product as yellow oil (10.8 mg, 10%).

MS (ESI⁺) for C₂₃H₂₉ClN₄O₃ m/z 445 (M+H⁺), HRMS found: 444,1938 calculated: 444,1928

¹H NMR (270 MHz, Chloroform-d) ppm 1.17-1.25 (m, 1H); 1.71-1.85 (m, 3H); 1.99-2.15 (m, 4H); 2.31-2.41 (m, 1H); 2.76 (s, 3H); 2.81-2.98 (m, 1H); 3.65-3.74 (m, 1H); 3.83 (d, J=6.07, 6H); 4.00-4.18 (b, 1H); 6.68-6.80 (m, 3H); 7.05-7.23 (m, 1H); 7.65 (d, J=10.03, 1H); 8.05 (d, J=6.59, 1H); 8.87 (s, 1H); 9.35 (s, 1H)

### EXAMPLE 202

### N-[(3aS*,6R*,7aS*)-3a-(3,4-dimethoxyphenyl)-1-methyloctahydro-1H-indol-6-yl]-N′-quinolin-4-ylurea

Quinoline-4-carboxylic acid (45 mg, 0.26 mmol) was suspended in toluene (1 ml) and cooled on ice, under N₂. Et₃N (0.03 ml, 0.26 mmol) and diphenylphosphoryl azide (0.06 ml, 0.26 mmol) were added, the reaction mixture was stirred at r.t. for 1.5 hrs, heated at 80° C. for 1.5 hrs and cooled to ambient temperature. (3aS*,6R*,7aS*)-3a-(3,4-dimethoxyphenyl)-1-methyloctahydro-1H-indol-6-amine (Comparative Example 7; 50 mg, 0.17 mmol), in anhydrous CH₂Cl₂ (1 ml), was added and the reaction mixture was stirred at r.t for 18 hrs. CH₂Cl₂ was added and the crude mixture was extracted with 1M aq. HCl, washed with H₂O/sat. aq. NaCl, dried over MgSO₄, and concentrated. The crude product was purified by preparative HPLC, followed by filtration through silica (CH₂Cl₂:MeOH 4:1) to give clean title compound, 4.4 mg (5.6%). HRMS (EI) calc: 460.2474 found: 460.2447

### EXAMPLE 203

### N-(4-chloropyridin-2-yl)-N′-[(3aS*,6R*,7aS*)-3a-(3,4-dimethoxyphenyl)-1-methyloctahydro-1H-indol-6-yl]urea

4-chloropicolinic acid (20 mg, 0.13 mmol) was suspended in toluene (1 ml) and cooled on ice, under N₂. Et₃N (0.02 ml, 0.13 mmol) and diphenylphosphoryl azide (0.03 ml, 0.13 mmol) were added and the reaction mixture was stirred at r.t. for 2 h, heated at 80° C. for 1.5 h, and cooled to ambient temperature. (3aS*,6R*,7aS*)-3a-(3,4-dimethoxyphenyl)-1-methyloctahydro-1H-indol-6-amine (Comparative Example 7; 25 mg, 0.09 mmol), in anhydrous CH₂Cl₂ (1 ml), was added and the reaction mixture was heated at 80° C. for 4 h. CH₂Cl₂ was added and the crude mixture was extracted with 1M aq. HCl, washed with H₂O/sat. aq. NaCl, dried over MgSO₄, and concentrated. The crude product was purified by preparative HPLC, followed by filtration through silica (CH₂Cl₂:MeOH 4:1) to give clean title compound, 1.2 mg (3%). HRMS (EI) calc: 444.1928 found: 444.1916

### EXAMPLE 204

### N-(6-bromopyridin-2-yl)-N′-[(3aS*,6R*,7aS*)-3a-(3,4-dimethoxyphenyl)-1-methyloctahydro-1H-indol-6-yl]urea

6-Bromo-2-aminopyridine (21 mg, 0.12 mmol) was dissolved in anhydrous CH₂Cl₂ (0.5 ml) under N₂. Et₃N (0.03 ml, 0.24 mmol) was added, followed by triphosgene (15 mg, 0.05 mmol), in anhydrous CH₂Cl₂ (1 ml). The reaction mixture was stirred for 10 min, then (3aS*,6R*,7aS*)-3a-(3,4-dimethoxyphenyl)-1-methyloctahydro-1H-indol-6-amine (Comparative Example 7; 33 mg, 0.11 mmol), in anhydrous CH₂Cl₂ (1 ml), was added. The reaction mixture was stirred at ambient temperature for 18 hrs. The crude mixture was purified by column chromatography on silica (CH₂Cl₂/MeOH 4:1) to give the product, mixed with Et₃N-salt. Dissolve in CH₂Cl₂, extract with 1M aqueous HCl, neutralise with sat. aq. Na₂CO₃ to precipitate the title compound as clean product, 12 mg (22%). HRMS (EI) calc: 488.1423 found: 488.1403

### EXAMPLE 205

### N-[(3aS*,6R*,7aS*)-3a-(3,4-dimethoxyphenyl)-1-methyloctahydro-1H-indol-6-yl]-N′-(3-nitropyridin-2-yl)urea

3-Nitro-2-aminopyridine (17 mg, 0.12 mmol) was dissolved in anhydrous CH₂Cl₂ (0.5 ml) under N₂. Et₃N (0.03 ml, 0.24 mmol) was added, followed by triphosgene (15 mg, 0.05 mmol), in anhydrous CH₂Cl₂ (1 ml). The reaction mixture was stirred for 10 min, then (3aS*,6R*,7aS*)-3a-(3,4-dimethoxyphenyl)-1-methyloctahydro-1H-indol-6-amine (Comparative Example 7; 33 mg, 0.11 mmol), in anhydrous CH₂Cl₂ (1 ml), was added. The reaction mixture was stirred at ambient temperature for 18 hrs. The crude mixture was purified by column chromatography on silica (CH₂Cl₂/MeOH 4:1) to give product, mixed with Et₃N-salt. The mixture was dissolved in CH₂Cl₂, extracted with sat. aq. Na₂CO₃, dried over MgSO₄, and concentrated to give the title compound as clean product, 2 mg (4%).

HRMS (EI) calc: 455.2169 found: 455.2169

### EXAMPLE 206

### N-[(3aS*,6R*,7aS*)-3a-(3,4-dimethoxyphenyl)-1-methyloctahydro-1H-indol-6-yl]-N′-[6-(trifluoromethyl)pyridin-3-yl]urea

6-Trifluoromethyl-3-aminopyridine (19 mg, 0.12 mmol) was dissolved in anhydrous CH₂Cl₂ (0.5 ml) under N₂. Et₃N (0.03 ml, 0.24 mmol) was added, followed by triphosgene (15 mg, 0.05 mmol), in anhydrous CH₂Cl₂ (1 ml). The reaction mixture was stirred for 10 min, then (3aS*,6R*,7aS*)-3a-(3,4-dimethoxyphenyl)-1-methyloctahydro-1H-indol-6-amine (Comparative Example 7; 33 mg, 0.11 mmol), in anhydrous CH₂Cl₂ (1 ml), was added. The reaction mixture was stirred at ambient temperature for 18 hrs. The crude mixture was purified by column chromatography on silica (CH₂Cl₂/MeOH 4:1) to give product, mixed with Et₃N-salt. The mixture was dissolved in CH₂Cl₂, extracted with sat. aq. Na₂CO₃, dried over MgSO₄, and concentrated to give the title compound, 1 mg (2%).

HRMS (EI) calc: 478.2192 found: 478.2174

### EXAMPLE 207

### N-(6-chloropyridin-3-yl)-N′-[(3aS*,6R*,7aS*)-3a-(3,4-dimethoxyphenyl)-1-methyloctahydro-1H-indol-6-yl]urea

6-C1-3-Aminopyridine (15 mg, 0.12 mmol) was dissolved in anhydrous CH₂Cl₂ (0.5 ml) under N₂. Et₃N (0.03 ml, 0.24 mmol) was added, followed by triphosgene (15 mg, 0.05 mmol), in anhydrous CH₂Cl₂ (1 ml). The reaction mixture was stirred for 10 min, then (3aS*,6R*,7aS*)-3a-(3,4-dimethoxyphenyl)-1-methyloctahydro-1H-indol-6-amine (Comparative Example 7; 33 mg, 0.11 mmol), in anhydrous CH₂Cl₂ (1 ml), was added. The reaction mixture was stirred at ambient temperature for 18 hrs. The crude mixture was purified by column chromatography on silica (CH₂Cl₂/MeOH 4:1) to give product, mixed with Et₃N-salt. The mixture was dissolved in CH₂Cl₂, extracted with sat. aq. Na₂CO₃, dried over MgSO₄, and concentrated to give the title compound, 2 mg (4%). HRMS (EI) calc: 444.1928 found: 444.1908

### EXAMPLE 208

### N-[(3aS*,6R*,7aS*)-3a-(3,4-diethoxyphenyl)-1-methyloctahydro-1H-indol-6-yl]-N′-(3,4-difluoro]henyl)urea trifluoroacetate

Cs₂CO₃ (0.046 g, 0.14 mmol) was added to a solution of N-(3,4-difluorophenyl)-N′-[(3aS*,6R*,7aS*)-3a-(3,4-dihydroxyphenyl)-1-methyloctahydro-1H-indol-6-yl]urea hydrobromide (Example 322) (0.015 g, 0.036 mmol) in DMSO (0.5 ml) and stirred for 1 minute before ethylbromide (0.015 g, 0.14 mmol) was added. The mixture was stirred for 16 h and diluted with diethyl ether, washed with water (3×), dried (MgSO₄) and evaporated. The crude product was purified by reversed phase HPLC. Isolated as the TFA salt. Yield 4.4 mg (21%). White solid. HRMS for C₂₆H₃₃F₂N₃O₃: Calcd, 473.2490; found, 473.2492.

### EXAMPLE 209

### N-[(3aS*,6R*,7aS*)-3a-(3,4-dimethoxyphenyl)-1-methyloctahydro-1H-indol-6-yl]-N′-(2-methoxypyridin-3-yl)urea

2-Methoxy-3-aminopyridine (15 mg, 0.12 mmol)) was dissolved in anhydrous CH₂Cl₂ (0.5 ml) under N₂. Et₃N (0.03 ml, 0.24 mmol) was added, followed by triphosgene (15 mg, 0.05 mmol), in anhydrous CH₂Cl₂ (1 ml). The reaction mixture was stirred for 10 min, then (3aS*,6R*,7aS*)-3a-(3,4-dimethoxyphenyl)-1-methyloctahydro-1H-indol-6-amine (Comparative Example 7; 33 mg, 0.11 mmol), in anhydrous CH₂Cl₂ (1 ml), was added. The reaction mixture was stirred at ambient temperature for 18 hrs. The crude mixture was purified by column chromatography on silica (CH₂Cl₂/MeOH 4:1) to give product, mixed with Et₃N-salt. The mixture was dissolved in CH₂Cl₂, extracted with sat. aq. Na₂CO₃, dried over MgSO₄, and concentrated to give the title compound, 5.5 mg (10%). HRMS (EI) calc: 440.2424 found: 440.2445

### EXAMPLE 210

### N-[(3aS*,6R*,7aS*)-3a-(3,4-dimethoxyphenyl)-1-methyloctahydro-1H-indol-6-yl]-N′-(4-ethoxypyridin-3-yl)urea

4-Ethoxy-3-aminopyridine (16 mg, 0.12 mmol) was dissolved in anhydrous CH₂Cl₂ (0.5 ml) under N₂. Diisopropylethyl amine (0.04 ml, 0.24 mmol) was added, followed by triphosgene (15 mg, 0.05 mmol), in anhydrous CH₂Cl₂ (1 ml). The reaction mixture was stirred for 10 min, then (3aS*,6R*,7aS*)-3a-(3,4-dimethoxyphenyl)-1-methyloctahydro-1H-indol-6-amine (Comparative Example 7; 33 mg, 0.11 mmol), in anhydrous CH₂Cl₂ (1 ml), was added. The reaction mixture was stirred at ambient temperature for 18 hrs. The crude mixture was purified by column chromatography on silica (CH₂Cl₂/MeOH 4:1) to give the title compound, 8.2 mg (15%) HRMS (EI) calc: 454.2580 found: 454.2566

### EXAMPLE 211

### N-(3,4-difluorophenyl)-N′-[(3aS*,6R*,7aS*)-3a-(3,4-dipropoxyphenyl) 1-methyloctahydro-1H-indol-6-yl]urea trifluoroacetate

Cs₂CO₃ (0.065 g, 0.2 mmol) was added to a solution of N-(3,4-difluorophenyl)-N′-[(3aS*,6R*,7aS*)-3a-(3,4-dihydroxyphenyl)-1-methyloctahydro-1H-indol-6-yl]urea hydrobromide (Example 322) (0.025 g, 0.050 mmol) in DMF (2 ml) and stirred for 1 minute before propylbromide (0.014 ml, 0.15 mmol) was added. The mixture was stirred for 18 h and diluted with diethyl ether, washed with water (3×), dried (MgSO₄) and evaporated. The crude product was purified by reversed phase HPLC. Yield 3.4 mg (11%). Brown oil. HRMS for C₂₈H₃₇F₂N₃O₃: Calcd, 501.2803; found, 501.2821.

### EXAMPLE 212

### N-(3,4-difluorophenyl)-N′-[(3aS*,6R*,7aS*)-3a-(3,4-dihydro-2H-1,5-benzodioxepin-7-yl)-1-methyloctahydro-1H-indol-6-yl]urea trifluoroacetate

Cs₂CO₃ (0.070 g, 0.2 mmol) was added to a solution of N-(3,4-difluorophenyl)-N′-[(3aS*,6R*,7aS*)-3a-(3,4-dihydroxyphenyl)-1-methyloctahydro-1H-indol-6-yl]urea hydrobromide (Example 322) (0.025 g, 0.050 mmol) in DMSO (2 ml) and stirred for 5 minutes before dibromopropane (0.008 ml, 0.14 mmol) was added. The mixture was stirred at 40° C. for 3 h and diluted with diethyl ether, washed with water (2×), dried (MgSO₄) and evaporated. The crude product was purified by reversed phase HPLC. Isolated as the TFA salt. Yield 7.2 mg (25%). Colourless oil. MS (ionspray; [M+H]⁺) m/z: 458.2. HRMS for C₂₅H₂₉F₂N₃O₃: Calcd, 457.2177; found, 457.2169.

### EXAMPLE 213

### N-(3,4-difluorophenyl)-N′-[(3aS*,6R*,7aS*)-3a-(3,4-diisopropoxyphenyl)-1-methyloctahydro-1H-indol-6-yl]urea trifluoroacetate

Cs₂CO₃ (0.033 g, 0.1 mmol) was added to a solution of N-(3,4-difluorophenyl)-N′-[(3aS*,6R*,7aS*)-3a-(3,4-dihydroxyphenyl)-1-methyloctahydro-1H-indol-6-yl]urea hydrobromide (Example 322) (0.010 g, 0.020 mmol) in DMF (2 ml) and stirred for 1 minute before isopropyliodide (0.017 g, 0.10 mmol) was added. The mixture was stirred for 3 h and diluted with diethyl ether, washed with water (2×), dried (MgSO₄) and evaporated. The crude product was purified by reversed phase HPLC. Isolated as the TFA salt. Yield 2.0 mg (16%). White solid. MS (ionspray; [M+H]⁺) m/z: 502.2. HRMS for C₂₈H₃₇F₂N₃O₃: Calcd, 501.2803; found, 501.2785.

### EXAMPLE 214

### N-[(3aS*,6R*,7aS*)-3a-(3,4-dimethoxyphenyl)-1-methyloctahydro-1H-indol-6-yl]-N′-(2,6-dimethylpyridin-4-yl)urea

2,6-Dimethyl-4-aminopyridine (6 mg, 0.05 mmol) was dissolved in anhydrous CH₂Cl₂ (0.5 ml) under N₂. Diisopropylethyl amine (0.02 ml, 0.12 mmol) was added, followed by triphosgene (6 mg, 0.02 mmol), in anhydrous CH₂Cl₂ (0.5 ml). The reaction mixture was stirred for 10 min, then (3aS*,6R*,7aS*)-3a-(3,4-dimethoxyphenyl)-1-methyloctahydro-1H-indol-6-amine (Comparative Example 7; 15 mg, 0.05 mmol), in anhydrous CH₂Cl₂ (0.5 ml), was added. The reaction mixture was stirred at ambient temperature for 18 hrs. The crude mixture was purified by column chromatography on silica (CH₂Cl₂/MeOH 4:1) to give the title compound, 4 mg (18%). HRMS (EI) calc: 438.2631 found: 438.2645

### EXAMPLE 215

### N-(3,4-difluorophenyl)-N′-[(3aS*,6R*,7aS*)-3a-(2,3-dihydro-1,4-benzodioxin-6-yl)-1-methyloctahydro-1H-indol-6-yl]urea trifluoroacetate

Cs₂CO₃ (0.080 g, 0.25 mmol) was added to a solution of N-(3,4-difluorophenyl)-N′-[(3aS*,6R*,7aS*)-3a-(3,4-dihydroxyphenyl)-1-methyloctahydro-1H-indol-6-yl]urea hydrobromide (Example 322) (0.025 g, 0.05 mmol) in DMSO (2 ml) and stirred for 2 minutes before dibromoethane (0.016 g, 0.055 mmol) was added. The mixture was stirred at 60° C. for 20 h and diluted with diethyl ether, washed with water (2×), dried (MgSO₄) and evaporated. The crude product was purified by reversed phase HPLC. Isolated as the TFA salt. Yield 2.2 mg (8%). White solid. MS (ionspray; [M+H]⁺) m/z: 444.2. HRMS for C₂₄H₂₇F₂N₃O₃: Calcd, 443.2020; found, 443.2030.

### EXAMPLE 216

### N-[(3aS*,6R*,7aS*)-3a-(3,4-dimethoxyphenyl)-1-methyloctahydro-1H-indol-6-yl]-N′-(4,6-dimethylpyridin-2-yl)urea

4,6-Dimethyl-2-aminopyridine (15 mg, 0.12 mmol) was dissolved in anhydrous CH₂Cl₂ (0.5 ml) under N₂. Et₃N (0.03 ml, 0.24 mmol) was added, followed by triphosgene (15 mg, 0.05 mmol), in anhydrous CH₂Cl₂ (1 ml). The reaction mixture was stirred for 10 min, then (3aS*,6R*,7aS*)-3a-(3,4-dimethoxyphenyl)-1-methyloctahydro-1H-indol-6-amine (Comparative Example 7; 33 mg, 0.11 mmol), in anhydrous CH₂Cl₂ (1 ml), was added. The reaction mixture was stirred at ambient temperature for 18 hrs. The crude mixture was purified by column chromatography on silica (CH₂Cl₂/MeOH 4:1) to give the title compound, 3 mg (6%). MS (ESI+) for C₂₅H₃₄N₄O₃ m/z 439 (M+1)

### EXAMPLE 217

### N-cano-N′-[(3aS*,6R*,7aS*)-3a-(3,4-dimethoxyphenyl)-1-methyloctahydro-1H-indol-6-yl]-N″-(2,6-dimethoxypyridin-3-yl)guanidine

Phenyl N-cyano-N′-(2,6-dimethoxypyridin-3-yl)imidocarbamate (21 mg, 0.07 mmol) and (3aS*,6R*,7aS*)-3a-(3,4-dimethoxyphenyl)-1-methyloctahydro-1H-indol-6-amine (Comparative Example 7; 30 mg, 0.10 mmol) were mixed in anhydrous DMF (1.5 ml) and heated in microwave at 200° C. for 300 s. The reaction mixture was diluted with CH₂Cl₂ and extracted several times with H₂O, dried over MgSO₄, and concentrated. The crude product was purified by column chromatography on silica (CH₂Cl₂/MeOH 9:1) to give the title compound, 15 mg (43%). HRMS (EI) calc: 494.2642 found: 494.2648

### EXAMPLE 218

### N-(6-chloropyridin-3-yl)-N′-cano-N″-[(3aS*,6R*,7aS*)-3a-(3,4-dimethoxyphenyl)-1-methyloctahydro-1H-indol-6-yl]guanidine

Phenyl N-(6-chloropyridin-3-yl)-N′-cyanoimidocarbamate (19 mg, 0.07 mmol) and (3aS*,6R*,7aS*)-3a-(3,4-dimethoxyphenyl)-1-methyloctahydro-1H-indol-6-amine (Comparative Example 7; 30 mg, 0.10 mmol) were mixed in anhydrous DMF (1.5 ml) and heated in microwave at 200° C. for 300 s. The reaction mixture was diluted with CH₂Cl₂ and extracted several times with H₂O, dried over MgSO₄, and concentrated. The crude product was purified by column chromatography on silica (CH₂Cl₂/MeOH 9:1) to give the title compound, 20 mg (61%) HRMS (EI) calc: 468.2041 found: 468.2045

### EXAMPLE 219

### N-cyano-N′-[(3aS*,6R*,7aS*)-3a-(3,4-dimethoxyphenyl)-1-methyloctahydro-1H-indol-6-yl]-N″-(2-methoxypyridin-3-yl)guanidine

Phenyl N-cyano-N′-(2-methoxypyridin-3-yl)imidocarbamate (18 mg, 0.07 mmol) and (3aS*,6R*,7aS*)-3a-(3,4-dimethoxyphenyl)-1-methyloctahydro-1H-indol-6-amine (Comparative Example 7; 30 mg, 0.10 mmol) were mixed in anhydrous DMF (1.5 ml) and heated in microwave at 200° C. for 300 s. The reaction mixture was diluted with CH₂Cl₂ and extracted several times with H₂O, dried over MgSO₄, and concentrated. The crude product was purified by column chromatography on silica (CH₂Cl₂/MeOH 9:1) to give the title compound, 20 mg (62%) HRMS (EI) calc: 464.2536 found: 464.2540

### EXAMPLE 220

### N-biphenyl-2-yl-N′-[(3aS*,6R*,7aS*)-3a-(3,4-dimethoxyphenyl)-1-methyloctahydro-1H-indol-6-yl]urea trifluoroacetate

Reagent: 2-biphenylyl isocyanate

Synthetic procedure: Scheme E

Yield: 12.5 mg (61%)

Measured mass: 485.2662

Calc. mass: 485.2678

### EXAMPLE 221

### N-[(3aS*,6R*,7aS*)-3a-(3,4-dimethoxyphenyl)-1-methyloctahydro-1H-indol-6-yl]-N′-(3-methylphenyl)urea trifluoroacetate

Reagent: 3-methylphenyl isocyanate

Synthetic procedure: Scheme E

Yield: 17.1 mg (93%)

Measured mass: 423.2516

Calc. mass: 423.2522

### EXAMPLE 222

### ethyl 2-[({[(3aS*,6R*,7aS*)-3a-(3,4-dimethoxyphenyl)-1-methyloctahydro-1H-indol-6-yl]amino}carbonyl)amino]benzoate trifluoroacetate

Reagent: ethyl 2-isocyanatobenzoate

Synthetic procedure: Scheme E

Yield: 13.8 mg (67%)

Measured mass: 481.2573

Calc. mass: 481.2577

### EXAMPLE 223

### N-(2-tert-butylphenyl)-N′-[(3aS*,6R*,7aS*)-3a-(3,4-dimethoxyphenyl)-1-methyloctahydro-1H-indol-6-yl]urea trifluoroacetate

Reagent: 2-tert-butylphenyl isocyanate

Synthetic procedure: Scheme E

Yield: 12.6 mg (63%)

Measured mass: 465.2975

Calc. mass: 465.2991

### EXAMPLE 224

### N-[(3aS*,6R*,7aS*)-3a-(3,4-dimethoxyphenyl)-1-methyloctahydro-1H-indol-6-yl]-N′-[2-(methylthio)phenyl]urea trifluoroacetate

Reagent: 2-(methylthio)phenyl isocyanate

Synthetic procedure: Scheme E

Yield: 16 mg (82%)

Measured mass: 455.2243

Calc. mass: 455.2243

### EXAMPLE 225

### N-[(3aS*,6R*,7aS*)-3a-(3,4-dimethoxyphenyl)-1-methyloctahydro-1H-indol-6-yl]-N′-(2-methoxyphenyl)urea trifluoroacetate

Reagent: 2-methoxyphenyl isocyanate

Synthetic procedure: Scheme E

Yield: 10.5 mg (55%)

Measured mass: 439.2462

Calc. mass: 439.2471

### EXAMPLE 226

### N-[(3aS*,6R*,7aS*)-3a-(3,4-dimethoxyphenyl)-1-methyloctahydro-1H-indol-6-yl]-N′-(2-nitrophenyl)urea trifluoroacetate

Reagent: 2-nitrophenyl isocyanate

Synthetic procedure: Scheme E

Yield: 8.8 mg (45%)

Measured mass: 454.2211

Calc. mass: 454.2216

### EXAMPLE 227

### N-[(3aS*,6R*,7aS*)-3a-(3,4-dimethoxyphenyl)-1-methyloctahydro-1H-indol-6-yl]-N′-(3-phenoxyphenyl)urea trifluoroacetate

Reagent: 3-phenoxyphenyl isocyanate

Synthetic procedure: Scheme E

Yield: 18.5 mg (87%)

Measured mass: 501.2633

Calc. mass: 501.2628

### EXAMPLE 228

### N-[(3aS,6R,7aS)-3a-(3,4-dimethoxyphenyl)-1-methyloctahydro-1H-indol-6-yl]-N′-[2-fluoro-3-(trifluoromethyl)phenyl]urea

A solution of the chiral amine (3aS,6R,7aS)-3a-(3,4-dimethoxyphenyl)-1-methyloctahydro-1H-indol-6-amine (Comparative Example 10; 0.58 g; 2.0 mmol) in dry THF (15.0 ml) was stirred at RT and treated with 2-fluoro-3-trifluoromethylphenylisocyanate (0.41 g; 2.0 mmol) and the resulting solution stirred at RT overnight.

The mixture was evaporated to afford a gum, which was flash-chromatographed over silica. Elution with ethyl acetate gave the product, which was treated with a 4M solution of hydrogen chloride in dioxane then evaporated to give the corresponding HCl salt as a colourless solid.

Yield=535 mg (54%)

Measured mass: 495.2135

Calc. mass: 495.2145

### EXAMPLE 229

### N-[(3aS*,6R*,7aS*)-3a-(3,4-dimethoxyphenyl)-1-methyloctahydro-1H-indol-6-yl]-N′-(2-iodophenyl)urea trifluoroacetate

Reagent: 2-iodophenyl isocyanate

Synthetic procedure: Scheme E

Yield: 14 mg (63%)

Measured mass: 535.1329

Calc. mass: 535.1332

### EXAMPLE 230

### N-(3aS*,6R*,7aS*)-3a-(3,4-dimethoxyphenyl)-1-methyloctahydro-1H-indol-6-yl-N′-(3-iodophenyl)urea trifluoroacetate

Reagent: 3-iodophenyl isocyanate

Synthetic procedure: Scheme E

Yield: 13.7 mg (61%)

Measured mass: 535.1334

Calc. mass: 535.1332

### EXAMPLE 231

### N-[(3aS*,6R*,7aS*)-3a-(3,4-dimethoxyphenyl)-1-methyloctahydro-1H-indol-6-yl]-N′-(4-iodophenyl)urea trifluoroacetate

Reagent: 4-iodophenyl isocyanate

Synthetic procedure: Scheme E

Yield: 17.9 mg (80%)

Measured mass: 535.1343

Calc. mass: 535.1332

### EXAMPLE 232

### N-[(3aS*,6R*,7aS*)-3a-(3,4-dimethoxyphenyl)-1-methyloctahydro-1H-indol-6-yl]-N′-[4-(methylthio)phenyl]urea trifluoroacetate

Reagent: 4-(methylthio)phenyl isocyanate

Synthetic procedure: Scheme E

Yield: 12.3 mg (63%)

Measured mass: 455.2243

Calc. mass: 455.2243

### EXAMPLE 233

### N-[(3aS*,6R*,7aS*)-3a-(3,4-dimethoxyphenyl)-1-methyloctahydro-1H-indol-6-yl]-N′-(3-fluorophenyl)urea trifluoroacetate

Reagent: 3-fluorophenyl isocyanate

Synthetic procedure: Scheme E

Yield: 13.1 g (70%)

Measured mass: 427.2273

Calc. mass: 427.2271

### EXAMPLE 234

### N-[(3aS*,6R*,7aS*)-3a-(3,4-dimethoxyphenyl)-1-methyloctahydro-1H-indol-6-yl]-N′-(2-ethylphenyl)urea trifluoroacetate

Reagent: 2-ethylphenyl isocyanate

Synthetic procedure: Scheme E

Yield: 14.1 mg (74%)

Measured mass: 437.2680

Calc. mass: 437.2678

### EXAMPLE 235

### N-(2-cyanophenyl)-N′-[(3aS*,6R*,7aS*)-3a-(3,4-dimethoxyphenyl)-1-methyloctahydro-1H-indol-6-yl]urea trifluoroacetate

Reagent: 2-cyanophenyl isocyanate

Synthetic procedure: Scheme E

Yield: 10.6 mg (56%)

Measured mass: 434.2314

Calc. mass: 434.2318

### EXAMPLE 236

### N-(4-butyl-2-methylphenyl)-N′-[(3aS*,6R*,7aS*)-3a-(3,4-dimethoxyphenyl)-1-methyloctahydro-1H-indol-6-yl]urea trifluoroacetate

Reagent: 4-butyl-2-methylphenyl isocyanate

Synthetic procedure: Scheme E

Yield: 11.3 mg (55%)

Measured mass: 479.3150

Calc. mass: 479.3148

### EXAMPLE 237

### N-[(3aS*,6R*,7aS*)-3a-(3,4-dimethoxyphenyl)-1-methyloctahydro-1H-indol-6-yl]-N′-[4-fluoro-2-(trifluoromethyl)phenyl]urea trifluoroacetate

Reagent: 4-fluoro-2-(trifluoromethyl)phenyl isocyanate

Synthetic procedure: Scheme E

Yield: 10.9 mg (52%)

Measured mass: 495.2150

Calc. mass: 495.2145

### EXAMPLE 238

### N-(2-bromo-4,6-difluorophenyl)-N′-[(3aS*,6R*,7aS*)-3a-(3,4-dimethoxyphenyl)-1-methyloctahydro-1H-indol-6-yl]urea trifluoroacetate

Reagent: 2-bromo-4,6-difluorophenyl isocyanate

Synthetic procedure: Scheme E

Yield: 11.2 mg (51%)

Measured mass: 523.1278

Calc. mass: 523.1282

### EXAMPLE 239

### N-(2,4-dibromo-6-fluorophenyl)-N′-[(3aS*,6R*,7aS*)-3a-(3,4-dimethoxyphenyl)-1-methyloctahydro-1H-indol-6-yl]urea trifluoroacetate

Reagent: 2,4-dibromo-6-fluorophenyl isocyanate

Synthetic procedure: Scheme E

Yield: 10.0 mg (42%)

Measured mass: 583.0482

Calc. mass: 583.0481

### EXAMPLE 240

### N-(4-bromo-2,6-difluorophenyl)-N′-[(3aS*,6R*,7aS*)-3a-(3,4-dimethoxyphenyl)-1-methyloctahydro-1H-indol-6-yl]urea trifluoroacetate

Reagent: 4-bromo-2,6-difluorophenyl isocyanate

Synthetic procedure: Scheme E

Yield: 14.5 mg (66%)

Measured mass: 523.1276

Calc. mass: 523.1282

### EXAMPLE 241

### N-[(3aS*,6R*,7aS*)-3a-(3,4-dimethoxyphenyl)-1-methyloctahydro-1H-indol-6-yl]-N′-(2,3,4-trifluorophenyl)urea trifluoroacetate

Reagent: 2,3,4-trifluorophenyl isocyanate

Synthetic procedure: Scheme E

Yield: 8.1 mg (41%)

Measured mass: 463.2067

Calc. mass: 463.2083

### EXAMPLE 242

### N-[2-(difluoromethoxy)phenyl]-N′-[(3aS*,7aS*)-3a-(3,4-dimethoxyphenyl)-1-methyloctahydro-1H-indol-6-yl]urea trifluoroacetate

Reagent: 2-(difluoromethoxy)phenyl isocyanate

Synthetic procedure: Scheme E

Yield: 14.0 mg (69%)

Measured mass: 475.2283

Calc. mass: 475.2283

### EXAMPLE 243

### N-[4-chloro-2-(trifluoromethyl)phenyl]-N′-[(3aS*,6R*,7aS*)-3a-(3,4-dimethoxyphenyl)-1-methyloctahydro-1H-indol-6-yl]urea trifluoroacetate

Reagent: 4-chloro-2-(trifluoromethyl)phenyl isocyanate

Synthetic procedure: Scheme E

Yield: 13.7 mg (64%)

Measured mass: 511.1875

Calc. mass: 511.1850

### EXAMPLE 244

### N-[(3aS*,6R*,7aS*)-3a-(3,4-dimethoxyphenyl)-1-methyloctahydro-1H-indol-6-yl]-N′-[4-(6-methyl-1,3-benzothiazol-2-yl)phenyl]urea trifluoroacetate

Reagent: 4-(6-methyl-2-benzothiazolyl)phenyl isocyanate

Synthetic procedure: Scheme E

Yield: 26.2 (113%)

Measured mass: 556.2507

Calc. mass: 556.2508

### EXAMPLE 245

### N-[(3aS*,6R*,7aS*)-3a-(3,4-dimethoxyphenyl)-1-methyloctahydro-1H-indol-6-yl]-N′-9H-fluoren-2-ylurea trifluoroacetate

Reagent: 9H-fluoren-2-yl isocyanate

Synthetic procedure: Scheme E

Yield: 15.8 mg (75%)

Measured mass: 497.2672

Calc. mass: 497.2678

### EXAMPLE 246

### N-[4-(benzyloxy)phenyl]-N′-[(3aS*,6R*,7aS*)-3a-(3,4-dimethoxyphenyl)-1-methyloctahydro-1H-indol-6-yl]urea trifluoroacetate

Reagent: 4-(benzyloxy)phenyl isocyanate

Synthetic procedure: Scheme E

Yield: 16.9 mg (78%)

Measured mass: 515.2774

Calc. mass: 515.2784

### EXAMPLE 247

### N-[(3aS*,6R*,7aS*)-3a-(3,4-dimethoxyphenyl)-1-methyloctahydro-1H-indol-6-yl]-N′-[2-fluoro-5-(trifluoromethyl)phenyl]urea trifluoroacetate

Reagent: 2-fluoro-5-(trifluoromethyl)phenyl isocyanate

Synthetic procedure: Scheme E

Yield: 17.0 mg (81%)

Measured mass: 495.2150

Calc. mass: 495.2145

### EXAMPLE 248

### N-(4-butylphenyl)-N′-[(3aS*,6R*,7aS*)-3a-(3,4-dimethoxyphenyl)-1-methyloctahydro-1H-indol-6-yl]urea trifluoroacetate

Reagent: 4-butylphenyl isocyanate

Synthetic procedure: Scheme E

Yield: 18.3 mg (92%)

Measured mass: 465.2989

Calc. mass: 465.2991

### EXAMPLE 249

### N-(3,5-dimethoxyphenyl)-N′-[(3aS*,6R*,7aS*)-3a-(3,4-dimethoxyphenyl)-1-methyloctahydro-1H-indol-6-yl]urea trifluoroacetate

Reagent: 3,5-dimethoxyphenyl isocyanate

Synthetic procedure: Scheme E

Yield: 16.0 mg (80%)

Measured mass: 469.2559

Calc. mass: 469.2577

### EXAMPLE 250

### N{(3aS*,6R*,7aS*)-3a-[4-(benzyloxy)-3-methoxyphenyl]-1-methyloctahydro-1H-indol-6-yl}-N′-(3,4-difluorophenyl)urea

Step 1

Br₂ (2.71 ml, 53.21 mmol) was added drop-wise to a solution of Ph₃P (13.96 g, 53.21 mmol) in DCM (150 ml) at 0° C. and stirred for 5 min. [4-(Benzyloxy)-3-methoxyphenyl]methanol (10.0 g, 40.93 mmol) was added portion wise and stirring continued for 45 min. Et₂O (1000 ml) was added, mixture filtered, and concentrated. The residue was dissolved in DMF (100 ml) and NaCN (2.40 g, 49.11 mmol) added. The mixture was stirred at ambient temperature for 3 h before water (100 ml) was added and the mixture extracted with Et₂O (3×250 ml). Combined organic layers were washed with brine and concentrated. The resulting intermediate [4-(benzyloxy)-3-methoxyphenyl]acetonitrile was isolated by flash chromatography using iso-hexanes/EtOAc 2:1 as eluent. Yield 10.01 g (83%); white solid.

¹H NMR (400 MHz, CDCl₃) δ ppm 3.68 (s, 2H) 3.91 (s, 3H) 5.17 (s, 2H) 6.80 (dd, J=8.2, 2.1 Hz, 1H) 6.81-6.89 (m, 2H) 7.33-7.46 (m, 5H). ¹³C NMR (100 MHz, CDCl₃) δ 23.06, 55.94, 70.92, 111.37, 114.16, 118.04, 120.05, 122.63, 127.15, 127.83, 128.47, 136.70, 147.75, 149.95.

Step 2

LiNH₂ (1.04 g, 45.60 mmol) was suspended in DME (50 ml) at ambient temperature and [4-(benzyloxy)-3-methoxyphenyl]acetonitrile (4.28 g, 16.89 mmol) in DME (50 ml) was added. The mixture was heated to 80° C. After 30 min of heating a deep green colour developed and 1-bromo-2-chloroethane (1.55 ml, 18.58 mmol) in DME (10 ml) was added carefully. The green colour disappeared and mixture turned light brown. The mixture was heated at 80° C. overnight. Water (50 ml) and Et₂O (100 ml) was added and aqueous layer extracted with DCM. Combined organic layer were evaporated and the intermediate 1-[4-(benzyloxy)-3-methoxyphenyl]cyclopropanecarbonitrile isolated by flash chromatography using 10-20% EtOAc in iso-hexanes. Yield 3.27 g (69%); yellow solid. Mp. 84° C.

¹H NMR (400 MHz, CDCl₃) δ ppm 1.32 (m, 2H) 1.64 (m, 2H) 3.90 (s, 3H) 5.13 (s, 2H) 6.71 (dd, J=8.53, 2.26 Hz, 1H) 6.81 (m, 1H) 6.89 (d, J=2.0 Hz, 1H) 7.35 (m, 5H) ³C NMR (100 MHz, CDCl₃) δ 13.46, 17.35, 56.04, 70.97, 110.57, 113.98, 118.23, 122.86, 127.14, 127.85, 128.51, 128.85, 136.77, 147.72, 149.77.

Step 3

DIBAL-H (4.17 ml, 29.35 mmol, 20 wt % in toluene) was added to a solution of 1-[4-(benzyloxy)-3-methoxyphenyl]cyclopropanecarbonitrile (4.10 g, 14.67 mmol) in THF (50 ml) and the mixture stirred for 3 h before the mixture was pored carefully into 2 M HCl (50 ml) and extracted with Et₂O (3×100 ml). The combined organic extracts were dried (MgSO₄) and evaporated to give the desired intermediate 1-[4-(benzyloxy)-3-methoxyphenyl]cyclopropanecarbaldehyde 3.65 g (88%); yellow oil. ¹H NMR (400 MHz, CDCl₃) δ 1.35-1.37 (m, 2H) 1.51-1.54 (m, 2H) 3.89 (s, 3H) 5.15 (s, 2H) 6.77 (dd, J=8.16, 2.13 Hz, 1H) 6.84 (d, J=1.51 Hz, 1H) 6.86 (s, 1H) 7.29-7.44 (m, 5H) 9.24 (s, 1H). ¹³C NMR (100 MHz; CDCl₃) δ 16.28, 37.27, 56.03, 71.00, 113.81, 113.87, 122.19, 127.20, 127.83, 128.54, 130.52, 137.10, 147.78, 149.51, 201.30.

Step 4

Methylamine (13 ml, 26.0 mmol, 2 M in THF) was added to a suspension of 1-[4-(benzyloxy)-3-methoxyphenyl]cyclopropanecarbaldehyde (3.64 g, 12.89 mmol) and Na₂SO₄ (10 g) in DCE (13 ml) and the mixture stirred at ambient temperature overnight. Mixture was filtered and salts washed with Et₂O to give N-{1-[4-(benzyloxy)-3-methoxyphenyl]cyclopropyl}methylene)-N-methylamine Yield 3.73 g (98%) yellow oil.

¹H NMR (400 MHz, CDCl₃) δ 1.11-1.14 (m, 2H) 1.23-1.25 (m, 2H) 3.22 (d, J=1.51 Hz, 3H) 3.88 (s, 3H) 5.13 (s, 2H) 6.79-6.86 (m, 3H) 7.28-7.44 (m, 5H) 7.54 (d, J=1.51 Hz, 1 H). ¹³C NMR (100 MHz, CDCl₃) δ 14.91, 29.96, 47.58, 55.95, 70.98, 113.61, 113.69, 121.72, 127.19, 127.73, 128.48, 134.70, 137.30, 147.13, 149.31, 169.28.

Step 5

N-{1-[4-(benzyloxy)-3-methoxyphenyl]cyclopropyl}methylene)-N-methylamine (2.42 g, 8.19 mmol) in DCM (20 ml) was added 1 M HCl in Et₂O (40 ml) and the mixture evaporated to dryness. The residue was dissolved in acetonitrile (50 ml) and methyl vinyl ketone (820 μl, 9.83 mmol) and Na₂SO₄ (10 g) was added. The mixture refluxed overnight and filtered. Half of mixture was distributed between 2 M HCl and Et₂O. Aqueous layer was made alkaline using 2 M NaOH and extracted with DCM. The DCM layer was concentrated. Yield 0.67 g (22%); yellow oil. Rest of material was concentrated and partitioned between 2 M NaOH and DCM. The organic layer was concentrated and the product isolated by flash chromatography using hexanes/EtOAc 2:1 and 3% Et₃N in EtOAc to give the intermediate (3aS*,7aS*)-3a-[4-(benzyloxy)-3-methoxyphenyl]-1-methyloctahydro-6H-indol-6-one. Yield 421 mg (14%), brownish oil. ¹H NMR (400 MHz, CDCl₃) δ 2.06-2.45 (m, 10H) 2.57 (dd, J=3.5, 1.8 Hz, 2H) 2.91 (t, J=3.5 Hz, 1H) 3.09-3.20 (m, 1H) 3.90 (s, 3H) 5.14 (s, 2H) 6.84 (s, 2H) 6.91 (s, 1H) 7.29-7.44 (m, 5H). ¹³C NMR (100 MHz, CDCl₃ δ 35.23, 36.20, 38.78, 40.04, 40.51, 47.48, 54.84, 56.22, 70.35, 71.01, 110.62, 113.69, 117.99, 127.21, 127.82, 128.53, 137.16, 140.78, 146.69, 149.58, 211.45. MS: 366 (M+1).

Step 6

NH₄OAc (9.90 g, 128 mmol) was added to a solution of (3aS*,7aS*)-3a-[4-(benzyloxy)-3-methoxyphenyl]-1-methyloctahydro-6H-indol-6-one (935 mg, 2.570 mmol) in MeOH (50 ml) and the mixture was heated at 60° C. for 2 h before NaCNBH₃ (404 mg, 6.425 mmol) was added and stirring continued for 1 h. Solvent was evaporated and 2 M NaOH (20 ml) was added. Aqueous phase was extracted with DCM, combined organic layers dried 3,4-Difluorophenyl isocyanate (261 μl, 2.218 mmol) was added to a solution of the crude amine (625 mg, 1.706 mmol) in DCM (10 ml) and mixture stirred for 30 min at ambient temperature before solvent was evaporated and the products isolated by flash chromatography using 5-10% MeOH in DCM as eluent.

First product eluted: N-{(3aS*,6S*,7aS*)-3a-[4-(benzyloxy)-3-methoxyphenyl]-1-methyloctahydro-1H-indol-6-yl}-N′-(3,4-difluorophenyl)urea: 318 mg (36%); white solid.

¹H NMR (400 MHz, CDCl₃) δ 1.37-2.30 (m, 9H) 2.21 (s, 3H) 2.75 (s, 1H) 3.13-3.15 (m, 1H) 3.91 (s, 3H) 4.09 (m, 1H) 5.15 (s, 2H) 6.80-7.46 (m, 13H). ¹³C NMR (100 MHz, CDCl₃) δ 26.62, 27.32, 29.75, 40.66, 44.60, 47.38, 53.46, 56.13, 68.26, 70.93, 110.83, 110.91, 113.37, 117.01, 117.18, 118.49, 127.21, 127.76, 128.45, 137.09, 146.45, 149.30, 155.11. MS (ESI+) for C₃₀H₃₃F₂N₃O₃ M/Z 522 (M+1). HRMS (EI) calcd for C₃₀H₃₃F₂N₃O₃: 521.249, found 521.2493.

Second product eluted: N-{(3aS*,6R*,7aS*)-3a-[4-(benzyloxy)-3-methoxyphenyl]-1-methyloctahydro-1H-indol-6-yl}-N′-(3,4-difluorophenyl)urea: 261 mg (29%); white solid.

¹H NMR (400 MHz, CDCl₃) δ 0.87-2.21 (m, 9H) 2.24 (s, 3H) 2.65 (m, 1H) 3.09-3.13 (m, 1H) 3.75 (m, 3H) 3.86-3.88 (m, 1H) 4.99 (s, 2H) 5.14 (d, J=6.8 Hz, 1H) 6.66-6.84 (m, 5H) 7.18-7.33 (m, 6H) 7.65 (s, 1H). ¹³C NMR (100 MHz, CDCl₃) δ 30.08, 30.71, 34.29, 40.11, 40.25, 44.60, 46.85, 53.71, 56.07, 69.14, 70.99, 108.56, 108.78, 110.98, 111.04, 113.35, 113.41, 116.69, 116.87, 118.69, 127.24, 127.36, 127.91, 128.14, 128.23, 128.49, 135.93, 135.95, 136.01, 136.04, 136.91, 146.25, 148.73, 149.18, 149.20, 155.22. MS (ESI+) for C₃₀H₃₃F₂N₃O₃ m/z 522 (M+1). HRMS (EI) calcd for C₃₀H₃₃F₂N₃O₃: 521.249, found 521.2493

The relative configuration of the products was confirmed by NMR.

### EXAMPLE 251

### N-(3,4-difluorophenyl)-N′-{(3aS*,6R*,7aS*)-3a-(3,4-dimethoxyphenyl)-1-[(dimethylamino)acetyl]octahydro-1H-indol-6-yl lurea

HOBt (0.007 g, 0.051 mmol), EDC (N-(dimethylaminopropyl)-N′-ethylcarbodiimide hydrochloride) (0.010 g, 0.051 mmol) and NEt₃ (0.008 ml, 0.051 mmol) were added to a solution of N-(3,4-difluorophenyl)-N′-[(3aS*,6R*,7aS*)-3a-(3,4-dimethoxyphenyl)octahydro-1H-indol-6-yl]urea (Example 175) (0.020 g, 0.046 mmol) in THF (1 ml) and N,N-dimethylglycine acid (0.0066 g, 0.063 mmol) was added. The reaction was stirred at 40° C. for 5 hours, filtered and evaporated. The crude product was purified by reversed phase HPLC. Isolated as the TFA salt. The mixture was made basic with 1N NaOH and extracted with dichloromethane (3×), dried (MgSO₄) and evaporated.

Yield 6.5 mg (27%). White solid. MS (ionspray; [M+H]⁺) m/z: 517.4. HRMS for C₂₇H₃₄F₂N₄O₄: Calcd, 516.2548; found, 516.2541.

### EXAMPLE 252

### N-(3,4-difluorophenyl)-N′-{(3aS*,6R*,7aS*)-3a-(3,4-dimethoxyphenyl)-1-[(1,3-dimethyl-1H-pyrazol-5-yl)methyl]octahydro-1H-indol-6-yl}urea trifluoroacetate

General procedure for the reductive amination procedure: N-(3,4-difluorophenyl)-N′-[(3aS*,6R*,7aS*)-3a-(3,4-dimethoxyphenyl)octahydro-1H-indol-6-yl]urea (Example 175) (0.020 g, 0.046 mmol) and the requisite aldehyde (0.23 mmol) were dissolved in MeOH (1 ml) and stirred for 30 minutes before NaCNBH₃ (0.029 g, 0.46 mmol) was added.

Reagent: 1,3-dimethyl-1H-pyrazole-5-carbaldehyde

The mixture was stirred for 18 h and filtered. The crude product was purified by reversed phase HPLC. Isolated as the TFA salt. Yield 26 mg (87%). MS (ionspray; [M+H]⁺) m/z: 540.4. HRMS for C₂₉H₃₅F₂N₅O₃: Calcd, 539.2708; found, 539.2724.

### EXAMPLE 253

### N-(3,4-difluorophenyl)-N′-{(3aS*,6R*,7aS*)-3a-(3,4-dimethoxyphenyl)-1-[(3,5-dimethylisoxazol-4-yl)methyl]octahydro-1H-indol-6-yl}urea trifluoroacetate

General procedure for reductive amination from Example 252

Reagent: 3,5-dimethyl-4-isoxazolecarbaldehyde

The mixture was stirred for 18 h and filtered. The crude product was purified by reversed phase HPLC. Isolated as the TFA salt. Yield 23 mg (77%). MS (ionspray; [M+H]⁺) m/z: 541.6. HRMS for C₂₉H₃₄F₂N₄O₄: Calcd, 540.2548; found, 540.2564.

### EXAMPLE 254

### N-(3,4-difluorophenyl)-N′-[(3aS*,6R*,7aS*)-3a-(3,4-dimethoxyphenyl)-1-(1,3-thiazol-2-ylmethyl)octahydro-1H-indol-6-yl]urea trifluoroacetate

General procedure for reductive amination from Example 252.

Reagent: 2-formylthiazole

The mixture was stirred for 18 h and filtered. The crude product was purified by reversed phase HPLC. Isolated as the TFA salt. Yield 21.4 mg (73%). Yellow solid. MS (ionspray; [M+H]⁺) m/z: 529.0. HRMS for C₂₇H₃₀F₂N₄O₃S: Calcd, 528.2007; found, 528.1992.

### EXAMPLE 255

### N-(3,4-difluorophenyl)-N′-{(3aS*,6R*,7aS*)-3a-(3,4-dimethoxyphenyl)-1-[(1-methyl-1H-imidazol-2-yl)methyl]octahydro-1H-indol-6-yl}urea trifluoroacetate

General procedure for reductive amination from Example 252.

Reagent: 1-methyl-2-imidazolecarboxaldehyde

The mixture was stirred for 18 h and filtered. The crude product was purified by reversed phase HPLC. Isolated as the TFA salt. Yield 24 mg (82%). Colourless oil. MS (ionspray; [M+H]⁺) m/z: 526.2. HRMS for C₂₈H₃₃F₂N₅O₃: Calcd, 525.2551; found, 525.2545

### EXAMPLE 256

### N-(3,4-difluorophenyl)-N′-{(3aS*,6R*,7aS*)-3a-(3,4-dimethoxyphenyl)-1-[(2-methyl-1H-imidazol-4-yl)methyl]octahydro-1H-indol-6-yl}urea trifluoroacetate

N-(3,4-difluorophenyl)-N′-[(3aS*,6R*,7aS*)-3a-(3,4-dimethoxyphenyl)octahydro-1H-indol-6-yl]urea (Example 175) (0.010 g, 0.023 mmol) and 2-methyl-1H-imidazole-4-carboxaldehyde (0.115 mmol) were dissolved in MeOH (0.7 ml) and stirred for 30 minutes before NaCNBH₃ (0.015 g, 0.23 mmol) was added. The mixture was stirred for 16 h and filtered. The crude product was purified by reversed phase HPLC. Isolated as the TFA salt.

Yield 13 mg (87%). White solid. MS (ionspray; [M+H]⁺) m/z: 526.2. HRMS for C₂₈H₃₃F₂N₅O₃: Calcd, 525.2551; found, 525.2540.

### EXAMPLE 257

### N-(3,4-difluorophenyl)-N′-[(3aS*,6R*,7aS*)-3a-(4-ethoxy-3-methoxyphenyl)-1-methyloctahydro-1H-indol-6-yl]urea trifluoroacetate

Ethyl iodide (26 L, 0.33 mmol) was added to a suspension of N-(3,4-difluorophenyl)-N′-[(3aS*,6R*,7aS*)-3a-(4-hydroxy-3-methoxyphenyl)-1-methyloctahydro-1H-indol-6-yl]urea hydrochloride (Example 323) (29 mg, 0.053 mmol) and Cs₂CO₃ (50 mg, 0.142 mmol) in DMF (3 ml). The mixture was stirred overnight. Solvent evaporated and product isolated by preparative HPLC. Yield 12.5 mg (41%), colourless oil. MS (ESI+) for C₂₅H₃₁F₂N₃O₃ m/z 460 (M+1). HRMS (EI) calcd for C₂₅H₃₁F₂N₃O₃: 459.2333, found 459.2317.

### EXAMPLE 258

### N-[(3aS*,6R*,7aS*)-1-(2-azidoethyl)-3a-(3,4-dimethoxyphenyl)octahydro-1H-indol-6-yl]-N′-(3,4-difluorophenyl)urea

To a solution of N-(3,4-difluorophenyl)-N′-[(3aS*,6R*,7aS*)-3a-(3,4-dimethoxyphenyl)octahydro-1H-indol-6-yl]urea (Example 175) (170 mg, 0.394 mmol) in MeOH (1 mL) was added acetic acid (50 mg, 0.8 mmol) and chloroacetaldehyde (300 μL of a 40% aq. soln, 1.52 mmol). The resulting solution was stirred at rt during one minute, and NaBH₃CN (50 mg, 0.796 mmol) was then added portionwise. The mixture was stirred for another 5 min until HPLC indicated full conversion of the starting material. The reaction mixture was concentrated in vacuo and the residue was dissolved in 20% aq. NH₄Cl (5 mL) and extracted with EtOAc (2×10 mL). The combined organic phases were washed (sat. aq. NaCl, 5 mL), dried (Na₂SO₄) and evaporated to give the intermediate N-[(3aS*,6R*,7aS*)-1-(2-chloroethyl)-3a-(3,4-dimethoxyphenyl)octahydro-1H-indol-6-yl]-N′-(3,4-difluorophenyl)urea (200 mg) as a colorless oil, which was used without further purification.

¹H NMR (270 MHz, CDCl₃) δ ppm 0.92-1.15 (m, 1H); 1.25-1.41 (m, 1H); 1.65-2.15 (m, 4H); 2.33-2.67 (m, 1H); 3.05-3.37 (m, 2H); 3.59 (dd, J=5.8, 1.9 Hz, 1H); 3.84 (s, 6H); 3.92-4.07 (m, 1H); 4.80 (d, J=3.0 Hz, 1H); 6.73-7.10 (m, 5H); 7.26-7.32 (m, 1H). MS (ESI+) for C₂₅H₃₀ClF₂N₃O₃ m/z 494 (M+1).

A solution of the crude alkyl chloride (0.394 mmol, assuming 100% yield in the previous reaction) in EtOH (3 mL) was transferred to a solution of LiN₃ (100 mg, 2 mmol) and NH₄Cl (100 mg, 1.9 mmol) in 50% aq. EtOH (2 mL) and the resulting solution was heated at reflux overnight. The reaction mixture was evaporated and the residue was partitionated between EtOAc (10 mL) and H₂O (5 mL). The aqueous phase was extracted once with EtOAc (10 mL) and the combined organics were washed (sat. aq. NaCl), dried (MgSO₄) and evaporated. The crude azide was purified using column chromatography (silica, CHCl₃ sat with NH₃) to give the title compound (135 mg, 65%) as a colorless oil, which solidified upon standing.

¹H NMR (270 MHz, CDCl₃) δ ppm 0.95-1.15 (m, 1H); 1.25-1.45 (m, 1H); 1.65-2.22 (m, 6H); 2.28-2.57 (m, 3H); 3.00-3.52 (m, 5H); 3.83 (s, 6H); 3.88-4.12 (m, 1H); 4.88 (d, J=8.7 Hz, 1H); 6.74-7.05 (m, 5H); 7.16-7.38 (m, 2H).

MS (ESI+) for C₂₅H₃₀F₂N₆O₃ m/z 501 (M+1).

HRMS (EI): found: 500.2351; calc.: 500.2347.

### EXAMPLE 259

### N-(3,4-difluorophenyl)-N′-{(3aS*,6R*,7aS*)-3a-(3,4-dimethoxyphenyl)-1-[(4,5-dimethyl-2-furyl)methyl]octahydro-1H-indol-6-yl}urea trifluoroacetate

N-(3,4-difluorophenyl)-N′-[(3aS*,6R*,7aS*)-3a-(3,4-dimethoxyphenyl)octahydro-1H-indol-6-yl]urea (Example 175) (0.010 g, 0.023 mmol) and 4,5-dimethylfurfural (0.115 mmol) were dissolved in MeOH (0.7 ml) and stirred for 30 minutes before NaCNBH₃ (0.015 g, 0.23 mmol) was added. The mixture was stirred for 16 h and filtered. The crude product was purified by reversed phase HPLC. Isolated as the TFA salt. Yield 3.0 mg (20%). Yellow oil. MS (ionspray; [M+H]⁺) m/z: 540.4. HRMS for C₃₀H₃₅F₂N₃O₄: Calcd, 539.2596; found, 539.2583.

### EXAMPLE 260

### N-(3,4-difluorophenyl)-N′-[(3aS*,6R*,7aS*)-3a-(3,4-dimethoxyphenyl)-1-(3-furylmethyl)octahydro-1H-indol-6-yl]urea trifluoroacetate

General procedure for the reductive amination:

A stock solution was made by dissolving N-(3,4-difluorophenyl)-N′-[(3aS*,6R*,7aS*)-3a-(3,4-dimethoxyphenyl)octahydro-1H-indol-6-yl]urea (Example 175) (0.130 g, 0.30 mmol) in MeOH (9.5 ml). 0.7 ml (0.023 mmol) of this solution was added into each vial along with the requisite aldehyde (0.115 mmol) and stirred for 30 minutes before NaCNBH₃ (0.015 g, 0.23 mmol) was added.

Reagent: 3-furaldehyde

The mixture was stirred for 18 h and filtered. The crude product was purified by reversed phase HPLC. Yield 10.3 mg (70%). Light brown liquid. MS (ionspray; [M+H]⁺) m/z: 512.4. HRMS for C₂₈H₃₁F₂N₃O₄: Calcd, 511.2283; found, 511.2282.

### EXAMPLE 261

### N-(3,4-difluorophenyl)-N′-[(3aS*,6R*,7aS*)-3a-(3,4-dimethoxyphenyl)-1-isopropyloctahydro-1H-indol-6-yl]urea trifluoroacetate

Procedure for reductive amination, see Example 260.

Reagent: acetone

The mixture was stirred for 18 h and filtered. The crude product was purified by reversed phase HPLC. Isolated as the TFA salt. Yield 9.9 mg (73%) Colourless oil. MS (ionspray; [M+H]⁺) m/z: 474.0. HRMS for C₂₆H₃₃F₂N₃O₃: Calcd, 473.2490; found, 473.2484.

### EXAMPLE 262

### N-(3,4-difluorophenyl)-N′-[(3aS*,6R*,7aS*)-3a-(3,4-dimethoxyphenyl)-1-(pyridin-4-ylmethyl)octahydro-1H-indol-6-yl]urea bis(trifluoroacetate)

Procedure for reductive amination, see Example 260.

Reagent: 4-pyridinecarboxaldehyde

The mixture was stirred for 18 h and filtered. The crude product was purified by reversed phase HPLC. Isolated as the TFA salt. Yield 15.5 mg (88%). White solid. MS (ionspray; [M+H]⁺) m/z: 523.2. HRMS for C₂₉H₃₂F₂N₄O₃: Calcd, 522.2442; found, 522.2445.

### EXAMPLE 263

### N-(3,4-difluorophenyl)-N′-[(3aS*,6R*,7aS*)-3a-(3,4-dimethoxyphenyl)-1-(pyridin-3-ylmethyl)octahydro-1H-indol-6-yl]urea bis(trifluoroacetate)

Procedure for reductive amination, see Example 260.

Reagent: 3-pyridinecarboxaldehyde

The mixture was stirred for 18 h and filtered. The crude product was purified by reversed phase HPLC. Isolated as the TFA salt. Yield 16.8 mg (97%). White solid. MS (ionspray; [M+H]⁺) In/z: 523.2. HRMS for C₂₉H₃₂F₂N₄O₃: Calcd, 522.2442; found, 522.2463.

### EXAMPLE 264

### N-(3,4-difluorophenyl)-N′-{(3aS*,6R*,7aS*)-3a-(3,4-dimethoxyphenyl)-1-[(5-methyl-2-furyl)methyl]octahydro-1H-indol-6-yl}urea trifluoroacetate

Procedure for reductive amination, see Example 260.

Reagent: 5-methylfurfural

The mixture was stirred for 18 h and filtered. The crude product was purified by reversed phase HPLC. Isolated as the TFA salt. Yield 6.2 mg (42%). Light brown liquid. MS (ionspray; [M+H]⁺) m/z: 526.2. HRMS for C₂₉H₃₃F₂N₃O₄: Calcd, 525.2439; found, 525.2429.

### EXAMPLE 265

### N-(3,4-difluorophenyl)-N′-[(3aS*,6R*,7aS*)-3a-(3,4-dimethoxyphenyl)-1-(3-thienylmethyl)octahydro-1H-indol-6-yl]urea trifluoroacetate

Procedure for reductive amination, see Example 260.

Reagent: 3-thiophenecarboxaldehyde

The mixture was stirred for 18 h and filtered. The crude product was purified by reversed phase HPLC. Isolated as the TFA salt. Yield 8.1 mg (54%). Colourless liquid. MS (ionspray; [M+H]⁺) m/z: 528.0. HRMS for C₂₈H₃₁F₂N₃O₃S: Calcd, 527.2054; found, 527.2052

### EXAMPLE 266

### N-(3,4-difluorophenyl)-N′-[(3aS*,6R*,7aS*)-3a-(3,4-dimethoxyphenyl)-1-(4-methylbenzyl)octahydro-1H-indol-6-yl]urea trifluoroacetate

Procedure for reductive amination, see Example 260.

Reagent: p-tolualdehyde

The mixture was stirred for 18 h and filtered. The crude product was purified by reversed phase HPLC. Isolated as the TFA salt. Yield 1.1 mg (7.3%). Colourless liquid. MS (ionspray; [M+H]⁺) m/z: 536.2. HRMS for C₃₁H₃₅F₂N₃O₃: Calcd, 535.2646; found, 535.2644.

### EXAMPLE 267

### N-(3,4-difluorophenyl)-N′-[(3aS*,6R*,7aS*)-3a-(3,4-dimethoxyphenyl)-1-(4-fluorobenzyl)octahydro-1H-indol-6-yl]urea trifluoroacetate

Procedure for reductive amination, see Example 260.

Reagent: 4-fluorobenzaldehyde

The mixture was stirred for 18 h and filtered. The crude product was purified by reversed phase HPLC. Isolated as the TFA salt. Yield 8.4 mg (56%). Colourless oil. MS (ionspray; [M+H]⁺) m/z: 540.4. HRMS for C₃₀H₃₂F₃N₃O₃: Calcd, 539.2396; found, 539.2390.

### EXAMPLE 268

### N-(3,4-difluorophenyl)-N′-{(3aS*,6R*,7aS*)-3a-(3,4-dimethoxyphenyl)-1-[(5-methyl-2-thienyl)methyl]octahydro-1H-indol-6-yl}urea trifluoroacetate

Procedure for reductive amination, see Example 260.

Reagent: 5-methyl-2-thiophenecarboxaldehyde

The mixture was stirred for 18 h and filtered. The crude product was purified by reversed phase HPLC. Isolated as the TEA salt. Yield 7.6 mg (51%). Yellow oil. MS (ionspray; [M+H]⁺) m/z: 542.2. HRMS for C₂₉H₃₃F₂N₃O₃S: Calcd, 541.2211; found, 541.2212

### EXAMPLE 269

### N-(3,4-difluorophenyl)-N′-{(3aS*,6R*,7aS*)-3a-(3,4-dimethoxyphenyl)-1-[(2-ethyl-4-methyl-1H-imidazol-5-yl)methyl]octahydro-1H-indol-6-yl}urea trifluoroacetate

Procedure for reductive amination, see Example 260.

Reagent: 2-ethyl-5-formyl-4-methylimidazole

The mixture was stirred for 18 h and filtered. The crude product was purified by reversed phase HPLC. Isolated as the TFA salt. Yield 10.3 mg (67%). Colourless oil. MS (ionspray; [M+H]⁺) m/z: 554.0. HRMS for C₃₀H₃₇F₂N₅O₃: Calcd, 553.2864; found, 553.2867.

### EXAMPLE 270

### N-[(3aS*,6R*,7aS*)-1-[(5-chloro-1,3-dimethyl-1H-pyrazol-4-yl)methyl]-3a-(3,4-dimethoxyphenyl)octahydro-1H-indol-6-yl]-N′-(3,4-difluorophenyl)urea trifluoroacetate

Procedure for reductive amination, see Example 260.

Reagent: 5-chloro-1,3-dimethyl-1H-pyrazole-4-carbaldehyde

The mixture was stirred for 18 h and filtered. The crude product was purified by reversed phase HPLC. Isolated as the TFA salt. Yield 5.5 mg (35%). Colourless oil. MS (ionspray; [M+H]⁺) m/z: 574.2. HRMS for C₂₉H₃₄ClF₂N₅O₃: Calcd, 573.2318; found, 573.2329.

### EXAMPLE 271

### N-(3,4-difluorophenyl)-N′-{(3aS*,6R*,7aS*)-3a-(3,4-dimethoxyphenyl)-1-[(2-ethyl-1H-imidazol-4-yl)methyl]octahydro-1H-indol-6-yl}urea trifluoroacetate

Procedure for reductive amination, see Example 260.

Reagent: 2-ethyl-1H-imidazole-5-carbaldehyde

The mixture was stirred for 18 h and filtered. The crude product was purified by reversed phase HPLC. Isolated as the TFA salt. Yield 7.2 mg (48%). Colourless oil. MS (ionspray; [M+H]⁺) m/z: 540.4.

### EXAMPLE 272

### N-(3,4-difluorophenyl)-N′-{(3aS*,6R*,7aS*)-3a-(3,4-dimethoxyphenyl)-1-[2-(dimethylamino)ethyl]octahydro-1H-indol-6-yl}urea bis(trifluoroacetate)

NaCNBH₃ (0.020 g, 0.32 mmol) was added to a solution of N-(3,4-difluorophenyl)-N′-[(3aS*,6R*,7aS*)-3a-(3,4-dimethoxyphenyl)octahydro-1H-indol-6-yl]urea (Example 175) (0.020 g, 0.046 mmol) and chloroacetaldehyde (0.036 g, 0.21 mmol) and stirred overnight. 1 ml of dimethylamine was added and the mixture was heated to 70° C. for 20 minutes, cooled and evaporated. The crude product was purified by reversed phase HPLC. Isolated as the TFA salt. Yield 3.2 mg (10%) Colourless oil. MS (ionspray; [M+H]⁺) m/z: 503.2.

HRMS for C₂₇H₃₆F₂N₄O₃: Calcd, 502.2755; found, 502.2750.

### EXAMPLE 273

### N-(3,4-difluorophenyl)-N′-[(3aS*,6R*,7aS*)-3a-(3-methoxy-4-propoxyphenyl)-1-methyloctahydro-1H-indol-6-yl]urea trifluoroacetate

1-Iodopropane (43 μL, 0.448 mmol) was added to a suspension of N-(3,4-difluorophenyl)-N′-[(3aS*,6R*,7aS*)-3a-(4-hydroxy-3-methoxyphenyl)-1-methyloctahydro-1H-indol-6-yl]urea hydrochloride (Example 323) (42 mg, 0.089 mmol) and Cs₂CO₃ (150 mg, 0.46 mmol) in DMF (1 ml). The mixture was stirred overnight at ambient temperature. Solvent evaporated and product isolated by preparative HPLC. Yield 27.1 mg (52%). MS (ESI+) for C₂₆H₃₃F₂N₃O₃ m/z 474 (M+1). HRMS (EI) calcd for C₂₆H₃₃F₂N₃O₃: 473.249, found 473.2516.

### EXAMPLE 274

### N-(3,4-difluorophenyl)-N′-[(3aS*,6R*,7aS*)-3a-(4-isopropoxy-3-methoxyphenyl)-1-methyloctahydro-1H-indol-6-yl]urea trifluoroacetate

2-Bromopropane (42 μL, 0.448 mmol) was added to a suspension of N-(3,4-difluorophenyl)-N′-[(3aS*,6R*,7aS*)-3a-(4-hydroxy-3-methoxyphenyl)-1-methyloctahydro-1H-indol-6-yl]urea hydrochloride (Example 323) (42 mg, 0.089 mmol) and Cs₂CO₃ (150 mg, 0.46 mmol) in DMF (1 ml). The mixture was stirred overnight at ambient temperature. Solvent evaporated and product isolated by preparative HPLC. Yield 46.9 mg (90%). MS (ESI+) for C₂₆H₃₃F₂N₃O₃ M/Z 474 (M+1). HRMS (EI) calcd for C₂₆H₃₃F₂N₃O₃: 473.249, found 473.2489.

### EXAMPLE 275

### N-(3,4-difluorophenyl)-N′-{(3aS*,6R*,7aS*)-3a-(3,4-dimethoxyphenyl)-1-[(4-methyl-1H-imidazol-5-yl)methyl]octahydro-1H-indol-6-yl}urea

Procedure for reductive amination, see Example 260.

Reagent: 4-methyl-5-imidazolecarboxaldehyde

The mixture was stirred for 30 h and filtered. The white crystals were washed with MeOH and dried. Yield 5.7 mg (48%). White solid. MS (ionspray; [M+H]⁺) m/z: 526.2.

### EXAMPLE 276

### N-(3,4-difluorophenyl)-N′-[(3aS*,6R*,7aS*)-3a-(3,4-dimethoxyphenyl)-1-methyl-6-propyloctahydro-1H-indol-6-yl]urea and

### EXAMPLE 277

### N-(3,4-difluorophenyl)-N′-[(3aS*,6S*,7aS*)-3a-(3,4-dimethoxyphenyl)-1-methyl-6-propyloctahydro-1H-indol-6-yl]urea

(3aS*,7aS*)-3a-(3,4-Dimethoxyphenyl)-1-methyloctahydro-6H-indol-6-one (Comparative Example 4; 605 mg, 2.09 mmol) dissolved in toluene (15 mL) was heated to reflux for 2 h with benzylamine (0.228 mL, 2.09 mmol) using a Dean-Stark condensor. The solution was taken to dryness and the residue was dissolved in THF (5 mL) and added to Mg (60 mg, 2.5 mmol). The mixture was cooled in ice under N₂. Allyl bromide (278 mg, 2.3 mmol) was added. After 5 min the mixture was warmed to room temp and stirred for 1.5 h. Aq. saturated NH₄Cl (1 mL) was added and the mixture was extraced with diethyl ether. The crude product was purified by chromatography on Lichroprep RP-18 using 20-40% CH₃CN-0.2% TFA in water as eluent. The resulting TFA-salt, dissolved in MeOH, was passed through an anion exchange resin, Dowex 1×8 in hydroxide form. HPLC of this product (310 mg) showed the presence of three peaks in the ratio 1:1.38:0.21. This product was dissolved in 95% EtOH (20 mL) and was hydrogenated over 10% Pd/C (34 mg) for 6 h. After filtration and evaporation of EtOH the product was purified by flash chromatography on silica gel with 4% MeOH, 0.8% Et₃N/DCM as eluent. Two fractions were collected: First eluted: (42 mg, 6% yield)

MS ES+m/z 333.3. ¹H NMR (CDCl₃) δ 0.91 (t, J=6.9 Hz, 3H), 1.00-1.20 (m, 3H), 1.21-1.39 (m, 2H), 1.39-1.61 (m, 3H), 1.72-2.04 (m, 6H), 2.13-2.31 (m, 4H), 2.71 (m, 1H), 3.02 (m, 1H), 3.83, 3.85 (s, 6H), 6.72-6.81 (m, 1H), 6.85-6.95 (m, 2H). ¹³C NMR (CDCl₃) δ 14.81, 16.69, 33.87, 35.55, 36.67, 39.96, 40.27, 42.70, 47.69, 50.84, 54.22, 55.86, 55.78, 68.73, 110.44, 110.64, 118.42, 140.77, 146.83, 148.60.

Second eluted: (65 mg, 9% yield)

MS ES+m/z 333.3. ¹H NMR (CDCl₃) δ 0.86 (t, J=6.2 Hz, 3H), 1.05-1.39 (m, 6H), 1.45 (dd, J=14.8, 3.5 Hz, 1H), 1.63-2.00 (m, 4H), 2.08-2.28 (m, 2H), 2.34 (s, 3H), 2.66 (bs, 1H), 2.90 (bs, 2H), 3.18 (m, 1H), 3.82, 3.84 (s, 6H), 6.67-6.92 (m, 3H). ¹³C NMR (CDCl₃) δ 14.63, 15.91, 31.35, 33.28, 35.12, 40.59, 40.69, 47.26, 47.54, 51.21, 53.93, 55.70, 55.84, 68.76, 110.30, 110.60, 118.39, 139.25, 146.88, 148.57.

The first eluted fraction (42 mg, 0.126 mmol) dissolved in DCM (1 mL) and 3,4-difluorophenyl isocyanate (29 mg, 0.188 mmol) was kept at room temp for 5 h. The solution was added to PS-trisamine (ca 0.18 g) in DCM and stirred for 2 h. The resin was filtered and washed with DCM and the filtrate was evaporated. Flash-chromatography using 10% MeOH/CHCl₃ as eluent gave the title compound (49 mg, 86%).

MS ES+m/z 488.1. ¹H NMR (CDCl₃) δ 0.92 (t, J=7.2 Hz, 3H), 1.12-2.43 (m, 18H), 2.74 (m, 1H), 3.04 (m, 1H), 3.84 (s, 6H), 4.20 (bs, 1H), 5.99 (bs, 1H), 6.72-7.07 (m, 5H), 7.28-7.38 (m, 1H).

Second eluated fraction (65 mg, 0.195 mmol) dissolved in DCM (1 mL) and 3,4-difluorophenyl isocyanate (36 mg, 0.234 mmol) was kept at room temp for 5 h. The solution was added to PS-Trisamin (ca 0.18 g) in DCM and stirred for 2 h. The resin was filtered and washed with DCM and the filtrate was evaporated to give the title compound (90 mg, 95%).

MS ES+m/z 488.1. ¹H NMR (CDCl₃) δ 0.88 (t, J=6.9 Hz, 3H), 1.07 (m, 1H), 1.15-1.45 (m, 4H), 1.76-2.09 (m, 5H), 2.10-2.45 (m, 6H), 2.79 (bs, 1H), 3.15 (m, 1H), 3.86, 3.87 (s, 6H), 5.87 (bs, 1H), 6.72-6.87 (m, 3H), 6.87-6.97 (m, 1H), 6.99-7.14 (m, 1H), 7.27-7.38 (m, 1H), 7.49 (bd, 1H).

### EXAMPLE 278

### (3aS*,6R*,7aS*)-6-([(3,4-difluorophenyl)amino]carbonyl}amino)-3a-(3,4-dimethoxyphenyl)-1,1-dimethyloctahydro-1H-indolium trifluoroacetate

Methyl iodide (MeI) (0.0043 ml, 0.06 mmol) was added to a suspension of N-(3,4-difluorophenyl)-N′-[(3aS*,6R*,7aS*)-3a-(3,4-dimethoxyphenyl)octahydro-1H-indol-6-yl]urea (Example 175) (0.010 g, 0.023 mmol) and K₂CO₃ (0.010 g, 0.07 mmol) in DMF (1 ml). The mixture was stirred overnight and additional MeI (0.0043 ml, 0.06 mmol) and K₂CO₃ (0.010 g, 0.07 mmol) was added and the mixture was heated to 50° C. overnight. The crude product was purified by reversed phase HPLC. Isolated as the TA salt. Yield 2.8 mg (21%). Colourless liquid. MS (ionspray; [M+H]⁺) m/z: 460.4. HRMS for C₂₅H₃₂F₂N₃O₃: Calcd, 460.2412; found, 460.2417.

### EXAMPLE 279

### N-(3,4-difluorophenyl)-N′-[(3aS*,6R*,7aS*)-3a-(3,4-dimethoxyphenyl)-1-isobutyloctahydro-1H-indol-6-yl]urea trifluoroacetate

Procedure for reductive amination, see Example 260.

Reagent: isobutyraldehyde

The mixture was stirred for 18 h and filtered. The crude product was purified by reversed phase HPLC. Isolated as the TFA salt. Yield 12.5 mg (90%). Colourless oil. MS (ionspray; [M+H]⁺) m/z: 488.2. HRMS for C₂₇H₃₅F₂N₃O₃: Calcd, 487.2646; found, 487.2659.

### EXAMPLE 280

### N-[(3aS*,6R*,7aS*)-1-cyclopentyl-3a-(3,4-dimethoxyphenyl)octahydro-1H-indol-6-yl]-N′-(3,4-difluorophenyl)urea trifluoroacetate

Procedure for reductive amination, see Example 260.

Reagent: cyclopentanone

The mixture was stirred for 18 h and filtered. The crude product was purified by reversed phase HPLC. Isolated as the TFA salt. Yield 10.3 mg (74%). Colourless oil. MS (ionspray; [M+H]⁺) m/z: 500.2. HRMS for C₂₈H₃₅F₂N₃O₃: Calcd, 499.2646; found, 499.2637.

### EXAMPLE 281

### N-(3,4-difluorophenyl)-N′-[(3aS*,6R*,7aS*)-3a-(3,4-dimethoxyphenyl)-1-(3-methylbutyl)octahydro-1H-indol-6-yl]urea trifluoroacetate

Procedure for reductive amination, see Example 260.

Reagent: isovaleraldehyde

The mixture was stirred for 18 h and filtered. The crude product was purified by reversed phase HPLC. Isolated as the TFA salt. Yield 9.6 mg (69%). Colourless oil. MS (ionspray; [M+H]⁺) m/z: 502.2. HRMS for C₂₈H₃₇F₂N₃O₃: Calcd, 501.2803; found, 501.2817.

### EXAMPLE 282

### N-[(3aS*,6R*,7aS*)-1-(cyclopropylmethyl)-3a-(3,4-dimethoxyphenyl)octahydro-1H-indol-6-yl]-N′-(3,4-difluorophenyl)urea trifluoroacetate

Procedure for reductive amination, see Example 260.

Reagent: cyclopropanecarboxaldehyde

The mixture was stirred for 17 h and filtered. The crude product was purified by reversed phase HPLC. Isolated as the TFA salt. Yield 9.3 mg (67%). Colourless liquid. MS (ionspray; [M+H]⁺) m/z: 486.4. HRMS for C₂₇H₃₃F₂N₃O₃: Calcd, 485.2490; found, 485.2506.

### EXAMPLE 283

### N-(3,4-difluorophenyl)-N′-[(3aS*,6R*,7aS*)-3a-(3,4-dimethoxyphenyl)-1-(2-ethylbutyl)octahydro-1H-indol-6-yl]urea trifluoroacetate

Procedure for reductive amination, see Example 260.

Reagent: 2-ethylbutyraldehyde

The mixture was stirred for 18 h and filtered. The crude product was purified by reversed phase HPLC. Isolated as the TFA salt. Yield 10.7 mg (74%). MS (ionspray; [M+H]⁺) m/z: 516.4. HRMS for C₂₉H₃₉F₂N₃O₃: Calcd, 515.2959; found, 515.2945.

### EXAMPLE 284

### N-(3,4-difluorophenyl)-N′-[(3aS*,6R*,7aS*)-3a-(3,4-dimethoxyphenyl)-1-(3,3-dimethylbutyl)octahydro-1H-indol-6-yl]urea trifluoroacetate

Procedure for reductive amination, see Example 260.

Reagent: 3,3-dimethylbutyraldehyde

The mixture was stirred for 17 h and filtered. The crude product was purified by reversed phase HPLC. Isolated as the TFA salt. Yield 7.1 mg (49%). MS (ionspray; [M+H]⁺) m/z: 516.2. HRMS for C₂₉H₃₉F₂N₃O₃: Calcd, 515.2959; found, 515.2947.

### EXAMPLE 285

### N-(3,4-difluorophenyl)-N′-[(3aS*,6R*,7aS*)-3a-(3,4-dimethoxyphenyl)-1-(3,3,3-trifluoropropyl)octahydro-1H-indol-6-yl]urea trifluoroacetate

Procedure for reductive amination, see Example 260.

Reagent: 3,3,3-trifluoropropanal

The mixture was stirred for 4 days and filtered. The crude product was purified by reversed phase HPLC. Isolated as the TFA salt. Yield 0.8 mg (5%). MS (ionspray; [M+H]⁺) m/z: 528.2. HRMS for C₂₆H₃₀F₅N₃O₃: Calcd, 527.2207; found, 527.2191.

### EXAMPLE 286

### N-(3,4-difluorophenyl)-N′-[(3aS*,6R*,7aS*)-3a-(3,4-dimethoxyphenyl)-1-(pyridin-2-ylmethyl)octahydro-1H-indol-6-yl]urea bis(trifluoroacetate)

Procedure for reductive amination, see Example 260.

Reagent: 2-pyridinecarboxaldehyde

The mixture was stirred for 17 h and filtered. The crude product was purified by reversed phase HPLC. Isolated as the TFA salt. Yield 7.1 mg (42%). Colourless oil. MS (ionspray; [M+H]⁺) m/z: 523.2. HRMS for C₂₉H₃₂F₂N₄O₃: Calcd, 522.2442; found, 522.2434.

### EXAMPLE 287

### N-(3,4-difluorophenyl)-N′-{(3aS*,6R*,7aS*)-3a-(3,4-dimethoxyphenyl)-1-[3-(dimethylamino)-2,2-dimethylpropyl]octahydro-1H-indol-6-yl}urea bis(trifluoroacetate)

Procedure for reductive amination, see Example 260.

Reagent: 3-dimethylamino-2,2-dimethylpropionaldehyde

The mixture was stirred for 4 days and filtered. The crude product was purified by reversed phase HPLC. Isolated as the TFA salt. Yield 2.6 mg (14%). White solid. MS (ionspray; [M+H]⁺) m/z: 545.2. HRMS for C₃₀H₄₂F₂N₄O₃: Calcd, 544.3225; found, 544.3233.

### EXAMPLE 288

### N-(3,4-difluorophenyl)-N′-[(3aS*,6R*,7aS*)-3a-(3,4-dimethoxyphenyl)-1-(tetrahydro-2H-pyran-4-yl)octahydro-1H-indol-6-yl]urea trifluoroacetate

Procedure for reductive amination, see Example 260.

Reagent: tetrahydro-4H-pyran-4-one

The mixture was stirred for 16 h and filtered. The crude product was purified by reversed phase HPLC. Isolated as the TFA salt. Yield 4.6 mg (32%). White solid. MS (ionspray; [M+H]⁺) m/z: 516.2. HRMS for C₂₈H₃₅F₂N₃O₄: Calcd, 515.2596; found, 515.2583.

### EXAMPLE 289

### N-(3,4-difluorophenyl)-N′-[(3aS*,6R*,7aS*)-3a-(3,4-dimethoxyphenyl)-1-(2-fluoroethyl)octahydro-1H-indol-6-yl]urea trifluoroacetate

N-(3,4-difluorophenyl)-N′-[(3aS*,6R*,7aS*)-3a-(3,4-dimethoxyphenyl)octahydro-1H-indol-6-yl]urea (Example 175) (0.010 g, 0.023 mmol), 1-bromo-2-fluoroethane (0.19 g, 0.25 mmol) and triethylamine (0.06 ml, 0.46 mmol) were mixed and heated at 50° C. in a sealed vial for 15 days. The mixture was evaporated and the crude product was purified by reversed phase HPLC. Isolated as the TFA salt. Yield 5.2 mg (38%). White solid. MS (ionspray; [M+H]⁺) m/z: 478.2. HRMS for C₂₅H₃₀F₃N₃O₃: Calcd, 477.2239; found, 477.2256.

### EXAMPLE 290

### N-(3,4-difluorophenyl)-N′-((3aS*,6R*,7aS*)-3a-(3,4-dimethoxyphenyl)-1-{2-[4-(trimethylsilyl)-1H-1,2,3-triazol-1-yl]ethyl}octahydro-1H-indol-6-yl)urea

A tube was charged with a magnetic stirbar, Example 258 (25 mg, 0.050 mmol), and trimethylsilylacetylene (0.5 mL). The tube was sealed tightly and the contents were stirred at 100° C. for 24 hrs. The reaction mixture was evaporated at reduced pressure and the residue was subjected to column chromatography (silica, CHCl₃ sat. with NH₃), to give the title compound (28 mg, 93%) as a colorless oil. The regiochemistry was determined using NOE spectroscopy.

¹H NMR (270 MHz, CDCl₃) δ ppm 0.27 (s, 9H); 0.90-2.8 (m, 11H); 2.90-3.70 (m, 5H); 3.82 (s, 6H); 4.35-5.10 (m, 2H); 6.65-7.15 (m, 5H); 6.74-7.05 (m, 5H); 7.40-7.72 (m, 1H); 7.89 (s, 1H).

MS (ESI+) for m/z 599 (M+1).

HRMS (EI) Calc for C₃₀H₄₀F₂N₆O₃Si: 598.2899; found: 598.2922.

### EXAMPLE 291

### N-(3,4-difluorophenyl)-N′-[(3aS*,6R*,7aS*)-3a-(3,4-dimethoxyphenyl)-1-(2-{5-[(4-methylphenyl)sulfonyl]-1H-tetrazol-1-yl}ethyl)octahydro-1H-indol-6-yl]urea

A tube was charged with a magnetic stirbar, Example 258 (25 mg, 0.050 mmol), and p-toluenesulfonyl cyanide (100 mg, 0.55 mmol). The tube was sealed tightly and the contents were stirred at 100° C. for 24 hrs. Column chromatography (silica, heptane:CHCl₃ sat. with NH₃) 25:75 to 0:100) then afforded the title compound (27 mg, 79%) as a colorless oil.

MS (ESI+) for m/z 682 (M+1).

HRMS (EI) Calc for C₃₃H₃₇F₂N₇O₅S: 681.2545: Found 681.2567.

### EXAMPLE 292

### N-[(3aS*,6R*,7aS*)-3a-(3,4-dimethoxyphenyl)-1-methyloctahydro-1H-indol-6-yl]-N′-(3-methylisothiazol-5-yl)urea trifluoroacetate

5-Amino-3-methylisothiazole (21 mg, 0.14 mmol) was dissolved in 1 mL dry CH₂Cl₂ under N₂. Triethylamine (24 mg, 0.14 mmol) was added followed by drop-wise addition of triphosgene (20 mg, 0.06 mmol) in 1 mL of dry CH₂Cl₂. Stirred at room temperature for 2 hrs, and then (3aS*,6R*,7aS*)-3a-(3,4-dimethoxyphenyl)-1-methyloctahydro-1H-indol-6-amine (Comparative Example 7; 35 mg, 0.12 mmol) was added. Stirred at room temperature overnight and then concentrated. Purification using preparative HPLC gave the product as yellow oil (8.3 mg, 16%).

MS (ESI⁺) for C₂₂H₃₀N₄O₃S m/z 431 (M+H⁺), HRMS found: 430,2040 calculated: 430,2039

¹HNMR (270 MHz, Chloroform-d) ppm 1.20-1.35 (m, 1H); 1.86-2.01 (m, 4H); 2.42-2.59 (m, 4H); 3.05 (s, 3H); 3.48 (s, 1H); 3.62-3.72 (m, 1H); 3.88 (s, 9H); 4.15-4.25 (m, 2H); 5.66 (w, 1H); 6.42-6.53 (m, 1H); 6.77-6.94 (m, 2H); 7.63 (s, 1H)

### EXAMPLE 293

### N-[(3aS*,6R*,7aS*)-3a-(3,4-dimethoxyphenyl)-1-methyloctahydro-1H-indol-6-yl]-N′-isoxazol-3-ylurea trifluoroacetate

3-Aminoisooxazole (12 mg, 0.114 mmol) was dissolved in 1 mL dry CH₂Cl₂ under N₂. Triethylamine (24 mg, 0.14 mmol) was added followed by drop-wise addition of triphosgene (20 mg, 0,06 mmol) in 1 mL of dry CH₂Cl₂. Stirred at room temperature for 2 hrs, and then (3aS*,6R*,7aS*)-3a-(3,4-dimethoxyphenyl)-1-methyloctahydro-1H-indol-6-amine (Comparative Example 7; 35 mg, 0.12 mmol) was added. Stirred at room temperature overnight and then concentrated. Purification using preparative HPLC gave the product as light yellow oil (5.0 mg, 10%).

MS (ESI+) for C₂₁H₂₈N₄O₄ m/z 401 (M+H⁺), HRMS found: 400,2094 calculated: 400,2111

¹HNMR (270 MHz, Chloroform-d) ppm 1.25-1.40 (m, 1H); 1.85-2.15 (m, 3H); 2.18-2.85 (m, 3H); 2.58 (d, J=13.12, 1H); 2.77 (s, 1H); 3.01 (s, 3H); 3.76 (s, 1H); 3.83 (s, 1H); 3.89 (s, 6H); 4.26 (b, 2H); 6.21 (s, 1H); 6.77-6.89 (m, 3H); 8.16 (s, 1H); 9.17 (s, 1H)

### EXAMPLE 294

### N-(3,4-difluorophenyl)-N′-((3aS*,6R*,7aS*)-3a-(3,4-dimethoxyphenyl)-1-{2-[4-(methoxymethyl)-1H-1,2,3-triazol-1-yl]ethyl}octahydro-1H-indol-6-yl)urea trifluoroacetate and

### EXAMPLE 295

### N-(3,4-difluorophenyl)-N′-((3aS*,6R*,7aS*)-3a-(3,4-dimethoxyphenyl)-1-{2-[5-(methoxymethyl)-1H-1,2,3-triazol-1-yl]ethyl}octahydro-1H-indol-6-yl)urea trifluoroacetate

A tube was charged with a magnetic stirbar, Example 258 (25 mg, 0.050 mmol), and methyl propargyl ether (0.5 mL). The tube was sealed tightly and the contents were stirred at 100° C. for 24 hrs. The reaction mixture was evaporated at reduced pressure and the residue was then subjected to column chromatography (silica, CHCl₃ sat. with NH₃), to give approximately a 1:1 mixture of the title compounds (25 mg, 88%) as a colorless oil. The single regioisomers were isolated by preparative HPLC, to give as a colorless gum (8 mg, 28%).

MS (ESI+) for C₂₉H₃₆F₂N₆O₄ M/z 571 (M+1).

HRMS(EI): Found: 570.2766, Calc.: 570.2766.

From the slower eluting fraction was isolated a colorless gum (6 mg, 21%).

MS (ESI+) for C₂₉H₃₆F₂N₆O₄ m/z 571 (M+1).

HRMS(EI): Found: 570.2777, Calc.: 570.2766.

### EXAMPLE 296

### N-(4-bromo-3-chlorophenyl)-N′-[(3aS*,6R*,7aS*)-3a-(3,4-dimethoxyphenyl)-1-methyloctahydro-1H-indol-6-yl]urea trifluoroacetate

Synthetic procedure: scheme F

Reagent: 4-bromo-3-chloroaniline

Measured mass: 521.1075

Calc. mass: 521.1081

Yield: (43 mg; 68%)

### EXAMPLE 297

### N-(3-chloro-4-cyanophenyl)-N′-[(3aS*,6R*,7aS*)-3a-(3,4-dimethoxyphenyl)-1-methyloctahydro-1H-indol-6-yl]urea trifluoroacetate

Synthetic procedure: scheme F

Reagent: 4-amino-2-chlorobenzonitrile

Measured mass: 468.1925

Calc. mass: 468.1928

Yield: (38 mg; 66%)

### EXAMPLE 298

### N-[(3aS*,6R*,7aS*)-3a-(3,4-dimethoxyphenyl)-1-methyloctahydro-1H-indol-6-yl]-N′-quinolin-6-ylurea trifluoroacetate

Synthetic procedure: scheme F

Reagent: quinolin-6-amine

Measured mass: 460.2488

Calc. mass: 460.2474

Yield: (13 mg; 23%)

### EXAMPLE 299

### N-[(3aS*,6R*,7aS*)-3a-(3,4-dimethoxyphenyl)-1-methyloctahydro-1H-indol-6-yl]-N′-(2-methylquinolin-6-yl)urea trifluoroacetate

Synthetic procedure: scheme F

Reagent: 2-methylquinolin-6-amine

Measured mass: 474.2647

Calc. mass: 474.2631

Yield: (41 mg; 69%)

### EXAMPLE 300

### N-[(3aS*,6R*,7aS*)-3a-(3,4-dimethoxyphenyl)-1-methyloctahydro-1H-indol-6-yl]-N′-(2-methyl-1H-indol-5-yl)urea trifluoroacetate

Synthetic procedure: scheme F

Reagent: 2-methyl-1H-indol-5-amine

Measured mass: 462.2637

Calc. mass: 462.2631

Yield: (9 mg; 16%)

### EXAMPLE 301

### N-[(3aS*,6R*,7aS*)-3a-(3,4-dimethoxy henyl)-1-methyloctahydro-1H-indol-6-yl]-N′-(2-methyl-1,3-benzothiazol-5-yl)urea trifluoroacetate

Synthetic procedure: scheme F

Reagent: 2-methyl-1,3-benzothiazol-5-amine

Measured mass: 480.2184

Calc. mass: 480.2195

Yield: (34 mg; 58%)

### EXAMPLE 302

### N-(3,5-dibromo-4-methylphenyl)-N′-[(3aS*,6R*,7aS*)-3a-(3,4-dimethoxyphenyl)-1-methyloctahydro-1H-indol-6-yl]urea trifluoroacetate

Synthetic procedure: scheme F

Reagent: 3,5-dibromo-4-methylaniline

Measured mass: 579.0752

Calc. mass: 579.0732

Yield: (38 mg; 55%)

### EXAMPLE 303

### N-1H-1,2,3-benzotriazol-5-yl-N′-[(3aS*,6R*,7aS*)-3a-(3,4-dimethoxyphenyl)-1-methyloctahydro-1H-indol-6-yl]urea trifluoroacetate

Synthetic procedure: scheme F

Reagent: 1H-1,2,3-benzotriazol-5-amine

Measured mass: 450.2365

Calc. mass: 450.2379

Yield: (6 mg; 11%)

### EXAMPLE 304

### N-[(3aS*,6R*,7aS*)-3a-(3,4-dimethoxy henyl)-1-methyloctahydro-1H-indol-6-yl]-N′-[4-(1,2,3-thiadiazol-4-yl)phenyl]urea trifluoroacetate

Synthetic procedure: scheme F

Reagent: 4-(1,2,3-thiadiazol-4-yl)aniline

Measured mass: 493.2138

Calc. mass: 493.2148

Yield: (39 mg; 64%)

### EXAMPLE 305

### N-[4-(4-bromo-1-methyl-1H-pyrazol-3-yl)phenyl]-N′-[(3aS*,6R*,7aS*)-3a-(3,4-dimethoxyphenyl)-1-methyloctahydro-1H-indol-6-yl]urea trifluoroacetate

Synthetic procedure: scheme F

Reagent: 4-(4-bromo-1-methyl-1H-pyrazol-3-yl)aniline

Measured mass: 567.1884

Calc. mass: 567.1845

Yield: (47 mg; 69%)

### EXAMPLE 306

### N-(3,4-dicyanophenyl)-N′-[(3aS*,6R*,7aS*)-3a-(3,4-dimethoxyphenyl)-1-methyloctahydro-1H-indol-6-yl]urea trifluoroacetate

Synthetic procedure: scheme F

Reagent: 4-aminophthalonitrile

Measured mass: 459.2277

Calc. mass: 459.2270

Yield: (34 mg; 60%)

### EXAMPLE 307

### N-(3,4-difluorophenyl)-N′-{(3aS*,6R*,7aS*)-3a-(3,4-dimethoxyphenyl)-1-[(propylamino)acetyl]octahydro-1H-indol-6-yl}urea trifluoroacetate

N-(3,4-difluorophenyl)-N′-[(3aS*,6R*,7aS*)-3a-(3,4-dimethoxyphenyl)octahydro-1H-indol-6-yl]urea, Example 175, (0.010 g, 0.023 mmol) and chloroacetylchloride (0.0022 ml, 0.028 mmol) were dissolved in CH₂Cl₂ (1 ml) and one drop triethylamine was added. The mixture was stirred at room temperature for 2 hours and evaporated. The crude material was dissolved in CH₂Cl₂ (1 ml) and propylamine (0.1 g, 1.7 mmol) was added. The mixture was heated at 40° C. for 18 hours and evaporated. The crude product was purified by reversed phase HPLC. Isolated as the TFA salt. Yield 4.2 mg (28%). White solid. MS (ionspray; [M+H]⁺) m/z: 531.2. HRMS for C₂₈H₃₆F₂N₄O₄: Calcd, 530.2705; found, 530.2715.

### EXAMPLE 308

### N-[(3aS*,6R*,7aS*)-1-cyclohexyl-3a-(3,4-dimethoxyphenyl)octahydro-1H-indol-6-yl]-N′-(3,4-difluorophenyl)urea trifluoroacetate

N-(3,4-difluorophenyl)-N′-[(3aS*,6R*,7aS*)-3a-(3,4-dimethoxyphenyl)octahydro-1H-indol-6-yl]urea, Example 175, (0.010 g, 0.023 mmol) and cyclohexanone (0.0113 g, 0.115 mmol) were dissolved in MeOH (0.7 ml) and shaked for 30 minutes before MP-cyanoborohydride (0.115 g, 0.23 mmol) was added. The mixture was shaked overnight and filtered. The crude product was purified by reversed phase HPLC. Isolated as the TFA salt.

Yield 4.8 mg (33%). White solid. MS (ionspray; [M+H]⁺) m/z: 514.4. HRMS for C₂₉H₃₇F₂N₃O₃: Calcd, 513.2803; found, 513.2803

### EXAMPLE 309

### (3aS*,6R*,7aS*)-N-cyano-6-({[(3,4-difluorophenyl)amino]carbonyl}-amino)-3a-(3,4-dimethoxyphenyl)-N′-ethyloctahydro-1H-indole-1-carboximidamide

N-(3,4-difluorophenyl)-N′-[(3aS*,6R*,7aS*)-3a-(3,4-dimethoxyphenyl)octahydro-1H-indol-6-yl]urea, Example 175, (0.020 g, 0.046 mmol) and cyanocarbonimidate (0.0116 g, 0.048 mmol) were dissolved in acetonitrile (1 ml) and heated at 40° C. overnight. Additional cyanocarbonimidate (0.005 g, 0.021 mmol) was added and the mixture was stirred for 3 hours and filtered through a SCX column (1 g) with MeOH/CH₂Cl₂ (6 ml) 1:1 as the eluent. The mixture was evaporated and dissolved in THF (1 ml) and 2N ethylamine in THF (0.1 ml, 0.2 mmol) was added and the mixture was stirred at 40° C. overnight and evaporated. The crude product was purified by reversed phase HPLC. Yield 5.3 mg (23%). White solid. MS (ionspray; [M+H]⁺) m/z: 527.2. HRMS for C₂₇H₃₂F₂N₆O₃: Calcd, 526.2504; found, 526.2496.

### EXAMPLE 310

### N-(3,4-difluorophenyl)-N′-[(3aS*,6R*,7aS*)-3a-(3,4-dimethoxyphenyl)-1-(1-methylpiperidin-4-yl)octahydro-1H-indol-6-yl]urea bis(trifluoroacetate)

General procedure for reductive amination, see Example 260.

Reagent: 1-methyl-4-piperidone (0.014 g, 0.115 mmol)

The mixture was stirred for 16 hours and filtered. The crude product was purified by reversed phase HPLC. Isolated as the TFA salt. Yield 1.7 mg (10%). White solid. MS (ionspray; [M+H]⁺) m/z: 529.2. HRMS for C₂₉H₃₈F₂N₄O₃: Calcd, 528.2912; found, 528.2902.

### EXAMPLE 311

### N-(3,4-difluorophenyl)-N′-[(3aS*,6R*,7aS*)-3a-(3,4-dimethoxyphenyl)-1-(tetrahydro-3-thienyl)octahydro-1H-indol-6-yl]urea

General procedure for reductive amination, see Example 260.

Reagent: tetrahydrothiophen-3-one (0.012 g, 0.115 mmol)

The mixture was stirred for 16 hours and filtered. The white crystals were washed with MeOH (2×) and dried. Yield 2.1 mg (18%). White solid. MS (ionspray; [M+H]⁻) m/z: 516.2. HRMS for C₂₇H₃₃F₂N₃O₃: Calcd, 517.2211; found, 517.2217.

### EXAMPLE 312

### N-[(3aS*,6R*,7aS*)-1-cyclobutyl-3a-(3,4-dimethoxyphenyl)octahydro-1H-indol-6-yl]-N′-(3,4-difluorophenyl)urea trifluoroacetate

General procedure for reductive amination, see Example 260.

Reagent: cyclobutanone (0.009 g, 0.115 mmol)

The mixture was stirred for 17 hours and filtered. The crude product was purified by reversed phase HPLC. Isolated as the TFA salt. Yield 1.7 mg (10%). Colorless liquid. MS (ionspray; [M+H]⁺) m/z: 486.4. HRMS for C₂₇H₃₃F₂N₃O₃: Calcd, 485.2490; found, 485.2478.

### EXAMPLE 313

### N-[(3aS*,6R*,7aS*)-3a-(3,4-dimethoxyphenyl)-1-methyloctahydro-1H-indol-6-yl]-N′-[4-methoxy-3-(trifluoromethyl)phenyl]urea trifluoroacetate

General method for urea formation:

Triethyl amine was added (33 μL, 0.24 mmol) to a solution of a phenylamine (0.14 mmol) in dry CH₂Cl₂ (1.5 mL) under an atmosphere of nitrogen. Triphosgene (14 mg, 0.048 mmol) was dissolved in dry CH₂Cl₂ (0.5 mL) and added drop-wise. The reaction was stirred at room temperature for 2 hrs. (3aS*,6R*,7aS*)-3a-(3,4-dimethoxyphenyl)-1-methyloctahydro-1H-indol-6-amine (Comparative Example 7) (40 mg, 0.14 mmol) dissolved in dry CH₂Cl₂ (1.0 mL) was added and the reaction was stirred over night at room temperature. After solvent evaporation, the crude product was dissolved in CH₃CN/H₂O and purified using preparative HPLC.

Reagent: 4-methoxy-3-trifluoromethyl-phenylamine (25 mg). The crude product was dissolved in CH₃CN (1.5 mL)/H₂O (1.0 mL) and purified using preparative HPLC to give 60 mg (69%) of the title compound as a colorless solid.

MS (ESI+) for C₂₆H₃₂F₃N₃O₄ m/z 508 (M+H)⁺.

HRMS (EI) calcd for C₂₆H₂₉F₃N₄O₃: 507.2345, found 507.2343.

### EXAMPLE 314

### N-(3-chloro-4-morpholin-4-ylphenyl)-N′-[(3aS*,6R*,7aS*)-3a-(3,4-dimethoxyphenyl)-1-methyloctahydro-1H-indol-6-yl]urea trifluoroacetate

Synthesis was performed from 3-chloro-4-morpholin-4-yl-phenylamine (30 mg) using the general method described in Example 313. The crude product was dissolved in CH₃CN (3.0 mL)/H₂O (0.5 mL) and purified using preparative HPLC to give 50 mg (55%) of the title compound as a colorless solid.

MS (ESI+) for C₂₈H₃₇Cl N₄ m/z 529 (M+H)⁺.

HRMS (EI) calcd for C₂₈H₃₇Cl N₄: 528.2503, found 528.2496.

### EXAMPLE 315

### N-[(3aS*,6R*,7aS*)-3a-(3,4-dimethoxyphenyl)-1-methyloctahydro-1H-indol-6-yl]-N′-[3-methoxy-5-(trifluoromethyl)phenyl]urea trifluoroacetate

General method for urea formation from Example 313.

Reagent: 3-ethoxy-5-trifluoromethyl-phenylamine (27 mg).

The crude product was dissolved in CH₃CN (2.0 mL)/H₂O (0.5 mL) and purified using preparative HPLC to give 63 mg (72%) of the title compound as a colorless solid.

MS (ESI+) for C₂₆H₃₂F₃N₃O₄ m/z 508 (M+H)⁺.

HRMS (EI) calcd for C₂₆H₂₉F₃N₄O₃: 507.2345, found 507.2363.

### EXAMPLE 316

### N-[4-cyano-3-(trifluoromethyl)phenyl]-N′-[(3aS*,6R*,7aS*)-3a-(3,4-dimethoxyphenyl)-1-methyloctahydro-1H-indol-6-yl]urea trifluoroacetate

General method for urea formation from Example 313.

Reagent: 4-methylamino-2-trifluoromethylbenzonitrile (26 mg). The crude product was dissolved in CH₃CN (1.0 mL)/H₂O (0.5 mL) and purified using preparative HPLC to give 36 mg (42%) of the title compound as a colorless solid.

MS (ESI+) for C₂₆H₂₉F₃N₄O₃ m/z 503 (M+H)⁺.

HRMS (EI) calcd for C₂₆H₂₉F₃N₄O₃: 502.2192, found 502.2207.

### EXAMPLE 317

### N-[(3aS*,6R*,7aS*)-3a-(3,4-dimethoxyphenyl)-1-methyloctahydro-1H-indol-6-yl]-N′-(3,4,5-trifluorophenyl)urea trifluoroacetate

General method for urea formation from Example 313.

Reagent: methyl-(3,4,5-trifluoro-phenyl)amine (21 mg). The crude product was dissolved in CH₃CN (1.0 mL)/H₂O (0.5 mL) and purified using preparative HPLC to give 36 mg (74%) of the title compound as a colorless solid.

MS (ESI+) for C₂₄H₂₈F₃N₃O₃ m/z 464 (M+H)⁺.

HRMS (EI) calcd for C₂₆H₂₉F₃N₄O₃: 463.2083, found 463.2065.

### EXAMPLE 318

### N-[(3aS*,6R*,7aS*)-3a-(3,4-dimethoxyphenyl)-1-methyloctahydro-1H-indol-6-yl]-N′-[3-(1H-tetrazol-5-yl)phenyl]urea trifluoroacetate

General method for urea formation from Example 313.

Reagent: 3-(2H-tetrazol-5-yl)-phenylamine (23 mg). The crude product was dissolved in CH₃CN (1.5 mL)/H₂O (0.5 mL) and purified using preparative HPLC to give 70 mg (83%) of the title compound as a colorless solid.

¹H NMR (500 MHz, MeOH-d₄)

MS (ESI+) for C₂₅H₃₁N₇O₃ m/z 478 (M+H)⁺.

HRMS (EI) calcd for C₂₆H₂₉F₃N₄O₃: 477.2488, found 477.2485.

### EXAMPLE 319

### N-[(3aS*,6R*,7aS*)-3a-(3,4-dimethoxyphenyl)-1-methyloctahydro-1H-indol-6-yl]-N′-{3-[(trifluoromethyl)sulfonyl]phenyl}urea trifluoroacetate

Synthesis was performed from 3-trifluoromethanesulfonyl-phenylamine (32 mg) using the general method described in Example 313. The crude product was dissolved in CH₃CN (1.5 mL)/H₂O (0.5 mL) and purified using preparative HPLC to give 72 mg (78%) of the title compound as a colorless solid.

¹H NMR (500 MHz, CDCl₃-d) δ 1.05-1.35 (m, 1H) 1.80-2.12 (m, 3H) 2.15-2.35 (m, 3H) 2.40-2.85 (m, 2H) 3.01-3.25 (m, 4H) 3.75-3.92 (m, 1H) 3.85 (s, 3H) 3.85 (s, 3H) 4.01-4.17 (m, 1H) 6.63-6.73 (m, 2H) 6.74-6.80 (m, 1H) 7.47-7.67 (m, 2H) 8.49-8.70 (m, 1H) 8.90-9.07 (m, 1H).

MS (ESI+) for C₂₅H₃₀F₃N₃O₅ m/z 542 (M+H)⁺.

HRMS (EI) calcd for C₂₅H₃₀F₃N₃O₅: 541.1858, found 541.1842.

### EXAMPLE 320

### N-1,3-benzothiazol-6-yl-N′-[(3aS*,6R*,7aS*)-3a-(3,4-dimethoxyphenyl)-1-methyloctahydro-1H-indol-6-yl]urea trifluoroacetate

Synthesis was performed from benzothiazol-6-ylamine (21 mg) using the general method described in Example 313. The crude product was dissolved in CH₃CN (1.0 mL)/H₂O (1.5 mL) and purified using preparative HPLC to give 55 mg (68%) of the title compound as a colorless solid.

MS (ESI+) for C₂₅H₃₀N₄O₃ m/z 467 (M+H)⁺.

HRMS (EI) calcd for C₂₅H₃₀N₄O₃: 466.2039, found 466.2037.

### EXAMPLE 321

### N-[(3aS*,6R*,7aS*)-3a-(3,4-dimethoxyphenyl)-1-methyloctahydro-1H-indol-6-yl]-N′-[4-(methylsulfonyl)phenyl]urea trifluoroacetate

Triethyl amine was added (50 μL, 0.36 mmol) to a solution of 4-methanesulfonyl-phenylamine hydrochloride (29 mg, 0.14 mmol) in dry CH₂Cl₂ (1.5 mL) under an atmosphere of nitrogen. Triphosgene (14 mg, 0.048 mmol) dissolved in dry CH₂Cl₂ (0.5 mL) was added drop-wise where after the reaction was stirred at room temperature for 2 hrs. (3aS*,6R*,7aS*)-3a-(3,4-dimethoxyphenyl)-1-methyloctahydro-1H-indol-6-amine (Comparative Example 7) (40 mg, 0.14 mmol) dissolved in dry CH₂Cl₂ (1.0 mL) was added and the reaction was stirred over night at room temperature. After solvent evaporation, the crude product was dissolved in CH₃CN (1.5 mL)/H₂O (1.5 mL) and purified using preparative HPLC to give 69 mg (82%) of the title compound as a colorless solid.

MS (ESI+) for C₂₅H₃₃N₃O₅S m/z 488 (M+H)⁺.

HRMS (EI) calcd for C₂₅H₃₃N₃O₅S: 487.2141, found 487.2145.

### EXAMPLE 322

### N-(3,4-difluorophenyl)-N′-[(3aS*,6R*,7aS*)-3a-(3,4-dihydroxyphenyl)-1-methyloctahydro-1H-indol-6-yl]urea hydrobromide

BBr₃ (2.0 ml, 2.0 mmol) was added to a solution of N-(3,4-difluorophenyl)-N′-[(3aS*,6R*,7aS*)-3a-(3,4-dimethoxyphenyl)-1-methyloctahydro-1H-indol-6-yl]urea 2-methoxyphenyl benzoate (2-methoxyphenyl benzoate salt of Example 117) (0.7 g, 1.6 mmol) in CH₂Cl₂ (40 ml). (A white precipitate was formed). After 30 minutes additional BBr₃ (2.0 ml, 2.0 mmol) was added and the mixture was stirred for additional 2 hours. MeOH was added dropwise and the mixture was evaporated. The residue (orange oil) was taken up in EtOAc and a white powder precipitated. The mixture was filtered and the white crystals were washed with CH₂Cl₂ and EtOAc. Isolated as the hydrobromide.

Yield 0.7 g (90%). ¹H NMR (400 MHz, DMSO-D6) δ ppm 0.95-1.11 (m, 1H) 1.54-1.82 (m, 4H) 1.98 (s, 1H) 1.99-2.15 (m, 3H) 2.29 (d, J=15.06 Hz, 1H) 2.96 (d, J=4.52 Hz, 3H) 3.16-3.28 (m, 1H) 3.66-3.79 (m, 1H) 3.89-4.00 (m, 2H) 6.31-6.42 (m, 1H) 6.67 (dd, J=8.41, 2.13 Hz, 1H) 6.72-6.74 (m, 1H) 6.80 (d, J=2.01 Hz, 1H) 6.96-7.05 (m, 1H) 7.20-7.30 (m, 1H) 7.56-7.65 (m, 1H) 8.65 (s, 1H) 9.53 (s, 1H). MS (ionspray; [M+H]⁺) m/z: 418.2.

### EXAMPLE 323

### N-(3,4-Difluorophenyl)-N′-[(3aS*,6R*,7aS*)-3a-(4-hydroxy-3-methoxyphenyl)-1-methyloctahydro-1H-indol-6-yl]urea hydrochloride

N-{(3aS*,6R*,7aS*)-3a-[4-(benzyloxy)-3-methoxyphenyl]-1-methyloctahydro-1H-indol-6-yl}-N′-(3,4-difluorophenyl)urea (Example 250) (340 mg, 0.652 mmol) was dissolved in EtOH (15 ml), 1 M HCl in Et₂O (3 ml) and AcOH (3 ml) was added. 10% Palladium on charcoal (spatula) was added and mixture shaken in Parr Apparatus (−5 bar H₂) overnight. HPLC indicated 77% conversion. 1 M HCl in Et₂O (4 ml) and more 10% palladium on charcoal (spatula) was added, and mixture shaken in Parr apparatus for 2.5 h. Mixture filtered through Celite and washed with EtOH. Et₂O added to filtrate and white material precipitated. Yield 162 mg (53%); white solid. ¹H NMR (400 MHz, DMSO-d₆) δ 1.02-1.06 (m, 1H) 1.56-2.31 (m, 8H) 2.93 (d, J=3.5 Hz, 3H) 3.79 (s, 3H) 3.754-10 (m, 3H) 6.51 (d, J=7.3 Hz, 1H) 6.76-76.82 (m, 1H) 6.93 (s, 1H) 7.02 (s, 1H) 7.20-7.28 (m, 1H) 7.59-7.64 (m, 1H) 8.97 (s, 1H) 9.02 (s, 1H) 10.16 (s, 1H). MS (ESI+) for C₂₃H₂₇F₂N₃O₃ m/z 432 (M+1). HRMS (EI) calcd for C₂₃H₂₇F₂N₃O₃: 431.202, found 431.2034.

### EXAMPLE 324

### N-2,1,3-benzothiadiazol-4-yl-N′-[(3aS*,6R*,7aS*)-3a-(3,4-dimethoxyphenyl)-1-methyloctahydro-1H-indol-6-yl]urea trifluoroacetate

Reagent: 2,1,3-benzothiadiazol-4-yl isocyanate

Synthetic procedure: Scheme E

Yield: 4.3 mg (21%)

Measured mass: 467.1998

Calc. mass: 467.1991

### EXAMPLE 325

### N-[(3aS*,6R*,7aS*)-3a-(3,4-dimethoxyphenyl)-1-methyloctahydro-1H-indol-6-yl]-N′-[3-(trifluoromethyl)phenyl]thiourea trifluoroacetate

Reagent: 3-(trifluoromethyl)phenyl isothiocyanate

Synthetic procedure: Scheme E

Yield: 6.1 mg (29%)

Measured mass: 493.2005

Calc. mass: 493.2011

### EXAMPLE 326

### N-[(3aS*,6S*,7aS*)-3a-(1,3-benzodioxol-5-yl)-1-benzyloctahydro-1H-indol-6-yl]-N′-(3-chlorophenyl)urea and

### EXAMPLE 327

### N-[(3aS*,6R*,7aS*)-3a-(1,3-benzodioxol-5-yl)-1-benzyloctahydro-1H-indol-6-yl]-N′-(3-chlorophenyl)urea

LiNH₂ (7.5 g, 328 mmol) was suspended in DME (200 mL) at ambient temperature and (3,4-methylenedioxy)phenylacetonitrile (20 g, 124 mmol) in DME (50 mL) was added portionwise over 15 min. The mixture was heated at 80° C. for 30 min, whereupon its color changed to green, before a solution of 1-bromo-2-chloroethane (11.3 mL, 136 mmol) in DME (50 mL) was added over a period of 20 min. During the course of the addition, the green color of the mixture changed to light brown. The mixture was heated at 80° C. overnight, or until GC indicated >95% consumption of the starting material. The mixture was cooled on an ice/water bath, and water (200 mL) and Et₂O (400 ml) was then added to destroy the excess of strong base. The mixture was extracted with DCM (2×100 mL), and the combined organic extracts were washed with H₂O (100 mL), dried (MgSO₄) and evaporated. The residue was purified by flash chromatography (silica, 10-20% EtOAc in n-heptane) to yield 1-(3,4-methylenedioxyphenyl)cyclopropanecarbonitrile (19.0 g, 82%) as a yellowish oil. ¹H NMR (270 MHz, CDCl₃) δ ppm 1.18-1.32 (m, 2H); 1.57-1.66 (m, 2H); 5.93 (s, 2H); 6.71-6.83 (m, 3H).

1-(3,4-methylenedioxyphenyl)cyclopropanecarbonitrile (19.0 g of crude material, 101 mmol), was dissolved in dry toluene (800 mL) and cooled on an ice/water bath. A solution of DIBAL (1M in toluene, 140 mL, 140 mmol) was added dropwise via an addition funnel, over a period of 30 min. The resulting mixture was heated at 50° C. overnight. The reaction mixture was cooled to 0° C. and cautiously transferred, in small portions and with swirling, to a separatory funnel containing ice-cold aq. HCl (4M, 0.5 L). The aqueous layer was extracted once with EtOAc (400 mL) and the combined organic portions were washed with water (1×300 mL) and brine (1×200 mL), dried (MgSO₄) and concentrated to give the aldehyde (19 g) as a yellowish oil, which was used in the subsequent step without further purification. ¹H NMR (270 MHz, CDCl₃) δ 1.32-1.37 (m, 2H); 1.49-1.55 (m, 2H); 5.95 (s, 2H); 6.69-6.83 (m, 3H); 9.18 (s, 1H).

To a solution of the aldehyde (19 g of crude material, assumed to be 101 mmol) in dry THF (800 m-L) was added benzylamine (11.9 g, 111 mmol) and an excess of MgSO₄ (50 g) and the resulting mixture was stirred at rt during 24 h. The mixture was filtered and evaporated to give the imine (28 g), which was used in the next step without further purification. ¹H NMR (270 MHz, CDCl₃) δ 1.12-1.17 (m, 2H); 1.30-1.36 (m, 2H); 4.56 (s, 2H); 5.93 (s, 2H); 6.69-6.89 (m, 3H); 7.14-7.40 (m, 5H); 7.70 (s, 1H).

To a solution of imine (10 g of crude material, assumed to be 53 mmol) and benzylamine hydrochloride (10 g, 68 mmol) in MeCN (400 mL) was added Na₂SO₄ (20 g) and but-3-en-2-one (3.7 g, 53 mmol). The mixture was heated at reflux for 5 h and then cooled to rt. The drying agent was filtered off and the filtrate was evaporated to dryness. The residue was partitioned between EtOAc (200 mL) and saturated aqueous NaHCO₃ (100 mL), and the aqueous layer was extracted with EtOAc (2×100 mL). The combined organic portions were washed with brine (100 mL), dried (MgSO₄) and concentrated to give 3a-benzo[1,3]dioxol-5-yl-1-benzyl-octahydroindol-6-one as a colorless oil (29%) after purification by column chromatography (silica/hexanes: EtOAc 70:30).

¹H NMR (270 MHz, CDCl₃) δ 1.85-2.36 (m, 6H); 2.40-2.82 (m, 3H); 2.87-2.98 (m, 1H); 3.10 (d, 1H, J=12.1 Hz); 3.21-3.26 (m, 1H); 4.08 (d, 1H, J=12.1 Hz); 5.93 (s, 2H); 6.71-6.95 (m, 3H); 7.17-7.41 (m, 5H). ¹³C NMR (67.9 MHz, CDCl₃) δ 34.86, 36.08, 38.45, 40.36, 47.24, 51.46, 57.30, 68.24, 100.88, 106.73, 107.86, 118.48, 126.75, 128.02, 128.57, 138.78, 141.52, 145.63, 147.86, 210.96.

To a solution of 3a-benzo[1,3]dioxol-5-yl-1-benzyl-octahydroindol-6-one (1.5 g, 4.30 mmol) and ammonium formate (2 g, 38 mmol) in MeOH (100 mL) was added NaBH₃CN (2 g, 32 mmol) in portions during 5 min. The resulting mixture was stirred at rt for 4 h and then evaporated. The residue was partitioned between EtOAc (100 mL) and saturated aqueous NaHCO₃ (50 mL). The aqueous phase was extracted with EtOAc (2×50 mL) and the combined organic fractions were washed with brine (50 mL), dried (MgSO₄) and evaporated to give 3a-benzo[1,3]dioxol-5-yl-1-benzyl-octahydro-indol-6-ylamine as a colorless oil (1.3 g, 87%), which was used in the next step without further purification. ¹H NMR indicated the formation of approximately a 1:1 mixture of diastereomers (at C-6).

¹H NMR (270 MHz, CDCl₃) δ 0.89-2.42 (m, 11H); 2.83-3.30 (m, 3H); 4.18 (d, 0.5H, J=13.7 Hz); 4.30 (d, 0.5H, J=13.0 Hz); 5.88 (s, 2H); 6.64-7.00 (m, 3H); 7.10-7.53 (m, 5H).

To a solution of 3a-benzo[1,3]dioxol-5-yl-1-benzyl-octahydro-indol-6-ylamine (50 mg, 0.143 mmol) in CHCl₃ (2 mL) was added 3-chlorophenylisocyanate (0.025 mL; 0.20 mmol) and the resulting solution was stirred overnight at rt. The two diastereoisomers were separated by column chromatography (silica/CHCl₃ sat. with NH₃), affording, in order of elution, N-[(3aS*,6S*,7aS*)-3a-(1,3-benzodioxol-5-yl)-1-benzyloctahydro-1H-indol-6-yl]-N′-(3-chlorophenyl)urea (24 mg, 33%) and N-[(3aS*,6R*,7aS*)-3a-(1,3-benzodioxol-5-yl)-1-benzyloctahydro-1H-indol-6-yl]-N′-(3-chlorophenyl)urea (21 mg, 29%) as colorless oils. Assignments of relative stereochemistry were made by nÖe NMR spectroscopy.

N-[(3aS*,6S*,7aS*)-3a-(1,3-benzodioxol-5-yl)-1-benzyloctahydro-1H-indol-6-yl]-N′-(3-chlorophenyl)urea (eluting first on silica gel): ¹H NMR (500 MHz, CHLOROFORM-D) δ ppm 1.13 (m, 1H) 1.43 (ddd, J=14.65, 11.96, 2.93 Hz, 1H) 1.74 (m, J=12.70, 11.48, 6.10 Hz, 1H) 1.79 (ddd, J=12.94, 8.55, 5.13 Hz, 1H) 1.89 (m, 1H) 1.99 (m, J=13.92 Hz, 1H) 2.23 (td, J=13.79, 2.93 Hz, 1H) 2.28 (m, 2H) 3.05 (td, J=9.03, 6.35 Hz, 1H) 3.07 (s, 1H) 3.19 (d, J=12.94 Hz, 1H) 4.19 (m, 1H) 4.24 (d, J=13.18 Hz, 1H) 4.33 (d, J=7.81 Hz, 1H) 5.94 (s, 2H) 6.14 (s, 1H) 6.77 (d, J=8.30 Hz, 1H) 6.83 (dd, J=8.06, 1.71 Hz, 1H) 6.87 (d, J=1.71 Hz, 1H) 6.99 (ddd, J=7.87, 1.77, 1.10 Hz, 1H) 7.10 (d, J=8.79 Hz, 1H) 7.16 (t, J=7.93 Hz, 1H) 7.23 (m, J=7.32, 7.32 Hz, 1H) 7.32 (m, 2H) 7.34 (m, 1H) 7.40 (m, J=7.32 Hz, 2H).

MS (ESI+) m/z 504 (M+1).

HRMS (EI) calc for C₂₉H₃₀ClN₃O₃: 503.1976; found 503.1985.

N-[(3aS*,6R*,7aS*)-3a-(1,3-benzodioxol-5-yl)-1-benzyloctahydro-1H-indol-6-yl]-N′-(3-chlorophenyl)urea (eluting slower on silica gel): ¹H NMR (500 MHz, CDCl₃) δ ppm 1.13 (m, 1H) 1.43 (ddd, J=14.65, 11.96, 2.93 Hz, 1H) 1.74 (m, J=12.70, 11.48, 6.10 Hz, 1H) 1.79 (ddd, J=12.94, 8.55, 5.13 Hz, 1H) 1.89 (m, 1H) 1.99 (m, J=13.92 Hz, 1H) 2.23 (td, J=13.79, 2.93 Hz, 1H) 2.28 (m, 2H) 3.05 (td, J=9.03, 6.35 Hz, 1H) 3.07 (s, 1H) 3.19 (d, J=12.94 Hz, 1H) 4.19 (m, 1H) 4.24 (d, J=13.18 Hz, 1H) 4.33 (d, J=7.81 Hz, 1H) 5.94 (s, 2H) 6.14 (s, 1H) 6.77 (d, J=8.30 Hz, 1H) 6.83 (dd, J=8.06, 1.71 Hz, 1H) 6.87 (d, J=1.71 Hz, 1H) 6.99 (ddd, J=7.87, 1.77, 1.10 Hz, 1H) 7.10 (d, J=8.79 Hz, 1H) 7.16 (t, J=7.93 Hz, 1H) 7.23 (m, J=7.32, 7.32 Hz, 1H) 7.32 (m, 2H) 7.34 (m, 1H) 7.40 (m, J=7.32 Hz, 2H).

MS (ESI+) m/z 504 (M+1).

HRMS (EI) calc for C₂₉H₃₀ClN₃O₃: 503.1976; found 503.1990.

### EXAMPLE 328

### N-[(3aS*,6R*,7aS*)-3a-(1,3-benzodioxol-5-yl)-1-methyloctahydro-1H-indol-6-yl]-N′-[3-(trifluoromethyl)phenyl]urea

To a solution of 3a-benzo[1,3]dioxol-5-yl-1-benzyl-octahydro-indol-6-ylamine, intermediate from Example 327 (20 mg, 0.057 mmol) in THF (1 mL) was added 4-chloro-3-(trifluoromethyl)phenyl isocyanate (15 mg, 0.068 mmol) and the resulting mixture was stirred at rt overnight. The reaction mixture was then transferred to a flask containing MeOH (2 mL) and 10% Pd/C (50 mg). The resulting suspension was vigorously stirred under H₂ (1 atm) for 24 hrs. The catalyst was then filtered off, before formaldehyde (0.5 mL of a 40% aq solution) and NaBH₃CN (50 mg, 0.8 mmol) were added. The resulting solution was stirred another 30 min, and then concentrated at reduced pressure. The residue was partitioned between EtOAc (5 mL) and 5% aq. NaHCO₃ (2 mL). The aqueous phase was extracted with CHCl₃ (2 mL) and the combined organics were dried (MgSO₄) and evaporated. The residue was purified by column chromatography (silica/CHCl₃ sat. with NH₃) to give the title as a colorless oil (8 mg, 30%). The relative stereochemistry was determined by nÖe NMR spectroscopy.

¹H NMR (500 MHz, DMSO-D6) δ ppm 1.00 (qd, J=12.17, 4.27 Hz, 1H) 1.36 (ddd, J=14.40, 11.23, 3.17 Hz, 1H) 1.64 (m, 1H) 1.65 (ddd, J=12.21, 11.47, 4.88 Hz, 1H) 1.82 (ddd, J=12.70, 8.79, 6.10 Hz, 1H) 1.95 (d, J=9.28 Hz, 1H) 1.99 (m, 1H) 2.02 (m, 1H) 2.24 (m, 1H) 2.28 (s, 3H) 2.66 (m, 1H) 3.13 (td, J=9.09, 4.76 Hz, 1H) 3.78 (m, 1H) 5.97 (s, 2H) 6.09 (d, J=8.06 Hz, 1H) 6.84 (s, 2H) 6.95 (s, 1H) 7.18 (d, J=7.57 Hz, 1H) 7.40 (t, J=7.93 Hz, 1H) 7.45 (m, 1H) 7.93 (s, 1H) 8.56 (s, 1H).

MS (ESI+) m/z 462 (M+1).

### COMPARATIVE EXAMPLE 329

### (3aS*,6R*,7aS*)-3a-(3,4-dimethoxyphenyl)-1-(1-methylpiperidin-4-yl)octahydro-1H-indol-6-amine

A solution of tert-butyl [(3aS*,6R*,7aS*)-3a-(3,4-dimethoxyphenyl)octahydro-1H-indol-6-yl]carbamate acetate, intermediate from Comparative Example 10, (380 mg; 1.0 mmol) and an aldehyde or ketone (1.0 mmol) in dichloroethane (10 ml) was treated with sodium triacetoxyborohydride (297 mg; 1.4 mmol) and the mixture stirred at room temperature overnight. The resulting solution was treated with water (5.0 ml) then 2M sodium hydroxide (5.0 ml) then extracted with ether to afford a colourless gum. The gum was treated with a 50% v/v solution of trifluoroacetic acid in dichloromethane (10.0 ml) and allowed to stand for 30-40 min. The mixture was evaporated and the residue partitioned between 2M sodium hydroxide and ethyl acetate. Evaporation of the organic phase gave the product.

Thus, 1-methylpiperidin-4-one gave (3aS*,6R*,7aS*)-3a-(3,4-dimethoxyphenyl)-1-(1-methylpiperidin-4-yl)octahydro-1H-indol-6-amine. Yield=220 mg (47%) used without further purification

### COMPARATIVE EXAMPLE 330

### (3aS*,6R*,7aS*)-3a-(3,4-dimethoxyphenyl)-1-isopropyloctahydro-1H-indol-6-amine

Procedure as in Comparative Example 329.

Acetone gave (3aS*,6R*,7aS*)-3a-(3,4-dimethoxyphenyl)-1-isopropyloctahydro-1H-indol-6-amine. Yield=260 mg (82%) used without further purification

### COMPARATIVE EXAMPLE 331

### (3aS*,6R*,7aS*)-1-cyclohexyl-3a-(3,4-dimethoxyphenyl)octahydro-1H-indol-6-amine

Procedure as in Comparative Example 329.

Cyclohexanone gave (3aS*,6R*,7aS*)-1-cyclohexyl-3a-(3,4-dimethoxyphenyl)octahydro-1H-indol-6-amine. Yield=215 mg (60%) used without further purification.

### COMPARATIVE EXAMPLE 332

### (3aS*,6R*,7aS*)-3a-(3,4-dimethoxyphenyl)-1-(tetrahydro-2H-pyran-4-yl)octahydro-1H-indol-6-amine

Procedure as in Comparative Example 329.

Tetrahydropyran-4-one gave (3aS*,6R*,7aS*)-3a-(3,4-dimethoxyphenyl)-1-(tetrahydro-2H-pyran-4-yl)octahydro-1H-indol-6-amine. Yield=295 mg (82%) used without further purification.

### COMPARATIVE EXAMPLE 333

### (3aS*,6R*,7aS*)-1-cyclobutyl-3a-(3,4-dimethoxyphenyl)octahydro-1H-indol-6-amine

Procedure as in Comparative Example 329.

Cyclobutanone gave (3aS*,6R*,7aS*)-1-cyclobutyl-3a-(3,4-dimethoxyphenyl)octahydro-1H-indol-6-amine. Yield=230 mg (70%) used without further purification.

### COMPARATIVE EXAMPLE 334

### (3aS*,6R*,7aS*)-3a-(3,4-dimethoxyphenyl)-1-propyloctahydro-1H-indol-6-amine

Procedure as in Comparative Example 329.

Propan-1-al gave (3aS*,6R*,7aS*)-3a-(3,4-dimethoxyphenyl)-1-propyloctahydro-1H-indol-6-amine. Yield=241 mg (78%), used without further purification.

### COMPARATIVE EXAMPLE 335

### (3aS*,6R*,7aS*)-1-cyclopropylmethyl-3a-(3,4-dimethoxyphenyl)octahydro-1H-indol-6-amine

Procedure as in Comparative Example 329.

Cyclopropylcarboxaldehyde gave (3aS*,6R*,7aS*)-1-cyclopropylmethyl-3a-(3,4-dimethoxyphenyl)octahydro-1H-indol-6-amine. Yield=209 mg (63%), used without further purification.

### EXAMPLE 336

### N-(3-bromophenyl)-N′-[(3aS*,6R*,7aS*)-3a-(3,4-dimethoxyphenyl)-1-isopropyloctahydro-1H-indol-6-yl]urea trifluoroacetate

A solution of (3aS*,6R*,7aS*)-3a-(3,4-dimethoxyphenyl)-1-isopropyloctahydro-1H-indol-6-amine, Comparative Example 330, (0.03 mmol) in tetrahydrofuran (2.0 ml) was treated with the appropriate arylisocyanate (0.03 mmol) and allowed to stand at room temperature overnight. The solvent was removed by evaporation and the product isolated by preparative HPLC (YMC column: gradient 20-65% MeCN in water containing 0.1% trifluoroacetic acid).

Reagent: 3-bromophenylisocyanate

Yield: 7.5 mg

Calculated mass: 515.1784

Measured mass: 515.1774

### EXAMPLE 337

### N-[(3aS*,6R*,7aS*)-3a-(3,4-dimethoxyphenyl)-1-isopropyloctahydro-1H-indol-6-yl]-N′-(2,3,4-trifluorophenyl)urea trifluoroacetate

Procedure as in Example 336

Reagent: 2,3,4-trifluorophenylisocyanate

Yield: 4.3 mg

Calculated mass: 491.2396

Measured mass: 491.2380

### EXAMPLE 338

### N-(3,5-difluorophenyl)-N′-[(3aS*,6R*,7aS*)-3a-(3,4-dimethoxyphenyl)-1-isopropyloctahydro-1H-indol-6-yl]urea trifluoroacetate

Procedure as in Example 336

Reagent: 3,5-difluorophenylisocyanate

Yield: 9.8 mg

Calculated mass: 473.2490

Measured mass: 473.2488

### EXAMPLE 339

### N-[3-chloro-4-(trifluoromethyl)phenyl]-N′-[(3aS*,6R*,7aS*)-3a-(3,4-dimethoxyphenyl)-1-isopropyloctahydro-1H-indol-6-yl]urea trifluoroacetate

Procedure as in Example 336

Reagent: 3-chloro-4-(trifluoromethyl)phenylisocyanate

Yield: 9.1 mg

Calculated mass: 539.2163

Measured mass: 539.2164

### EXAMPLE 340

### N-[(3aS*,6R*,7aS*)-3a-(3,4-dimethoxyphenyl)-1-isopropyloctahydro-1H-indol-6-yl]-N′-(3,5-dimethylphenyl)urea trifluoroacetate

Procedure as in Example 336

Reagent: 3,5-dimethylphenylisocyanate

Yield: 4.8 mg

Calculated mass: 465.2991

Measured mass: 465.2968

### EXAMPLE 341

### N-[(3aS*,6R*,7aS*)-3a-(3,4-dimethoxyphenyl)-1-isopropyloctahydro-1H-indol-6-yl]-N′-[4-(trifluoromethyl)phenyl]urea trifluoroacetate

Procedure as in Example 336

Reagent: 4-(trifluoromethyl)phenylisocyanate

Yield: 4.8 mg

Calculated mass: 505.2552

Measured mass: 505.2533

### EXAMPLE 342

### N-[(3aS*,6R*,7aS*)-3a-(3,4-dimethoxyphenyl)-1-isopropyloctahydro-1H-indol-6-yl]-N′-[3-(trifluoromethyl)phenyl]urea trifluoroacetate

Procedure as in Example 336

Reagent: 3-(trifluoromethyl)phenylisocyanate

Yield: 6.4 mg

Calculated mass: 505.2552

Measured mass: 505.2559

### EXAMPLE 343

### N-[(3aS*,6R*,7aS*)-3a-(3,4-dimethoxyphenyl)-1-isopropyloctahydro-1H-indol-6-yl]-N′-[4-fluoro-3-(trifluoromethyl)phenyl]urea trifluoroacetate

Procedure as in Example 336

Reagent: 4-fluoro-3-(trifluoromethyl)phenylisocyanate

Yield: 0.3 mg

Calculated mass: 523.2458

Measured mass: 523.2435

### EXAMPLE 344

### N-[(3aS*,6R*,7aS*)-3a-(3,4-dimethoxyphenyl)-1-isopropyloctahydro-1H-indol-6-yl]-N′-[3-fluoro-5-(trifluoromethyl)phenyl]urea trifluoroacetate

Procedure as in Example 336

Reagent: 3-fluoro-5-(trifluoromethyl)phenylisocyanate

Yield: 7.1 mg

Calculated mass: 523.2458

Measured mass: 523.2354

### EXAMPLE 345

### N-(5-chloro-2-methylphenyl)-N′-[(3aS*,6R*,7aS*)-3a-(3,4-dimethoxyphenyl)-1-isopropyloctahydro-1H-indol-6-yl]urea trifluoroacetate

Procedure as in Example 336

Reagent: 5-chloro-2-methylphenylisocyanate

Yield: 6.5 mg

Calculated mass: 485.2445

Measured mass: 485.2433

### EXAMPLE 346

### N-[4-chloro-3-(trifluoromethyl)phenyl]-N′-[(3aS*,6R*,7aS*)-3a-(3,4-dimethoxyphenyl)-1-isopropyloctahydro-1H-indol-6-yl]urea trifluoroacetate

Procedure as in Example 336

Reagent: 4-chloro-3-(trifluoromethyl)phenylisocyanate

Yield: 8.3 mg

Calculated mass: 539.2163

Measured mass: 539.2161

### EXAMPLE 347

### N-(2,4-difluorophenyl)-N′-[(3aS*,6R*,7aS*)-3a-(3,4-dimethoxyphenyl)-1-isopropyloctahydro-1H-indol-6-yl]urea trifluoroacetate

Procedure as in Example 336

Reagent: 2,4-difluorophenylisocyanate

Yield: 3.3 mg

Calculated mass: 473.2490

Measured mass: 473.2470

### EXAMPLE 348

### N-[(3aS*,6R*,7aS*)-3a-(3,4-dimethoxyphenyl)-1-isopropyloctahydro-1H-indol-6-yl]-N′-(4-fluorophenyl)urea trifluoroacetate

Procedure as in Example 336

Reagent: 4-fluorophenylisocyanate

Yield: 7.6 mg

Calculated mass: 455.2584

Measured mass: 455.2581

### EXAMPLE 349

### N-[(3aS*,6R*,7aS*)-3a-(3,4-dimethoxyphenyl)-1-isopropyloctahydro-1H-indol-6-yl]-N′-[2-fluoro-5-(trifluoromethyl)phenyl]urea trifluoroacetate

Procedure as in Example 336

Reagent: 2-fluoro-5-(trifluoromethyl)phenylisocyanate

Yield: 9.9 mg

Calculated mass: 523.2458

Measured mass: 523.2442

### EXAMPLE 350

### N-[(3aS*,6R*,7aS*)-3a-(3,4-dimethoxyphenyl)-1-isopropyloctahydro-1H-indol-6-yl]-N′-(3-fluoro-4-methylphenyl)urea trifluoroacetate

Procedure as in Example 336

Reagent: 3-fluoro-4-methylphenylisocyanate

Yield: 6.7 mg

Calculated mass: 469.2741

Measured mass: 469.2732

### EXAMPLE 351

### N-[(3aS*,6R*,7aS*)-1-cyclobutyl-3a-(3,4-dimethoxyphenyl)octahydro-1H-indol-6-yl]-N′-[2-fluoro-3-(trifluoromethyl)phenyl]urea trifluoroacetate

A solution of (3aS*,6R*,7aS*)-1-cyclobutyl-3a-(3,4-dimethoxyphenyl)octahydro-1H-indol-6-amine, Comparative Example 333, (0.03 mmol) in tetrahydrofuran (2.0 ml) was treated with the appropriate arylisocyanate (0.03 mmol) and allowed to stand at room temperature overnight. The solvent was removed by evaporation and the product isolated by preparative HPLC (YMC column: gradient 20-65% MeCN in water containing 0.1% trifluoroacetic acid).

Reagent: 2-fluoro-3-(trifluoromethyl)phenylisocyanate

Yield: 12.1 mg

Calculated mass: 535.2458

Measured mass: 535.2436

### EXAMPLE 352

### N-[(3aS*,6R*,7aS*)-1-cyclobutyl-3a-(3,4-dimethoxyphenyl)octahydro-1H-indol-6-yl]-N′-[4-(trifluoromethyl)phenyl]urea trifluoroacetate

Procedure as in Example 351

Reagent: 4-(trifluoromethyl)phenylisocyanate

Yield: 9.6 mg

Calculated mass: 517.2552

Measured mass: 517.2548

### EXAMPLE 353

### N-[(3aS*,6R*,7aS*)-1-cyclobutyl-3a-(3,4-dimethoxyphenyl)octahydro-1H-indol-6-yl]-N′-(3-fluorophenyl)urea trifluoroacetate

Procedure as in Example 351

Reagent: 3-fluorophenylisocyanate

Yield: 11.4 mg

Calculated mass: 467.2584

Measured mass: 467.2597

### EXAMPLE 354

### N-[(3aS*,6R*,7aS*)-1-cyclobutyl-3a-(3,4-dimethoxyphenyl)octahydro-1H-indol-6-yl]-N′-(2,5-difluorophenyl)urea trifluoroacetate

Procedure as in Example 351

Reagent: 2,5-difluorophenylisocyanate

Yield: 11.1 mg

Calculated mass: 485.2490

Measured mass: 485.2505

### EXAMPLE 355

### N-[(3aS*,6R*,7aS*)-1-cyclobutyl-3a-(3,4-dimethoxyphenyl)octahydro-1H-indol-6-yl]-N′-[3-(trifluoromethyl)phenyl]urea trifluoroacetate

Procedure as in Example 351

Reagent: 3-(trifluoromethyl)phenylisocyanate

Yield: 10.2 mg

Calculated mass: 517.2552

Measured mass: 517.2548

### EXAMPLE 356

### N-(3-chloro-4-fluorophenyl)-N′-[(3aS*,6R*,7aS*)-1-cyclobutyl-3a-(3,4-dimethoxyphenyl)octahydro-1H-indol-6-yl]urea trifluoroacetate

Procedure as in Example 351

Reagent: 3-chloro-4-fluorophenylisocyanate

Yield: 11.4 mg

Calculated mass: 501.2194

Measured mass: 501.2211

### EXAMPLE 357

### N-[(3aS*,6R*,7aS*)-1-cyclobutyl-3a-(3,4-dimethoxyphenyl)octahydro-1H-indol-6-yl]-N′-(2,3,4-trifluorophenyl)urea trifluoroacetate

Procedure as in Example 351

Reagent: 2,3,4-trifluorophenylisocyanate

Yield: 2.6 mg

Calculated mass: 503.2396

Measured mass: 503.2379

### EXAMPLE 358

### N-[(3aS*,6R*,7aS*)-1-cyclobutyl-3a-(3,4-dimethoxyphenyl)octahydro-1H-indol-6-yl]-N′-[4-fluoro-3-(trifluoromethyl)phenyl]urea trifluoroacetate

Procedure as in Example 351

Reagent: 4-fluoro-3-(trifluoromethyl)phenylisocyanate

Yield: 1.3 mg

Calculated mass: 535.2458

Measured mass: 535.2450

### EXAMPLE 359

### N-(3-bromophenyl)-N′-[(3aS*,6R*,7aS*)-1-cyclobutyl-3a-(3,4-dimethoxyphenyl)octahydro-1H-indol-6-yl]urea trifluoroacetate

Procedure as in Example 351

Reagent: 3-bromophenylisocyanate

Yield: 11.9 mg

Calculated mass: 527.1784

Measured mass: 527.1779

### EXAMPLE 360

### N-[(3aS*,6R*,7aS*)-1-cyclobutyl-3a-(3,4-dimethoxyphenyl)octahydro-1H-indol-6-yl]-N′-[3-fluoro-5-(trifluoromethyl)phenyl]urea trifluoroacetate

Procedure as in Example 351

Reagent: 3-fluoro-5-(trifluoromethyl)phenylisocyanate

Yield: 9.3 mg

Calculated mass: 535.2458

Measured mass: 535.2457

### EXAMPLE 361

### N-(5-chloro-2-methylphenyl)-N′-[(3aS*,6R*,7aS*)-1-cyclobutyl-3a-(3,4-dimethoxyphenyl)octahydro-1H-indol-6-yl]urea trifluoroacetate

Procedure as in Example 351

Reagent: 5-chloro-2-methylphenylisocyanate

Yield: 10.4 mg

Calculated mass: 497.2445

Measured mass: 497.2442

### EXAMPLE 362

### N-(3-chlorophenyl)-N′-[(3aS*,6R*,7aS*)-1-cyclobutyl-3a-(3,4-dimethoxyphenyl)octahydro-1H-indol-6-yl]urea trifluoroacetate

Procedure as in Example 351

Reagent: 3-chlorophenylisocyanate

Yield: 9.9 mg

Calculated mass: 483.2289

Measured mass: 483.2304

### EXAMPLE 363

### N-[(3aS*,6R*,7aS*)-3a-(3,4-dimethoxyphenyl)-1-(tetrahydro-2H-pyran-4-yl)octahydro-1H-indol-6-yl]-N′-[2-fluoro-3-(trifluoromethyl)phenyl]urea trifluoroacetate

A solution of (3aS*,6R*,7aS*)-3a-(3,4-dimethoxyphenyl)-1-(tetrahydro-2H-pyran-4-yl)octahydro-1H-indol-6-amine, Comparative Example 332, (0.03 mmol) in tetrahydrofuran (2.0 ml) was treated with the appropriate arylisocyanate (0.03 mmol) and allowed to stand at room temperature overnight. The solvent was removed by evaporation and the product isolated by preparative HPLC (YMC column: gradient 20-65% MeCN in water containing 0.1% trifluoroacetic acid).

Reagent: 2-fluoro-3-(trifluoromethyl)phenylisocyanate

Yield: 9.7 mg

Calculated mass: 565.2564

Measured mass: 565.2576

### EXAMPLE 364

### N-[(3aS*,6R*,7aS*)-3a-(3,4-dimethoxyphenyl)-1-(tetrahydro-2H-pyran-4-yl)octahydro-1H-indol-6-yl]-N′-[4-(trifluoromethyl)phenyl]urea trifluoroacetate

Procedure as in Example 363

Reagent: 4-(trifluoromethyl)phenylisocyanate

Yield: 12.4 mg

Calculated mass: 547.2658

Measured mass: 547.2637

### EXAMPLE 365

### N-[(3aS*,6R*,7aS*)-3a-(3,4-dimethoxyphenyl)-1-(tetrahydro-2H-pyran-4-yl)octahydro-1H-indol-6-yl]-N′-(3-fluorophenyl)urea trifluoroacetate

Procedure as in Example 363

Reagent: 3-fluorophenylisocyanate

Yield: 1.1 mg

Calculated mass: 497.2690

Measured mass: 497.2697

### EXAMPLE 366

### N-(2,5-difluorophenyl)-N′-[(3aS*,6R*,7aS*)-3a-(3,4-dimethoxyphenyl)-1-(tetrahydro-2H-pyran-4-yl)octahydro-1H-indol-6-yl]urea trifluoroacetate

Procedure as in Example 363

Reagent: 2,5-difluorophenylisocyanate

Yield: 8.2 mg

Calculated mass: 515.2596

Measured mass: 515.2600

### EXAMPLE 367

### N-[(3aS*,6R*,7aS*)-3a-(3,4-dimethoxyphenyl)-1-(tetrahydro-2H-pyran-4-yl)octahydro-1H-indol-6-yl]-N′-[3-(trifluoromethyl)phenyl]urea trifluoroacetate

Procedure as in Example 363

Reagent: 3-(trifluoromethyl)phenylisocyanate

Yield: 10.8 mg

Calculated mass: 547.2658

Measured mass: 547.2637

### EXAMPLE 368

### N-(3-chloro-4-fluorophenyl)-N′-[(3aS*,6R*,7aS*)-3a-(3,4-dimethoxyphenyl)-1-(tetrahydro-2H-pyran-4-yl)octahydro-1H-indol-6-yl]urea trifluoroacetate

Procedure as in Example 363

Reagent: 3-chloro-4-fluorophenylisocyanate

Yield: 9.8 mg

Calculated mass: 531.2300

Measured mass: 531.2293

### EXAMPLE 369

### N-[(3aS*,6R*,7aS*)-3a-(3,4-dimethoxyphenyl)-1-(tetrahydro-2H-pyran-4-yl)octahydro-1H-indol-6-yl]-N′-(2,3,4-trifluorophenyl)urea trifluoroacetate

Procedure as in Example 363

Reagent: 2,3,4-trifluorophenylisocyanate

Yield: 10.7 mg

Calculated mass: 533.2501

Measured mass: 533.2526

### EXAMPLE 370

### N-[(3aS*,6R*,7aS*)-3a-(3,4-dimethoxyphenyl)-1-(tetrahydro-2H-pyran-4-yl)octahydro-1H-indol-6-yl]-N′-[4-fluoro-3-(trifluoromethyl)phenyl]urea trifluoroacetate

Procedure as in Example 363

Reagent: 4-fluoro-3-(trifluoromethyl)phenylisocyanate

Yield: 9.8 mg

Calculated mass: 565.2564

Measured mass: 565.2572

### EXAMPLE 371

### N-(3-bromophenyl)-N′-[(3aS*,6R*,7aS*)-3a-(3,4-dimethoxyphenyl) 1-(tetrahydro-2H-pyran-4-yl)octahydro-1H-indol-6-yl]urea trifluoroacetate

Procedure as in Example 363

Reagent: 3-bromophenylisocyanate

Yield: 8.9 mg

Calculated mass: 557.1889

Measured mass: 557.1898

### EXAMPLE 372

### N-[(3aS*,6R*,7aS*)-3a-(3,4-dimethoxyphenyl)-1-(tetrahydro-2H-pyran-4-yl)octahydro-1H-indol-6-yl]-N′-[3-fluoro-5-(trifluoromethyl)phenyl]urea trifluoroacetate

Procedure as in Example 363

Reagent: 3-fluoro-5-(trifluoromethyl)phenylisocyanate

Yield: 7.0 mg

Calculated mass: 565.2564

### EXAMPLE 373

### N-(5-chloro-2-methylphenyl)-N′-[(3aS*,6R*,7aS*)-3a-(3,4-dimethoxyphenyl)-1-(tetrahydro-2H-pyran-4-yl)octahydro-1H-indol-6-yl]urea trifluoroacetate

Procedure as in Example 363

Reagent: 5-chloro-2-methylphenylisocyanate

Yield: 10.1 mg

Calculated mass: 527.2551

Measured mass: 527.2567

### EXAMPLE 374

### N-(3-chlorophenyl)-N′-[(3aS*,6R*,7aS*)-3a-(3,4-dimethoxyphenyl)-1-(tetrahydro-2H-pyran-4-yl)octahydro-1H-indol-6-yl]urea trifluoroacetate

Procedure as in Example 363

Reagent: 3-chlorophenylisocyanate

Yield: 13.8 mg

Calculated mass: 513.2394

Measured mass: 513.2402

### EXAMPLE 375

### N-[(3aS*,6R*,7aS*)-1-cyclohexyl-3a-(3,4-dimethoxyphenyl)octahydro-1H-indol-6-yl]-N′-[2-fluoro-3-(trifluoromethyl)phenyl]urea trifluoroacetate

A solution of (3aS*,6R*,7aS*)-1-cyclohexyl-3a-(3,4-dimethoxyphenyl)octahydro-1H-indol-6-amine, Comparative Example 331, (0.03 mmol) in tetrahydrofuran (2.0 ml) was treated with the appropriate arylisocyanate (0.03 mmol) and allowed to stand at room temperature overnight. The solvent was removed by evaporation and the product isolated by preparative HPLC (YMC column: gradient 20-65% MeCN in water containing 0.1% trifluoroacetic acid).

Reagent: 2-fluoro-3-(trifluoromethyl)phenylisocyanate

Yield: 10.9 mg

Calculated mass: 563.2771

Measured mass: 563.2762

### EXAMPLE 376

### N-[(3aS*,6R*,7aS*)-1-cyclohexyl-3a-(3,4-dimethoxyphenyl)octahydro-1H-indol-6-yl]-N′-[4-(trifluoromethyl)phenyl]urea trifluoroacetate

Procedure as in Example 375

Reagent: 4-(trifluoromethyl)phenylisocyanate

Yield: 6.4 mg

Calculated mass: 545.2865

Measured mass: 545.2871

### EXAMPLE 377

### N-[(3aS*,6R*,7aS*)-1-cyclohexyl-3a-(3,4-dimethoxyphenyl)octahydro-1H-indol-6-yl]-N′-(3-fluorophenyl)urea trifluoroacetate

Procedure as in Example 375

Reagent: 3-fluorophenylisocyanate

Yield: 11.4 mg

Calculated mass: 495.2897

Measured mass: 495.2911

### EXAMPLE 378

### N-[(3aS*,6R*,7aS*)-1-cyclohexyl-3a-(3,4-dimethoxyphenyl)octahydro-1H-indol-6-yl]-N′-(2,5-difluorophenyl)urea trifluoroacetate

Procedure as in Example 375

Reagent: 2,5-difluorophenylisocyanate

Yield: 9.2 mg

Calculated mass: 513.2803

Measured mass: 513.2814

### EXAMPLE 379

### N-[(3aS*,6R*,7aS*)-1-cyclohexyl-3a-(3,4-dimethoxyphenyl)octahydro-1H-indol-6-yl]-N′-[3-(trifluoromethyl)phenyl]urea trifluoroacetate

Procedure as in Example 375

Reagent: 3-(trifluoromethyl)phenylisocyanate

Yield: 12.2 mg

Calculated mass: 545.2865

Measured mass: 545.2883

### EXAMPLE 380

### N-(3-chloro-4-fluorophenyl)-N′-[(3aS*,6R*,7aS*)-1-cyclohexyl-3a-(3,4-dimethoxyphenyl)octahydro-1H-indol-6-yl]urea trifluoroacetate

Procedure as in Example 375

Reagent: 3-chloro-4-fluorophenylisocyanate

Yield: 9.4 mg

Calculated mass: 529.2507

Measured mass: 529.2513

### EXAMPLE 381

### N-[(3aS*,6R*,7aS*)-1-cyclohexyl-3a-(3,4-dimethoxyphenyl)octahydro-1H-indol-6-yl]-N′-(2,3,4-trifluorophenyl)urea trifluoroacetate

Procedure as in Example 375

Reagent: 2,3,4-trifluorophenylisocyanate

Yield: 12.6 mg

Calculated mass: 531.2709

Measured mass: 531.2691

### EXAMPLE 382

### N-[(3aS*,6R*,7aS*)-1-cyclohexyl-3a-(3,4-dimethoxyphenyl)octahydro-1H-indol-6-yl]-N′-[4-fluoro-3-(trifluoromethyl)phenyl]urea trifluoroacetate

Procedure as in Example 375

Reagent: 4-fluoro-3-(trifluoromethyl)phenylisocyanate

Yield: 11.7 mg

Calculated mass: 563.2771

Measured mass: 563.2700

### EXAMPLE 383

### N-(3-bromophenyl)-N′-[(3aS*,6R*,7aS*)-1-cyclohexyl-3a-(3,4-dimethoxyphenyl)octahydro-1H-indol-6-yl]urea trifluoroacetate

Procedure as in Example 375

Reagent: 3-bromophenylisocyanate

Yield: 11.5 mg

Calculated mass: 555.2097

Measured mass: 555.2111

### EXAMPLE 384

### N-[(3aS*,6R*,7aS*)-1-cyclohexyl-3a-(3,4-dimethoxyphenyl)octahydro-1H-indol-6-yl]-N′-[3-fluoro-5-(trifluoromethyl)phenyl]urea trifluoroacetate

Procedure as in Example 375

Reagent: 3-fluoro-5-(trifluoromethyl)phenylisocyanate

Yield: 12.6 mg

Calculated mass: 563.2771

Measured mass: 563.2750

### EXAMPLE 385

### N-(5-chloro-2-methylphenyl)-N′-[(3aS*,6R*,7aS*)-1-cyclohexyl-3a-(3,4-dimethoxyphenyl)octahydro-1H-indol-6-yl]urea trifluoroacetate

Procedure as in Example 375

Reagent: 5-chloro-2-methylphenylisocyanate

Yield: 12.3 mg

Calculated mass: 525.2758

Measured mass: 525.2757

### EXAMPLE 386

### N-(3-chlorophenyl)-N′-[(3aS*,6R*,7aS*)-1-cyclohexyl-3a-(3,4-dimethoxyphenyl)octahydro-1H-indol-6-yl]urea trifluoroacetate

Procedure as in Example 375

Reagent: 3-chlorophenylisocyanate

Yield: 11.0 mg

Calculated mass: 511.2602

Measured mass: 511.2613

### EXAMPLE 387

### N-[(3aS*,6R*,7aS*)-3a-(3, 4-dimethoxyphenyl)-1-(1-methylpiperidin-4-yl)octahydro-1H-indol-6-yl]-N′-[2-fluoro-3-(trifluoromethyl)phenyl]urea trifluoroacetate

A solution of (3aS*,6R*,7aS*)-3a-(3,4-dimethoxyphenyl)-1-(1-methylpiperidin-4-yl)octahydro-1H-indol-6-amine, Comparative Example 329, (0.03 mmol) in tetrahydrofuran (2.0 ml) was treated with the appropriate arylisocyanate (0.03 mmol) and allowed to stand at room temperature overnight. The solvent was removed by evaporation and the product isolated by preparative HPLC (YMC column: gradient 20-65% MeCN in water containing 0.1% trifluoroacetic acid).

Reagent: 2-fluoro-3-(trifluoromethyl)phenylisocyanate

Yield: 8.6 mg

Calculated mass: 578.2880

Measured mass: 578.2888

### EXAMPLE 388

### N-[(3aS*,6R*,7aS*)-3a-(3,4-dimethoxyphenyl)-1-(1-methylpiperidin-4-yl)octahydro-1H-indol-6-yl]-N′-[4-(trifluoromethyl)phenyl]urea trifluoroacetate

Procedure as in Example 387

Reagent: 4-(trifluoromethyl)phenylisocyanate

Yield: 9.5 mg

Calculated mass: 560.2974

Measured mass: 560.2987

### EXAMPLE 389

### N-[(3aS*,6R*,7aS*)-3a-(3,4-dimethoxyphenyl)-1-(1-methylpiperidin-4-yl)octahydro-1H-indol-6-yl]-N′-(3-fluorophenyl)urea trifluoroacetate

Procedure as in Example 387

Reagent: 3-fluorophenylisocyanate

Yield: 6.6 mg

Calculated mass: 510.3006

Measured mass: 510.3004

### EXAMPLE 390

### N-(2,5-difluorophenyl)-N′-[(3aS*,6R*,7aS*)-3a-(3,4-dimethoxyphenyl) 1-(1-methylpiperidin-4-yl)octahydro-1H-indol-6-yl]urea trifluoroacetate

Procedure as in Example 387

Reagent: 2,5-difluorophenylisocyanate

Yield: 7.0 mg

Calculated mass: 528.2912

Measured mass: 528.2912

### EXAMPLE 391

### N-[(3aS*,6R*,7aS*)-3a-(3,4-dimethoxyphenyl)-1-(1-methylpiperidin-4-yl)octahydro-1H-indol-6-yl]-N′-[3-(trifluoromethyl)phenyl]urea trifluoroacetate

Procedure as in Example 387

Reagent: 3-(trifluoromethyl)phenylisocyanate

Yield: 7.9 mg

Calculated mass: 560.2974

Measured mass: 560.2996

### EXAMPLE 392

### N-(3-chloro-4-fluoro henyl)-N′-[(3aS*,6R*,7aS*)-3a-(3,4-dimethoxyphenyl)-1-(1-methylpiperidin-4-yl)octahydro-1H-indol-6-yl]urea trifluoroacetate

Procedure as in Example 387

Reagent: 3-chloro-4-fluorophenylisocyanate

Yield: 7.7 mg

Calculated mass: 544.2616

Measured mass: 544.2629

### EXAMPLE 393

### N-[(3aS*,6R*,7aS*)-3a-(3,4-dimethoxyphenyl)-1-(1-methylpiperidin-4-yl)octahydro-1H-indol-6-yl]-N′-(2,3,4-trifluorophenyl)urea trifluoroacetate

Procedure as in Example 387

Reagent: 2,3,4-trifluorophenylisocyanate

Yield: 7.6 mg

Calculated mass: 546.2818

Measured mass: 546.2819

### EXAMPLE 394

### N-[(3aS*,6R*,7aS*)-3a-(3,4-dimethoxyphenyl)-1-(1-methylpiperidin-4-yl)octahydro-1H-indol-6-yl]-N′-[4-fluoro-3-(trifluoromethyl)phenyl]urea trifluoroacetate

Procedure as in Example 387

Reagent: 4-fluoro-3-(trifluoromethyl)phenylisocyanate

Yield: 7.3 mg

Calculated mass: 578.288

Measured mass: 578.289

### EXAMPLE 395

### N-(3-bromophenyl)-N′-[(3aS*,6R*,7aS*)-3a-(3,4-dimethoxyphenyl)-1-(1-methylpiperidin-4-yl)octahydro-1H-indol-6-yl]urea trifluoroacetate

Procedure as in Example 387

Reagent: 3-bromophenylisocyanate

Yield: 9.5 mg

Calculated mass: 570.2206

Measured mass: 570.2211

### EXAMPLE 396

### N-[(3aS*,6R*,7aS*)-3a-(3,4-dimethoxyphenyl)-1-(1-methylpiperidin-4-yl)octahydro-1H-indol-6-yl]-N′-[3-fluoro-5-(trifluoromethyl)phenyl]urea trifluoroacetate

Procedure as in Example 387

Reagent: 3-fluoro-5-(trifluoromethyl)phenylisocyanate

Yield: 10.0 mg

Calculated mass: 578.288

Measured mass: 578.2881

### EXAMPLE 397

### N-(5-chloro-2-methylphenyl)-N′-[(3aS*,6R*,7aS*)-3a-(3,4-dimethoxyphenyl)-1-(1-methylpiperidin-4-yl)octahydro-1H-indol-6-yl]urea trifluoroacetate

Procedure as in Example 387

Reagent: 5-chloro-2-methylphenylisocyanate

Yield: 10.0 mg

Calculated mass: 540.2867

Measured mass: 540.2879

### EXAMPLE 398

### N-(3-chlorophenyl)-N′-[(3aS*,6R*,7aS*)-3a-(3,4-dimethoxyphenyl)-1-(1-methylpiperidin-4-yl)octahydro-1H-indol-6-yl]urea trifluoroacetate

Procedure as in Example 387

Reagent: 3-chlorophenylisocyanate

Yield: 8.8 mg

Calculated mass: 526.2711

Measured mass: 526.2722

### EXAMPLE 399

### N-[(3aS*,6R*,7aS*)-1-(cyclopropylmethyl)-3a-(3,4-dimethoxyphenyl)octahydro-1H-indol-6-yl]-N′-[2-fluoro-3-(trifluoromethyl)phenyl]urea trifluoroacetate

A solution of (3aS*,6R*,7aS*)-1-cyclopropylmethyl-3a-(3,4-dimethoxyphenyl)octahydro-1H-indol-6-amine, Comparative Example 335, (0.03 mmol) in tetrahydrofuran (2.0 ml) was treated with the appropriate arylisocyanate (0.03 mmol) and allowed to stand at room temperature overnight. The solvent was removed by evaporation and the product isolated by preparative HPLC (YMC column: gradient 20-65% MeCN in water containing 0.1% trifluoroacetic acid).

Reagent: 2-fluoro-3-(trifluoromethyl)phenylisocyanate

Yield: 11.2 mg

Calculated mass: 535.2458

Measured mass: 535.2484

### EXAMPLE 400

### N-[(3aS*,6R*,7aS*)-1-(cyclopropylmethyl)-3a-(3,4-dimethoxyphenyl)octahydro-1H-indol-6-yl]-N′-[4-(trifluoromethyl)phenyl]urea trifluoroacetate

Procedure as in Example 399

Reagent: 4-(trifluoromethyl)phenylisocyanate

Yield: 13.2 mg

Calculated mass: 517.2552

MS (ionspray; [M+H]⁺) m/z: 518

### EXAMPLE 401

### N-[(3aS*,6R*,7aS*)-1-(cyclopropylmethyl)-3a-(3,4-dimethoxyphenyl)octahydro-1H-indol-6-yl]-N′-(3-fluorophenyl)urea trifluoroacetate

Procedure as in Example 399

Reagent: 3-fluorophenylisocyanate

Yield: 12.5 mg

Calculated mass: 467.2584

Measured mass: 467.2594

### EXAMPLE 402

### N-[(3aS*,6R*,7aS*)-1-(cyclopropylmethyl)-3a-(3,4-dimethoxyphenyl)octahydro-1H-indol-6-yl]-N′-(2,5-difluorophenyl)urea trifluoroacetate

Procedure as in Example 399

Reagent: 2,5-difluorophenylisocyanate

Yield: 10.5 mg

Calculated mass: 485.2490

Measured mass: 485.2471

### EXAMPLE 403

### N-[(3aS*,6R*,7aS*)-1-(cyclopropylmethyl)-3a-(3,4-dimethoxyphenyl)octahydro-1H-indol-6-yl]-N′-[3-(trifluoromethyl)phenyl]urea trifluoroacetate

Procedure as in Example 399

Reagent: 3-(trifluoromethyl)phenylisocyanate

Yield: 11.3 mg

Calculated mass: 517.2552

Measured mass: 517.2555

### EXAMPLE 404

### N-(3-chloro-4-fluorophenyl)-N′-[(3aS*,6R*,7aS*)-1-(cyclopropylmethyl)-3a-(3,4-dimethoxyphenyl)octahydro-1H-indol-6-yl]urea trifluoroacetate

Procedure as in Example 399

Reagent: 3-chloro-4-fluorophenylisocyanate

Yield: 3.5 mg

Calculated mass: 501.2194

Measured mass: 501.2215

### EXAMPLE 405

### N-[(3aS*,6R*,7aS*)-3a-(3,4-dimethoxyphenyl)-1-(1-methylpiperidin-4-yl)octahydro-1H-indol-6-yl]-N′-(2,3,4-trifluorophenyl)urea trifluoroacetate

Procedure as in Example 399

Reagent: 2,3,4-trifluorophenylisocyanate

Yield: 9.8 mg

Calculated mass: 503.2396

Measured mass: 503.2397

### EXAMPLE 406

### N-[(3aS*,6R*,7aS*)-1-(cyclopropylmethyl)-3a-(3,4-dimethoxyphenyl)octahydro-1H-indol-6-yl]-N′-[4-fluoro-3-(trifluoromethyl)phenyl]urea trifluoroacetate

Procedure as in Example 399

Reagent: 4-fluoro-3-(trifluoromethyl)phenylisocyanate

Yield: 10.6 mg

Calculated mass: 535.2458

Measured mass: 535.2476

### EXAMPLE 407

### N-(3-bromophenyl)-N′-[(3aS*,6R*,7aS*)-1-(cyclopropylmethyl)-3a-(3,4-dimethoxyphenyl)octahydro-1H-indol-6-yl]urea trifluoroacetate

Procedure as in Example 399

Reagent: 3-bromophenylisocyanate

Yield: 12.8 mg

Calculated mass: 527.1784

Measured mass: 527.1780

### EXAMPLE 408

### N-[(3aS*,6R*,7aS*)-1-(cyclopropylmethyl)-3a-(3,4-dimethoxyphenyl)octahydro-1H-indol-6-yl]-N′-[3-fluoro-5-(trifluoromethyl)phenyl]urea trifluoroacetate

Procedure as in Example 399

Reagent: 3-fluoro-5-(trifluoromethyl)phenylisocyanate

Yield: 10.4 mg

Calculated mass: 535.2458

Measured mass: 535.2467

### EXAMPLE 409

### N-(5-chloro-2-methylphenyl)-N′-[(3aS*,6R*,7aS*)-1-(cyclopropylmethyl)-3a-(3,4-dimethoxyphenyl)octahydro-1H-indol-6-yl]urea trifluoroacetate

Procedure as in Example 399

Reagent: 5-chloro-2-methylphenylisocyanate

Yield: 13.4 mg

Calculated mass: 497.2445

Measured mass: 497.2453

### EXAMPLE 410

### N-(3-chlorophenyl)-N′-[(3aS*,6R*,7aS*)-1-(cyclopropylmethyl)-3a-(3,4-dimethoxyphenyl)octahydro-1H-indol-6-yl]urea trifluoroacetate

Procedure as in Example 399

Reagent: 3-chlorophenylisocyanate

Yield: 12.1 mg

Calculated mass: 483.2289

Measured mass: 483.2297

### EXAMPLE 411

### N-[(3aS*,6R*,7aS*)-3a-(3,4-dimethoxyphenyl)-1-propyloctahydro-1H-indol-6-yl-N′-[2-fluoro-3-(trifluoromethyl)phenyl]urea trifluoroacetate

A solution of (3aS*,6R*,7aS*)-3a-(3,4-dimethoxyphenyl)-1-propyloctahydro-1H-indol-6-amine, Comparative Example 334, (0.03 mmol) in tetrahydrofuran (2.0 ml) was treated with the appropriate arylisocyanate (0.03 mmol) and allowed to stand at room temperature overnight. The solvent was removed by evaporation and the product isolated by preparative HPLC (YMC column: gradient 20-65% MeCN in water containing 0.1% trifluoroacetic acid).

Reagent: 2-fluoro-3-(trifluoromethyl)phenylisocyanate

Yield: 8.2 mg

Calculated mass: 523.2458

Measured mass: 523.2432

### EXAMPLE 412

### N-[(3aS*,6R*,7aS*)-3a-(3,4-dimethoxyphenyl)-1-propyloctahydro-1H-indol-6-yl]-N′-[4-(trifluoromethyl)phenyl]urea trifluoroacetate

Procedure as in Example 411

Reagent: 4-(trifluoromethyl)phenylisocyanate

Yield: 10.8 mg

Calculated mass: 505.2552

Measured mass: 505.2563

### EXAMPLE 413

### N-[(3aS*,6R*,7aS*)-3a-(3,4-dimethoxyphenyl)-1-propyloctahydro-1H-indol-6-yl]-N′-(3-fluorophenyl)urea trifluoroacetate

Procedure as in Example 411

Reagent: 3-fluorophenylisocyanate

Yield: 10.8 mg

Calculated mass: 455.2584

Measured mass: 455.2570

### EXAMPLE 414

### N-(2,5-difluorophenyl)-N′-[(3aS*,6R*,7aS*)-3a-(3,4-dimethoxyphenyl)-1-propyloctahydro-1H-indol-6-yl]urea trifluoroacetate

Procedure as in Example 411

Reagent: 2,5-difluorophenylisocyanate

Yield: 6.8 mg

Calculated mass: 473.249

Measured mass: 473.2469

### EXAMPLE 415

### N-[(3aS*,6R*,7aS*)-3a-(3,4-dimethoxyphenyl)-1-propyloctahydro-1H-indol-6-yl]-N′-[3-(trifluoromethyl)phenyl]urea trifluoroacetate

Procedure as in Example 411

Reagent: 3-(trifluoromethyl)phenylisocyanate

Yield: 6.5 mg

Calculated mass: 505.2552

Measured mass: 505.2547

### EXAMPLE 416

### N-(3-chloro-4-fluorophenyl)-N′-[(3aS*,6R*,7aS*)-3a-(3,4-dimethoxyphenyl)-1-propyloctahydro-1H-indol-6-yl]urea trifluoroacetate

Procedure as in Example 411

Reagent: 3-chloro-4-fluorophenylisocyanate

Yield: 5.4 mg

Calculated mass: 489.2194

Measured mass: 489.2179

### EXAMPLE 417

### N-(3aS*,6R*,7aS*)-3a-(3,4-dimethoxyphenyl)-1-propyloctahydro-1H-indol-6-yl-N′-(2,3,4-trifluorophenyl)urea trifluoroacetate

Procedure as in Example 411

Reagent: 2,3,4-trifluorophenylisocyanate

Yield: 7.2 mg

Calculated mass: 491.2396

Measured mass: 491.2400

### EXAMPLE 418

### N-(3aS*,6R*,7aS*)-3a-(3,4-dimethoxyphenyl)-1-propyloctahydro-1H-indol-6-yl]-N′-[4-fluoro-3-(trifluoromethyl)phenyl]urea trifluoroacetate

Procedure as in Example 411

Reagent: 4-fluoro-3-(trifluoromethyl)phenylisocyanate

Yield: 6.8 mg

Calculated mass: 523.2458

Measured mass: 523.2468

### EXAMPLE 419

### N-(3-bromophenyl)-N′-[(3aS*,6R*,7aS*)-3a-(3,4-dimethoxyphenyl)-1-propyloctahydro-1H-indol-6-yl]urea trifluoroacetate

Procedure as in Example 411

Reagent: 3-bromophenylisocyanate

Yield: 5.8 mg

Calculated mass: 515.1784

Measured mass: 515.1801

### EXAMPLE 420

### N-[(3aS*,6R*,7aS*)-3a-(3,4-dimethoxyphenyl)-1-propyloctahydro-1H-indol-6-yl]-N′-[3-fluoro-5-(trifluoromethyl)phenyl]urea trifluoroacetate

Procedure as in Example 411

Reagent: 3-fluoro-5-(trifluoromethyl)phenylisocyanate

Yield: 6.2 mg

Calculated mass: 523.2458

Measured mass: 523.2469

### EXAMPLE 421

### N-(5-chloro-2-methylphenyl)-N′-[(3aS*,6R*,7aS*)-3a-(3,4-dimethoxyphenyl)-1-propyloctahydro-1H-indol-6-yl]urea trifluoroacetate

Procedure as in Example 411

Reagent: 5-chloro-2-methylphenylisocyanate

Yield: 5.6 mg

Calculated mass: 485.2445

Measured mass: 485.2433

### EXAMPLE 422

### N-(3-chlorophenyl)-N′-[(3aS*,6R*,7aS*)-3a-(3,4-dimethoxyphenyl)-1-propyloctahydro-1H-indol-6-yl]urea trifluoroacetate

Procedure as in Example 411

Reagent: 3-chlorophenylisocyanate

Yield: 8.6 mg

Calculated mass: 471.2289

Measured mass: 471.2304

### EXAMPLE 423

### N-2,1,3-benzothiadiazol-4-yl-N′-[(3aS*,6R*,7aS*)-3a-(3,4-dimethoxyphenyl)-1-methyloctahydro-1H-indol-6-yl]urea trifluoroacetate

Synthetic Procedure: Scheme E

Reagent: N-2,1,3-benzothiadiazol-4-yl-isocyanate

Yield: 4.3 mg

Calculated mass: 467.1991

Measured mass: 467.1998

### EXAMPLE 424

### N-[(3aS*,6R*,7aS*)-3a-(3,4-dimethoxyphenyl)-1-methyloctahydro-1H-indol-6-yl]-N′-[3-(1,1,2,2-tetrafluoroethoxy)phenyl]urea trifluoroacetate

Reagent: 3-(1,1,2,2-tetrafluoroethoxy)aniline

Synthetic Procedure: Scheme F

Yield: 6.4 mg

Calculated mass: 525.2251

Measured mass: 525.2261

### EXAMPLE 425

### N-[(3aS*,6R*,7aS*)-3a-(3,4-dimethoxyphenyl)-1-methyloctahydro-1H-indol-6-yl]-N′-[4-nitro-3-(trifluoromethyl)phenyl]urea trifluoroacetate

Reagent: 4-nitro-3-(trifluoromethyl)aniline

Synthetic Procedure: Scheme F

Yield: 7.2 mg

Calculated mass: 522.2090

Measured mass: 522.2094

### EXAMPLE 426

### N-butyl-3-[({[(3aS*,6R*,7aS*)-3a-(3,4-dimethoxyphenyl)-1-methyloctahydro-1H-indol-6-yl]amino}carbonyl)amino]benzenesulfonamide trifluoroacetate

Reagent: 3-amino-N-butylbenzenesulphonamide

Synthetic Procedure: Scheme F

Yield: 4.6 mg

Calculated mass: 544.2719

Measured mass: 544.2703

### EXAMPLE 427

### N-(2,2-difluoro-1,3-benzodioxol-5-yl)-N′-[(3aS*,6R*,7aS*)-3a-(3,4-dimethoxyphenyl)-1-methyloctahydro-1H-indol-6-yl]urea trifluoroacetate

Reagent: 5-amino-2,2-difluoro-1,3-benzodioxole

Synthetic Procedure: Scheme F

Yield: 8.2 mg

Calculated mass: 489.2075

Measured mass: 489.2059

### EXAMPLE 428

### N-[(3aS*,6R*,7aS*)-3a-(3,4-dimethoxyphenyl)-1-methyloctahydro-1H-indol-6-yl]-N′-(1,1-dioxido-1-benzothien-6-yl)urea trifluoroacetate

Reagent: 6-amino-1,1-dioxido-1-benzothiophene

Synthetic Procedure: Scheme F

Yield: 1.2 mg

Calculated mass: 497.1984

Measured mass: 497.2077

### EXAMPLE 429

### N-(2,5-difluorophenyl)-N′-[(3aS,6R,7aS)-3a-(3,4-dimethoxyphenyl)-1-methyloctahydro-1H-indol-6-yl]urea hydrochloride

A solution of (3aS,6R,7aS)-1-methyl-3a-(3,4-dimethoxyphenyl)octahydro-1H-indol-6-amine, Comparative Example 10, (1.74 g; 6.0 mmol) in dichloromethane (20.0 ml) was treated dropwise with a solution of 2,5-difluorophenylisocyanate (0.93 g; 6.0 mmol) in DCM (5.0 ml). The resulting solution was stirred at room temperature for 24 h.

The mixture was evaporated and the colourless solid treated with MeOH (30 ml) then acidified with 4M HCl in Dioxane (5.0 ml). The mixture was evaporated and the residue crystallised from EtOH to afford the product as a colourless solid 2.64 g (91%)

Calculated mass: 445.2177

Measured mass: 445.2177

Optical rotation [α]²⁰_{D}=+8.2

### EXAMPLE 430

### N-(3-chloro-4-fluorophenyl)-N′-[(3aS,6R,7aS)-3a-(3,4-dimethoxyphenyl)-1-methyloctahydro-1H-indol-6-yl]urea hydrochloride

A solution of (3aS,6R,7aS)-1-methyl-3a-(3,4-dimethoxyphenyl)octahydro-1H-indol-6-amine, Comparative Example 10, (130 mg; 0.45 mmol) in tetrahydrofuran (5.0 ml) was treated with 3-chloro-4-fluorophenylisocyanate (77 mg; 0.45 mmol). The resulting solution was stirred at room temperature for 24 h.

The mixture was evaporated and the residue flash-chromatographed over silica. Elution with ethyl acetate gave the product which was converted to the hydrochloride salt by treatment with 2M HCl in dioxane followed by evaporation of the solvent to afford a colourless solid 186 mg (83%)

Calculated mass: 461.1881

Measured mass: 461.1898

Optical rotation [α]²⁰_{D}=+4.5

### EXAMPLE 431

### N-[(3aS,6R,7aS)-3a-(3,4-dimethoxyphenyl)-1-methyloctahydro-1H-indol-6-yl]-N′-[3-fluoro-5-(trifluoromethyl)phenyl]urea hydrochloride

A solution of the (3aS,6R,7aS)-1-methyl-3a-(3,4-dimethoxyphenyl)octahydro-1H-indol-6-amine, Comparative Example 10, (377 mg; 1.3 mmol) in tetrahydrofuran (10.0 ml) was treated with 3-fluoro-5-(trifluoromethyl)phenylisocyanate (205 mg; 1.3 mmol). The resulting solution was stirred at room temperature for 24 h.

The mixture was evaporated and the residue flash-chromatographed over silica. Elution with ethyl acetate gave the free-base which was converted to the hydrochloride salt by treatment with 4M HCl in dioxane. The solvent was evaporated and the residue triturated with diethyl ether to afford a colourless solid 320 mg (46%)

Calculated mass: 495.2145

Measured mass: 495.2138

Optical rotation [α]²⁰_{D}=+5.2

### EXAMPLE 432

### N-[(3aS,6R,7aS)-3a-(3,4-dimethoxyphenyl)-1-methyloctahydro-1H-indol-6-yl]-N′-(3-fluorophenyl)urea hydrochloride

A solution of (3aS,6R,7aS)-1-methyl-3a-(3,4-dimethoxyphenyl)octahydro-1H-indol-6-amine, Comparative Example 10, (130 mg; 0.45 mmol) in tetrahydrofuran (5.0 ml) was treated with 3-fluorophenylisocyanate (61 mg; 0.45 mmol). The resulting solution was stirred at room temperature for 24 h.

The mixture was evaporated and the residue flash-chromatographed over silica. Elution with ethyl acetate gave the product which was converted to the hydrochloride salt by treatment with 2M HCl in dioxane followed by evaporation of the solvent to afford a colourless solid 147 mg (71%)

Calculated mass: 427.2271

Measured mass: 427.2283

Optical rotation [α]²⁰_{D}=+4.8

### EXAMPLE 433

### N-[(3aS,6R,7aS)-1-(cyclopropylmethyl)-3a-(3,4-dimethoxyphenyl)octahydro-1H-indol-6-yl]-N′-(2,3,4-trifluorophenyl)urea hydrochloride

A solution of (3aS,6R,7aS)-1-(cyclopropylmethyl)-3a-(3,4-dimethoxyphenyl)octahydro-1H-indol-6-amine prepared in Example 438 (step 1), (600 mg; 1.8 mmol) in DCM (10 ml) was treated with 2,3,4-trifluorophenylisocyanate (329 mg; 1.9 mmol) and stirred at RT overnight. The mixture was evaporated and the residue flash-chromatographed over silica. Elution with chloroform (saturated with NH₃ gas) gave the crude product which was treated with 4M HCl in dioxane and crystallised from ethanol-light petroleum (40-60) to afford the product as a colourless solid. Yield 150 mg (15%)

Calculated mass: 503.2396

Measured mass: 503.2385

### EXAMPLE 434

### N-(3,4-difluorophenyl)-N′-[(1S,10S,12R)-4,5-dimethoxy-9-azatetracyclo[7.5.2.0˜1,10˜.0˜2,7˜]hexadeca-2,4,6-trien-12-yl]urea trifluoroacetate

N-(3,4-difluorophenyl)-N′-[(3aS*,6R*,7aS*)-3a-(3,4-dimethoxyphenyl)octahydro-1H-indol-6-yl]urea, Example 175, (0.010 g, 0.023 mmol) was dissolved in MeOH (0.3 ml) and formaldehyde 37% (0.5 ml) was added. The mixture was stirred for 15 minutes and 8N HCl (0.8 ml) was added. The mixture was allowed to stand overnight at r.t. and then pH was adjusted to 10 with 2N NaOH, extracted with CH₂Cl₂ (2×) dried (MgSO₄) and evaporated. The crude product was purified by reversed phase HPLC using acetonitrile-water (containing 0.1% TFA), gradient 5-20%. Yield 7.7 mg (60%). Colorless liquid.

MS (ionspray; [M+H]⁺) m/z: 444.2. HRMS for C₂₄H₂₇F₂N₃O₃: Calcd, 443.2020; found, 443.2018.

### EXAMPLE 435

### N-(3,4-difluorophenyl)-N′-[(3aS,6R,7aS)-3a-(3,4-dimethoxyphenyl)-1-butyloctahydro-1H-indol-6-yl]urea hydrochloride

Tert-butyl [(3aS,6R,7aS)-3a-(3,4-dimethoxyphenyl)octahydro-1H-indol-6-yl]carbamate, intermediate from Comparative Example 10, (286 mg, 670 μmol) was dissolved in dichloromethane (8 mL). Butyraldehyde (200 μL, 160 mg, 2.22 mmol) was added and the mixture stirred at room temperature for 1 hour. Sodium triacetoxyborohydride (400 mg, 1.89 mmol) was added and the reaction stirred for 3 days. The mixture was then diluted with dichloromethane (100 mL), washed with sodium hydroxide solution (3M, 100 mL) and the organic phase separated and dried over sodium sulphate. The solvent was reduced to a volume of ca. 10 mL, and TFA (2 mL) was added. After stirring at room temperature for 4 hours, the solvent was removed under reduced pressure, and the crude material dissolved in dichloromethane (100 mL). This was washed with sodium hydroxide solution (3M, 3×50 mL), dried over sodium sulfate and the solvent reduced to a volume of ca. 5 mL under reduced pressure. This was then treated with 3,4-difluorophenylisocyanate (113 mg, 727 μmol) and the reaction stirred at room temperature overnight. Solvent was then removed and the crude product was purified by column chromatography (SiO₂; dichloromethane as eluent) to give the desired product as a clear gum (60 mg, 18%). This was then converted to the hydrochloride salt by dissolving in dichloromethane (10 mL) and adding HCl in ether (20 mL of a 2M solution). The solution was evaporated to give the desired salt of the title compound as an off-white solid.

Calculated mass: 487.2646

Measured mass: 487.2662

### EXAMPLE 436

### N-(3,4-difluorophenyl)-N′-[(3aS,6R,7aS)-3a-(3,4-dimethoxyphenyl)-1-isopropyloctahydro-1H-indol-6-yl]urea hydrochloride

Tert-butyl [(3aS,6R,7aS)-3a-(3,4-dimethoxyphenyl)octahydro-1H-indol-6-yl]carbamate, intermediate from Comparative Example 10, (250 mg, 664 μmol) was dissolved in dichloromethane (8 mL). Acetone (158 mg, 2.72 mmol) was added and the mixture stirred at room temperature for 1 hour. Sodium triacetoxyborohydride (400 mg, 1.89 mmol) was added and the reaction stirred for 3 days. LCMS after this time showed ca. 40% completion. Further aliquots of acetone and borohydride were added and the reaction allowed to proceed to completion. The mixture was then diluted with dichloromethane (100 mL), washed with sodium hydroxide solution (3M, 100 mL) and the organic phase separated and dried over sodium sulphate. The solvent was reduced to a volume of ca. 10 mL, and TFA (2 mL) was added. After stirring at room temperature for 4 hours, the solvent was removed under reduced pressure, and the crude material dissolved in dichloromethane (100 mL). This was washed with sodium hydroxide solution (3M, 3×50 mL), dried over sodium sulfate and the solvent reduced to a volume of ca. 5 mL under reduced pressure. This was then treated with 3,4-difluorophenylisocyanate (159 mg, 1.0 mmol) and the reaction stirred at room temperature overnight. Solvent was then removed and the crude product was purified by column chromatography (SiO₂; ethyl acetate:pentane 1:1 as eluent) to give the desired product as an off-white solid (120 mg, 38%). This was then converted to the hydrochloride salt by dissolving in dichloromethane (10 mL) and adding HCl in ether (20 mL of a 2M solution). The solution was evaporated to give the desired salt as an off-white solid.

### EXAMPLE 437

### N-(3,4-difluorophenyl)-N′-[(3aS,6R,7aS)-3a-(3,4-dimethoxyphenyl)-1-propyloctahydro-1H-indol-6-yl]urea hydrochloride

Step 1: Tert-butyl [(3aS,6R,7aS)-3a-(3,4-dimethoxyphenyl)octahydro-1H-indol-6-yl]carbamate, intermediate from Comparative Example 10, as the AcOH salt (300 mg, 0.69 mmol) and propan-1-al (0.06 ml, 0.83 mmol) were mixed in dichloro ethane (10 ml). NaBH(OAc)₃ (220 mg, 1.04 mmol) was added and the reaction mixture was stirred at ambient temperature for 2.5 hrs. The mixture was concentrated, 2M NaOH (10 ml) was added and the mixture was extracted with EtOAc, dried over Na₂SO₄ and concentrated. TFA, 50% vol. in CH₂Cl₂ (10 ml) was added and the reaction mixture was stirred at ambient temperature for 45 min. The crude mixture was basified with 2M NaOH, extracted with EtOAc, dried over Na₂SO₄, and concentrated to give the product, 170 mg (77%), which was immediately used in the next step without further purification.

Step 2: (3aS,6R,7aS)-3a-(3,4-dimethoxyphenyl)-1-propyloctahydro-1H-indol-6-amine (438 mg, 1.38 mmol) and 3,4-difluorophenyl isocyanate (213 mg, 1.38 mmol) were mixed in anhydrous CH₂Cl₂ (5 ml) under N₂. Stirred at ambient temperature over night. The crude mixture was concentrated and purified by column chromatography on silica (EtOAc) to give the product. 2M HCl in diethylether was added followed by evaporation of the diethylether to give the title compound, 82 mg. A 1 mg/ml solution of the title compound in methanol gave Optical rotation [α]²⁰_{D}=+15

### EXAMPLE 438

### N-[(3aS,6R,7aS)-1-(cyclopropylmethyl)-3a-(3,4-dimethoxyphenyl)octahydro-1H-indol-6-yl]-N′-(3,4-difluorophenyl)urea hydrochloride

Step 1: Tert-butyl [(3aS,6R,7aS)-3a-(3,4-dimethoxyphenyl)octahydro-1H-indol-6-yl]carbamate, (as the AcOH salt) intermediate from Comparative Example 10, (300 mg, 0.69 mmol) and cyclopropanecarboxaldehyde (0.06 ml, 0.83 mmol) were mixed in dichloro ethane (10 ml). NaBH(OAc)₃ (220 mg, 1.04 mmol) was added and the reaction mixture was stirred at ambient temperature for 2.5 hrs. The mixture was concentrated, 2M NaOH (10 ml) was added and the mixture was extracted with EtOAc, dried over Na₂SO₄ and concentrated. TFA, 50% vol. in CH₂Cl₂ (10 ml) was added and the reaction mixture was stirred at ambient temperature for 45 min. The crude mixture was basified with 2M NaOH, extracted with EtOAc, dried over Na₂SO₄, and concentrated to give the product, 182 mg (80%), which was immediately used in the next step without further purification.

Step 2: (3aS,6R,7aS)-1-(cyclopropylmethyl)-3a-(3,4-dimethoxyphenyl)octahydro-1H-indol-6-amine prepared in step 1, (182 mg, 0.55 mmol) and 3,4-difluorophenyl isocyanate (128 mg, 0.83 mmol) were mixed in anhydrous CH₂Cl₂ (5 ml) under N₂. Stirred at ambient temperature over night. The crude mixture was concentrated and purified by column chromatography on silica (EtOAc) to give the product which was converted to the HCl salt by treatment with a 2 M solution of HCl in diethyl ether followed by evaporation of the diethyl ether to give the title compound, 80 mg (28%). A 1 mg/ml solution of the title compound in methanol gave optical rotation [α]²⁰_{D}=+10

### EXAMPLE 439

### N-[4-cyano-3-(trifluoromethyl)phenyl]-N′-[(3aS,6R,7aS)-3a-(3,4-dimethoxyphenyl)-1-methyloctahydro-1H-indol-6-yl]urea hydrochloride

Triethylamine (83 μL, 0.60 mmol), was added to a solution of 4-amino-2-trifluoromethylbenzonitrile (67 mg, 0.36 mmol) in dry CH₂Cl₂ (3 mL) under an atmosphere of nitrogen. Triphosgene (36 mg, 0.12 mmol) dissolved in dry CH₂Cl₂ (1 mL) was added drop-wise and the reaction was stirred at ambient temperature for 2 hrs. (3aS,6R,7aS)-3a-(3,4-dimethoxyphenyl)-1-methyloctahydro-1H-indol-6-amine, Comparative Example 10, (87 mg, 0.30 mmol) dissolved in dry CH₂Cl₂ (3 mL) was added and the reaction was stirred over night at ambient temperature. After solvent evaporation, the crude was precipitated in EtOAc/MeOH (95:5), filtered, dried in vacuo, dissolved in CH₂Cl₂ (10 mL) and treated with HCl in Et₂O (2.0M, 0.4 mL, 0.8 mmol). 50 mL of dry Et₂O was added to precipitate the HCl salt. The white solid formed was filtered off and dried in vacuo to yield 60.6 mg (37%) of the product as a white powder.

¹H NMR (500 MHz, MeOH-d₄) ppm 1.13-1.39 (m, 1H) 1.67-1.98 (m, 3H) 2.10-2.35 (m, 2H) 2.35-2.61 (m 2H) 3.12 (s, 3H) 3.18-3.47 (m, 1H) 3.78-4.04 (2H) 3.83 (s, 3H) 3.86 (s, 3H) 4.06-4.19 (m, 1H) 6.90-7.08 (m, 3H) 7.59-7.71 (dd, J=2,0 Hz, 8.5 Hz, 1H) 7.76-7.86 (d, J=8.5 Hz, 1H) 8.06-8.15 (d, J=2.0 Hz, 1H).

HRMS (EI) calcd for C₂₆H₂₉F₃N₄O₃: 502.2192, found 502.2208.

optical rotation [α]²⁰_{D}=+17,5

### EXAMPLE 440

### N-[(3aS,6R,7aS)-3a-(3,4-dimethoxyphenyl)-1-methyloctahydro-1H-indol-6-yl]-N′-(3,4,5-trifluorophenyl)urea hydrochloride

Triethylamine (1.57 mL, 11.3 mmol) was added to a solution of 3,4,5-trifluorophenylamine (1.0 g, 6.8 mmol) in dry CH₂Cl₂ (40 mL) under an atmosphere of nitrogen. Triphosgene (672 mg, 2.3 mmol) dissolved in dry CH₂Cl₂ (20 mL) was added drop-wise and the reaction was stirred at ambient temperature for 2 hrs. (3aS,6R,7aS)-3a-(3,4-dimethoxyphenyl)-1-methyloctahydro-1H-indol-6-amine, Comparative Example 10, (1.64 mg, 5.7 mmol) dissolved in dry CH₂Cl₂ (40 mL) was added and the reaction was stirred over night at ambient temperature. After solvent evaporation, the crude was purified by flash chromatography (start with 100% EtOAc, then EtOAc/MeOH (95:5)) filtered and dry in vacuo to yield 1.09 g of the free base as a white solid. The free base was dissolved in CH₂Cl₂ (20 mL) and treated with HCl in Et₂O (2.0M, 2.35 mL, 4.7 mmol). 300 mL of dry Et₂O was added to precipitate the HCl salt. The white solid formed was filtered off, dried in vacuo and recrystallized from ethanol. The white crystals were filtered off and dried in vacuo to yield 482 mg (20%) of the product.

¹H NMR (500 MHz, DMSO-D6) ppm 0.99-1.12 (m, 1H) 1.61 (ddd, J=15.1, 12.0, 3.2 Hz, 1H) 1.70 (d, J=11.1 Hz, 1H) 1.90 (t, J=13.2 Hz, 1H) 2.00-2.16 (m, 2H) 2.21 (d, J=14.5 Hz, 1H) 2.31 (d, J=14.4 Hz, 1H) 2.95 (d, J=3.2 Hz, 3H) 3.16-3.29 (m, 1H) 3.71-3.81 (m, J=20.4 Hz, 1H) 3.76 (s, 3H) 3.80 (s, 3H) 4.05 (d, J=7.4 Hz, 1H) 4.02-4.19 (m, 1H) 6.62 (d, J=7.6 Hz, 1H) 6.90-6.98 (m, 2H) 6.99 (s, 1H) 7.31 (dd, J=9.0, 5.9 Hz, 2H) 9.23 (s, 1H) 10.12-10.34 (m, 1H).

HRMS (EI) calcd for C₂₄H₂₈F₃N₃O₃: 463.2083, found 463.2083.

Anal. Calcd for C₂₄H₂₈F₃N₃O₃ x HCl: C, 57.6; H, 5.9; N, 8.4. Found: C, 57.4; H, 5.9; N, 8.4.

optical rotation [α]²⁰_{D}+4.75

### EXAMPLE 441

### N-[(3aS,6R,7aS)-3a-(3,4-dimethoxyphenyl)-1-methyloctahydro-1H-indol-6-yl]-N′-[4-methoxy-3-(trifluoromethyl)phenyl]urea hydrochloride

Triethylamine (83 μL, 0.60 mmol) was added to a solution of 4-methoxy-3-trifluoromethyl-phenylamine (69 mg, 0.36 mmol) in dry CH₂Cl₂ (3 mL) under an atmosphere of nitrogen. Triphosgene (36 mg, 0.12 mmol) dissolved in dry CH₂Cl₂ (1 mL) was added drop-wise and the reaction was stirred at ambient temperature for 2 hrs. (3aS,6R,7aS)-3a-(3,4-dimethoxyphenyl)-1-methyloctahydro-1H-indol-6-amine, Comparative Example 10, (87 mg, 0.30 mmol) dissolved in dry CH₂Cl₂ (3 mL) was added and the reaction was stirred over night at ambient temperature. After solvent evaporation, the crude was precipitated in EtOAc/MeOH (95:5), filtered and dried in vacuo to yield 137 mg of the free base as a white solid. The free base was dissolved in CH₂Cl₂ (5 mL) and treated with HCl in Et₂O (2.0M, 0.4 mL, 0.8 mmol). 50 mL of dry Et₂O was added to precipitate the HCl salt. The white solid formed was filtered off and dried in vacuum to yield 102.9 mg (31%) as a white powder. The product contained 2.3 molecules of triethylamine hydrochloride as demonstrated by ¹H NMR (270 MHz, MeOH-d) δ 1.15-1.44 (m, 1H), 1.31 (t, J=7.2 Hz, 3H, 2.5 Et₃N) 1.68-1.93 (m, 3H) 2.10-2.31 (m, 2H) 2.31-2.57 (m 2H) 3.10 (s, 3H) 3.13-3.43 (m, 1H) 3.20 (q, J=7.2 Hz, 2H, 2.3 Et₃N) 3.77-4.00 (m, 2H) 3.83 (s, 3H) 3.84 (s, 3H) 3.86 (s, 3H) 4.01-4.18 (m, 1H) 6.91-7.02 (m, 3H) 7.02-7.13 (m, 1H) 7.34-7.49 (m, 1H) 7.58-7.72 (m, 1H).

MS (ESI+) for C₂₆H₃₂F₃N₃O₄ m/z 508 (M+H)⁺.

optical rotation [α]²⁰_{D}=+6.67

### EXAMPLE 442

### (3aS,6R,7aS)-6-({[(3,4-difluorophenyl)aminolcarbonyl]amino)-3a-(3,4-dimethoxyphenyl)-1,1-dimethyloctahydro-1H-indolium chloride

MeI (207 μL, 3.39 mmol) and K₂CO₃ (340 mg, 2.49 mmol) were added to N-(3,4-difluorophenyl)-N′-[(3aS,6R,7aS)-3a-(3,4-dimethoxyphenyl)-1-methyloctahydro-1H-indol-6-yl]urea, free base of Example 12, (185 mg, 0.42 mmol) in MeCN (10 mL). The reaction mixture was stirred at 50° C. for 2 h, allowed to cool to ambient temperature, and filtrered through celite. After concentration, the crude product was purified by reversed phase HPLC to give 125.8 mg of the free base as an uncolored oil. The free base was dissolved in CH₂Cl₂ (1 mL) and treated with HCl in Et₂O (2M, 0.22 mL, 0.438 mmol) and the mixture diluted with Et₂O (70 mL). The resulting HCl salt was filtered, washed with Et₂O, and dried in vacuo at 30° C. to give 68.1 mg (35%) of the title compound as a white solid.

¹H NMR (270 MHz, METHANOL-D3) δ ppm 1.06-1.30 (m, 1H) 1.71-1.86 (m, 1H) 1.91-2.15 (m, 2H) 2.22-2.37 (m, 1H) 2.51-2.62 (m, 2H) 2.62-2.74 (m, 1H) 3.26 (s, 3H) 3.33 (s, 3H) 3.64-3.78 (m, 1H) 3.78-4.00 (m, 2H) 3.83 (s, 3H) 3.88 (s, 3H) 4.30-4.41 (m, 1H) 6.92-7.02 (m, 4H) 7.06-7.20 (m, 1H) 7.45-7.57 (m, 1H).

HRMS (EI) calcd for C₂₅H₃₂N₃O3. Cl: 460.2412, found 460.2422.

optical rotation [α]²⁰_{D}=+33.2

### EXAMPLE 443

### N-(3-cyano-4-fluorophenyl)-N′-[(3aS*,6R*,7aS*)-3a-(3,4-dimethoxyphenyl)-1-methyloctahydro-1H-indol-6-yl]urea trifluoroacetate

Triethylamine (33 μL, 0.24 mmol) was added to a solution of 5-Amino-2-fluoro-benzonitrile (19 mg, 0.14 mmol) in dry CH₂Cl₂ (1.5 mL) under an atmosphere of nitrogen. Triphosgene (14 mg, 0.048 mmol) dissolved in dry CH₂Cl₂ (0.5 mL) was added drop-wise and the reaction was stirred at ambient temperature for 2 hrs. (3aS*,6R*,7aS*)-3a-(3,4-dimethoxyphenyl)-1-methyloctahydro-1H-indol-6-amine, Comparative Example 7, (40 mg, 0.14 mmol) dissolved in dry CH₂Cl₂ (1.0 mL) was added and the reaction was stirred over night at ambient temperature. After concentration, the crude product was purified using preparative HPLC to give 27 mg (34%) of the product as a colorless solid.

¹H NMR (270 MHz, MeOH-d₄): 1.08-1.26 (m, 1H) 1.61-1.82 (m, 3H) 2.08-2.21 (m, 2H) 2.25-2.46 (m, 2H) 3.01 (s, 3H) 3.22-3.34 (m, 1H) 3.73 (s, 3H) 3.76 (s, 3H) 3.69-3.88 (m, 2H) 3.97-4.05 (m, 1H) 6.85-6.91 (m, 3H) 7.14 (t, J=8.9 Hz, 1H) 7.40-7.49 (m, 1H) 7.72-7.79 (m, 1H).

MS (ESI+) for C₂₅H₂₉FN₄O₃ m/z 453 (M+H)⁺.

HRMS (EI) calcd for C₂₅H₂₉F N₄O₃: 452.2224, found 452.2210.

### EXAMPLE 444

### N-[(3aS*,6R*,7aS*)-3a-(3,4-dimethoxyphenyl)-1-methyloctahydro-1H-indol-6-yl]-N′-[3-(1,3-oxazol-5-yl)phenyl]urea trifluoroacetate

Triethylamine (33 μL, 0.24 mmol) was added to a solution of 3-Oxazol-5-yl-phenylamine (19 mg, 0.14 mmol) in dry CH₂Cl₂ (1.5 mL) under an atmosphere of nitrogen. Triphosgene (14 mg, 0.048 mmol) dissolved in dry CH₂Cl₂ (0.5 mL) was added drop-wise and the reaction was stirred at ambient temperature for 2 hrs. (3aS*,6R*,7aS*)-3a-(3,4-dimethoxyphenyl)-1-methyloctahydro-1H-indol-6-amine, Comparative Example 7, (40 mg, 0.14 mmol) dissolved in dry CH₂Cl₂ (1.0 mL) was added and the reaction stirred over night at ambient temperature. After concentration, the crude product was purified using preparative HPLC to give 33 mg (47%) of the product as a colorless solid.

¹H NMR (270 MHz, MeOH-d₄): 1.11-1.38 (m, 1H) 1.671-1.95 (m, 3H) 2.12-2.32 (m, 2H) 2.34-2.60 (m, 2H) 3.12 (s, 3H) 3.28-3.45 (m, 1H) 3.83 (s, 3H) 3.86 (s, 3H) 3.74-4.01 (m, 2H) 4.05-4.17 (m, 1H) 6.89-7.06 (m, 3H) 7.20-7.38 (m, 3H) 7.41-7.56 (m, 1H) 7.78-7.89 (m, 1H) 8.17-8.29 (m, 1H).

HRMS (EI) calcd for C₂₅H₂₉F N₄O₃: 476.2424, found 476.2416.

### EXAMPLE 445

### N-[(3aS*,6R*,7aS*)-3a-(3,4-dimethoxyphenyl)-1-methyloctahydro-1H-indol-6-yl]-N′-[8-fluoro-2-(trifluoromethyl)quinolin-4-yl]urea trifluoroacetate

Step 1: To a suspension of 8-fluoro-2-trifluoromethyl-quinolin-4-ol (23 mg, 1.0 mmol) in dry toluene (3 mL) was added tosyl isocyanate (0.35 mL, 2.3 mmol) in one portion and the reaction mixture was heated at reflux for 60 h. 90% H₂SO₄ (0.4 mL) was added to the stirred solution, cooled to 0° C. The two-phase mixture was warmed to 60° C. with stirring for 12 h. After cooling to 0° C., crushed ice (0.5 g) was added followed by 46% aqueous NaOH (0.95 mL). The precipitated Na₂SO₄ was filtered off and the aqueous phase washed thoroughly with CH₂Cl₂. The organic phase was washed with brine and dried (Na₂SO₄).

Step 2:Triethylamine (33 μL, 0.24 mmol) was added to a solution of crude 8-fluoro-2-(trifluoromethyl)quinolin-4-amine (50 mg from step 1) in dry CH₂Cl₂ (1.5 mL) under an atmosphere of nitrogen. Triphosgene (14 mg, 0.048 mmol) dissolved in dry CH₂Cl₂ (0.5 mL) was added drop-wise and the reaction was stirred at ambient temperature for 2 hrs. (3aS*,6R*,7aS*)-3a-(3,4-dimethoxyphenyl)-1-methyloctahydro-1H-indol-6-amine, Comparative Example 7 (40 mg, 0.14 mmol) dissolved in dry CH₂Cl₂ (1.0 mL) was added and the reaction was stirred over night at ambient temperature. After concentration, the crude product was purified using preparative HPLC to give 21.3 mg (32%) of the product as a colorless solid.

¹H NMR (270 MHz, MeOH-d₄) δ 1.19-1.42 (m, 1H) 1.74-2.00 (m, 3H) 2.14-2.38 (m, 2H) 2.38-2.73 (m, 2H) 3.14 (s, 3H) 3.31-3.47 (m, 1H) 3.78-4.11 (m, 2H) 3.84 (s, 3H) 3.87 (s, 3H) 4.11-4.20 (m, 1H) 6.90-7.07 (m, 3H) 7.48-7.72 (m, 2H) 7.86-7.98 (m, 1H) 8.67-8.76 (m, 1H).

HRMS (EI) calcd for C₂₈H₃₀F₄N₄: 546.2254, found 546.2236.

### EXAMPLE 446

### N-[(3aR,6S,7aR)-3a-(3,4-dimethoxyphenyl)-1-methyloctahydro-1H-indol-6-yl]-N′-[3-fluoro-5-(trifluoromethyl)phenyl]urea trifluoroacetate

DIPEA (21 μL, 0.12 mmol) and 1-fluoro-3-isocyanato-5-trifluoromethyl-benzene (25 mg, 0.12 mmol) in CH₂Cl₂ (1 mL) were added to a solution of (3aR,6S,7aR)-3a-(3,4-dimethoxyphenyl)-1-methyloctahydro-1H-indol-6-amine, Comparative Example 11 (29 mg, 0.10 mmol) in CH₂Cl₂ (1 mL). The reaction mixture was stirred 2 h. After concentration the crude product was purified using preparative HPLC to give 48.1 mg (79%) of the product as a colorless solid.

¹H NMR (270 MHz, METHANOL-D4) ppm 1.14-1.40 (m, 1H) 1.69-1.94 (m, 3H) 2.15-2.32 (m, 2H) 2.36-2.58 (m, 2H) 3.11 (s, 3H) 3.32-3.43 (m, 1H) 3.81-3.98 (m, 2H) 3.83 (s, 3H) 3.86 (s, 3H) 4.07-4.14 (m, 1H) 6.98 (s, 4H) 7.44-7.55 (m, 2H)

HRMS (EI) calcd for C₂₅H₂₉F₄N₃O₃: 495.2145, found 495.2140.

Optical rotation [α]²⁰_{D}=−5.40

### EXAMPLE 447

### N-(4-bromophenyl)-N′-[(3aR,6S,7aR)-3a-(3,4-dimethoxyphenyl)-1-methyloctahydro-1H-indol-6-yl]urea trifluoroacetate

DIPEA (21 μL, 0.12 mmol) and 4-bromophenylisocyanate (24 mg, 0.12 mmol) in CH₂Cl₂ (1 mL) were added to a solution of (3aR,6S,7aR)-3a-(3,4-dimethoxyphenyl)-1-methyloctahydro-1H-indol-6-amine, Comparative Example 11, (29 mg, 0.10 mmol) in CH₂Cl₂ (1 mL). The reaction mixture was stirred for 2 h. After concentration the crude product was purified using preparative HPLC to give 1.5 mg (2%) of the product as a colorless solid.

¹H NMR (270 MHz, METHANOL-D4) ppm 1.16-1.34 (m, 1H) 1.74-1.94 (m, 3H) 2.16-2.33 (m, 2H) 2.35-2.59 (m, 2H) 3.11 (s, 3H) 3.32-3.46 (m, 1H) 3.83 (s, 3H) 3.81-3.98 (m, 2H) 3.84-3.88 (m, 3H) 4.05-4.16 (m, 1H) 6.93-7.05 (m, 3H) 7.22-7.41 (m, 4H)

HRMS (EI) calcd for C₂₄H₃₀BrN₃O₃: 487.1471, found 487.1459.

### EXAMPLE 448

### N-(2,4-difluorophenyl)-N′-[(3aR,6S,7aR)-3a-(3,4-dimethoxyphenyl)-1-methyloctahydro-1H-indol-6-yl]urea trifluoroacetate

DIPEA (21 μL, 0.12 mmol) and 2,4-difluorophenyl isocyanate (19 mg, 0.12 mmol) in CH₂Cl₂ (1 mL) were added to a solution of (3aR,6S,7aR)-3a-(3,4-dimethoxyphenyl)-1-methyloctahydro-1H-indol-6-amine, Comparative Example 11, (29 mg, 0.10 mmol) in CH₂Cl₂ (1 mL). The reaction mixture was stirred 2 h. After concentration the crude product was purified using preparative HPLC to give 42 mg (75%) of the product as a colorless solid.

¹H NMR (270 MHz, METHANOL-D4) ppm 1.11-1.32 (m, 1H) 1.67-1.93 (m, 3H) 2.12-2.33 (m, 2H) 2.34-2.58 (m, 2H) 3.10 (s, 3H) 3.32-3.42 (m, 1H) 3.80-3.98 (m, 2H) 3.83 (s, 3H) 3.84-3.88 (m, 3H) 4.06-4.15 (m, 1H) 6.81-6.91 (m, 1H) 6.91-7.02 (m, 4H) 7.76-7.88 (m, 1H)

HRMS (EI) calcd for C₂₄H₂₉F₂N₃O₃: 445.2177, found 445.2164.

Optical rotation [α]²⁰_{D}=−2.71

### EXAMPLE 449

### N-(2,5-difluorophenyl)-N′-[(3aR,6S,7aR)-3a-(3,4-dimethoxyphenyl)-1-methyloctahydro-1H-indol-6-yl]urea trifluoroacetate

DIPEA (21 μL, 0.12 mmol) and 2,5-difluorophenyl isocyanate (19 mg, 0.12 mmol) in CH₂Cl₂ (1 mL) were added to a solution of (3aR,6S,7aR)-3a-(3,4-dimethoxyphenyl)-1-methyloctahydro-1H-indol-6-amine, Comparative Example 11, (29 mg, 0.10 mmol) in CH₂Cl₂ (1 mL). The reaction mixture was stirred 2 h. After concentration the crude product was purified using preparative HPLC to give 40.3 mg (72%) of the product as a colorless solid.

¹H NMR (270 MHz, METHANOL-D4) ppm 1.09-1.33 (m, 1H) 1.69-1.94 (m, 3H) 2.13-2.33 (m, 2H) 2.34-2.61 (m, 2H) 3.11 (s, 3H) 3.31-3.44 (m, 1H) 3.78-3.99 (m, 2H) 3.83 (s, 3H) 3.86 (s, 3H) 4.06-4.15 (m, 1H) 6.57-6.73 (m, 1H) 6.92-7.01 (m, 3H) 7.00-7.13 (m, 1H) 7.80-7.93 (m, 1H)

HRMS (EI) calcd for C₂₄H₂₉F₂N₃O₃: 445.2177, found 445.2179.

optical rotation [α]²⁰_{D}=−6.10

### EXAMPLE 450

### N-[(3aR,6S,7aR)-3a-(3,4-dimethoxyphenyl)-1-methyloctahydro-1H-indol-6-yl]-N′-[4-methoxy-3-(trifluoromethyl)phenyl]urea trifluoroacetate

Triethylamine (28 μL, 0.20 mmol) was added to a solution of 4-methoxy-2-trifluoromethyl aniline (23 mg, 0.12 mmol) in dry CH₂Cl₂ (1.0 mL) under an atmosphere of nitrogen. Triphosgene (12 mg, 0.040 mmol) dissolved in dry CH₂Cl₂ (0.5 mL) was added drop-wise and the reaction was stirred at ambient temperature for 2 hrs. (3aR,6S,7aR)-3a-(3,4-dimethoxyphenyl)-1-methyloctahydro-1H-indol-6-amine, Comparative Example 11, (29 mg, 0.10 mmol) dissolved in dry CH₂Cl₂ (1.0 mL) was added and the reaction was stirred over night at ambient temperature. After concentration, the crude product was purified using preparative HPLC to give 40.4 mg (65%) of the product as a colorless solid.

¹H NMR (270 MHz, METHANOL-D4) ppm 1.13-1.36 (m, 1H) 1.69-1.94 (m, 3H) 2.14-2.32 (m, 2H) 2.34-2.59 (m, 2H) 3.10 (s, 3H) 3.31-3.44 (m, 1H) 3.80-3.99 (m, 2H) 3.83 (s, 3H) 3.84 (s, 3H) 3.86 (s, 3H) 4.04-4.17 (m, 1H) 6.95-7.01 (m, 3H) 7.07 (d, J=8.9 Hz, 1H) 7.42 (dd, J=8.9, 2.7 Hz, 1H) 7.67 (d, J=2.5 Hz, 1H).

HRMS (EI) calcd for C₂₆H₃₂F₃N₃O₄: 507.2345, found 507.2341.

Optical rotation [α]²⁰_{D}=−6.04

### EXAMPLE 451

### N-[4-cyano-3-(trifluoromethyl)phenyl]-N′-[(3aR,6S,7aR)-3a-(3,4-dimethoxyphenyl)-1-methyloctahydro-1H-indol-6-yl]urea trifluoroacetate

Triethylamine (28 μL, 0.20 mmol) was added to a solution of 4-amino-2-trifluoromethyl-benzonitrile (22 mg, 0.12 mmol) in dry CH₂Cl₂ (1.0 mL) under an atmosphere of nitrogen. Triphosgene (12 mg, 0.040 mmol) dissolved in dry CH₂Cl₂ (0.5 mL) was added drop-wise and the reaction was stirred at ambient temperature for 2 hrs. (3aR,6S,7aR)-3a-(3,4-dimethoxyphenyl)-1-methyloctahydro-1H-indol-6-amine, Comparative Example 11, (29 mg, 0.10 mmol) dissolved in dry CH₂Cl₂ (1.0 mL) was added and the reaction was stirred over night at ambient temperature. After concentration, the crude product was purified using preparative HPLC to give 20.5 mg (33%) of the product as a colorless solid.

¹H NMR (270 MHz, METHANOL-D4) ppm 1.17-1.36 (m, 1H) 1.68-1.94 (m, 3H) 2.17-2.30 (m, 2H) 2.37-2.57 (m, 2H) 3.12 (s, 3H) 3.32-3.44 (m, 1H) 3.80-4.00 (m, 2H) 3.83 (s, 3H) 3.86 (s, 3H) 4.07-4.17 (m, 1H) 6.94-7.03 (m, 3H) 7.65 (dd, J=8.5, 2.1 Hz, 1H) 7.81 (d, J=8.5 Hz, 1H) 8.11 (d, J=2.2 Hz, 1H).

HRMS (EI) calcd for C₂₆H₂₉F₃N₄O₃: 502.2192, found 502.2178.

optical rotation [α]²⁰_{D}=−8.30

### EXAMPLE 452

### N-[(3aR,6S,7aR)-3a-(3,4-dimethoxyphenyl)-1-methyloctahydro-1H-indol-6-yl]-N′-(3,4,5-trifluorophenyl)urea trifluoroacetate

Triethylamine (28 μL, 0.20 mmol) was added to a solution of 3,4,5-trifluorophenyl amine (18 mg, 0.12 mmol) in dry CH₂Cl₂ (1.0 mL) under an atmosphere of nitrogen. Triphosgene (12 mg, 0.040 mmol) dissolved in dry CH₂Cl₂ (0.5 mL) was added drop-wise and the reaction was stirred at ambient temperature for 2 hrs. (3aR,6S,7aR)-3a-(3,4-dimethoxyphenyl)-1-methyloctahydro-1H-indol-6-amine, Comparative Example 11, (29 mg, 0.10 mmol) dissolved in dry CH₂Cl₂ (1.0 mL) was added and the reaction was stirred over night at ambient temperature. After concentration, the crude product was purified using preparative HPLC to give 41.5 mg (73%) of the product as a colorless solid.

¹H NMR (270 MHz, METHANOL-D4) ppm 1.14-1.34 (m, 1H) 1.69-1.92 (m, 3H) 2.14-2.31 (m, 2H) 2.35-2.55 (m, 2H) 3.11 (s, 3H) 3.31-3.43 (m, 1H) 3.80-3.97 (m, 2H) 3.83 (s, 3H) 3.86 (s, 3H) 4.06-4.15 (m, 1H) 6.95-7.02 (m, 3H) 7.10-7.23 (m, 2H)

HRMS (EI) calcd for C₂₄H₂₈F₃N₃O₃: 463.2083, found 463.2084.

Optical rotation [α]²⁰_{D}=−2.80

### EXAMPLE 453

### N-(3-chloro-4-cyanophenyl)-N′-[(3aR,6S,7aR)-3a-(3,4-dimethoxyphenyl)-1-methyloctahydro-1H-indol-6-yl]urea trifluoroacetate

Triethylamine (28 μL, 0.20 mmol) was added to a solution of 4-amino-2-chloro-benzonitrile (18 mg, 0.12 mmol) in dry CH₂Cl₂ (1.0 mL) under an atmosphere of nitrogen. Triphosgene (12 mg, 0.040 mmol) dissolved in dry CH₂Cl₂ (0.5 mL) was added drop-wise and the reaction was stirred at ambient temperature for 2 hrs. (3aR,6S,7aR)-3a-(3,4-dimethoxyphenyl)-1-methyloctahydro-1H-indol-6-amine, Comparative Example 11, (29 mg, 0.10 mmol) dissolved in dry CH₂Cl₂ (1.0 mL) was added and the reaction was stirred over night at ambient temperature. After concentration, the crude product was purified using preparative HPLC to give 31.1 mg (53%) of the product as a colorless solid.

¹H NMR (270 MHz, METHANOL-D4) ppm 1.15-1.37 (m, 1H) 1.70-1.93 (m, 3H) 2.13-2.34 (m, 2H) 2.36-2.57 (m, 2H) 3.11 (s, 3H) 3.30-3.42 (m, 1H) 3.78-4.00 (m, 2H) 3.83 (s, 3H) 3.86 (s, 3H) 4.07-4.14 (m, 1H) 6.94-7.01 (m, 3H) 7.32 (dd, J=8.7, 2.2 Hz, 1H) 7.61 (d, J=8.7 Hz, 1H) 7.85 (d, J=2.0 Hz, 1H)

HRMS (EI) calcd for C₂₄H₂₈F₃N₃O₃: 468.1928, found 468.1925.

Optical rotation [α]²⁰_{D}=−10.4

### EXAMPLE 454

### N-[(3aS*,6R*,7aS*)-1-(chloroacetyl)-3a-(3,4-dimethoxyphenyl)octahydro-1H-indol-6-yl]-N′-(3,4-difluorophenyl)urea

N-(3,4-difluorophenyl)-N′-[(3aS*,6R*,7aS*)-3a-(3,4-dimethoxyphenyl)octahydro-1H-indol-6-yl]urea, Example 175, (0.4 g, 0.93 mmol) was dissolved in CH₂Cl₂ (8 ml) and triethylamine (0.173 ml, 1.2 mmol). Chloroacetylchloride (0.077 ml, 0.97 mmol) was added. The mixture was stirred for 20 minutes at room temperature and evaporated. The residue was dissolved in CH₂Cl₂ and water and pH was adjusted to 3 with 1N HCl (4 drops). The organic phase was washed with water (2×) dried (MgSO₄) and evaporated. The crude product was purified by column chromatography on silica gel using 2.5% to 5% MeOH in CH₂Cl₂. Yield 0.18 g (38%). White/green solid. MS (ionspray; [M+H]⁺) m/z: 508.2. HRMS for C₂₅H₂₈ClF₂N₃O₄: Calcd, 507.1736; found, 507.1739.

¹H NMR (400 MHz, CHLOROFORM-D) δ ppm 1.59-1.87 (m, 3H) 1.88-2.17 (m, 4H) 2.25-2.46 (m, 2H) 3.40-3.70 (m, 2H) 3.76 (s, 3H) 3.84 (s, 3H) 3.96 (dd, 2H) 4.55 (s, 1H) 5.55 (s, 1H) 6.65-6.83 (m, 3H) 6.91-7.03 (m, 2H) 7.32-7.52 (m, 2H).

### EXAMPLE 455

### N-(3,4-difluorophenyl)-N′-{(3aS*,6R*,7aS*)-3a-(3,4-dimethoxyphenyl)-1-[(isopropylamino)acetyl]octahydro-1H-indol-6-yl}urea trifluoroacetate

N-[(3aS*,6R*,7aS*)-1-(chloroacetyl)-3a-(3,4-dimethoxyphenyl)octahydro-1H-indol-6-yl]-N′-(3,4-difluorophenyl)urea, Example 454, (0.010 g, 0.023 mmol) was dissolved in CH₂Cl₂ (1 ml) and isopropylamine (0.010 ml, 0.12 mmol) was added. The mixture was heated 40° C. and stirred for 20 hours. After filtration and evaporation the crude product was purified by reversed phase HPLC Isolated as the TFA salt. Yield 4.7 mg (19%). Colorless gum. MS (ionspray; [M+H]⁺) m/z: 531.2. HRMS for C₂₈H₃₆F₂N₄O₄: Calcd, 530.2705; found, 530.2709.

### EXAMPLE 456

### N-[(3aS*,6R*,7aS*)-1-(anilinoacetyl)-3a-(3,4-dimethoxyphenyl)octahydro-1H-indol-6-yl]-N′-(3,4-difluorophenyl)urea

N-[(3aS*,6R*,7aS*)-1-(chloroacetyl)-3a-(3,4-dimethoxyphenyl)octahydro-1H-indol-6-yl]-N′-(3,4-difluorophenyl)urea, Example 454, (0.010 g, 0.023 mmol) was dissolved in chloroform (1 ml) and aniline (0.003 ml, 0.03 mmol) and 1 drop of triethylamine was added. The mixture was heated to 65° C. and heated overnight. Additional aniline (0.005 ml, 0.005 mmol) was added and the mixture was stirred for additional 2 days then evaporated. The crude product was purified by reversed phase HPLC. Yield 2.1 mg (19%). Light yellow solid. HRMS for C₃₁H₃₄F₂N₄O₄: Calcd, 564.2548; found, 564.2796.

### EXAMPLE 457

### N-(3,4-difluorophenyl)-N′-{(3aS*,6R*,7aS*)-3a-(3,4-dimethoxyphenyl)-1-[(methylamino)acetyl]octahydro-1H-indol-6-yl}urea trifluoroacetate

N-[(3aS*,6R*,7aS*)-1-(chloroacetyl)-3a-(3,4-dimethoxyphenyl)octahydro-1H-indol-6-yl]-N′-(3,4-difluorophenyl)urea, Example 454, (0.015 g, 0.03 mmol) was dissolved in THF (1 ml) and a solution of 40% methylamine in water (0.5 ml) was added. The mixture was stirred for 10 minutes and evaporated. The crude product was purified by reversed phase HPLC. Isolated as the TFA salt. Yield 10.4 mg (57%). Light yellow liquid. MS (ionspray; [M+H]⁺) m/z: 503.2. HRMS for C₂₆H₃₂F₂N₄O₄: Calcd, 502.2392; found, 502.2372.

### EXAMPLE 458

### N-[(3aS*,6R*,7aS*)-1-(aminoacetyl)-3a-(3,4-dimethoxyphenyl)octahydro-1H-indol-6-yl]-N′-(3,4-difluorophenyl)urea

N-[(3aS*,6R*,7aS*)-1-(chloroacetyl)-3a-(3,4-dimethoxyphenyl)octahydro-1H-indol-6-yl]-N′-(3,4-difluorophenyl)urea, Example 454, (0.015 g, 0.03 mmol) was dissolved in chloroform saturated with NH₃ (3 ml). The mixture was heated to 60° C. in a sealed vial overnight. The crude product was purified by reversed phase. Isolated as the TFA salt.

Yield 5.6 mg (39%). White solid. MS (ionspray; [M+H]⁺) m/z: 489.2. HRMS for C₂₅H₃₀F₂N₄O₄: Calcd, 488.2235; found, 488.2232.

### EXAMPLE 459

### N-(3,4-difluorophenyl)-N′-((3aS*,6R*,7aS*)-3a-(3,4-dimethoxyphenyl)-1-{[(2-furylmethyl)amino]acetyl}octahydro-1H-indol-6-yl)urea trifluoroacetate

N-[(3aS*,6R*,7aS*)-1-(chloroacetyl)-3a-(3,4-dimethoxyphenyl)octahydro-1H-indol-6-yl]-N′-(3,4-difluorophenyl)urea, Example 454, (0.015 g, 0.03 mmol) was dissolved in THF and furfurylamine (0.010 g, 0.1 mmol) was added.

The mixture was stirred at room temperature overnight and evaporated. The crude product was purified by reversed phase HPLC. Isolated as the TFA salt. Yield 13 mg (65%). White solid. MS (ionspray; [M+H]⁺) m/z: 569.2. HRMS for C₃₀H₃₄F₂N₄O₅: Calcd, 568.2497; found, 568.2487.

### EXAMPLE 460

### N-(3,4-difluorophenyl)-N′-((3aS*,6R*,7aS*)-3a-(3,4-dimethoxyphenyl)-1-{[(pyridin-4-ylmethyl)amino]acetyl}octahydro-1H-indol-6-yl)urea bis(trifluoroacetate)

N-[(3aS*,6R*,7aS*)-1-(chloroacetyl)-3a-(3,4-dimethoxyphenyl)octahydro-1H-indol-6-yl]-N′-(3,4-difluorophenyl)urea, Example 454, (0.015 g, 0.03 mmol) was dissolved in THF and 4-picolylamine (0.0108 g, 0.1 mmol) was added.

The mixture was stirred at room temperature overnight and evaporated. The crude product was purified by reversed phase HPLC. Isolated as the bis TFA salt. Yield 10 mg (42%). Light brown gum. MS (ionspray; [M+H]⁺) m/z: 580.4. HRMS for C₃₁H₃₅F₂N₅O₄: Calcd, 579.2657; found, 579.2673.

### EXAMPLE 461

### N-[(3aS*,6R*,7aS*)-1-{[(4-chlorobenzyl)amino]acetyl}-3a-(3,4-dimethoxyphenyl)octahydro-1H-indol-6-yl]-N′-(3,4-difluorophenyl)urea trifluoroacetate

N-[(3aS*,6R*,7aS*)-1-(chloroacetyl)-3a-(3,4-dimethoxyphenyl)octahydro-1H-indol-6-yl]-N′-(3,4-difluorophenyl)urea (0.015 g, 0.03 mmol), Example 454, was dissolved in THF and 4-chlorobenzylamine (0.0146 g, 0.1 mmol) was added.

The mixture was stirred at room temperature overnight and the temperature was raised to 45° C. and stirred for additional 3 days and evaporated. The crude product was purified by reversed phase HPLC using. Isolated as the TFA salt. Yield 12 mg (55%). White solid. MS (ionspray; [M+H]⁺) m/z: 613.2. HRMS for C₃₂H₃₅ClF₂N₄O₄: Calcd, 612.2315; found, 612.2318.

### EXAMPLE 462

### N-(3,4-difluorophenyl)-N′-{(3aS*,6R*,7aS*)-3a-(3,4-dimethoxyphenyl)-1-[(ethylamino)acetyl]octahydro-1H-indol-6-yl}urea trifluoroacetate

N-[(3aS*,6R*,7aS*)-1-(chloroacetyl)-3a-(3,4-dimethoxyphenyl)octahydro-1H-indol-6-yl]-N′-(3,4-difluorophenyl)urea, Example 454, (0.015 g, 0.03 mmol) was dissolved in THF and ethylamine 2N in THF (0.005 g, 0.1 mmol) was added. The mixture was stirred at room temperature overnight and the temperature was raised to 45° C. and stirred for additional 3 days and evaporated. The crude product was purified by reversed phase HPLC. Isolated as the TFA salt. Yield 10.4 mg (55%). Yellow gum. MS (ionspray; [M+H]⁺) m/z: 517.4. HRMS for C₂₇H₃₄F₂N₄O₄: Calcd, 516.2548; found, 516.2534.

### EXAMPLE 463

### N-(3,4-difluorophenyl)-N′-{(3aS*,6R*,7aS*)-3a-(3,4-dimethoxyphenyl)-1-[(4-methylpiperidin-1-yl)acetyl]octahydro-1H-indol-6-yl}urea trifluoroacetate

N-[(3aS*,6R*,7aS*)-1-(chloroacetyl)-3a-(3,4-dimethoxyphenyl)octahydro-1H-indol-6-yl]-N′-(3,4-difluorophenyl)urea, Example 454, (0.015 g, 0.03 mmol) was dissolved in THF and 4-methyl-piperidine (0.010 g, 0.1 mmol) was added. The mixture was stirred at room temperature overnight and evaporated. The crude product was purified by reversed phase HPLC. Isolated as the TFA salt. Yield 16.0 mg (78%). White solid. MS (ionspray; [M+H]⁺) m/z: 571.2. HRMS for C₃₁H₄₀F₂N₄O₄: Calcd, 570.3018; found, 570.3017.

### EXAMPLE 464

### N-(3,4-difluorophenyl)-N′-[(3aS*,6R*,7aS*)-3a-(3,4-dimethoxyphenyl)-1-(pyrrolidin-1-ylacetyl)octahydro-1H-indol-6-yl]urea trifluoroacetate

N-[(3aS*,6R*,7aS*)-1-(chloroacetyl)-3a-(3,4-dimethoxyphenyl)octahydro-1H-indol-6-yl]-N′-(3,4-difluorophenyl)urea, Example 454, (0.015 g, 0.03 mmol) was dissolved in THF and pyrrolidine (0.007 g, 0.1 mmol) was added. The mixture was stirred at room temperature overnight and evaporated. The crude product was purified by reversed phase HPLC. Isolated as the TFA salt. Yield 13.3 mg (68%). Colorless gum. MS (ionspray; [M+H]⁺) m/z: 543.4.

### EXAMPLE 465

### N-(3,4-difluorophenyl)-N′-((3aS*,6R*,7aS*)-3a-(3,4-dimethoxyphenyl)-1-{[(2-hydroxyethyl)amino]acetyl}octahydro-1H-indol-6-yl)urea trifluoroacetate

N-[(3aS*,6R*,7aS*)-1-(chloroacetyl)-3a-(3,4-dimethoxyphenyl)octahydro-1H-indol-6-yl]-N′-(3,4-difluorophenyl)urea, Example 454, (0.015 g, 0.03 mmol) was dissolved in THF and ethanolamine (0.006 g, 0.1 mmol) was added.

The mixture was stirred at room temperature overnight and evaporated. The crude product was purified by reversed phase HPLC. Yield 11.0 mg (57%). Colorless liquid. MS (ionspray; [M+H]⁺) m/z: 533.2. HRMS for C₂₇H₃₄F₂N₄O₅: Calcd, 532.2497; found, 532.2517.

### EXAMPLE 466

### N-(3,4-difluorophenyl)-N′-[(3aS*,6R*,7aS*)-3a-(3,4-dimethoxyphenyl)-1-(3,3,5,5-tetramethylcyclohexyl)octahydro-1H-indol-6-yl]urea trifluoroacetate

N-(3,4-difluorophenyl)-N′-[(3aS*,6R*,7aS*)-3a-(3,4-dimethoxyphenyl)octahydro-1H-indol-6-yl]urea, Example 175, (0.010 g, 0.023 mmol) was dissolved in MeOH (0.5 ml) and 3,3,5,5-tetramethylcyclohexanone (0.018 g, 0.115 mmol) was added. After 5 minutes NaCNBH₃ (0.15 g, 0.24 mmol) was added all in one portion.

The mixture was stirred for 17 h and filtered. The crude product was purified by reversed phase HPLC. Isolated as the TFA salt. Yield 11.0 mg (70%). Colourless liquid. MS (ionspray; [M+H]⁺) m/z: 570.4. HRMS for C₃₃H₄₅F₂N₃O₃: Calcd, 569.3429; found, 569.3450.

### EXAMPLE 467

### N-[(3aS*,6R*,7aS*)-1-bicyclo[2.2.1]hept-2-yl-3a-(3,4-dimethoxyphenyl)octahydro-1H-indol-6-yl]-N′-(3,4-difluorophenyl)urea trifluoroacetate

N-(3,4-difluorophenyl)-N′-[(3aS*,6R*,7aS*)-3a-(3,4-dimethoxyphenyl)octahydro-1H-indol-6-yl]urea, Example 175, (0.010 g, 0.023 mmol) was dissolved in MeOH (0.5 ml) and 2-norcamphor (0.013 g, 0.115 mmol) was added. After 5 minutes NaCNBH₃ (0.15 g, 0.24 mmol) was added all in one portion.

The mixture was stirred for 17 h and filtered. The crude product was purified by reversed phase HPLC. Isolated as the TFA salt. Yield 8.0 mg (54%). White solid. MS (ionspray; [M+H]⁺) m/z: 526.4. HRMS for C₃₀H₃₇F₂N₃O₃: Calcd, 525.2803; found, 525.2806.

### EXAMPLE 468

### N-(3,4-difluorophenyl)-N′-[(3aS*,6R*,7aS*)-3a-(3,4-dimethoxyphenyl)-1-(4-methylcyclohexyl)octahydro-1H-indol-6-yl]urea trifluoroacetate

N-(3,4-difluorophenyl)-N′-[(3aS*,6R*,7aS*)-3a-(3,4-dimethoxyphenyl)octahydro-1H-indol-6-yl]urea, Example 175, (0.010 g, 0.023 mmol) was dissolved in MeOH (0.5 ml) and 4-methylcyclohexanone (0.013 g, 0.115 mmol) was added. After 5 minutes NaCNBH₃ (0.15 g, 0.24 mmol) was added all in one portion.

The mixture was stirred for 18 h and filtered. The crude product was purified by reversed phase. Isolated as the TFA salt. Yield 8.0 mg (54%). White solid. MS (ionspray; [M+H]⁺) m/z: 528.4. HRMS for C₃₀H₃₇F₂N₃O₃: Calcd, 527.2959; found, 527.2959.

### EXAMPLE 469

### N-[(3aS*,6R*,7aS*)-1-allyl-3a-(3,4-dimethoxyphenyl)octahydro-1H-indol-6-yl]-N′-(3,4-difluorophenyl)urea trifluoroacetate

Allylbromide (0.0024 g, 0.028 mmol) was added to a solution of N-(3,4-difluorophenyl)-N′-[(3aS*,6R*,7aS*)-3a-(3,4-dimethoxyphenyl)octahydro-1H-indol-6-yl]urea, Example 175 (0.010 g, 0.023 mmol) in THF (1.0 ml). The mixture was heated at 50° C. overnight and evaporated. The crude product was purified by reversed phase HPLC. Isolated as the TFA salt. Yield 4.7 mg (35%). Colorless oil. MS (ionspray; [M+H]⁺) m/z: 472.2.

### EXAMPLE 470

### 2-[(3aS*,6R*,7aS*)-6-({[(3,4-difluorophenyl)amino]carbonyl}amino)-3a-(3,4-dimethoxyphenyl)octahydro-1H-indol-1-yl]-N,N-dimethylacetamide trifluoroacetate

N-(3,4-difluorophenyl)-N′-[(3aS*,6R*,7aS*)-3a-(3,4-dimethoxyphenyl)octahydro-1H-indol-6-yl]urea, Example 175 (0.010 g, 0.023 mmol), 2-chloro-N,N-dimethylacetamide (0.003 ml, 0028 mmol) and 1 drop of triethylamine was dissolved in THF (1 ml) and the mixture was heated 50° C. overnight. Additional dimethylacetamide (0.003 ml, 0.028 mmol) was added and the mixture was heated for an additional 8 hours. The mixture was evaporated and purified by reversed phase HPLC. Isolated as the TFA salt. Yield 8.3 mg (57%). Colorless gum. MS (ionspray; [M+H]⁺) m/z: 517.4. HRMS for C₂₇H₃₄F₂N₄O₄: Calcd, 516.2548; found, 516.2533.

### EXAMPLE 471

### Ethyl {[(3aS*,6R*,7aS*)-6-({[(3,4-difluorophenyl)amino]carbonyl}amino)-3a-(3,4-dimethoxyphenyl)octahydro-1H-indol-1-yl]carbonothioyl}carbamate

Ethoxycarbonylisothiocyanate (0.014 ml, 0.12 mmol) was added to a solution of N-(3,4-difluorophenyl)-N′-[(3aS*,6R*,7aS*)-3a-(3,4-dimethoxyphenyl)octahydro-1H-indol-6-yl]urea, Example 175, (0.049 g, 0.113 mmol) in CH₂Cl₂ (3 ml). The mixture was stirred for 1 hour and diluted with CH₂Cl₂ and water. pH was adjusted to 2 with 1N HCl, washed with water (2×), dried (MgSO₄) and evaporated. Yield: 56 mg (88%). Grey solid. MS (ionspray; [M+H]⁺) m/z: 563.4.

### EXAMPLE 472

### (3aS*,6R*,7aS*)-6-({[(3,4-difluorophenyl)amino]carbonyl}amino)-3a-(3,4-dimethoxyphenyl)octahydro-1H-indole-1-carbothioamide

Ethyl {[(3aS*,6R*,7aS*)-6-({[(3,4-difluorophenyl)amino]carbonyl}amino)-3a-(3,4-dimethoxyphenyl)octahydro-1H-indol-1-yl]carbonothioyl}carbamate, Example 471, (0.056 g, 0.1 mmol) and a 2N solution of NaOH (2 ml) were dissolved in MeOH (2 ml). The mixture was heated at 80° C. (homogenous mixture) overnight and evaporated. The solid was partitioned between CH₂Cl₂ and water, the organic phase was dried (MgSO₄) and evaporated. The crude product was purified by reversed phase HPLC. Yield 28 mg (57%). Light yellow solid. MS (ionspray; [M+H]⁺) m/z: 491.2. HRMS for C₂₄H₂₈F₂N₄O₃S: Calcd, 490.1850; found, 490.1852.

### EXAMPLE 473

### N-(3,4-difluorophenyl)-N′-[(3aS*,6R*,7aS*)-3a-(3,4-dimethoxyphenyl)-1-(4-methyl-1,3-thiazol-2-yl)octahydro-1H-indol-6-yl]urea

(3aS*,6R*,7aS*)-6-([(3,4-difluorophenyl)amino]carbonyl}amino)-3a-(3,4-dimethoxyphenyl)octahydro-1H-indole-1-carbothioamide, Example 472, (0.015 g, 0.031 mmol) and chloroacetone (0.0027 ml, 0,034 mmol) were dissolved in DMF (0.6 ml) and heated at 70° C. overnight. The mixture was cooled and evaporated. The crude product was purified by reversed phase HPLC. Isolated as the TFA salt. Yield 8.0 mg (50%). Light yellow liquid. MS (ionspray; [M+H]⁺) m/z: 529.2. HRMS for C₂₇H₃₀F₂N₄O₃S: Calcd, 528.2007; found, 528.2027.

### EXAMPLE 474

### N-(3,4-difluorophenyl)-N′-{(3aS*,6R*,7aS*)-3a-(3,4-dimethoxyphenyl)-1-[(4-methylpiperazin-1-yl)carbonyl]octahydro-1H-indol-6-yl}urea trifluoroacetate

N-(3,4-difluorophenyl)-N′-[(3aS*,6R*,7aS*)-3a-(3,4-dimethoxyphenyl)octahydro-1H-indol-6-yl]urea, Example 175, (0.010 g, 0.023 mmol), 4-methyl-1-piperazine-carbonylchloride hydrochloride (0.0051 g, 0.025 mmol) and 1 drop of triethylamine was dissolved in THF (1 ml) and stirred at 40° C. for 5 hours and evaporated. The crude product was purified by reversed phase HPLC. Isolated as the TFA salt. Yield 10.0 mg (65%). Light brown gum. MS (ionspray; [M+H]⁺) m/z: 558.2. HRMS for C₂₉H₃₇F₂N₅O₄: Calcd, 557.2814; found, 557.2826.

### EXAMPLE 475

### 4-[(3aS*,6R*,7aS*)-6-({[(3,4-difluorophenyl)amino]carbonyl}amino)-3a-(3,4-dimethoxyphenyl)octahydro-1H-indol-1-yl]-4-oxobutanoic acid

N-(3,4-difluorophenyl)-N′-[(3aS*,6R*,7aS*)-3a-(3,4-dimethoxyphenyl)octahydro-1H-indol-6-yl]urea, Example 175, (0.010 g, 0.023 mmol) and succinic anhydride (0.0025 g, 0.025 mmol) and 1 drop of triethylamine was dissolved in CH₂Cl₂ (1 ml) and stirred for 5 hours. The mixture was acidified to pH 3 with 1N HCl and washed with water (2×), dried (MgSO₄) and evaporated. Yield 11.5 mg (94%). Light yellow solid. MS (ionspray; [M+H]⁺) m/z: 532.2. HRMS for C₂₇H₃₁F₂N₃O₆: Calcd, 531.2181; found, 531.2174.

### EXAMPLE 476

### (3aS*,6R*,7aS*)-6-({[(3,4-difluorophenyl)amino]carbonyl}amino)-3a-(3,4-dimethoxyphenyl)octahydro-1H-indole-1-carboxamide

BrCN (0.0025 g, 0.025 mmol) was added to a suspension of N-(3,4-difluorophenyl)-N′-[(3aS*,6R*,7aS*)-3a-(3,4-dimethoxyphenyl)octahydro-1H-indol-6-yl]urea, Example 175 (0.010 g, 0.023 mmol) and NaHCO₃ (0.0084 g, 0.1 mmol) in EtOH (1 ml). The mixture was stirred for 2 hours at room temperature and evaporated. The crude product was purified by reversed phase HPLC. During which procedure the cyano-compund hydrolyzed to the corresponding urea. Yield 1.5 mg (14%). White solid. MS (ionspray; [M+H]⁺) m/z: 475.2. HRMS for C₂₄H₂₈F₂N₄O₄: Calcd, 474.2079; found, 474.2075.

### EXAMPLE 477

### N-(3,4-difluorophenyl)-N′-[(3aS*,6R*,7aS*)-3a-(3,4-dimethoxyphenyl)-1-pyrimidin-2-yloctahydro-1H-indol-6-yl]urea

N-(3,4-difluorophenyl)-N′-[(3aS*,6R*,7aS*)-3a-(3,4-dimethoxyphenyl)octahydro-1H-indol-6-yl]urea, Example 175, (0.010 g, 0.023 mmol), 2-chloropyrimidine (0.0053 g, 0.046) and K₂CO₃ (0.016 g, 0.115 mmol) was dissolved in DMF (1 ml). The mixture was heated at 100° C. for 4 hours and partitioned between water and diethylether, dried (MgSO₄) and evaporated. The crude product was purified by reversed phase HPLC. Isolated as the TFA salt. Yield 3.0 mg (26%). White solid. MS (ionspray; [M+H]⁺) m/z: 510.4. HRMS for C₂₇H₂₉F₂N₅O₃: Calcd, 509.2238; found, 509.2236

### EXAMPLE 478

### N-(3,4-difluorophenyl)-N′-((3aS*,6R*,7aS*)-3a-(3,4-dimethoxyphenyl)-1-{[2-(4-methylpiperidin-1-yl)ethyl]sulfonyl}octahydro-1H-indol-6-yl)urea trifluoroacetate

Triethylamine (0.010 ml, 0.1 mmol) was added to a solution of N-(3,4-difluorophenyl)-N′-[(3aS*,6R*,7aS*)-3a-(3,4-dimethoxyphenyl)octahydro-1H-indol-6-yl]urea, Example 175, (0.010 g, 0.023 mmol) and 2-chloroethanesulfonylchloride (0.0073 ml, 0.069 mmol) in CH₂Cl₂ (1 ml). The mixture was stirred for 30 minutes before 4-methylpiperidine (0.1 ml, 10 mmol) was added and the mixture was allowed to stir for 1 hour and evaporated. The crude product was purified by reversed phase HPLC. Isolated as the TFA salt. Yield 6.5 mg (19%). Colorless liquid. MS (ionspray; [M+H]⁺) m/z: 621.4. HRMS for C₃H₄₂F₂N₄O₅: Calcd, 620.2844; found, 620,2838.

### EXAMPLE 479

### N-(3,4-difluorophenyl)-N′-[(3aS*,6R*,7aS*)-3a-(3,4-dimethoxyphenyl) 1-(3-piperidin-1-ylpropyl)octahydro-1H-indol-6-yl]urea bis(trifluoroacetate)

1-(3-chloropropyl)-piperidine hydrochloride (0.010 g, 0.05 mmol) and 1 drop of triethylamine were added to a solution of N-(3,4-difluorophenyl)-N′-[(3aS*,6R*,7aS*)-3a-(3,4-dimethoxyphenyl)octahydro-1H-indol-6-yl]urea, Example 175, (0.015 g, 0.035 mmol) in THF (1 ml). The mixture was stirred overnight and evaporated. The crude product was purified by reversed phase HPLC. Isolated as the TFA salt. Yield 10.0 mg (28%). Colorless liquid. MS (ionspray; [M+H]⁺) m/z: 557.2. HRMS for C₃₁H₄₂F₂N₄O₃: Calcd, 556,3225; found, 556.3230.

### EXAMPLE 480

### N-(3,4-difluorophenyl)-N′-[(3aS*,6R*,7aS*)-3a-(3,4-dimethoxyphenyl)-1-(2-piperidin-1-ylethyl)octahydro-1H-indol-6-yl]urea trifluoroacetate

N-(2-chloroethyl)-piperidine hydrochloride (0.0092 g, 0.05 mmol) and 1 drop of triethylamine were added to a solution of N-(3,4-difluorophenyl)-N′-[(3aS*,6R*,7aS*)-3a-(3,4-dimethoxyphenyl)octahydro-1H-indol-6-yl]urea, Example 175, (0.015 g, 0.035 mmol) in THF (1 ml). The mixture was stirred overnight and evaporated. The crude product was purified by reversed phase HPLC. Isolated as the TFA salt. Yield 6.4 mg (28%). Brown solid. MS (ionspray; [M+H]⁺) m/z: 543.2. HRMS for C₃₁H₄₂F₂N₄O₃: Calcd, 542.3068; found, 542.3049.

### EXAMPLE 481

### N-[(3aS*,6R*,7aS*)-1-[(chloromethyl)sulfonyl]-3a-(3,4-dimethoxyphenyl)octahydro-1H-indol-6-yl]-N′-(3,4-difluorophenyl)urea

Chloromethanesulfonylchloride (0.0125 g, 0.14 mmol) and triethylamine (0.020 ml, 0.138 mmol) were added to a solution of N-(3,4-difluorophenyl)-N′-[(3aS*,6R*,7aS*)-3a-(3,4-dimethoxyphenyl)octahydro-1H-indol-6-yl]urea, Example 175, (0.050 g, 0.116 mmol) in THF (2 ml). The mixture was stirred for 10 minutes and additional triethylamine (0.020 ml, 0.138 mmol) was added and the mixture was allow to stir for 1 hour and evaporated. The residue was partitioned between water and CH₂Cl₂. The organic phase was dried (MgSO₄) and evaporated. The crude product was filtered through a plug of silica using 5% MeOH in CH₂Cl₂ as the eluent. Yield 44 mg (70%). White solid. MS (ionspray; [M+H]⁺) m/z: 542.2.

### EXAMPLE 482

### N-(3,4-difluorophenyl)-N′-[(3aS*,6R*,7aS*)-3a-(3,4-dimethoxyphenyl)-1-phenyloctahydro-1H-indol-6-yl]urea

1-(3,4-dimethoxyphenyl)cyclopropanecarbaldehyde (0.5 g, 2.46 mmol), aniline hydrochloride (0.43 g, 3.3 mmol), sodium sulfate (0.7 g, 49 mmol) and methylvinylketone (0.26 ml, 3.17 mmol) were dissolved in MeCN (8 ml) and DMSO (1.5 ml) and stirred at 100° C. for 2 hours. The mixture was cooled and diluted with diethylether. The mixture was washed with 1N NaOH and water, and the organic phase was dried (MgSO₄) and evaporated. The crude product was purified by column chromatography on silica gel with CH₂Cl₂ as the eluent. Yield 0.205 g of the expected ketone intermediate, purity 85%. Ammonium acetate (1.34 g, 17.4 mmol) was added to a solution of this ketone (0.205 g, 0.58 mmol) in MeOH (3 ml) and the mixture was stirred at 70° C. for 30 minutes then treated with NaCN(BH3) (0.183 g, 2.9 mmol) and stirred at this temperature for an additional 30 mins. Then evaporated. The crude product was partitioned between water and CH₂Cl₂, washed with water (2×), dried (MgSO₄) and evaporated. The crude product was purified by column chromatography on silica gel using CHCl₃ saturated with NH₃(g) as the eluent. Yield 64 mg of a colorless oil. This oil was dissolved in CH₂Cl₂ (1 ml) and 3,4-difluoroisocyanate (0.023 ml, 0.20 mmol) was added. The mixture was stirred for 10 minutes and evaporated. The crude product was purified by reversed phase HPLC. Isolated as the TFA salt. Yield 2.0 mg (2.1%). White solid. MS (ionspray; [M+H]⁺) m/z: 508.2.

HRMS for C₂₉H₃₁F₂N₃O₃: Calcd, 507.2333; found, 507.2320.

### EXAMPLE 483

### N-(3,4-difluorophenyl)-N′-[(3aS*,6R*,7aS*)-3a-(3,4-dimethoxyphenyl)-1-formyloctahydro-1H-indol-6-yl]urea

N-(3,4-difluorophenyl)-N′-[(3aS*,6R*,7aS*)-3a-(3,4-dimethoxyphenyl)octahydro-1H-indol-6-yl]urea, Example 175 (0.025 g, 0.06 mmol) was dissolved in vinyl formate (3 ml) and heated in a microwave oven at 100° C. for 5 minutes. The mixture was evaporated and dissolved in methanol (1 ml). 1 drop of water was added and white crystals were formed. The crystals were filtered and washed with cold methanol and dried in vacuo.

Yield 6.6 mg (24%). MS (ionspray; [M+H]⁺) m/z: 460.4.

### EXAMPLE 484

### N-[(3aS*,6R*,7aS*)-1-cyclopropyl-3a-(3,4-dimethoxyphenyl)octahydro-1H-indol-6-yl]-N′-(3,4-difluorophenyl)urea trifluoroacetate

N-(3,4-difluorophenyl)-N′-[(3aS*,6R*,7aS*)-3a-(3,4-dimethoxyphenyl)octahydro-1H-indol-6-yl]urea, Example 175, (0.020 g, 0.046 mmol) was dissolved in MeOH (1 ml), and acetic acid (0.028 ml, 0.47 mmol), 1-ethoxycyclopropoxytrimethylsilane (0.044 ml, 0.22 mmol) and 3 Å molecular sieves were added. The mixture was stirred at 40° C. for 20 hours, filtered and the crude product was purified by reversed phase HPLC. Isolated as the TFA salt. Yield 10.0 mg (37%). White solid. MS (ionspray; [M+H]⁺) m/z: 472.2. HRMS for C₂₆H₃₁F₂N₃O₃: Calcd, 471.2333; found, 471.2326.

### COMPARATIVE EXAMPLE 485

### (3aS*,6R*,7aS*)-3a-(2-fluoro-3,4-dimethoxyphenyl)-1-methyloctahydro-1H-indol-6-amine

Step 1:

MeI (4.64 ml, 75.0 mmol) was added to a solution of 2-fluoro-6-methoxyphenol (10 g, 70.3 mmol) and CsCO₃ (91.7 g, 280 mmol) in DMSO (400 ml). The mixture was stirred for 1 hour and additional MeI (0.5 ml, 8 mmol) was added. After additional 20 minutes the mixture was partitioned between water and diethylether. The organic phase was washed with water (3×), dried (MgSO₄) and evaporated to give 1-fluoro-2,3-dimethoxybenzene. Yield 9.9 g (91%). Colorless liquid. 1H NMR (400 MHz, CHLOROFORM-D) δ ppm 3.86 (s, 3H) 3.90-3.93 (m, J=1.00 Hz, 3H) 6.65-6.74 (m, 1H) 6.90-6.98 (m, 2H).

Step 2:

1-fluoro-2,3-dimethoxybenzene (9.5 g, 61 mmol) and formaldehyde (37%, 11 ml) were dissolved in glacial acetic acid (38 ml) and HCl (g) was bubbled through the solution for 5 hours (no starting material left). The mixture was poured onto crushed ice and extracted twice with diethylether. The combined organics were washed with water (3×), dried (MgSO₄) and evaporated. The residue was dissolved in toluene and evaporated to get rid of acetic acid and afford 1-(chloromethyl)-2-fluoro-3,4-dimethoxybenzene. Yield 12.3 g (99%). White solid. 1H NMR (400 MHz, CHLOROFORM-D) δ ppm 3.87 (s, 3H) 3.93 (s, 3H) 4.59 (s, 2H) 6.67 (dd, J=8.66, 1.63 Hz, 1H) 7.04 (t, J=8.28 Hz, 1H).

Step 3:

NaCN (3.43 g, 70 mmol) was added to a solution of 1-(chloromethyl)-2-fluoro-3,4-dimethoxybenzene (12.3 g, 60 mmol) in DMSO at room temperature. The mixture was stirred for 1 h and additional NaCN (0.5 g, 10.2 mmol) was added. The mixture was stirred for additional 30 minutes and diluted with diethylether.

The organic phase was washed with water, dried (MgSO₄) and evaporated to give (2-fluoro-3,4-dimethoxyphenyl)acetonitrile. Yield 10.2 g (87%). White solid. 1H NMR (400 MHz, CHLOROFORM-D) δ ppm 3.72 (s, 2H) 3.90 (s, 3H) 3.96 (s, 3H) 6.72 (dd, J=8.78, 1.76 Hz, 1H) 7.04-7.13 (m, 1H).

Step 4:

(2-fluoro-3,4-dimethoxyphenyl)acetonitrile (9.1 g, 47 mmol) was added to a suspention of LiNH₂ (5.19 g, 119 mmol) in DME (455 ml) and heated to 65° C. After 2 minutes 1-bromo-2-chloroethane was added and the mixture was stirred for 24 hours at the same temperature. The mixture was diluted with ether, and icewater was carefully added. The organic was washed with water (3×), dried (MgSO₄) and evaporated. The crude product was purified by column chromatography on silica gel to give 1-(2-fluoro-3,4-dimethoxyphenyl)cyclopropanecarbonitrile.

Yield 6.0 g (58%). Light yellow solid. 1H NMR (400 MHz, CHLOROFORM-D) δ ppm 1.26-1.35 (m, 2H) 1.59-1.67 (m, 2H) 3.85 (s, 3H) 3.93 (s, 3H) 6.63 (dd, J=8.66, 1.88 Hz, 1H) 6.91-6.98 (m, 1H).

Step 5:

DIBAL (53 ml 1N in toluene, 53 mmol) was added dropwise to a solution of 1-(2-fluoro-3,4-dimethoxyphenyl)cyclopropanecarbonitrile (5.9 g, 26.7 mmol) in THF (130 ml). The mixture was stirred for 1.5 hours and poured onto 1N HCl and crushed ice. The mixture was extracted with ether (2×) and the combined organics were washed with 1N HCl (2×), dried (MgSO₄) and evaporated to give 1-(2-fluoro-3,4-dimethoxyphenyl)cyclopropanecarbaldehyde. Yield 4.8 g (80%). Yellow liquid. MS (ionspray; [M+H]⁺) m/z: 225.2.

1H NMR (400 MHz, CHLOROFORM-D) δ ppm 1.28-1.39 (m, 2H) 1.54-1.65 (m, 2H) 3.84-3.87 (m, 3H) 3.93 (s, 3H) 6.65 (dd, J=8.66, 1.63 Hz, 1H) 6.82-6.88 (m, 1H) 9.07 (d, J=1.25 Hz, 1H).

Step 6:

1-(2-fluoro-3,4-dimethoxyphenyl)cyclopropanecarbaldehyde (4.0 g, 17.8 mmol), methylamine hydrochloride (3.54 g, 52.4 mmol), sodiumsulfate (5.6 g, 39 mmol) and DMSO (14 ml) were dissolved in acetonitrile (70 ml) followed by addition of methylvinylketone (2.2 ml, 26.3 mmol).

The mixture was heated at 100° C. for 23 hours, cooled and diluted with ether and 1N NaOH, extracted with ether (2×). The combined organics were washed with water (2×), dried (MgSO₄) and evaporated. The crude product was purified by column chromatography on silica gel using 1%-5% MeOH in CH₂Cl₂ as the eluant to give (3aS*,7aS*)-3a-(2-fluoro-3,4-dimethoxyphenyl)-1-methyloctahydro-6H-indol-6-one.

Yield: 1.3 g (24%). Colorless liquid. MS (ionspray; [M+H]⁺) m/z: 308.2.

1H NMR (400 MHz, CHLOROFORM-D) δ ppm 2.06-2.26 (m, 6H) 2.28 (s, 3H) 2.37-2.48 (m, 1H) 2.55-2.63 (m, 1H) 2.66-2.73 (m, 1H) 2.91 (t, J=3.51 Hz, 1H) 3.03-3.12 (m, 1H) 3.86 (s, 3H) 3.92 (s, 3H) 6.63 (dd, J=8.91, 1.63 Hz, 1H) 6.92 (t, J=8.66 Hz, 1H)

Step 7:

(3aS*,7aS*)-3a-(2-fluoro-3,4-dimethoxyphenyl)-1-methyloctahydro-6H-indol-6-one (1.3 g, 4.2 mmol), and ammoniumtrifluoroacetic acid (2.6 g, 19.8 mmol) were dissolved in THF (130 ml) and stirred for 40 minutes before NaCNBH₃ (0.36 g, 5.7 mmol) was added. The mixture was stirred for 20 minutes and 1N NaOH (28 ml) was added and the mixture was heated at 70° C. for 1 hour and evaporated. The residue was partitioned between CH₂Cl₂ and water, extracted with CH₂Cl₂ (2×), dried (MgSO₄) and evaporated. The crude product was purified by column chromatography on silica gel using CHCl₃ saturated with NH₄ as the eluent. This gave a cis/trans ratio of 3:1. The crude product was dissolved in dry acetonitrile (22 ml) and hydrocinnamic acid (0.33 g, 2.2 mmol) was added and the mixture was allowed to stir for 5 hours. The white crystals were filtered and washed with acetonitrile (2×). The crystals were dissolved in CH₂Cl₂ and shaken vigorously with 1N NaOH and the organic phase was dried (MgSO₄) and evaporated to yield 0.395 g (30%) of (3aS*,6R*,7aS*)-3a-(2-fluoro-3,4-dimethoxyphenyl)-1-methyloctahydro-1H-indol-6-amine as a white solid. MS (ionspray) [M+H]⁺) m/z: 309.2.

1H NMR (400 MHz, CHLOROFORM-D) δ ppm 0.84-0.97 (m, 1H) 1.21 (s, 2H) 1.33-1.47 (m, 1H) 1.53-1.66 (m, 1H) 1.80-1.97 (m, 2H) 2.02-2.22 (m, 4H) 2.31 (s, 3H) 2.57 (s, 1H) 2.91-3.04 (m, 1H) 3.12-3.22 (m, 1H) 3.82 (s, 3H) 3.88 (s, 3H) 6.58 (dd, J=8.78, 1.76 Hz, 1H) 6.93 (t, J=8.78 Hz, 1H).

### EXAMPLE 486

### N-(2,5-difluorophenyl)-N′-[(3aS*,6R*,7aS*)-3a-(2-fluoro-3,4-dimethoxyphenyl)-1-methyloctahydro-1H-indol-6-yl]urea trifluoroacetate

2,5-difluorophenylisocyanate (0.004 ml, 0.034 mmol) was added to a solution of (3aS*,6R*,7aS*)-3a-(2-fluoro-3,4-dimethoxyphenyl)-1-methyloctahydro-1H-indol-6-amine, Comparative Example 485, (0.010 g, 0.032 mmol) in CH₂Cl₂ (1 ml). The mixture was stirred for 50 minutes and evaporated. The The crude product was purified by reversed phase HPLC. Isolated as the TFA salt. Yield 15.0 mg (81%). Colorless liquid. MS (ionspray; [M+H]⁺) m/z: 464.2. HRMS for C₂₄H₂₈F₃N₃O₃: Calcd, 463.2083; found, 463.2089.

### EXAMPLE 487

### N-[(3aS*,6R*,7aS*)-3a-(2-fluoro-3,4-dimethoxyphenyl)-1-methyloctahydro-1H-indol-6-yl]-N′-[2-fluoro-3-(trifluoromethyl)phenyl]urea trifluoroacetate

2-fluoro-3-(trifluoromethyl)-phenyl isocyanate (0.005 ml, 0.035 mmol) was added to a solution of (3aS*,6R*,7aS*)-3a-(2-fluoro-3,4-dimethoxyphenyl)-1-methyloctahydro-1H-indol-6-amine, Comparative Example 485, (0.010 g, 0.032 mmol) in CH₂Cl₂ (1 ml). The mixture was stirred for 60 minutes and evaporated. The crude product was purified by reversed phase HPLC. Isolated as the TFA salt. Yield 6.7 mg (33%). Colorless liquid. MS (ionspray; [M+H]⁺) m/z: 514.2. HRMS for C₂₅H₂₈F₃N₃O₃: Calcd, 513.2051; found, 513.2050.

### EXAMPLE 488

### N-[(3aS*,6R*,7aS*)-3a-(2-fluoro-3,4-dimethoxyphenyl)-1-methyloctahydro-1H-indol-6-yl]-N′-[3-fluoro-5-(trifluoromethyl)phenyl]urea trifluoroacetate

3-fluoro-5-(trifluoromethyl)phenylisocyanate (0.005 ml, 0.035 mmol) was added to a solution of (3aS*,6R*,7aS*)-3a-(2-fluoro-3,4-dimethoxyphenyl)-1-methyloctahydro-1H-indol-6-amine, Comparative Example 485, (0.010 g, 0.032 mmol) in CH₂Cl₂ (1 ml). The mixture was stirred for 60 minutes and evaporated. The crude product was purified by reversed phase HPLC. Isolated as the TFA salt. Yield 11.0 mg (53%). Colorless liquid. MS (ionspray; [M+H]⁺) m/z: 514.2. HRMS for C₂₅H₂₈F₅N₃O₃: Calcd, 513.2051; found, 513.2052.

### EXAMPLE 489

### N-(3,4-difluorophenyl)-N′-[(3aS*,6R*,7aS*)-3a-(2-fluoro-3,4-dimethoxyphenyl)-1-methyloctahydro-1H-indol-6-yl]urea

3,4-difluorophenylisocyanate (0.021 ml, 0.018 mmol) was added to a solution of (3aS*,6R*,7aS*)-3a-(2-fluoro-3,4-dimethoxyphenyl)-1-methyloctahydro-1H-indol-6-amine, Comparative Example 485, (0.050 g, 0.16 mmol) in CH₂Cl₂ (1 ml). The mixture was stirred for 30 minutes and evaporated. The crude product was purified by column chromatography on silica gel using CHCl₃ saturated with NH₃ as the eluent. Yield: 12.2 mg (16%). White solid. MS (ionspray; [M+H]⁺) m/z: 464.2. HRMS for C₂₄H₂₈F₃N₃O₃: Calcd, 463.2083; found, 463.2073.

### EXAMPLE 490

### N-[(3aS*,6R*,7aS*)-3a-(2-fluoro-3,4-dimethoxyphenyl)-1-methyloctahydro-1H-indol-6-yl]-N′-(3,4,5-trifluorophenyl)urea trifluoroacetate

Triphosgene (0.0074 g, 0.0025 mmol) was added to a solution of (3aS*,6R*,7aS*)-3a-(2-fluoro-3,4-dimethoxyphenyl)-1-methyloctahydro-1H-indol-6-amine, Comparative Example 485, (0.019 g, 0.060 mmol) and triethylamine (0.017 ml, 0.120 mmol) in CH₂Cl₂ (1 ml). The mixture was stirred for 90 minutes before 3,4,5-trifluoro-aniline (0.009 g, 0.060 mmol) was added. The mixture was stirred overnight and evaporated. The crude product was dissolved in MeOH and purified by reversed phase HPLC. Isolated as the TFA salt. Yield 6.6 mg (18%). White solid. MS (ionspray; [M+H]⁺) m/z: 482.2. HRMS for C₂₇H₂₇F₄N₃O₃: Calcd, 481.1989; found, 481.1972.

### COMPARATIVE EXAMPLE 491

### (3aS*,6R*,7aS*)-1-tert-butyl-3a-(3,4-dimethoxyphenyl)octahydro-1H-indol-6-amine

Step 1:

1-(3,4-dimethoxyphenyl)cyclopropanecarboxaldehyde, Comparative Example 2, (6.0 g, 29.5 mmol), t-butyl hydrochloride (0.43 g, 3.3 mmol), sodium sulfate (4.4 g, 40 mmol) and methylvinylketone (3.2 ml, 38.3 mmol) were dissolved in MeCN (100 ml) and DMSO (20 ml) and stirred at 100° C. overnight. Additional t-butylamine hydrochloride (1.4 g, 13 mmol) and methylvinylketone (1.1 ml, 13 mmol) were added once a day for 3 days until HPLC showed consumption of the starting material. The mixture was cooled and diluted with diethylether and 1N NaOH (80 ml). The organic phase was washed with water (3×) and evaporated. The residue was dissolved in 1N HCl (150 ml) and washed with diethylether (1×). The aqueous phase was made basic with 1N NaOH (155 ml) and extracted with diethylether (2×), dried (MgSO₄) and evaporated. The crude product was purified by column chromatography on silica gel with 1% triethylamine in CH₂Cl₂ as the eluent. Yield 1.9 g (20%) of (3aS*,7aS*)-1-tert-butyl-3a-(3,4-dimethoxyphenyl)octahydro-6H-indol-6-one, as a light brown gum. MS (ionspray; [M+H]⁺) m/z: 332.2.

Step 2:

(3aS*,7aS*)-1-tert-butyl-3a-(3,4-dimethoxyphenyl)octahydro-6H-indol-6-one prepared in step 1, (1.5 g, 4.5 mmol) and ammonium trifluoroacetate (6.0 g, 46 mmol) were dissolved in DME (150 ml) and stirred for 40 minutes before NaCNBH₃ (0.314 g, 5.0 mmol) was added. The mixture was stirred for 3.5 hours and evaporated. The residue was partitioned between CH₂Cl₂ and 1N NaOH, extracted with CH₂Cl₂ (2×), dried (MgSO₄) and evaporated. The crude product was dissolved in acetonitrile (20 ml) and hydrocinnamic acid (0.27 g, 1.8 mmol) was added and the mixture was allowed to stir overnight and the white powder was filtered and washed with dry acetonitrile (2×). The crystals were dissolved in CH₂Cl₂ and shaken vigorously with 1N NaOH. The organic phase was dried (MgSO₄) and evaporated to yield 0.190 g (12.7%) of the title compound as a white solid. MS (ionspray) [M+H]⁺) m/z: 333.2.

1H NMR (400 MHz, CHLOROFORM-D) δ ppm 1.03-1.17 (m, 11H) 1.35-1.45 (m, 1H) 1.64-1.72 (m, 1H) 1.73-1.82 (m, 2H) 1.90-2.01 (m, 3H) 2.80-2.88 (m, 1H) 3.03 (dd, J=7.65, 2.89 Hz, 1H) 3.11-3.21 (m, 1H) 3.48 (t, J=3.89 Hz, 1H) 3.85 (s, 3H) 3.87 (s, 3H) 6.79 (d, J=8.03 Hz, 1H) 6.90-7.00 (m, 3H).

### EXAMPLE 492

### N-[(3aS*,6R*,7aS*)-1-tert-butyl-3a-(3,4-dimethoxyphenyl)octahydro-1H-indol-6-yl]-N′-[2-fluoro-3-(trifluoromethyl)phenyl]urea trifluoroacetate

2-fluoro-3-trifluoromethylphenylisocyanate (0.014 ml, 0.096 mmol) was added to a solution of (3aS*,6R*,7aS*)-1-tert-butyl-3a-(3,4-dimethoxyphenyl)octahydro-1H-indol-6-amine, Comparative Example 491, (0.015 g, 0.045 mmol) in CH₂Cl₂ (1 ml). The mixture was stirred for 30 minutes and evaporated. The crude product was purified by reversed phase HPLC and isolated as the TFA salt. Yield 15.1 mg (54%). Colorless gum. MS (ionspray; [M+H]⁺) m/z: 538.4. HRMS for C₂₈H₃₅F₄N₃O₃: Calcd, 537.2615; found, 537.2590.

### EXAMPLE 493

### N-[(3aS*,6R*,7aS*)-1-tert-butyl-3a-(3,4-dimethoxyphenyl)octahydro-1H-indol-6-yl]-N′-(2,5-difluorophenyl)urea trifluoroacetate

2,5-difluorophenylisocyanate (0.004 ml, 0.034 mmol) was added to a solution of (3aS*,6R*,7aS*)-1-tert-butyl-3a-(3,4-dimethoxyphenyl)octahydro-1H-indol-6-amine, Comparative Example 491, (0.010 g, 0.030 mmol) in CH₂Cl₂ (1 ml). The mixture was stirred for 60 minutes and evaporated. The crude product was purified by reversed phase HPLC. Isolated as the TFA salt. Yield 8.4 mg (47%). Light brown solid. MS (ionspray; [M+H]⁺) m/z: 488.2. HRMS for C₂₇H₃₅F₂N₃O₃: Calcd, 487.2635; found, 487.2635.

### EXAMPLE 494

### N-[(3aS*,6R*,7aS*)-1-tert-butyl-3a-(3,4-dimethoxyphenyl)octahydro-1H-indol-6-yl]-N′-[3-fluoro-5-(trifluoromethyl)phenyl]urea trifluoroacetate

3,5-difluorophenylisocyanate (0.005 ml, 0.035 mmol) was added to a solution of (3aS*,6R*,7aS*)-1-tert-butyl-3a-(3,4-dimethoxyphenyl)octahydro-1H-indol-6-amine, Comparative Example 491, (0.010 g, 0.030 mmol) in CH₂Cl₂ (1 ml). The mixture was stirred for 60 minutes and evaporated. The crude product was purified by reversed phase HPLC. Isolated as the TFA salt. Yield 11.0 mg (56%). Light brown solid. MS (ionspray; [M+H]⁺) m/z: 538.2. HRMS for C₂₈H₃₅F₄N₃O₃: Calcd, 537.2615; found, 537.2627.

### EXAMPLE 495

### N-[(3aS*,6R*,7aS*)-1-tert-butyl-3a-(3,4-dimethoxyphenyl)octahydro-1H-indol-6-yl]-N′-(3,4,5-trifluorophenyl)urea trifluoroacetate

Triphosgene (0.0074 g, 0.0025 mmol) was added to a solution of (3aS*,6R*,7aS*)-1-tert-butyl-3a-(3,4-dimethoxyphenyl)octahydro-1H-indol-6-amine, Comparative Example 491, (0.020 g, 0.060 mmol) and triethylamine (0.017 ml, 0.120 mmol) in CH₂Cl₂ (1 ml). The mixture was stirred for 90 minutes before 3,4,5-trifluoro-aniline (0.009 g, 0.060 mmol) was added. The mixture was stirred overnight and evaporated. The crude product was dissolved in MeOH and purified by reversed phase HPLC. Isolated as the TFA salt. Yield 4.2 mg (11%). Light red gum. MS (ionspray; [M+H]⁺) m/z: 506.2. HRMS for C₂₇H₃₄F₃N₃O₃: Calcd, 505.2552; found, 505.2542.

### COMPARATIVE EXAMPLE 496

### (3aS*,7aS*)-3a-[3-(benzyloxy)-4-methoxyphenyl]-1-methyloctahydro-6H-indol-6-one

Step 1:

Benzyl bromide (23.2 g, 0.136 mol) was added to a suspension of 3-hydroxy-4-methoxybenzyl alcohol (19.0 g, 0.123 mol) and Cs₂CO₃ (44.2 g, 0.136 mol) in acetone (300 mL). The mixture was stirred at 65° C. overnight (16 h). The suspension was filtered and the solid material was washed with DCM, concentration of the filtrate gave 37 g of crude material, which was dissolved in ether and addition of iso-hexane gave a solid which was collected to afford [3-(benzyloxy)-4-methoxyphenyl]methanol, (24.05 g, 80%) as a white fluffy solid. 1H NMR (400 MHz, CHLOROFORM-D) ppm 3.88 (s, 3H) 4.55-4.58 (m, 2H) 5.15 (s, 2H) 6.86 (d, J=8.03 Hz, 1H) 6.89-6.92 (m, 1H) 6.94-6.96 (m, 1H) 7.28-7.31 (m, 1H) 7.34-7.38 (m, 2H) 7.42-7.46 (m, 2H). GC-MS (EI) for C₁₅H₁₆O₃ m/z 244 (M+)

Step 2:

Br₂ (6.56 mL, 128 mmol) was added dropwise to an ice cold solution of PPh₃ (33.6 g, 128 mmol) in DCM (370 mL). The mixture was stirred for 5 min and [3-(benzyloxy)-4-methoxyphenyl]methanol prepared in step 1, (24.05 g, 98 mmol) in DCM (370 mL) was added dropwise and stirred at 0° C. for 30 min. Ether (2200 mL) was added and the resulting precipitated OPPh₃ was filtered off. The solvent was evaporated to give crude 2-benzyloxy-4-bromomethyl-1-methoxybenzene. NaCN (5.78 g, 118 mmol) was added to the bromo compound dissolved in DMF (110 mL) and stirred over night (17 h). Water (350 mL) was added and the mixture was extracted with ether (3×700 mL), the organic phases combined, dried and concentrated. The crude product was purified by flash column chromatography (iso-hexane/EtOAc 6:1→4:1→2:1). This gave the product (14.71 g, 59%) as a white solid. ¹H NMR (400 MHz, CHLOROFORM-D) ppm 3.63 (s, 2H) 3.87 (s, 3H) 5.14 (s, 2H) 6.83-6.84 (m, 1H) 6.86-6.87 (m, 2H) 7.30-7.33 (m, 1H) 7.35-7.39 (m, 2H) 7.42-7.45 (m, 2H). GC-MS (EI) for C₁₆H₁₅NO₂ m/z 244 (M+).

Step 3:

[3-(benzyloxy)-4-methoxyphenyl]acetonitrile, prepared in step 2, (14.71 g, 58.1 mmol) in DME (150 mL) was added to a stirred suspension of LiNH₂ (2.00 g, 87 mmol) in DME (150 mL) at 65° C. An additional portion of LiNH₂ (1.61 g, 70 mmol) was added. The mixture was stirred for 1 h 45 min at 65° C. (color turned to green) and 2-bromochloroethane (5.71 mL, 68.4 mmol) in DME (15 mL) was added dropwise over 20 min. The mixture was stirred at that temperature over night (14 hours) and the color turned to dark blue. The reaction was quenched with water (200 mL) and the mixture was extracted twice with ether. The organic phases were combined, dried and concentrated. The crude 1-[3-(benzyloxy)₄-methoxyphenyl]cyclopropanecarbonitrile (17 g) was purified by flash column chromatography (short plug, DCM/hexane 1:1) to give the product (13.6 g, 84%). ¹H NMR (400 MHz, CHLOROFORM-D) ppm 1.23-1.26 (m, 2H) 1.59-1.63 (m, 2H) 3.86 (s, 3H) 5.14 (s, 2H) 6.83-6.85 (m, 3H) 7.28-7.33 (m, 1H) 7.35-7.39 (m, 2H) 7.43-7.46 (m, 2H). GC-MS (EI) for C₁₈H₁₇NO₂ m/z 279 (M+).

Step 4:

DIBAL (1.0M in toluene, 97 mL, 97 mmol) was added to a solution of 1-[3-(benzyloxy)-4-methoxyphenyl]cyclopropanecarbonitrile, prepared in step 3, (13.6 g, 49 mmol) in dry THF (125 mL) under N₂ (g). The solution was stirred for 4 hours and poured into ice cold 2M aq. HCl (150 mL). The mixture was extracted twice with ether and the organic phases were combined, dried and concentrated to give 12.6 g (92%) of 1-[3-(benzyloxy)-4-methoxyphenyl]cyclopropanecarbaldehyde. ¹H NMR (400 MHz, CHLOROFORM-D) ppm 1.28-1.31 (m, 2H) 1.48-1.51 (m, 2H) 3.87 (s, 3H) 5.13 (s, 2H) 6.84-6.85 (m, 1H) 6.86-6.87 (m, 2H) 7.27-7.32 (m, 1H) 7.34-7.38 (m, 2H) 7.42-7.45 (m, 2H) 9.21 (s, 1H). GC-MS (EI) for C₁₈H₁₈O₃ m/z 279 (M+).

Step 5:

Methylamine (2.0M in THF, 44.6 mL, 89.3 mmol) was added to a mixture of 1-[3-(benzyloxy)-4-methoxyphenyl]cyclopropanecarbaldehyde, prepared in step 4, (12.60 g, 44.6 mmol) and MgSO₄ (17 g) in 1,2-dichloroethane (45 mL). The mixture was stirred at rt. over night for 22 h. The solid material was filtered off, rinsed with ether and the organic phase was concentrated to give ((1E)-{1-[3-(benzyloxy)-4-methoxyphenyl]cyclopropyl}methylene)methylamine (12.45 g, 95%) as a yellow solid. ¹H NMR (400 MHz, CHLOROFORM-D) ppm 1.05-1.08 (m, 2H) 1.20-1.22 (m, 2H) 3.20 (d, J=1.51 Hz, 3H) 3.86 (s, 3H) 5.13 (s, 2H) 6.82-6.85 (m, 1H) 6.86-6.89 (m, 2H) 7.27-7.32 (m, 1H) 7.34-7.38 (m, 2H) 7.42-7.46 (m, 2H) 7.48-7.51 (m, 1H). GC-MS (EI) for C₁₉H₂₁NO₂ m/z 295 (M+).

Step 6:

((1E)-{1-[3-(benzyloxy)-4-methoxyphenyl]cyclopropyl}methylene)methylamine, prepared in step 5, (2.00 g, 6.77 mmol) was dissolved in toluene which was then evaporated to get rid of water. The resulting material was dissolved in DCM (15 mL) and 2M HCl in ether (5.0 mL) was added while cooling on ice-bath and the resulting enamine precipitated. The solvent was evaporated, toluene was added and evaporated to get rid of water. The crude enamine was dissolved in CH₃CN (30 mL), whereafter N,O-bis(trimethyl-silyl)-acetamide (340 μL) and methylvinylketone (680 μL, 8.1 mmol) were added in that order. The solution was refluxed at 85° C. under N₂ (g) over night (16 h). The solvent was evaporated to give the HCl-salt of the product, which was dissolved in water/EtOAc (pH 1). The acidic water phase was made basic with aq. NaOH to pH 9-10 and the layers were separated. The EtOAc was evaporated to give 1.8 g of a crude product which was purified by flash column chromatography (hexane/3% NEt₃ in EtOAc 2:1→hexane/3% NEt₃ in EtOAc 1:1). Affording (3aS*,7aS*)-3a-[3-(benzyloxy)₄-methoxyphenyl]-1-methyloctahydro-6H-indol-6-one (0.86 g, 35%) as a yellow viscous liquid. ¹H NMR (400 MHz, CHLOROFORM-D) ppm 1.94-2.25 (m, 6H) 2.25 (s, 3H) 2.30-2.43 (m, 2H) 2.45-2.50 (m, 1H) 2.76 (t, J=3.51 Hz, 1H) 3.04-3.09 (m, 1H) 3.89 (s, 3H) 5.17 (s, 2H) 6.83-6.86 (m, 2H) 6.90-6.93 (m, 1H) 7.26-7.30 (m, 1H) 7.32-7.36 (m, 2H) 7.40-7.43 (m, 2H). MS (ESI+) for C₂₃H₂₇NO₃ m/z 366.2 (M+H)⁺.

### EXAMPLE 497

### N-{(3aS*,6R*,7aS*)-3a-[3-(benzyloxy)-4-methoxyphenyl]-1-methyloctahydro-1H-indol-6-yl}-N′-(3,4-difluorophenyl)urea and

### EXAMPLE 498

### N-{(3aS*,6S*,7aS*)-3a-[3-(benzyloxy)-4-methoxyphenyl]-1-methyloctahydro-1H-indol-6-yl}-N′-(3,4-difluorophenyl)urea

NH₄OAc (4.43 g, 58 mmol) was added to a solution of (3aS*,7aS*)-3a-[3-(benzyloxy)-4-methoxyphenyl]-1-methyloctahydro-6H-indol-6-one, Comparative Example 496, (422 mg, 1.15 mmol) in dry MeOH (25 mL). After the mixture had stirred for 2 h at 60° C. NaCNBH₃ (181 mg, 2.89 mmol) was added and stirring continued for 1 hour at the same temperature. The solvent was evaporated and the mixture was dissolved in DCM and quenched with 2M aq. NaOH. The phases were separated and the water phase was extracted once with DCM. The organic phases were combined, dried and concentrated. The crude amine was dissolved in dry DCM (10 mL) and 3,4-difluorophenyl isocyanate (175 μL, 1.50 mmol) was added. The reaction mixture was stirred for 1.5 h and the solvent was evaporated. The crude mixture of diastereomers were separated and purified using flash column chromatography (5% MeOH in DCM-10% MeOH in DCM). This gave the cis-product N-{(3aS*,6R*,7aS*)-3a-[3-(benzyloxy)-4-methoxyphenyl 1-methyloctahydro-1H-indol-6-yl}-N′-(3,4-difluorophenyl)urea (174 mg, 29%) as a colorless solid. ¹H NMR (400 MHz, CHLOROFORM-D) ppm 0.82-0.92 (m, 1H) 1.10-1.19 (m, 1H) 1.63-1.71 (m, 2H) 1.75-1.82 (m, 1H) 1.85-1.91 (m, 1H) 2.00-2.11 (m, 2H) 2.20-2.29 (m, 1H) 2.32 (s, 3H) 2.58 (br. s., 1H) 3.15-3.24 (m, 1H) 3.87 (s, 3H) 3.87-3.96 (m, 1H) 4.27 (d, J=8.53 Hz, 1H) 5.11-5.19 (m, 2H) 6.42 (br. s., 1H) 6.80-6.91 (m, 4H) 6.97-7.04 (m, 1H) 7.26-7.28 (m, 1H) 7.31-7.34 (m, 3H) 7.39-7.41 (m, 2H). MS (ESI+) for C₃₀H₃₃F₂N₃O₃ m/z 522.2 (M+H)⁺. HRMS (EI) calcd for C₃₀H₃₃F₂N₃O₃: 521.2490, found 521.2498.

The trans-product N-{(3aS*,6S*,7aS*)-3a-[3-(benzyloxy)-4-methoxyphenyl]-1-methyloctahydro-1H-indol-6-yl}-N′-(3,4-difluorophenyl)urea (220 mg, 37%) was obtained as a colorless solid. ¹H NMR (400 MHz, CHLOROFORM-D) ppm 1.16-1.25 (m, 1H) 1.51-1.61 (m, 2H) 1.63-1.84 (m, 5H) 1.87-1.96 (m, 1H) 2.14 (s, 3H) 2.15-2.25 (m, 1H) 2.55-2.60 (br. s., 1H) 3.05-3.13 (br. s., 1H) 3.87 (s, 3H) 3.95-4.00 (br. s., 1H) 5.11-5.18 (m, 2H) 6.10-6.16 (br. s., 1H) 6.74-6.76 (m, 1H) 6.81-6.86 (m, 2H) 6.89-6.94 (m, 1H) 7.05-7.12 (m, 1H) 7.26-7.30 (m, 2H) 7.31-7.36 (m, 2H) 7.37-7.41 (m, 2H). MS (ESI+) for C₃₀H₃₃F₂N₃O₃ m/z 522.2 (M+H)⁺.

The relative stereochemistries of both compounds were readily confirmed using ¹H-NOESY NMR

### EXAMPLE 499

### N-(3,4-difluorophenyl)-N′-[(3aS*,6R*,7aS*)-3a-(3-hydroxy-4-methoxyphenyl)-1-methyloctahydro-1H-indol-6-yl]urea

Acetic acid (5 mL), HCl (1.0M in ether, 4 mL) and Pd/C (10%, 1.5 g) were added to N-{(3aS*,6R*,7aS*)-3a-[3-(benzyloxy)-4-methoxyphenyl]-1-methyloctahydro-1H-indol-6-yl}-N′-(3,4-difluorophenyl)urea, Example 497, (330 mg, 0.63 mmol) in EtOH (30 mL) and hydrogenated in a Parr-apparatus (H₂ (g), 5 atm) for 6 h. The reaction mixture was filtered through celite which was rinsed several times with EtOH and MeOH. The filtrate was concentrated and the crude product was purified on a short silica column (5% MeOH in CHCl₃ sat. with NH₃ (g)) to obtain the title compound (183 mg, 67%) as a white solid. ¹H NMR (400 MHz, DMSO-D6) ppm 0.94-1.04 (m, 1H) 1.28-1.36 (m, 1H) 1.55-1.66 (m, 2H) 1.72-1.79 (m, 1H) 1.88-2.01 (m, 3H) 2.17-2.24 (m, 1H) 2.25 (s, 3H) 2.57-2.61 (m, 1H) 3.09-3.14 (m, 1H) 3.73 (s, 3H) 3.72-3.80 (m, 1H) 6.03 (d, J=8.03 Hz, 1H) 6.74 (dd, J=8.28, 2.26 Hz, 1H) 6.80 (d, J=2.01 Hz, 1H) 6.84 (d, J=8.53 Hz, 1H) 6.94-6.99 (m, 1H) 7.19-7.27 (m, 1H) 7.56-7.61 (m, 1H) 8.38 (s, 1H) 8.82 (br. s., 1H). MS (ESI+) for C₂₃H₂₇F₂N₃O₃ m/z 432.4 (M+H)⁺. HRMS (EI) calcd for C₂₃H₂₇F₂N₃O₃: 431.2021, found 431.2035.

### EXAMPLE 500

### N-(3,4-difluorophenyl)-N′-[(3aS*,6R*,7aS*)-3a-(3-isopropoxy-4-methoxyphenyl)-1-methyloctahydro-1H-indol-6-yl]urea trifluoroacetate

Isopropylbromide (30 mg, 23 μL, 0.24 mmol) was added to a suspension of Cs₂CO₃ (79 mg, 0.24 mmol) and N-(3,4-difluorophenyl)-N′-[(3aS*,6R*,7aS*)-3a-(3-hydroxy-4-methoxyphenyl)-1-methyloctahydro-1H-indol-6-yl]urea, Example 499, (21 mg, 0.05 mmol) in DMF (2 mL). The reaction mixture was stirred for 72 h, the solvent was evaporated and the product purified by preparative HPLC. To give (11 mg, 38%) of a colorless salt. ¹H NMR (400 MHz, METHANOL-D4) ppm 1.09-1.17 (m, 1H) 1.20 (s, 3H) 1.21 (s, 3H) 1.64-1.79 (m, 3H) 2.07-2.19 (m, 2H) 2.26-2.33 (m, 1H) 2.36-2.43 (m, 1H) 3.01 (s, 3H) 3.22-3.30 (m, 1H) 3.74 (s, 3H) 3.75-3.84 (m, 2H) 3.97-4.00 (m, 1H) 4.45-4.52 (m, 1H) 6.83-6.87 (m, 1H) 6.91 (s, 3H) 6.98-7.06 (m, 1H) 7.36-7.42 (m, 1H). MS (ESI+) for C₂₆H₃₃F₂N₃O₃ m/z 474 (M+H)⁺. HRMS (EI) calcd for C₂₆H₃₃F₂N₃O₃: 473.2490, found 473.2474

### EXAMPLE 501

### N-(3,4-difluorophenyl)-N′-[(3aS*,6R*,7aS*)-3a-(3-ethoxy-4-methoxyphenyl)-1-methyloctahydro-1H-indol-6-yl]urea trifluoroacetate

Ethyliodide (51 mg, 26 μL, 0.32 mmol) was added to a suspension of Cs₂CO₃ (106 mg, 0.32 mmol) and N-(3,4-difluorophenyl)-N′-[(3aS*,6R*,7aS*)-3a-(3-hydroxy-4-methoxyphenyl)-1-methyloctahydro-1H-indol-6-yl]urea, Example 499, (28 mg, 0.07 mmol) in DMF (2 mL). The reaction mixture was stirred for 24 h and the solvent was evaporated. The crude product was purified by preparative HPLC. This gave the product (8 mg, 21%) as a colorless salt. 1H NMR (400 MHz, METHANOL-D4) ppm 1.07-1.16 (m, 1H) 1.29 (t, J=7.03 Hz, 3H) 1.62-1.76 (m, 3H) 2.05-2.17 (m, 2H) 2.26-2.33 (m, 1H) 2.33-2.40 (m, J=13.30, 2.26 Hz, 1H) 2.99 (s, 3H) 3.22-3.27 (m, 1H) 3.72 (s, 3H) 3.74-3.82 (m, 2H) 3.95-4.00 (m, 1H) 3.97 (q, J=7.03 Hz, 2H) 6.80-6.85 (m, 1H) 6.87 (s, 3H) 6.96-7.04 (m, 1H) 7.34-7.40 (m, 1H).). MS (ESI+) for C₂₅H₃₁F₂N₃O₃ m/z 460 (M+H)⁺.

HRMS (EI) calcd for C₂₆H₃₃F₂N₃O₃: 459.2334, found 459.2319.

### EXAMPLE 502

### N-(3,4-difluorophenyl)-N′-[(3aS*,6R*,7aS*)-3a-(4-methoxy-3-propoxyphenyl)-1-methyloctahydro-1H-indol-6-yl]urea trifluoroacetate

Propyl iodide (53 mg, 31 μL, 0.31 mmol) was added to a suspension of Cs₂CO₃ (102 mg, 0.31 mmol) and N-(3,4-difluorophenyl)-N′-[(3aS*,6R*,7aS*)-3a-(3-hydroxy-4-methoxyphenyl)-1-methyloctahydro-1H-indol-6-yl]urea, Example 499, (27 mg, 0.06 mmol) in DMF (2 mL). The reaction mixture was stirred for 24 h and the solvent was evaporated. The crude product was purified by preparative HPLC. This gave the product (13 mg, 35%) as a colorless salt. 1H NMR (400 MHz, METHANOL-D4) ppm 0.96 (t, J=7.28 Hz, 3H) 1.10-1.20 (m, 1H) 1.64-1.78 (m, 5H) 2.07-2.20 (m, 2H) 2.28-2.35 (m, 1H) 2.35-2.43 (m, 1H) 3.01 (s, 3H) 3.23-3.30 (m, 1H) 3.74 (s, 3H) 3.76-3.84 (m, 2H) 3.89 (t, J=6.53 Hz, 2H) 3.98-4.02 (m, 1H) 6.83-6.87 (m, 1H) 6.88 (s, 3H) 6.97-7.05 (m, 1H) 7.35-7.41 (m, 1H).). MS (ESI+) for C₂₆H₃₃F₂N₃O₃ m/z 474 (M+H)⁺. HRMS (EI) calcd for C₂₆H₃₃F₂N₃O₃: 473.2490, found 473.2510.

### COMPARATIVE EXAMPLE 503

### (3aS*,7aS*)-3a-[4-(difluoromethoxy)-3-methoxyphenyl]-1-methyloctahydro-6H-indol-6-one

Step 1:

NaBH₄ (1.345 g, 35.4 mmol) was added portionwise to an ice cold solution of 4-(difluoromethoxy)-3-methoxy benzaldehyde (4.772 g, 23.6 mmol) in dry MeOH (70 mL). The mixture was allowed to attain rt. and all starting material was consumed after 1 h. 2M aq. HCl (10 mL) was added and MeOH was evaporated under reduced pressure. The residue was extracted with DCM (×2) and water. The organic layers were combined, dried and concentrated to give [4-(difluoromethoxy)-3-methoxyphenyl]methanol-4.772 g (99%).

1H NMR (400 MHz, CHLOROFORM-D) ppm 3.88 (s, 3H) 4.67 (s, 2H) 6.53 (t, J=75.29 Hz, 1H) 6.87-6.91 (m, J=8.03, 2.01 Hz, 1H) 7.00-7.03 (m, J=1.76 Hz, 1H) 7.13 (d, J=8.03 Hz, 1H).

Step 2:

PBr₃ (965 mg, 3.56 mmol) in dry diethyl ether (2 mL) was added dropwise to an ice cold solution of [4-(difluoromethoxy)-3-methoxyphenyl]methanol, prepared in step 1, (311 mg, 1.52 mmol) in dry diethylether (15 mL). The reaction was stirred at reflux for 2 hours, at room temperature then poured onto crushed ice. The mixture was extracted twice with diethyl ether and the organic layers combined, dried and concentrated to give 4-(bromomethyl)-1-(difluoromethoxy)-2-methoxybenzene-400 mg (99%) product. 1H NMR (400 MHz, CHLOROFORM-D) ppm 3.87-3.91 (m, 3H) 4.46 (s, 2H) 6.53 (t, J=75.04 Hz, 1H) 6.93-6.97 (m, 1H) 6.99 (d, J=2.01 Hz, 1H) 7.10 (d, J=8.28 Hz, 1H).

Step 3:

Sodium cyanide (2.40 g, 48.9 mmol) was added to a solution of 4-(bromomethyl)-1-(difluoromethoxy)-2-methoxybenzene, prepared in step 2, (10.89 g, 40.8 mmol) in dry DMF (100 mL). The solution was stirred at rt. Overnight (17 hours). Water (200 mL) was added and the mixture was extracted once with DCM (250 mL). The organic layer was dried and concentrated under reduced pressure to give [4-(difluoromethoxy)-3-methoxyphenyl]acetonitrile 8.22 g (94%) of crude product. 1H NMR (400 MHz, CHLOROFORM-D) ppm 3.74 (s, 2H) 3.90 (s, 3H) 6.53 (t, J=74.91 Hz, 1H) 6.87 (dd, J=8.16, 2.13 Hz, 1H) 6.93 (d, J=2.01 Hz, 1H) 7.15 (d, J=8.28 Hz, 1H).

Step 4:

To a suspension of LiNH₂ (4.11 g, 178 mmol) in dry DME (100 mL) were added [4-(difluoromethoxy)-3-methoxyphenyl]acetonitrile, prepared in step 3, (7.62 g, 35.7 mmol) in dry DME (100 mL), dry DMSO (5.08 mL, 71.5 mmol) and dropwise 1-bromo-2-chloroethane (5.96 mL, 71.5 mmol) at 65° C. The mixture was stirred overnight for 17 hours. Water (15 mL) was added and DME was removed under reduced pressure. Additional 200 mL water was added followed by extraction with DCM (2×200 mL). The organic layers were combined, dried and concentrated to give 7.5 g crude product. Purification by flash column chromatography (DCM/iso-hexane 1:1) afforded 1-[4-(difluoromethoxy)-3-methoxyphenyl]cyclopropanecarbonitrile (5.60 g, 65%) as a bone-white solid. 1H NMR (400 MHz, CHLOROFORM-D) ppm 1.36-1.41 (m, 2H) 1.69-1.75 (m, 2H) 3.90 (s, 3H) 6.52 (t, J=75.04 Hz, 1H) 6.75 (dd, J=8.41, 2.13 Hz, 1H) 6.99 (d, J=2.26 Hz, 1H) 7.11 (d, J=8.53 Hz, 1H). GC-MS (EI) for C₁₂H₁₁F₂NO₂ m/z 239 (M)⁺.

Step 5:

DIBAL (1.0M in toluene, 45.2 mL, 45.2 mmol) was added dropwise under N₂ to a stirred solution of 1-[4-(difluoromethoxy)-3-methoxyphenyl]cyclopropanecarbonitrile, prepared in step 4, (5.40 g, 22.6 mmol) in dry THF (100 mL). 100% conversion to product after 1 hour. The reaction mixture was slowly poured into 150 mL ice-cold 2M aq. HCl followed by extraction with DCM (2×150 mL). The organic layers were combined, dried and concentrated to give 1-[4-(difluoromethoxy)-3-methoxyphenyl]cyclopropanecarbaldehyde (4.54 g, 83%) as a white fluffy solid. GC-MS (EI) for C₁₂H₁₂F₂O₃ m/z 242 (M)⁺.

Step 6:

Methylamine (2.0 M in THF, 16.5 mL, 33 mmol) was added to a suspension of ((1-[4-(difluoromethoxy)-3-methoxyphenyl]cyclopropanecarbaldehyde, prepared in step 5, (4.01 g, 16.6 mmol) and Na₂SO₄ (30 g, 211 mmol) in dry 1,2-dichloroethane. Stirring at rt. for 6 hours gave 60% conversion. Additional Na₂SO₄ (10 g) and methylamine (5 mL) were added with continous stirring 17 hours over night. The mixture was filtered and concentrated. HNMR of the crude product showed ˜15% aldehyde. The crude product was again dissolved in 1,2-dichloroethane (60 mL) whereafter Na₂SO₄ (10 g) and methylamine (6 mL) were added. Stirring for 22 hours gave 100% conversion to imine. The mixture was filtered and the filtrate evaporated to give the ((1E)-{1-[4-(difluoromethoxy)-3-methoxyphenyl]cyclopropyl}methylene)methylamine (4.23 g, 100%). 1H NMR (400 MHz, CHLOROFORM-D) ppm 1.13-1.18 (m, 2H) 1.24-1.29 (m, 2H) 3.23 (d, J=1.51 Hz, 2H) 3.86 (s, 3H) 6.51 (t, J=75.29 Hz, 1H) 6.87 (dd, J=8.28, 2.01 Hz, 1H) 6.91 (d, J=2.01 Hz, 1H) 7.08 (d, J=8.03 Hz, 1H) 7.47 (q, J=1.51 Hz, 1H). GC-MS (EI) for C₁₃H₁₅F₂NO₂ m/z 255 (M)⁺.

Step 7:

HCl (1.0 M in ether, 25 mL, 25 mmol) was added drop wise with a syringe to a cooled (ice-bath) solution of ((1E)-{1-[4-(difluoromethoxy)-3-methoxyphenyl]cyclopropyl}methylene)methylamine, prepared in step 6, (4.23 g, 16.6 mmol) in dry DCM (50 mL). The mixture was stirred for 10 minutes, then the solvent was evaporated. The residue was dissolved in MeCN (70 mL) and Na₂SO₄ (s) (20 g, 141 mmol) and methylvinylketone (2.76 mL, 33.1 mmol) were added. The mixture was stirred at 85° C. for 15 hours. The solvent was evaporated and the residue was partitioned between water and DCM. The pH of the aqueous layer was adjusted to 10 with 1M aq. NaOH. The layers were separated and the water phase was extracted once more with DCM. The organic layers were combined, dried and concentrated, the residue was purified by flash column chromatography (DCM/CHCl₃ sat. with NH₃(g) 9:1). To afford (3aS*,7aS*)-3a-[4-(difluoromethoxy)-3-methoxyphenyl]-1-methyloctahydro-6H-indol-6-one (2.72 g, 50%).

1H NMR (400 MHz, CHLOROFORM-D) ppm 2.05-2.25 (m, 5H) 2.26-2.36 (m, 1H) 2.31 (s, 3H) 2.40-2.51 (m, 1H) 2.59 (d, J=3.76 Hz, 2H) 2.93 (t, J=3.51 Hz, 1H) 3.10-3.17 (m, J=5.90, 3.64 Hz, 1H) 3.89 (s, 3H) 6.54 (t, J=75.16 Hz, 1H) 6.91-6.98 (m, 2H) 7.13 (d, J=8.03 Hz, 1H). MS (ESI+) for C₁₇H₂₁F₂NO₃ m/z 326

### COMPARATIVE EXAMPLE 504

### (3aS*,7aS*)-3a-[4-(difluoromethoxy)-3-methoxyphenyl]-1-methyloctahydro-1H-indol-6-amine

NH₄OAc (19 g, 24.4 mmol) was added to a stirred solution of (3aS*,7aS*)-3a-[4-(difluoromethoxy)-3-methoxyphenyl]-1-methyloctahydro-6H-indol-6-one, Comparative Example 503, (2.65 g, 8.14 mmol) in dry MeOH (250 mL) at 65° C. The mixture was stirred for 30 minutes then NaBH₃CN (614 mg, 9.77 mmol) was added and the mixture stirred for 1 hour. The solvent was evaporated under reduced pressure and the residue was extracted twice with 1M aq. NaOH. and DCM. The organic layers were combined, dried and concentrated to give 2.1 g crude product which was purified by flash column chromatography (iso-hexane/CHCl₃ sat. with NH₃(g) 30:70). This gave after concentration 1.188 g (45%) of an approximately 1:1 cis/trans mixture of the title compound, which was used without further purification. 1H NMR (400 MHz, CHLOROFORM-D) ppm 0.91-1.03 (m, 1H) 1.26-1.35 (m, 1H) 1.36-1.43 (m, 1H) 1.44-1.54 (m, 1H) 1.56-1.66 (m, 1H) 1.69-1.83 (m, 4H) 1.85-2.11 (m, 6H) 2.20-2.32 (m, 3H) 2.32-2.34 (m, 3H) 2.37 (s, 3H) 2.62-2.67 (m, 1H) 2.67-2.73 (m, J=3.14, 3.14 Hz, 1H) 2.95-3.08 (m, 2H) 3.11-3.26 (m, 2H) 3.87 (s, 3H) 3.87 (s, 3H) 6.52 (t, J=75.42 Hz, 2H) 6.87-6.93 (m, 2H) 6.94 (d, J=2.26 Hz, 2H) 7.07 (dd, J=8.41, 2.38 Hz, 2H). MS (ESI+) for C₁₇H₂₄F₂N₂O₂ m/z 327 (M+H)⁺.

### EXAMPLE 505

### N-{(3aS*,6S*,7aS*)-3a-[4-(difluoromethoxy)-3-methoxyphenyl]-1-methyloctahydro-1H-indol-6-yl}-N′-[2-fluoro-3-(trifluoromethyl)phenyl]urea and

### EXAMPLE 506

### N-{(3aS*,6R*,7aS*)-3a-[4-(difluoromethoxy)-3-methoxyphenyl]-1-methyloctahydro-1H-indol-6-yl}-N′-[2-fluoro-3-(trifluoromethyl)phenyl]urea

2-Fluoro-3-trifluoromethylphenyl isocyanate (27 mg, 0.13 mmol) was added to (3aS*,7aS*)-3a-[4-(difluoromethoxy)-3-methoxyphenyl]-1-methyloctahydro-1H-indol-6-amine, Comparative Example 504, (29 mg, 0.089 mmol) in dry DCM (2 mL). The reaction mixture was stirred at rt. for 2 hours. The solvent was evaporated and the crude product was purified by flash column chromatography (iso-hexane/CHCl₃ sat. with NH₃(g) 60:40-50:50).

The trans-compound N-{(3aS*,6S*,7aS*)-3a-[4-(difluoromethoxy)-3-methoxyphenyl]-1-methyloctahydro-1H-indol-6-yl}-N′-[2-fluoro-3-(trifluoromethyl)phenyl]urea, Example 505, (16 mg, 34%) was eluted first: 1H NMR (400 MHz, CHLOROFORM-D) ppm 1.40 (tt, J=13.92, 3.05 Hz, 1H) 1.66-1.75 (m, 1H) 1.75-1.82 (m, 1H) 1.78-1.90 (m, 1H) 1.90-2.01 (m, 2H) 1.96-2.06 (m, 1H) 2.13 (td, J=14.25, 3.23 Hz, 1H) 2.33 (ddd, J=10.96, 9.92, 5.86 Hz, 1H) 2.35 (s, 3H) 2.74-2.80 (m, 1H) 3.29 (td, J=9.34, 5.49 Hz, 1H) 3.89 (s, 3H) 4.08-4.18 (m, 1H) 6.31 (d, J=2.81 Hz, 1H) 6.54 (t, J=75.32 Hz, 1H) 6.90 (dd, J=8.30, 2.20 Hz, 1H) 6.93 (d, J=2.20 Hz, 1H) 7.10 (d, J=8.30 Hz, 1H) 7.15-7.27 (m, 2H) 7.48 (d, J=6.96 Hz, 1H) 8.28 (td, J=7.69, 1.83 Hz, 1H). MS (ESI+) for C₂₅H₂₇F₆N₃O₃ m/z 532 (M+H)⁺. HRMS (EI) calcd for C₂₅H₂₇F₆N₃O₃: 531.196, found 531.195.

The cis-compound was then eluted N-{(3aS*,6R*,7aS*)-3a-[4-(difluoromethoxy)-3-methoxyphenyl]-1-methyloctahydro-1H-indol-6-yl}-N′-[2-fluoro-3-(trifluoromethyl)phenyl]urea, Example 506, (15 mg, 32%) 1H NMR (400 MHz, CHLOROFORM-D) ppm 1.02-1.16 (m, 1H) 1.39 (ddd, J=14.65, 11.96, 3.05 Hz, 1H) 1.77 (ddd, J=12.67, 11.20, 4.82 Hz, 1H) 1.78-1.87 (m, 1H) 1.89 (ddd, J=12.79, 8.82, 6.23 Hz, 1H) 2.03 (dt, J=14.04, 2.81, 2.56 Hz, 1H) 2.20 (td, J=13.86, 3.05 Hz, 1H) 2.21-2.28 (m, 1H) 2.31 (ddd, J=11.08, 9.61, 6.29 Hz, 1H) 2.35 (s, 3H) 2.70-2.75 (m, 1H) 3.23 (td, J=9.19, 4.82 Hz, 1H) 3.88 (s, 3H) 3.97-4.10 (m, 1H) 4.56 (d, J=7.81 Hz, 1H) 6.54 (t, J=75.38 Hz, 1H) 6.52-6.59 (m, 1H) 6.91 (dd, J=8.30, 2.08 Hz, 1H) 6.94 (d, J=2.08 Hz, 1H) 7.10 (d, J=8.30 Hz, 1H) 7.11-7.21 (m, 2H) 8.34 (td, J=7.66, 1.89 Hz, 1H). MS (ESI+) for C₂₅H₂₇F₆N₃O₃ m/z 532 (M+H)⁺. HRMS (EI) calcd for C₂₅H₂₇F₆N₃O₃: 531.196, found 531.195.

### EXAMPLE 507

### N-{(3aS*,6R*,7aS*)-3a-[4-(difluoromethoxy)-3-methoxyphenyl]-1-methyloctahydro-1H-indol-6-yl}-N′-(3,4-difluorophenyl)urea

3,4-difluoromethylphenyl isocyanate (21 mg, 0.14 mmol) was added to (3aS*,7aS*)-3a-[4-(difluoromethoxy)-3-methoxyphenyl]-1-methyloctahydro-1H-indol-6-amine, Comparative Example 504, (40 mg, 0.12 mmol) in dry DCM (2 mL). The reaction mixture was stirred at rt. for 2 hours. The solvent was evaporated and the crude product was purified by flash column chromatography (iso-hexane/CHCl₃ sat. with NH₃(g) 60:40-50:50), to give the title compound. 1H NMR (400 MHz, CHLOROFORM-D) ppm 0.94-1.07 (m, J=2.76 Hz, 1H) 1.25-1.36 (m, 1H) 1.70-1.80 (m, 2H) 1.81-1.91 (m, 1H) 1.96-2.03 (m, 1H) 2.08-2.22 (m, 2H) 2.23-2.30 (m, 1H) 2.32 (s, 3H) 2.66-2.72 (m, 1H) 3.15-3.23 (m, 1H) 3.86 (s, 3H) 3.93-4.03 (m, J=8.03, 3.76 Hz, 1H) 4.44 (d, J=8.03 Hz, 1H) 6.51 (t, J=75.42 Hz, 1H) 6.44 (s, 1H) 6.83-6.93 (m, 3H) 6.96-7.05 (m, 1H) 7.07 (d, J=8.28 Hz, 1H) 7.26-7.33 (m, 1H). MS (ESI+) for C₂₄H₂₇F₄N₃O₃ m/z 482 (M+H)⁺.)⁺. HRMS (EI) calcd for C₂₄H₂₇F₄N₃O₃: 481.199, found 481.199.

### EXAMPLE 508

### N-{(3aS*,6R*,7aS*)-3a-4-(difluoromethoxy)-3-methoxyphenyl]-1-methyloctahydro-1H-indol-6-yl}-N′-[3-fluoro-5-(trifluoromethyl)phenyl]urea and

### EXAMPLE 509

### N-{(3aS*,6S*,7aS*)-3a-[4-(difluoromethoxy)-3-methoxyphenyl]-1-methyloctahydro-1H-indol-6-yl}-N′-[3-fluoro-5-(trifluoromethyl)phenyl]urea

3-Fluoro-5-trifluoromethylphenyl isocyanate (28 mg, 0.14 mmol) was added to (3aS*,7aS*)-3a-[4-(difluoromethoxy)-3-methoxyphenyl-1-methyloctahydro-1H-indol-6-amine, Comparative Example 504, (40 mg, 0.12 mmol) in dry DCM (2 mL). The reaction mixture was stirred at rt. for 2 hours. The solvent was evaporated and the crude products isolated by flash column chromatography (iso-hexane/CHCl₃ sat. with NH₃(g) 60:40-50:50). The cis-compound N-{(3aS*,6R*,7aS*)-3a-[4-(difluoromethoxy)-3-methoxyphenyl]-1-methyloctahydro-1H-indol-6-yl}-N′-[3-fluoro-5-(trifluoromethyl)phenyl]urea (12 mg, 19): 1H NMR (400 MHz, CHLOROFORM-D) ppm 0.94-1.06 (m, 1H) 1.27-1.38 (m, 1H) 1.68-1.89 (m, 3H) 1.93-2.01 (m, 1H) 2.04-2.14 (m, 1H) 2.13-2.20 (m, 1H) 2.22-2.31 (m, 1H) 2.28 (s, 3H) 2.65-2.71 (m, 1H) 3.11-3.20 (m, 1H) 3.85 (s, 3H) 3.91-4.04 (m, 1H) 4.79 (d, J=8.03 Hz, 1H) 6.53 (t, J=75.29 Hz, 1H) 6.83-6.94 (m, 3H) 7.02-7.11 (m, 2H) 7.20 (s, 1H) 7.37-7.46 (m, 1H). MS (ESI+) for C₂₅H₂₇F₆N₃O₃ m/z 532 (M+H)⁺. HRMS (EI) calcd for C₂₅H₂₇F₆N₃O₃: 531.196, found 531.195.

Trans-compound N-{(3aS*,6S*,7aS*)-3a-[4-(difluoromethoxy)-3-methoxyphenyl]-1-methyloctahydro-1H-indol-6-yl}-N′-[3-fluoro-5-(trifluoromethyl)phenyl]urea (17 mg, 27%): 1H NMR (400 MHz, CHLOROFORM-D) ppm 1.32-1.43 (m, 1H) 1.65-1.72 (m, 1H) 1.74-2.01 (m, 5H) 2.03-2.13 (m, 1H) 2.27-2.36 (m, 1H) 2.32 (s, 3H) 2.73-2.77 (m, 1H) 3.21-3.29 (m, 1H) 3.87 (s, 3H) 4.09-4.15 (m, 1H) 6.54 (t, J=75.29 Hz, 1H) 6.59 (s, 1H) 6.86-6.89 (m, 1H) 6.89-6.91 (m, J=2.26 Hz, 1H) 6.94-6.98 (m, J=8.03 Hz, 1H) 7.09 (d, J=8.28 Hz, 1H) 7.29 (s, 1H) 7.42-7.49 (m, 1H) 7.50-7.56 (m, 1H). MS (ESI+) for C₂₅H₂₇F₆N₃O₃ m/z 532 (M+H)⁺. HRMS (EI) calcd for C₂₅H₂₇F₆N₃O₃: 531.196, found 531.194.

### EXAMPLE 510

### N-{(3aS*,6R*,7aS*)-3a-[4-(difluoromethoxy)-3-methoxyphenyl]-1-methyloctahydro-1H-indol-6-yl}-N′-(2,5-difluorophenyl)urea

2,5-difluoromethylphenyl isocyanate (21 mg, 0.14 mmol) was added to (3aS*,7aS*)-3a-[4-(difluoromethoxy)-3-methoxyphenyl]-1-methyloctahydro-1H-indol-6-amine, Comparative Example 504, (40 mg, 0.12 mmol) in dry DCM (2 mL). The reaction mixture was stirred at rt. for 2 hours. The solvent was evaporated and the crude-product was purified by flash column chromatography (iso-hexane/CHCl₃ sat. with NH₃(g) 60:40-50:50). This gave the title compound (11 mg, 19%): 1H NMR (400 MHz, CHLOROFORM-D) ppm 0.99-1.10 (m, 1H) 1.31-1.41 (m, 1H) 1.72-1.92 (m, 3H) 1.97-2.05 (m, 1H) 2.11-2.32 (m, 3H) 2.33 (s, 3H) 2.67-2.72 (m, 1H) 3.15-3.24 (m, 1H) 3.86 (s, 3H) 3.94-4.08 (m, 1H) 4.65 (d, J=8.03 Hz, 1H) 6.56 (t, J=75.29 Hz, 1H) 6.54-6.64 (m, 2H) 6.85-6.98 (m, 3H) 7.07 (d, J=8.28 Hz, 1H) 7.86-7.98 (m, 1H). MS (ESI+) for C₂₄H₂₇F₄N₃O₃ m/z 482 (M+H)⁺. HRMS (EI) calcd for C₂₄H₂₇F₄N₃O₃: 481.199, found 481.198.

### EXAMPLE 511

### N-{(3aS*,6R*,7aS*)-3a-[4-(difluoromethoxy)-3-methoxyphenyl]-1-methyloctahydro-1H-indol-6-yl}-N′-(3,4,5-trifluorophenyl)urea

Triphosgene (15 mg, 0.05 mmol) in dry DCM (0.5 mL) was added to a solution of (3aS*,7aS*)-3a-[4-(difluoromethoxy)-3-methoxyphenyl]-1-methyloctahydro-1H-indol-6-amine, Comparative Example 504, (40 mg, 0.12 mmol) and dry NEt₃ (34 μL, 0.25 mmol) in dry DCM (1.5 mL). The mixture was stirred at rt. for 3 h. 3,4,5-trifluoroaniline (18 mg, 0.12 mmol) was added and stirring continued over night (20 h). The solvent was evaporated and the crude product was purified by flash column chromatography (iso-hexane/CHCl₃ sat. with NH₃(g) 60:40-50:50). This gave the title compound (11 mg, 18%). 1H NMR (400 MHz, CHLOROFORM-D) ppm 0.94-1.06 (m, 1H) 1.26-1.37 (m, 1H) 1.70-1.79 (m, 2H) 1.81-1.90 (m, 1H) 1.93-2.02 (m, 1H) 2.06-2.20 (m, 2H) 2.23-2.30 (m, 1H) 2.31 (s, 3H) 2.69 (s, 1H) 3.12-3.22 (m, 1H) 3.86 (s, 3H) 3.91-4.03 (m, J=11.80, 4.02 Hz, 1H) 4.62 (d, J=8.03 Hz, 1H) 6.53 (t, J=75.42 Hz, 1H) 6.73 (s, 1H) 6.84-6.93 (m, 2H) 6.95-7.02 (m, 2H) 7.07 (d, J=8.03 Hz, 1H). MS (ESI+) for C₂₄H₂₆F₅N₃O₃ m/z 500 (M+H)⁺. HRMS (EI) calcd for C₂₄H₂₆F₅N₃O₃: 499.189, found 499.189.

### COMPARATIVE EXAMPLE 512

### (3aS*,7aS*)-3a-[4-methoxy-3-(trifluoromethoxy)phenyl]-1-methyloctahydro-6H-indol-6-one trifluoroacetate

Step 1:

Br₂ (1.47 mL, 26.6 mmol) in glacial acetic acid (20 mL) was added dropwise over 30 min to a solution of 2-trifluoromethoxyphenol (5.05 g, 26.4 mmol) in glacial acetic acid (100 mL). Stirring 24 h gave 95% conversion, and the stirring continued over weekend (total 92 h). The reaction was quenched with 5M aq. Na₂S₂O₃ until the color had disappered. The reaction mixture was diluted with DCM (150 mL) and extracted three timed with saturated aq. NaHCO₃. The organic layer was dried (Na₂SO₄) and concentrated to give 4-bromo-2-(trifluoromethoxy)phenol (7.092 g, 97%) as a light-green fluffy solid. GC-MS (EI) for C₇H₄BrF₃O₂ m/z 256 (M)⁺.

Step 2:

4-bromo-2-(trifluoromethoxy)phenol, prepared in step 1, (7.09 g, 27.6 mmol) was dissolved in acetone (120 mL). Methyl iodide (10.30 mL, 165.6 mmol) and K₂CO₃ (15.3 g, 110.4 mmol) were added and the reaction mixture heated at 60° C. for 18 h heated. The mixture was filtered, and acetone removed under reduced pressure with no heat applied (product found to be volatile). The residue was extracted with water (150 mL) and DCM (2×100 mL). The organic layers were combined, dried and concentrated to give 4-bromo-1-methoxy-2-(trifluoromethoxy)benzene (6,75 g, 90%) as a light red oil. 1H NMR (400 MHz, CHLOROFORM-D) ppm 3.86 (s, 3H) 6.82-6.91 (m, 1H) 7.34-7.40 (m, 2H). GC-MS (EI) for C₈H₆BrF₃O₂ m/z 270 (M)⁺.

Step 3:

Pd(PPh₃)₄ (1.44 g, 1.25 mmol) was added to a solution of 4-bromo-1-methoxy-2-(trifluoromethoxy)benzene, prepared in step 2, (6.75 g, 24.9 mmol) and tributyl vinylstannane (11.5 g, 36 mmol) in dry toluene (100 mL). A color change from light red to black was noticed. The mixture was stirred at 100° C. over night for 23 h. 20% of the starting material was unreacted. Additional Pd(PPh₃)₄ (0.75 g, 0.65 mmol) and tributyl vinylstannane (2.5 mL, 8.6 mmol) were added in portions over 25 hours until all starting material was reacted. The solvent was evaporated (40° C.) under reduced pressure and the crude product was purified by flash column chromatography (iso-hexane), to give 1-methoxy-2-(trifluoromethoxy)-4-vinylbenzene (3.85 g, 71%) as a colorless solid. 1H NMR (400 MHz, CHLOROFORM-D) ppm 3.87 (s, 3H) 5.20 (d, J=11.04 Hz, 1H) 5.62 (d, J=17.57 Hz, 1H) 6.61 (dd, J=17.57, 11.04 Hz, 1H) 6.93 (d, J=8.53 Hz, 1H) 7.26-7.31 (m, 2H). GC-MS (EI) for C₁₀H₉F₃O₂ m/z 218 (M)⁺.

Step 4:

OsO₄ (220 mg, 0.88 mmol) was added to a stirred solution of 1-methoxy-2-(trifluoromethoxy)₄-vinylbenzene, prepared in step 3, (3.85 g, 17.7 mmol) in THF:H₂O 120:60 mL. A color change from colorless to dark blue/black was noticed. After 10 min, NaIO₄ (9.45 g, 44.2 mmol) was added and a grey precipitation was formed during addition. Additional TBF and water (50:25 mL) were added to make the mixture more homogenous. The reaction mixture was stirred at rt. for 30 min, followed by extraction with DCM (2×150 mL) and aq. NaHCO₃ (200 mL, H₂O: sat. aq. NaHCO₃ 1:3). The organic layers were combined, dried and concentrated to give 4-methoxy-3-(trifluoromethoxy)benzaldehyde as a dark green oil, used without further purification. 1H NMR (400 MHz, CHLOROFORM-D) ppm 3.97 (s, 3H) 7.11 (d, J=8.53 Hz, 1H) 7.75-7.78 (m, 1H) 7.81 (dd, J=8.53, 2.01 Hz, 1H) 9.87 (s, 1H). GC-MS (EI) for C₉H₇F₃O₃ m/z 220 (M)⁺.

Step 5:

NaBH₄ (1.00 g, 26.4 mmol) was added portion-wise to 4-methoxy-3-(trifluoromethoxy)benzaldehyde, prepared in step 4, (3.87 g, 17.6 mmol) in dry MeOH (70 mL). The mixture was stirred for 30 min and quenched with 2M aq. HCl (20 mL). The solvent was evaporated and the residue was extracted with water (200 mL) and DCM (2×200 mL). The organic layers were combined, dried and concentrated to 3.8 g crude product. Purification was performed by flash column chromatography (DCM-1% MeOH in DCM), to give [4-methoxy-3-(trifluoromethoxy)phenyl]methanol (2.41 g, 62%) as colorless oil.

1H NMR (500 MHz, CHLOROFORM-D) ppm 1.65 (t, J=5.86 Hz, 1H) 3.88 (s, 3H) 4.64 (d, J=5.86 Hz, 2H) 6.98 (d, J=8.42 Hz, 1H) 7.24-7.27 (m, 1H) 7.25-7.27 (m, 1H). GC-MS (EI) for C₉H₉F₃O₃ m/z 222 (M)⁺.

Step 6:

Br₂ (0.99 mL, 19.3 mmol) was added to an ice-cold solution of PPh₃ (5.07 g, 19.3 mmol) in DCM (50 mL). The mixture was stirred for 15 min and [4-methoxy-3-(trifluoromethoxy)phenyl]methanol, prepared in step 5, (3.30 g, 14.9 mmol) in DCM (50 mL) was added drop-wise. The reaction mixture was stirred at 0° C. for 30 min. The solvent was evaporated and the residue purified on a short silica plug to give 4-(bromomethyl)-1-methoxy-2-(trifluoromethoxy)benzene as a colorless oil (3.58 g, 84%).

1H NMR (400 MHz, CHLOROFORM-D) ppm 3.87 (s, 3H) 4.45 (s, 2H) 6.94 (d, J=8.78 Hz, 1H) 7.26-7.30 (m, 2H). GC-MS (EI) for C₉H₈BrF₃O₂ m/z 284 (M)⁺.

Step 7:

NaCN (0.71 g, 14.6 mmol) was added to a solution of 4-(bromomethyl)-1-methoxy-2-(trifluoromethoxy)benzene, prepared in step 6, (3.48 g, 12.1 mmol) in dry DMF (30 mL). The mixture was stirred for 5.5 h at rt. followed by extraction with water (5×10 mL) and ether (100 mL). The ethereal layer was dried and concentrated to give [4-methoxy-3-(trifluoromethoxy)phenyl]acetonitrile (2.50 g, 90%) as a light brown oil. 1H NMR (400 MHz, CHLOROFORM-D) ppm 3.70 (s, 2H) 3.87-3.89 (m, J=1.25 Hz, 3H) 6.97-7.01 (m, J=8.28 Hz, 1H) 7.16-7.19 (m, 1H) 7.21-7.26 (m, 1H). GC-MS (EI) for C₁₀H₈F₃NO₂ m/z 231 (M)⁺.

Step 8:

[4-methoxy-3-(trifluoromethoxy)phenyl]acetonitrile, prepared in step 7, (1.24 g, 5.4 mmol) in dry DME (30 mL), dry DMSO (0.76 mL, 10.7 mmol) and 1-bromo-2-chloroethane (1.34 mL, 16.1 mmol) were added, in that order, to a stirred suspension of LiNH₂ (1.29 g, 53.6 mmol) in dry DME (30 mL) at 65° C. Stirring at that temperature over night for 17 h gave 100% conversion. The reaction was quenched with water (5 mL) and the solvent was evaporated. The residue was partitioned between water (150 mL) and DCM (2×150 mL). The organic layers were combined, dried then concentrated and the residue purified by flash column chromatography (DCM/isohexane) affording 1-[4-methoxy-3-(trifluoromethoxy)phenyl]cyclopropanecarbonitrile (1.38 g, 40%) as a light brown oil. 1H NMR (400 MHz, CHLOROFORM-D) ppm 1.31-1.36 (m, 2H) 1.68-1.72 (m, 2H) 3.87 (s, 3H) 6.96 (d, J=8.53 Hz, 1H) 7.09-7.12 (m, J=2.26, 1.25 Hz, 1H) 7.24-7.28 (m, 1H). GC-MS (EI) for C₁₂H₁₀F₃NO₂ m/z 257 (M)⁺.

Step 9:

DIBAL (1.0M in toluene, 14.9 mL, 14.9 mmol) was added dropwise at rt. to a solution of 1-[4-methoxy-3-(trifluoromethoxy)phenyl]cyclopropanecarbonitrile, prepared in step 8, (958 mg, 3.73 mmol) in dry THF (30 mL) under N₂ (g). The reaction was stirred for 1 hour then additional DIBAL (3.73 mL, 3.73 mmol) was added and the mixture stirred for 17 h. 2M aq. HCl was added and the mixture twice extracted with DCM. The organic layers were combined, dried and concentrated to give 1-[4-methoxy-3-(trifluoromethoxy)phenyl]cyclopropanecarbaldehyde (690 mg, 71%) as a yellow oil. 1H NMR (400 MHz, CHLOROFORM-D) ppm 1.34-1.41 (m, 2H) 1.53-1.58 (m, 2H) 3.87 (s, 3H) 6.97 (d, J=8.53 Hz, 1H) 7.14-7.28 (m, 2H) 9.14 (s, 1H).

Step 10:

Na₂SO₄ (520 mg, 3.66 mmol), methylamine hydrochloride (494 mg, 7.32 mmol) and methyl vinylketone (305 mg, 3.66 mmol) were added, in that order, to a solution of 1-[4-methoxy-3-(trifluoromethoxy)phenyl]cyclopropanecarbaldehyde, prepared in step 9, (635 mg, 2.44 mmol) in dry DMSO (2 mL) and dry MeCN (10 mL). A reflux condenser was attached to the reaction vessel and the mixture was heated to 80° C. and stirred for 23 hours. Additional methyl vinylketone (300 μL, 3.66 mmol) and Na₂SO₄ (400 mg, 2.88 mmol) were added in two portions over 4 hours with continuous stirring for 20 hours. The solvent was evaporated and the residue partitioned between DCM and water. The pH was adjusted to ˜9 with 2M aq. NaOH and the phases were separated. The aqueous layer was extracted once more with DCM. The organic layers were combined, dried and concentrated to give 1.3 g of a black viscous oil. Purification by flash column chromatography (DCM-5% MeOH in DCM) followed by preparative HPLC gave (3aS*,7aS*)-3a-[4-methoxy-3-(trifluoromethoxy)phenyl]-1-methyloctahydro-6H-indol-6-one trifluoroacetate (125 mg, 11.4%). MS (ESI+) for C₁₇H₂₀F₃NO₃ m/z 344 (M+H)⁺.

### COMPARATIVE EXAMPLE 513

### (3aS*,7aS*)-3a-[4-methoxy-3-(trifluoromethoxy)phenyl]-1-methyloctahydro-1H-indol-6-amine

(3aS*,7aS*)-3a-[4-methoxy-3-(trifluoromethoxy)phenyl]-1-methyloctahydro-6H-indol-6-one trifluoroacetate, prepared in Comparative Example 512, (39 mg, 0.083 mmol) was dissolved in MeOH saturated with NH₃ (g). The mixture was stirred for 10 minutes, then treated with NaCNBH₃ (11 mg, 0.17 mmol). The reaction was heated to 120° C. for 5 min in a microwave oven followed by extraction with water and DCM (×2). the organic layers were combined, dried and concentrated and purified by flash column chromatography (CHCl₃ sat. with NH₃ (g)/isohexane 1:1), to give the product (22 mg, 77%) as a mixture of two diastereomers. MS (ESI+) for C₁₇H₂₃F₃N₂O₂ m/z 345 (M+H)⁺.

### EXAMPLE 514

### N-[3-fluoro-5-(trifluoromethyl)phenyl]-N′-{(3aS*,6R*,7aS*)-3a-[4-methoxy-3-(trifluoromethoxy)phenyl]-1-methyloctahydro-1H-indol-6-yl}urea and

### EXAMPLE 515

### N-[3-fluoro-5-(trifluoromethyl)phenyl]-N′-{(3aS*,6S*,7aS*)-3a-[4-methoxy-3-(trifluoromethoxy)phenyl]-1-methyloctahydro-1H-indol-6-yl}urea

(3aS*,7aS*)-3a-[4-methoxy-3-(trifluoromethoxy)phenyl]-1-methyloctahydro-1H-indol-6-amine, Comparative Example 513, (22 mg, 0.064 mmol) and 3-fluoro-5-trifluoromethylphenylisocyanate (14 mg, 0.07 mmol) were dissolved in dry DCM (1.5 mL) and stirred at r.t. for 1.5 h. The solvent was evaporated and the residue purified by preparative HPLC to give the cis-compound N-[3-fluoro-5-(trifluoromethyl)phenyl]-N′-{(3aS*,6R*,7aS*)-3a-[4-methoxy-3-(trifluoromethoxy)phenyl]-1-methyloctahydro-1H-indol-6-yl}urea (4 mg, 11%) as a colorless solid.

MS (ESI+) for C₂₅H₂₆F₇N₃O₃ m/z 550 (M+H)⁺. HRMS (EI) calcd for C₂₅H₂₆F₇N₃O₃: 549.186, found 549.185.

The trans-compound N-[3-fluoro-5-(trifluoromethyl)phenyl]-N′-{(3aS*,6S*,7aS*)-3a-[4-methoxy-3-(trifluoromethoxy)phenyl]-1-methyloctahydro-1H-indol-6-yl}urea (7 mg, 20%) was also obtained as a colorless solid. MS (ESI+) for C₂₅H₂₆F₇N₃O₃ m/Z 550 (M+H)⁺.

### EXAMPLE 516

### N-[(3aS*,6R*,7aS*)-3a-(3,4-dimethoxyphenyl)-1-methyloctahydro-1H-indol-6-yl]-N′-(6-fluoro-1,3-benzothiazol-2-yl)urea trifluoroacetate

2-Amino-6-fluorobenzothiazole (24 mg, 0.14 mmol) was dissolved in 1 mL dry CH₂Cl₂ under N₂. Triethylamine (24 mg, 0.14 mmol) was added followed by drop wise addition of Triphosgene (20 mg, 0.06 mmol) in 1 mL of dry CH₂Cl₂. The mixture was stirred at room temperature for 2 hrs, and then (3aS*,6R*,7aS*)-3a-(3,4-dimethoxyphenyl)-1-methyloctahydro-1H-indol-6-amine, Comparative Example 7, (35 mg, 0.12 mmol) was added. The mixture was stirred at room temperature overnight and then concentrated. Purification using preparative HPLC-MS (5-50% MeCN) gave the product as light yellow oil (2 mg, 4%).

¹HNMR (270 MHz, Chloroform-d) ppm 1.23-1.36 (m, 1H); 2.00-2.34 (m, 8H); 2.61 (d, J=13.48. 3H); 3.47 (s, 1H); 3.69-3.79 (m, 1H); 3.89 (s, 6H); 4.23-4.33 (b, 2H); 6.75-6.88 (m, 3H); 7.11-7.19 (m, 1H); 7.38-7.47 (m, 1H); 7.54-7.61 (m, 1H); 7.63 (s, 1H)

MS (ESI⁺) for C₂₅H₂₉FN₄O₃S m/z 485 (M+H⁺), HRMS found: 484,1939 calculated: 484,1944

### EXAMPLE 517

### N-[(3aS*,6R*,7aS*)-3a-(3,4-dimethoxyphenyl)-1-methyloctahydro-1H-indol-6-yl]-N′-(4-methyl-1,3-thiazol-2-yl)urea trifluoroacetate

Procedure: as for Example 516

Reagent: 2-Amino-4-methylthiazole

Yield: 13 mg (25%).

¹H-NMR (270 MHz, Chloroform-d) ppm 1.20-1.35 (m, 1H); 1.86-2.59 (m, 8H); 2.99 (s, 3H); 3.48 (s, 1H); 3.62-3.72 (m, 1H); 3.89 (s, 9H); 4.32-4.45 (m, 2H); 5.66 (w, 1H); 6.42-6.53 (m, 1H); 6.77-6.94 (m, 2H); 7.64 (s, 1H)

MS (ESI⁺) for C₂₂H₃₀N₄O₃S m/z 431 (M+H⁺), HRMS found: 430,2041 calculated: 430,2039

### EXAMPLE 518

### N-(4-chloro-1,3-benzothiazol-2-yl)-N′-[(3aS*,6R*,7aS*)-3a-(3,4-dimethoxyphenyl)-1-methyloctahydro-1H-indol-6-yl]urea trifluoroacetate

Procedure: as for Example 516

Reagent: 2-Amino-4-chlorobenzothiazole

Yield: 17 mg, 28%

¹HNMR (270 MHz, Chloroform-d) ppm 1.24-1.39 (m, 1H); 1.90-2.35 (m, 6 h); 2.61 (d, J=12.87, 1H); 2.87 (s, 1H); 3.02 (s, 3H); 3.75 (s, 1H); 3.90 (s, 6H); 4.25-4.40 (m, 2H), 6.16 (b, 1H); 6.77-6.88 (m, 3H); 7.31-7.37 (m, 1H); 7.45 (d, J=8.91, 1H); 7.58 (d, J=8.66, 1H): 7.71 (s, 1H)

MS (ESI⁺) for C₂₅H₂₉ClN₄O₃S m/z 501 (M+H⁺), HRMS found: 500,1665 calculated 500,1649

### EXAMPLE 519

### N-[4-(2-chloro-6-fluorophenyl)-1,3-thiazol-2-yl]-N′-[(3aS*,6R*,7aS*)-3a-(3,4-dimethoxyphenyl)-1-methyloctahydro-1H-indol-6-yl]urea trifluoroacetate

Procedure: as for Example 516

Reagent: 4-(2-Chloro-6-fluorophenyl)-1,3-thiazole-2-amine

Yield: 2.7 mg, 4%

¹HNMR (270 MHz, Chloroform-d) ppm 1.27-1.39 (m, 1H); 1.88-2.87 (m, 8H); 2.58 (d, J=13.12, 1H); 3.03 (s, 3H); 3.75 (s, 1H); 3.89 (d, J=3.34, 6H); 4.34 (b, 2H); 6.05 (w, 1H); 6.76-6.89 (m, 2H); 7.01 (s, 1H); 7.11-7.19 (m, 1H); 7.31-7.46 (m, 2H); 7.63 (s, 1H)

MS (ESI⁺) for C₂₇H₃₀ClFN₄O₃S m/z 545 (M+H⁺), HRMS found: 544,1730 calculated: 544,1711

### EXAMPLE 520

### N-(6-chloro-1,3-benzothiazol-2-yl)-N′-[(3aS*,6R*,7aS*)-3a-(3,4-dimethoxyphenyl)-1-methyloctahydro-1H-indol-6-yl]urea trifluoroacetate

Procedure: as for Example 516

Reagent: 2-Amino-6-chlorobenzothiazole

Yield: 11.2 mg, 19%

¹HNMR (270 MHz, Chloroform-d) ppm 1.26-1.38 (m, 1H); 1.95-2.40 (m, 7H); 2.70 (d, J=13.36, 1H); 2.87 (s, 1H); 3.07 (s, 3H); 3.78 (s, 1H); 3.89 (s, 6H); 4.25-4.34 (m, 2H); 6.76-6.88 (m, 3H); 7.29-7.34 (m, 1H); 7.41 (d, J=7.42, 1H); 7.56 (d, J=7.91, 1H); 7.64 (s, 1H) MS (ESI+) for C₂₅H₂₉ClN₄O₃S m/z 501 (M+H⁺), HRMS found: 500,1653 calculated: 500,1649

### EXAMPLE 521

### N-[(3aS*,6R*,7aS*)-3a-(3,4-dimethoxyphenyl)-1-isopropyloctahydro-1H-indol-6-yl]-N′-(3-fluorophenyl)urea trifluoroacetate

Step 1:

3-Fluorophenylisocyanate (9.6 mg, 0.07 mmol) was added to a solution of (3aS*,6R*,7aS*)-1-benzyl-3a-(3,4-dimethoxyphenyl)octahydro-1H-indole-6-amine, Comparative Example 5, (25 mg, 0.07 mmol) in CH₂Cl₂ and stirred at RT for 2 hrs. The mixture was filtered and the precipitate was washed with CH₂Cl₂ and then dried to give N-[3aS*,6R*,7aS*)-1-benzyl-3a-(3,4-dimethoxyphenyl)octahydro-1H-indol-6-yl]-N′-(3-fluorophenyl)urea as a light beige solid.

MS (ESI+) for C₃₀H₃₄FN₃O₃ m/Z 504 (M+H⁺), HRMS calculated: 503,2584

Step 2:

Pd/C (10%, 2.5 mg) was added to a solution of N-[3aS*,6R*,7aS*)-1-benzyl-3a-(3,4-dimethoxyphenyl)octahydro-1H-indol-6-yl]-N′-(3-fluorophenyl)urea, prepared in step 1, (25 mg, 0.05 mmol) in MeOH/THF 1:1 (10 mL) and stirred under 1 atm of H₂. The mixture was stirred overnight and then concentrated to give N-(3-fluorophenyl)-N′-[3aS*,6R*,7aS*)-3a-(3,4-dimethoxyphenyl)octahydro-1H-indol-6-yl]-urea as a clear oil.

MS (ESI+) for C₂₃H₂₈FN₃O₃ m/z 414 (M+H⁺), HRMS calculated: 413,2584

Step 3:

Acetone (8.7 mg, 0.15 mmol) was added to a solution of N-(3-fluorophenyl)-N′-[3aS*,6R*,7aS*)-3a-(3,4-dimethoxyphenyl)octahydro-1H-indol-6-yl]-urea, prepared in step 2, (12 mg, 0.03 mmol) in 5 mL MeOH and stirred at room temperature for 30 minutes. Then NaCNBH₃ (19.7 mg, 0.3 mmol) was added and the mixture was stirred overnight at room temperature. The mixture was filtered, concentrated and purified using preparative HPLC-MS (20-70% MeCN) giving the title compound as light beige oil (5.9 mg, 43%).

¹HNMR (270 MHz, Chloroform-d) ppm 1.25 (d, J=6.43, 3H); 1.41 (d, J=6.19, 3H); 1.80-2.10 (m, 5H); 0.22-2.31 (m, 1H); 2.48 (d, J=9.16, 1H); 2.94 (s, 1H); 3.46 (b, 1H); 3.60-3.74 (m, 1H); 3.86 (d, J=1.73, 6H); 3.97 (b, 2H); 6.66-6.86 (m, 5H); 7.14-7.21 (m, 2H); 7.45 (d, J=11.14, 2H); 7.63 (s, 1H)

MS (ESI⁺) for C₂₆H₃₄FN₃O₃ m/z 456 (M+H⁺), HRMS found: 455,2581 calculated: 455,2584

### EXAMPLE 522

### N-(3-chloro-4-fluorophenyl)-N′-[(3aS*,6R*,7aS*)-3a-(3,4-dimethoxyphenyl)-1-isopropyloctahydro-1H-indol-6-yl]urea trifluoroacetate

Step 1:

(3aS*,6R*,7aS*)-1-benzyl-3a-(3,4-dimethoxyphenyl)octahydro-1H-indol-6-amine, Comparative Example 5, (50 mg, 0.14 mmol) was dissolved in 6 mL MeOH and Boc-anhydride (305 mg, 1.4 mmol) was added. Then 3.5 mL of 10% NaHCO₃(aq) (4.2 mmol) was added. Mixture stirred at room temperature overnight. More H₂O was added and the mixture was extracted with ethyl acetate, washed with brine and then dried with MgSO4. Finally concentrated to give tert-butyl [(3aS*,6R*,7aS*)-1-benzyl-3a-(3,4-dimethoxyphenyl)octahydro-1H-indol-6-yl]carbamate as a white solid.

MS (ESI⁺) for C₂₈H₃₈N₂O₄ m/z 467 (M+H⁺)

Step 2:

Pd/C (10%, 6 mg) was added to a solution of tert-butyl [(3aS*,6R*,7aS*)-1-benzyl-3a-(3,4-dimethoxyphenyl)octahydro-1H-indol-6-yl]carbamate, prepared in step 1, (60 mg, 0.13 mmol) in MeOH/THF 1:1 (1 (10 mL) and stirred under 1 atm of H₂. The mixture was stirred overnight and then concentrated to give tert-butyl [(3aS*,6R*,7aS*)-3a-(3,4-dimethoxyphenyl)octahydro-1H-indol-6-yl]carbamate as yellow oil.

MS (ESI⁺) for C₂₁H₃₂N₂O₄ m/z 377 (M+H⁺)

Step 3:

Acetone (36 mg, 0.625 mmol) was added to a solution of tert-butyl [(3aS*,6R*,7aS*)-3a-(3,4-dimethoxyphenyl)octahydro-1H-indol-6-yl]carbamate, prepared in step 2, (47 mg, 0.125 mmol) in 5 mL MeOH and stirred at room temperature for 30 minutes. Then NaCNBH₃ (82 mg, 1.25 mmol) was added and the mixture was stirred overnight at room temperature. Product is deprotected during the reaction course. The mixture was filtered then concentrated to give N-[(3aS*,6R*,7aS*)-3a-(3,4-dimethoxyphenyl)-1-isopropyloctahydro-1H-indol-6-yl]amine as clear oil.

MS (ESI⁺) for C₁₉H₃₀N₂O₂ m/z 319 (M+H⁺)

Step 4:

N-[(3aS*,6R*,7aS*)-3a-(3,4-dimethoxyphenyl)-1-isopropyloctahydro-1H-indol-6-yl]amine, prepared in step 3, (15 mg, 0.05 mmol) was dissolved in CH₂Cl₂ and 3-Chloro-4-fluorophenylisocyanate (8.1 mg, 0.05 mmol) was added. The mixture was stirred at room temperature overnight, then concentrated and purified with preparative HPLC-MS system 40-90% MeCN giving the product as a light beige oil (2 mg, 8%).

¹HNMR (270 MHz, Chloroform-d) ppm 1.25 (d, J=6.43, 3H); 1.41 (d, J=6.19, 3H); 1.80-2.10 (m, 5H); 0.22-2.31 (m, 1H); 2.48 (d, J=9.16, 1H); 2.94 (s, 1H); 3.44 (b, 1H); 3.60-3.74 (m, 1H); 3.86 (d, J=1.73, 6H); 3.97 (b, 2H); 6.66-6.86 (m, 4H); 7.14-7.21 (m, 2H); 7.45 (d, J=11.14, 2H); 7.63 (s, 1H)

MS (ESI⁺) for C₂₆H₃₃ClFN₃O₃ m/z 490 (M+H⁺)

### EXAMPLE 523

### N-(3′-cyanobiphenyl-4-yl)-N′-(3,4-difluorophenyl)-N-[(3aS*,6R*,7aS*)-3a-(3,4-dimethoxyphenyl)-1-methyloctahydro-1H-indol-6-yl]urea trifluoroacetate and

### EXAMPLE 524

### N-(3′-cyanobiphenyl-4-yl)-N′-(3,4-difluorophenyl)-N-[(3aS*,6S*,7aS*)-3a-(3,4-dimethoxyphenyl)-1-methyloctahydro-1H-indol-6-yl]urea trifluoroacetate

Step 1:

(3aS*,7aS*)-3a-(3,4-dimethoxyphenyl)-1-methyloctahydro-6H-indol-6-one, Comparative Example 4, (35 mg, 0.12 mmol) was dissolved in CH₂Cl₂ with 4′-Amino-biphenyl-3-carbonitrile (23 mg, 0.12 mmol). NaBH₄ (7.6 mg, 0.2 mmol) and a drop of acetic acid were added. The reaction was stirred at room temperature over night, then concentrated and purified using preparative HPLC-MS system 10-80% MeCN to give a mixture of cis- and trans-4′-{[(3aS*,7aS*)-3a-(3,4-dimethoxyphenyl)-1-methyloctahydro-1H-indol-6-yl]amino}biphenyl-3-carbonitrile as a brown oil.

MS (ESI⁺) for C₃₀H₃₃N₃O₂ m/z 468 (M+H⁺)

Step 2:

The cis/trans mixture of 4′-{[(3aS*,7aS*)-3a-(3,4-dimethoxyphenyl)-1-methyloctahydro-1H-indol-6-yl]amino}biphenyl-3-carbonitrile, prepared in step 1, (20 mg, 0.04 mmol) was dissolved in CH₂Cl₂ and 3,4-Difluorophenylisocyanate (7.2 mg, 0.05 mmol) was added. The mixture was stirred at room temperature overnight, then concentrated. Purification and separation of the diastereomers using preparative HPLC-MS system 20-70% MeCN gave the cis-product N-(3′-cyanobiphenyl-4-yl)-N′-(3,4-difluorophenyl)-N-[(3aS*,6R*,7aS*)-3a-(3,4-dimethoxyphenyl)-1-methyloctahydro-1H-indol-6-yl]urea trifluoroacetate as yellow oil (2.6 mg, 10%)

¹HNMR (270 MHz, Chloroform-d) ppm 1.21-1.28 (m, 1H); 1.80 (s, 1H): 2.21-2.42 (m, 4H); 2.60 (d, J=13.61, 1H); 2.82 (b, 1H); 2.97-3.01 (m, 1H) 3.04 (s, 3H); 3.70 (s, 1H); 3.86 (s, 6H); 4.26 (d, J=11.38, 2H); 6.33 (s, 1H); 6.73-6.86 (m, 3H); 6.92-7.08 (m, 2H); 7.32-7.44 (m, 2H); 7.58-7.69 (m, 4H); 7.78-7.88 (m, 3H)

MS (ESI⁺) for C₃₇H₃₆F₂N₄O₃ m/z 623 (M+H⁺)

Trans-product N-(3′-cyanobiphenyl-4-yl)-N′-(3,4-difluorophenyl)-N-[(3aS*,6S*,7aS*)-3a-(3,4-dimethoxyphenyl)-1-methyloctahydro-1H-indol-6-yl]urea trifluoroacetate as yellow oil (1.7 mg, 7%)

¹HNMR (270 MHz, Chloroform-d) ppm 1.29-1.35 (m, 1H); 1.81 (s, 1H): 2.23-2.43 (m, 4H); 2.57 (d, J=13.45, 1H); 2.82 (b, 1H); 2.96-3.00 (m, 1H) 3.04 (s, 3H); 3.70 (s, 1H); 3.86 (s, 6H); 4.26 (d, J=12.01, 2H); 6.33 (s, 1H); 6.73-6.86 (m, 3H); 6.92-7.08 (m, 2H); 7.32-7.44 (m, 2H); 7.58-7.69 (m, 4H); 7.78-7.88 (m, 3H)

MS (ESI+) for C₃₇H₃₆F₂N₄O₃ m/z 623 (M+H⁺)

### EXAMPLE 525

### N-[(3aS*,6R*,7aS*)-3a-(3,4-dimethoxyphenyl)-1-isopropyloctahydro-1H-indol-6-yl]-N′-[2-fluoro-3-(trifluoromethyl)phenyl]urea trifluoroacetate

Step 1:

2-Fluoro-3-(trifluoromethyl)phenylisocyanate (14.4 mg, 0.07 mmol) was added to a solution of (3aS*,6R*,7aS*)-1-benzyl-3a-(3,4-dimethoxyphenyl)octahydro-1H-indole-6-amine, Comparative Example 5, (25 mg, 0.07 mmol) in CH₂Cl₂ and stirred at RT for 2 hrs. The mixture was filtered and the precipitate was washed with CH₂Cl₂ and then dried to give N-[3aS*,6R*,7aS*)-1-benzyl-3a-(3,4-dimethoxyphenyl)octahydro-1H-indol-6-yl]-N′-[2-fluoro-3-(trifluoromethyl)phenyl]urea as a beige solid.

MS (ESI⁺) for C₃₁H₃₃F₄N₃O₃ m/z 572 (M+H⁺)

Step 2:

Pd/C (10%, 2.9 mg) was added to a solution of N-[3aS*,6R*,7aS*)-1-benzyl-3a-(3,4-dimethoxyphenyl)octahydro-1H-indol-6-yl]-N′-[2-fluoro-3-(trifluoromethyl)phenyl]urea, prepared in step 1, (29 mg, 0.05 mmol) in MeOH/THF 1:1 (10 mL) and stirred under 1 atm of H₂. The mixture was stirred overnight and then concentrated to give N-[2-fluoro-3-(trifluoromethyl)phenyl]-N′-[3aS*,6R*,7aS*)-3a-(3,4-dimethoxyphenyl)octahydro-1H-indol-6-yl]-urea as a clear oil.

MS (ESI⁺) for C₂₄H₂₇F₄N₃O₃ m/z 419 (M+H⁺)

Step 3:

Acetone (11.6 mg, 0.20 mmol) was added to a solution of N-[2-fluoro-3-(trifluoromethyl)phenyl]-N′-[3aS*,6R*,7aS*)-3a-(3,4-dimethoxyphenyl)octahydro-1H-indol-6-yl]-urea, prepared in step 2, (17 mg, 0.04 mmol) in 5 mL MeOH and stirred at room temperature for 30 minutes. Then NaCNBH₃ (27 mg, 0.4 mmol) was added and the mixture was stirred overnight at room temperature. The mixture was filtrated, concentrated and purified using preparative HPLC-MS (20-70% MeCN) giving the product as white oil (14 mg, 67%).

¹HNMR (270 MHz, Chloroform-d) ppm 1.24-1.31 (m, 1H); 1.38 (d, J=6.43, 2H); 1.45 (d, J=6.43, 2H); 1.77 (s, 1H); 1.86-1.96 (m, 3H); 2.10-2.27 (m, 3H); 2.36-2.41 (m, 2H); 2.55-2.63 (m, 1H); 3.35 (b, 2H); 3.81-3.89 (m, 7H); 4.10-4.16 (m, 2H); 6.74-6.89 (m, 3H); 7.16-7.23 (m, 1H); 7.30-7.38 (m, 2H); 7.84 (b, 1H)

MS (ESI⁺) for C₂₇H₃₃F₄N₃O₃ m/z 524 (M+H⁺), HRMS found: 523,2481 calculated: 523,2458

### EXAMPLE 526

### N-(2,5-difluorophenyl)-N′-[(3aS*,6R*,7aS*)-3a-(3,4-dimethoxyphenyl)-1-isopropyloctahydro-1H-indol-6-yl]urea trifluoroacetate

Procedure as for Example 525

2,5-Difluorophenyl isocyanate gave the product as white gum (11 mg, 73%).

¹HNMR (270 MHz, Chloroform-d) ppm 1.25 (d, J=6.93, 2H); 1.41 (d, J=5.94, 2H); 1.74-1.85 (m, 3H); 1.86-1.96 (m, 3H); 2.06-2.16 (m, 3H); 2.25 (s, 1H); 2.45-2.56 (m, 2H); 3.12 (b, 1H); 3.58-3.67 (b, 1H); 3.88 (d, J=3.96, 6H); 4.02-4.15 (m, 2H); 6.58-6.66 (m, 1H); 6.75-6.86 (m, 3H); 6.90-6.99 (m, 1H); 7.63-7.71 (m, 1H); 8.08 (b, 1H)

MS (ESI⁺) for C₂₆H₃₃F₂N₃O₃ m/z 474 (M+H⁺), HRMS found: 473,2505 calculated: 473,2490

### EXAMPLE 527

### N-(2-chloro-6-methoxypyridin-4-yl)-N′-[(3aS,6R,7aS)-3a-(3,4-dimethoxyphenyl)-1-methyloctahydro-1H-indol-6-yl]urea hydrochloride

2-Chloro-6-methoxyisonicotinic acid (100 mg, 0.53 mmol) was dissolved in 5 mL toluene and stirred at −0° C. under N₂. Triethylamine (53 mg, 0.53 mmol) were added and then drop wise addition of DPPA (Diphenylphosphorylazide) (142 mg, 0.53 mmol). The mixture was stirred at RT for 1.5 hrs and then heated at 80° C. for 2 hrs. After cooling to room temperature (3aS,6R,7aS)-3a-(3,4-dimethoxyphenyl)-1-methyloctahydro-1H-indol-6-amine, Comparative Example 10, (100 mg, 0.34 mmol) in 1 mL dry CH₂Cl₂ were added. The reaction was stirred at room temperature overnight. The mixture was concentrated and then purified using preparative HPLC-MS system 5-70% MeCN, which gave the product as a TFA-salt. The product was stirred in CH₂Cl₂ and excess 2M HCl in ether was added to give the hydrochloride salt as a light yellow solid (58 mg, 36%)

¹HNMR (270 MHz, Chloroform-d) ppm 1.16-1.30 (m, 1H); 1.83-2.00 (m, 3H); 2.19-2.3 (m, 2H); 2.49 (d, J=15.09, 1H); 2.75 (s, 1H); 2.99 (s, 3H); 3.05-3.11 (m, 2H); 3.71 (s, 1H); 3.80-3.97 (m, 9H); 4.03-4.14 (m, 2H); 6.67-6.76 (m, 2H); 6.82-7.19 (m, 3H); 7.63 (s, 1H)

MS (ESI⁺) for C₂₄H₃₁ClN₄O₄ m/z 475 (M+H⁺), HRMS found: 474,2026 calculated: 474,2034

Optical rotation [α]²⁰_{D}=+11

### EXAMPLE 528

### N-(2,3-difluoro-4-methyl]phenyl)-N′-[(3aS,6R,7aS)-3a-(3,4-dimethoxyphenyl)-1-methyloctahydro-1H-indol-6-yl]urea trifluoroacetate

2,3-Difluoro-4-methylbenzoic acid (17 mg, 0.1 mmol) was dissolved in 1 mL toluene and stirred at ˜0° C. under N₂. Triethylamine (10 mg, 0.1 mmol) was added followed by drop wise addition of DPPA (Diphenylphosphorylazide) (27 mg, 0.1 mmol). The mixture was stirred at RT for 1,5 hrs and then heated at 80° C. for 2 hrs. After cooling to room temperature (3aS,6R,7aS)-3a-(3,4-dimethoxyphenyl)-1-methyloctahydro-1H-indol-6-amine, Comparative Example 10, (20 mg, 0.07 mmol) in 1 mL dry CH₂Cl₂ was added. The reaction was stirred at room temperature overnight. The mixture was concentrated and then purified using preparative HPLC-MS system 10-50% MeCN, which gave the product as light yellow oil (9.3 mg, 29%).

¹HNMR (270 MHz, Chloroform-d) ppm 1.16-1.34 (m, 1H); 1.78-2.07 (m, 3H); 2.12-2.46 (m, 2H); 2.53 (d, J=12.37, 1H); 2.74 (s, 1H); 2.89 (s, 3H); 3.67 (b, 2H); 3.86 (s, 9H); 3.96-4.14 (m, 2H); 6.67-6.83 (m, 2H); 7.07 (d, J=6.56, 1H) 7.41-7.32 (m, 2H) 8.20 (b, 1H)

MS (ESI⁺) for C₂₅H₃₁F₂N₃O₃ m/z 460 (M+H⁺), HRMS found: 459.2325, calculated: 459.2333.

### EXAMPLE 529

### N-(3-chloro-5-fluorophenyl)-N′-[(3aS,6R,7aS)-3a-(3,4-dimethoxyphenyl)-1-methyloctahydro-1H-indol-6-yl]urea trifluoroacetate

Procedure as for Example 528

3-Chloro-5-fluorobenzoic acid gave the product as light yellow oil (11.5 mg, 36%).

¹HNMR (270 MHz, Chloroform-d) ppm 1.05-1.14 (m, 1H); 1.83-1.93 (m, 3H); 2.06-2.19 (m, 2H); 2.39 (d, J=11.38, 1H); 2.69 (s, 1H); 2.87 (s, 3H); 3.47 (b, 2H); 3.73 (d, J=9.53, 1H); 3.85 (s, 6H); 4.06-4.21 (m, 2H); 6.45 (b, 1H) 6.65 (d, J=8.04, 2H) 6.76 (d, J=8.41, 2H); 7.04-7.11 (m, 1H); 7.36 (s, 2H); 7.81 (b, 1H)

MS (ESI⁺) for C₂₄H₂₉ClFN₃O₃ m/z 462 (M+H⁺), HRMS found: 461.1863, calculated: 461.1881

### EXAMPLE 530

### N-[(3aS,6R,7aS)-3a-(3,4-dimethoxyphenyl)-1-methyloctahydro-1H-indol-6-yl]-N′-(4-fluoro-3-methoxyphenyl)urea trifluoroacetate

Procedure as for Example 528

4-Fluoro-3-methoxybenzoic acid gave the product as yellow oil (20.4 mg, 64%).

¹HNMR (270 MHz, Chloroform-d) ppm 1.12-1.24 (m, 1H); 1.82-1.95 (m, 3H); 2.08-2.17 (m, 2H); 2.44 (d, J=14.35, 1H); 2.62-2.70 (m, 1H) 2.85 (s, 3H); 3.54 (b, 2H) 3.76 (s, 3H); 3.86 (s, 6H); 3.92 (s, 2H); 4.15 (b, 1H); 6.66 (s, 1H) 6.78 (d, J=8.29, 1H) 6.85-6.96 (m, 1H); 7.05-7.12 (m, 1H); 7.16-7.27 (m, 2H) 8.65 (b, 1H)

MS (ESI⁺) for C₂₅H₃₂FN₃O₄ m/z 458 (M+H⁺), HRMS found: 457.2375 calculated: 457.2377

### EXAMPLE 531

### N-[3,5-difluoro-4-(trifluoromethyl)phenyl]-N′-[(3aS,6R,7aS)-3a-(3,4-dimethoxyphenyl)-1-methyloctahydro-1H-indol-6-yl]urea trifluoroacetate

3,5-Difluoro-4-trifluoromethylaniline (17 mg, 0.084 mmol) was dissolved in 1 mL dry CH₂Cl₂ under N₂. Triethylamine (14 mg, 0.14 mmol was added followed by drop-wise addition of Triphosgene (12.6 mg, 0.042 mmol) in 1 mL of dry CH₂Cl₂. The mixture was stirred at room temperature for 2 hrs, and then (3aS,6R,7aS)-3a-(3,4-dimethoxyphenyl)-1-methyloctahydro-1H-indol-6-amine, Comparative Example 10, (20 mg, 0.07 mmol) was added. The mixture was stirred at room temperature overnight and then concentrated. Purification using preparative HPLC-MS (20-70% MeCN) gave the product as light yellow oil (3.1 mg, 9%).

¹HNMR (270 MHz, Chloroform-d) ppm 1.19-1.33 (m, 1H); 1.84-1.96 (m, 3H); 2.00-2.07 (m, 1H); 2.12-2.29 (m, 2H); 2.51 (d, J=14.84, 1H); 2.78 (s, 1H); 2.93 (s, 3H); 2.98-3.07 (m, 2H); 3.64 (s, 1H); 3.86 (s, 6H); 4.02 (b, 2H); 4.79 (b, 1H); 6.47-6.55 (m, 1H); 6.66 (m, 2H); 6.80 (d, J=8.41, 2H)

MS (ESI⁺) for C₂₅H₂₈F₅N₃O₃ m/z 514 (M+H⁺), HRMS found: 513.2072, calculated: 513.2051

### EXAMPLE 532

### N-[2,4-difluoro-5-(trifluoromethyl)phenyl]-N′-[(3aS,6R,7aS)-3a-(3,4-dimethoxyphenyl)-1-methyloctahydro-1H-indol-6-yl]urea trifluoroacetate

Procedure as for Example 531

2,4-Difluoro-5-trifluoromethylaniline gave the product as light yellow oil (21 mg, 58%).

¹HNMR (270 MHz, Chloroform-d) ppm 1.23-1.32 (m, 1H); 1.85-1.99 (m, 3H); 2.11-2.27 (m, 2H); 2.46 (d, J=13.11, 1H); 2.73 (s, 1H); 2.93 (s, 3H); 3.06-3.16 (m, 2H); 3.73 (s, 1H); 3.85 (s, 6H); 3.99-4.11 (m, 2H); 6.61-6.71 (m, 2H); 6.77-6.89 (m, 3H); 8.21 (b, 1H)

MS (ESI⁺) for C₂₅H₂₈F₅N₃O₃ m/z 514 (M+H⁺)

### EXAMPLE 533

### N-[2,5-difluoro-4-(trifluoromethyl)phenyl]-N′-[(3aS,6R,7aS)-3a-(3,4-dimethoxyphenyl)-1-methyloctahydro-1H-indol-6-yl]urea trifluoroacetate

Procedure as for Example 531

2,5-Difluoro-4-trifluoromethylaniline gave the product as light yellow oil (3 mg, 9%).

¹HNMR (270 MHz, Chloroform-d) ppm 1.29-1.35 (m, 1H); 1.83-1.96 (m, 3H); 2.05-2.17 (m, 2H); 2.33 (b, 1H); 2.73 (s, 1H); 2.94 (s, 3H); 3.09-3.18 (m, 2H); 3.76 (s, 1H); 3.87 (s, 6H); 4.06-4.14 (m, 2H); 6.77-6.85 (m, 2H); 7.17-7.23 (m, 3H); 8.46 (b, 1H)

MS (ESI⁺) for C₂₅H₂₈F₅N₃O₃ m/z 514 (M+H⁺), HRMS found: 513.2044, calculated: 513.2051

### EXAMPLE 534

### N-(2-chloro-4-fluoro-5-methylphenyl)-N′-[(3aS,6R,7aS)-3a-(3,4-dimethoxyphenyl)-1-methyloctahydro-1H-indol-6-yl]urea trifluoroacetate

Procedure as for Example 531

2-Chloro-4-fluoro-5-methylaniline gave the product as light yellow oil (15.6 mg, 47%).

¹HNMR (270 MHz, Chloroform-d) ppm 1.23-1.33 (m, 1H); 1.89-2.00 (m, 3H); 2.06-2.25 (m, 6H); 2.54 (d, J=15.35, 1H); 2.74 (s, 1H); 2.89 (s, 3H); 3.07-3.13 (m, 2H); 3.74 (s, 1H); 3.87 (s, 6H); 3.97-4.14 (m, 2H); 6.70-6.76 (m, 2H); 6.82 (d, J=8.91, 1H); 6.99 (d, J=8.66, 1H); 7.89 (b, 1H)

MS (ESI⁺) for C₂₅H₃₁ClFN₃O₃ m/z 476 (M+H⁺), HRMS found: 475.2040, calculated: 475.2038

### EXAMPLE 535

### N-[(3aS,6R,7aS)-3a-(3,4-dimethoxyphenyl)-1-methyloctahydro-1H-indol-6-yl]-N′-(2,3,5-trifluorophenyl)urea trifluoroacetate

Procedure as for Example 531

2,3,5-Trifluoromethylaniline gave the product as a light yellow oil (15.8 mg, 49%).

¹HNMR (270 MHz, Chloroform-d) ppm 1.20-1.33 (m, 1H); 1.85-2.01 (m, 3H); 2.12-2.29 (m, 2H); 2.50 (d, J=14.60, 1H); 2.77 (s, 1H); 2.94 (s, 3H); 3.00-3.08 (m, 2H); 3.64 (s, 1H); 3.86 (s, 6H); 3.96-4.12 (m, 2H); 4.80 (b, 1H); 6.47-6.55 (m, 1H); 6.62-6.69 (m, 2H); 6.81 (d, J=8.42, 2H)

MS (ESI⁺) for C₂₅H₂₈F₃N₃O₃ m/z 464 (M+H⁺), HRMS found: 462.2089, calculated: 463.2083

### EXAMPLE 536

### N-(2-chloro-5-fluorophenyl)-N′-[(3aS,6R,7aS)-3a-(3,4-dimethoxyphenyl)-1-methyloctahydro-1H-indol-6-yl]urea trifluoroacetate

Procedure as for Example 531

2-Chloro-5-fluoroaniline gave the product as light yellow oil (20.7 mg, 64%).

¹HNMR (270 MHz, Chloroform-d) ppm 1.24-1.33 (m, 1H); 1.87-1.95 (m, 2H); 2.05-2.12 (m, 1H); 2.17-2.25 (m, 2H); 2.48 (d, J=13.61, 1H); 2.75 (s, 1H); 2.87 (s, 3H); 3.07-3.16 (m, 2H); 3.70 (s, 1H); 3.86 (s, 6H); 3.95-4.12 (m, 2H); 6.65-6.71 (m, 2H); 6.79 (d, J=8.41, 2H); 7.18-7.22 (m, 2H); 8.01 (b, 1H)

MS (ESI⁺) for C₂₄H₂₉ClFN₃O₃ m/z 462 (M+H⁺), HRMS found: 461.1903, calculated: 461.1881

### EXAMPLE 537

### N-biphenyl-3-yl-N′-[(3aS,6R,7aS)-3a-(3,4-dimethoxyphenyl)-1-methyloctahydro-1H-indol-6-yl]urea trifluoroacetate

Procedure as for Example 531

3-aminobiphenyl gave the product as beige solid (22 mg, 65%).

¹HNMR (270 MHz, Chloroform-d) ppm 1.24-1.33 (m, 1H); 1.87-1.95 (m, 2H); 2.05-2.12 (m, 1H); 2.17-2.25 (m, 2H); 2.48 (d, J=13.61, 1H); 2.75 (s, 1H); 2.87 (s, 3H); 3.07-3.16 (m, 2H); 3.70 (s, 1H); 3.86 (s, 6H); 3.95-4.12 (m, 2H); 6.22 (d, J=7.67, 1H); 6.49-6.68 (m, 2H); 7.19-7.41 (m, 4H); 7.49-7.58 (m, 3H); 7.89 (s, 2H); 9.03 (b, 1H)

MS (ESI⁺) for C₃₀H₃₅N₃O₃ m/z 486 (M+H⁺), HRMS found: 485.2702, calculated: 485.2678

### EXAMPLE 538

### N-(2-chloro-4,6-difluorophenyl)-N′-[(3aS,6R,7aS)-3a-(3,4-dimethoxyphenyl)-1-methyloctahydro-1H-indol-6-yl]urea trifluoroacetate

Procedure as for Example 531

2-Chloro-3,6-difluoroaniline gave the product as light yellow oil (11.8 mg, 35%).

¹HNMR (270 MHz, Chloroform-d) ppm 1.23-1.39 (m, 1H); 1.88-2.08 (m, 3H); 2.18-2.33 (m, 2H); 2.53 (d, J=14.35, 1H); 2.74 (s, 1H); 3.01 (s, 3H); 3.08-3.13 (m, 2H); 3.74 (s, 1H); 3.88 (s, 6H); 4.02-4.18 (m, 2H); 6.72-6.88 (m, 2H); 6.97-7.01 (m, 2H); 7.27-7.32 (m, 1H); 7.63 (b, 1H)

MS (ESI⁺) for C₂₄H₂₈ClF₂N₃O₃ m/z 480 (M+H⁺),

### EXAMPLE 539

### N-[(3aS,6R,7aS)-3a-(3,4-dimethoxyphenyl)-1-methyloctahydro-1H-indol-6-yl]-N′-[2-methyl-5-(trifluoromethyl)phenyl]urea trifluoroacetate

Procedure as for Example 531

2-Methyl-5-trifluoromethylaniline gave the product as light yellow oil (4.1 mg, 12%).

¹HNMR (270 MHz, Chloroform-d) ppm 1.19-1.26 (m, 1H); 1.83-2.03 (m, 3H); 2.12-2.26 (m, 2H); 2.29 (s, 3H); 2.52 (d, J=12.87, 1H); 2.66 (s, 1H); 2.92 (s, 3H); 2.97-3.05 (m, 2H); 3.61 (s, 1H); 3.85 (s, 6H); 4.07-4.16 (m, 2H); 6.60 (d, J=8.42, 2H); 6.65 (s, 2H); 6.75 (d, J=8.42, 2H); 7.91 (b, 1H)

MS (ESI+) for C₂₆H₃₂F₃N₃O₃ m/z 492 (M+H⁺), HRMS found: 491.2496, calculated: 491.2396

### EXAMPLE 540

### N-(2,2-difluoro-1,3-benzodioxol-5-yl)-N′-[(3aS,6R,7aS)-3a-(3,4-dimethoxyphenyl)-1-methyloctahydro-1H-indol-6-yl]urea trifluoroacetate

Procedure as for Example 531

5-Amino-2,2-difluoro-1,3-benzodioxole gave the product as a light yellow oil (1.9 mg, 6%).

¹HNMR (270 MHz, Chloroform-d) ppm 1.05-1.14 (m, 1H); 1.83-1.93 (m, 3H); 2.06-2.19 (m, 2H); 2.39 (d, J=11.38, 1H); 2.69 (s, 1H); 2.87 (s, 3H); 3.47 (b, 2H); 3.73 (d, J=9.53, 1H); 3.85 (s, 6H); 4.06-4.21 (m, 2H); 6.45 (b, 1H) 6.65 (d, J=8.04, 2H); 6.76 (d, J=8.41, 2H); 7.04-7.11 (m, 1H); 7.36 (s, 2H); 7.81 (b, 1H)

MS (ESI+) for C₂₅H₂₉F₂N₃O₅ m/z 490 (M+H⁺), HRMS found: 489.2094, calculated: 489.2075

### EXAMPLE 541

### N-[(3aS,6R,7aS)-3a-(3,4-dimethoxyphenyl)-1-methyloctahydro-1H-indol-6-yl]-N′-[3-methyl-5-(trifluoromethyl)phenyl]urea trifluoroacetate

Procedure as for Example 531

3-Methyl-5-trifluoromethylaniline gave the product as yellow oil (28.3 mg, 82%).

¹HNMR (270 MHz, Chloroform-d) ppm 1.06-1.20 (m, 1H); 1.75-2.01 (m, 3H); 2.07-2.23 (m, 2H); 2.28-2.39 (m, 4H); 2.60 (s, 1H); 2.97 (s, 3H); 3.06-3.15 (m, 2H); 3.62 (s, 1H); 3.84 (d, J=5.94, 6H); 3.95-4.05 (m, 2H); 6.47-6.52 (m, 1H); 6.65-6.72 (m, 2H); 6.99 (s, 1H) 7.31-7.40 (m, 1H); 7.94 (b, 1H)

MS (ESI⁺) for C₂₆H₃₂F₃N₃O₃ m/z 492 (M+H⁺), HRMS found: 491.2381 calculated: 491.2396

### EXAMPLE 542

### N-[(3aS,6R,7aS)-3a-(3,4-dimethoxyphenyl)-1-methyloctahydro-1H-indol-6-yl]-N′-[3-fluoro-4-(trifluoromethyl)phenyl]urea trifluoroacetate

Procedure as for Example 531

2-Fluoro-3-trifluoromethylaniline gave the product as light yellow oil (1.8 mg, 5%).

¹HNMR (270 MHz, Chloroform-d) ppm 1.03-1.11 (m, 1H); 1.81-2.00 (m, 3H); 2.10-2.27 (m, 2H); 2.47 (d, J=13.86, 1H); 2.76 (s, 1H); 2.98 (s, 3H); 3.04-3.10 (m, 2H); 3.47 (s, 1H); 3.85 (d, J=5.94, 6H); 4.01-4.14 (m, 2H); 6.63 (s, 2H); 6.74 (d, J=8.41, 1H); 7.41-7.47 (m, 1H); 7.71 (s, 1H); 7.76 (b, 1H)

MS (ESI⁺) for C₂₅H₂₉F₄N₃O₃ m/z 496 (M+H⁺), HRMS found: 495.2152 calculated: 495.2145

### EXAMPLE 543

### N-[(3aS,6R,7aS)-3a-(3,4-dimethoxyphenyl)-1-methyloctahydro-1H-indol-6-yl]-N′-{2-[(trifluoromethyl)thio]phenyl}urea trifluoroacetate)

Procedure as for Example 531

2-(Trifluoromethylthio)aniline gave the product as a light yellow oil (11.0 mg, 3%).

¹HNMR (270 MHz, Chloroform-d) ppm 1.13-1.28 (m, 1H); 1.85-2.06 (m, 3H); 2.09-2.25 (m, 2H); 2.58 (d, J=12.37, 1H); 2.71 (s, 1H); 2.98 (s, 3H); 3.03-3.10 (m, 2H); 3.68 (s, 1H); 3.89 (s, 6H); 4.12-4.24 (m, 2H); 6.73-6.82 (m, 3H); 7.05-7.13 (m, 1H); 7.40-7.40 (m, 1H); 7.63 (d, J=9.16, 2H); 7.93 (b, 1H)

MS (ESI⁺) for C₂₅H₃₀F₃N₃O₃S m/z 501 (M+H⁺), HRMS found: 509.1981 calculated: 509.1960

### EXAMPLE 544

### N-(2-chloro-3,6-difluorophenyl)-N′-[(3aS,6R,7aS)-3a-(3,4-dimethoxyphenyl)-1-methyloctahydro-1H-indol-6-yl]urea trifluoroacetate

Procedure as for Example 528

2-Chloro-3,6-difluorobenzoic acid gave the product as off-white solid (19.4 mg, 58%).

¹HNMR (270 MHz, Chloroform-d) ppm 1.19-1.31 (m, 1H); 1.85-2.06 (m, 3H); 2.13-2.27 (m, 2H); 2.56 (d, J=13.41, 1H); 2.65 (s, 1H); 2.97 (s, 3H); 3.03-3.09 (m, 2H); 3.73 (s, 1H); 3.87 (s, 6H); 4.06-4.14 (m, 2H); 6.72-6.84 (m, 2H); 6.90-6.99 (m, 2H); 7.15-7.24 (m, 1H) 7.64 (b, 1H)

MS (ESI⁺) for C₂₄H₂₈ClF₂N₃O₄ m/z 480 (M+H⁺), HRMS found: 479.1786 calculated: 479.1787

### EXAMPLE 545

### N-(3-chloro-2,6-difluorophenyl)-N′-[(3aS,6R,7aS)-3a-(3,4-dimethoxyphenyl)-1-methyloctahydro-1H-indol-6-yl]urea trifluoroacetate

Procedure as for Example 528:

3-Chloro-2,6-difluorobenzoic acid gave the product as off-white solid (1.7 mg, 5%).

¹HNMR (270 MHz, Chloroform-d) ppm 1.19-1.32 (m, 1H); 1.88-2.06 (m, 3H); 2.16-2.31 (m, 2H); 2.56 (d, J=13.09, 1H); 2.66 (s, 1H); 2.97 (s, 3H); 3.03-3.08 (m, 2H); 3.73 (s, 1H); 3.88 (m, 6H); 4.00-4.14 (m, 2H); 6.72-6.78 (m, 2H); 6.82-6.93 (m, 2H); 7.39-7.48 (m, 1H); 8.31 (b, 1H)

MS (ESI+) for C₂₄H₂₈ClF₂N₃O₄ m/z 480 (M+H⁺), HRMS found: 479.1779 calculated: 479.1787

### EXAMPLE 546

### N-[2,3-difluoro-4-(trifluoromethyl)phenyl]-N′-[(3aS,6R,7aS)-3a-(3,4-dimethoxyphenyl)-1-methyloctahydro-1H-indol-6-yl]urea trifluoroacetate

Procedure as for Example 531:

### 2,3-Difluoro-4-trifluoromethylaniline gave the product as a yellow oil (13.4 mg, 37%).

¹HNMR (270 MHz, Chloroform-d) ppm 1.20-1.33 (m, 1H); 1.85-2.05 (m, 3H); 2.11-2.27 (m, 2H); 2.53 (d, J=14.85, 1H); 2.65 (s, 1H); 2.91 (s, 3H); 3.01-3.15 (m, 2H); 3.66 (s, 1H); 3.86 (m, 6H); 3.99-4.10 (m, 2H); 6.59-6.67 (m, 1H); 6.70 (s, 1H); 6.80 (d, J=8.41, 2H); 7.14-7.21 (m, 1H); 7.89 (b, 1H)

MS (ESI⁺) for C₂₅H₂₈F₅N₃O₃ m/z 514 (M+H⁺), HRMS found: 513,2042 calculated: 513.2051

### EXAMPLE 547

### N-(4-cyano-2,5-difluorophenyl)-N′-[(3aS,6R,7aS)-3a-(3,4-dimethoxyphenyl)-1-methyloctahydro-1H-indol-6-yl]urea trifluoroacetate

Procedure as for Example 531:

4-amino-2,5-difluorobenzonitrile gave the product as a yellow oil (13.4 mg, 37%).

¹HNMR (270 MHz, Chloroform-d) ppm 1.19-1.25 (m, 1H); 1.85-1.99 (m, 3H); 2.11-2.27 (m, 2H); 2.54 (d, J=14.17, 1H); 2.67 (s, 1H); 2.92 (s, 3H); 3.06-3.16 (m, 2H); 3.73 (s, 1H); 3.85 (s, 6H); 3.99-4.11 (m, 2H); 6.61-6.71 (m, 2H); 6.77-6.89 (m, 3H); 8.21 (b, 1H)

MS (ESI⁺) for C₂₅H₂₈F₂N₄O₃ m/z 471 (M+H⁺), HRMS found: 470,2114 calculated: 470.2129

### EXAMPLE 548

### N-(2-chloro-6-methoxypyridin-4-yl)-N′-[(3aS,6R,7aS)-3a-(3,4-dimethoxyphenyl)-1-methyloctahydro-1H-indol-6-yl]urea trifluoroacetate

Procedure as for Example 528:

3-Chloro-5-methoxyisonicotinic acid gave the product as a yellow oil (13.1 mg, 39%).

¹HNMR (270 MHz, Chloroform-d) ppm 1.16-1.30 (m, 1H); 1.83-2.00 (m, 3H); 2.19-2.31 (m, 2H); 2.49 (d, J=14.85, 1H); 2.75 (s, 1H); 2.97 (s, 3H); 3.05-3.11 (m, 2H); 3.73 (s, 1H); 3.80-3.97 (m, 9H); 4.03-4.14 (m, 2H); 6.67-6.76 (m, 2H); 6.82-7.19 (m, 3H); 7.63 (s, 1H)

MS (ESI⁺) for C₂₄H₃₁ClN₄O₄ m/z 475 (M+H⁺), HRMS found: 474,2010 calculated: 474.2034

Preparation of a Pharmaceutical Composition

### EXAMPLE 549

### Preparation of Tablets

| | |
|---|---|
| Ingredients | mg/tablet |

| | | |
|---|---|---|
| 1. | Active compound of formula (I) | 10.0 |
| 2. | Cellulose, microcrystalline | 57.0 |
| 3. | Calcium hydrogen phosphate | 15.0 |
| 4. | Sodium starch glycolate | 5.0 |
| 5. | Silicon dioxide, colloidal | 0.25 |
| 6. | Magnesium stearate | 0.75 |

The active ingredient 1 is mixed with ingredients 2, 3, 4 and 5 for about 10 minutes. The magnesium stearate is then added, and the resultant mixture is mixed for about 5 minutes and compressed into tablet form with or without film-coating.

Biological Methods

The ability of a compound of the invention to bind or act at the MCH1R receptor can be determined using in vitro and in vivo assays known in the art. The biological activity of compounds prepared in the Examples was tested using different tests.

Binding Assay

The compounds according to the invention were evaluated for their binding to the human MCH1R receptor by the following method:

Materials and Methods

Materials

Compounds: MCH peptide was purchased from Phoenix pharmaceuticals. (Phe¹³, [¹²⁵I]Tyr¹⁹ Melanine-Concentrating Hormone (human, mouse, rat) ([¹²⁵I]-MCH) was obtained from NEN life Science Products. Inc. Boston, Mass. Wheat germ agglutinine SPA beads (RPNQ 0001) were obtained from Amersham-Pharmacia Biotech. All other reagents used are of highest purity from different resources available. Protein Kits, Micro BCA™ Protein Assay Reagent Kit (Cat No. 23235) were purchased from Piece, Rockford, Ill., USA.

Plastic wares: Cell culture flasks, dishes were from Decton Dickinson Labware, NJ, USA. Scintillation plate, white clear bottom were from Wallac, Finland.

Cells and Culture Conditions

CHO-K1 cells expressing hMCH1 receptor were purchased from Euroscreen.

CHO-K1 hMCHR1 (Euroscreen, Brussels, Belgium, # ES-370-C) were cultivated in Nutrient mixture Ham's F-12 with Glutamax I (Gibco-BRL #31765-027) supplemented with 10% heat-inactivated foetal calf serum (FCS, Gibco-BRL #10108-165) and 400 μg/ml geniticin (Gibco-BRL #1140-0359). The cells were sub-cultivated twice weekly with split ratio=1:20-1:30. For membrane preparation the cells were cultured in 500 mm² dishes and the cells were harvested when 90% confluent.

Membrane Preparation

When the cells reached more than 90% confluence, dishes (500 cm²) were rinsed twice with 20 ml PBS (Ca²⁺ and Mg²⁺ free). Buffer A, which contains Tris.HCl (15), MgCl₂.6H₂O (2), EDTA (0.3), EGTA (1) in mM with pH 7.5, 25 ml was added and cells were suspended using a window scraper. The cells were collected in 50 ml Falcon tube pre-cooled on ice and then centrifuged for 3 minutes at 1500 g at 4° C. The supernatant was discarded and the cells were suspended again with Buffer A. The cells were homogenized using a Polytron homogenizer at setting 4 for 4 times for 30 seconds with 1 minute pause between the cycles. The homogenized preparation was centrifuged at 40,000 g (18500 rpm with ss-34, No. 5 rotor in Sorvall centrifuge, RC5C, DuPont) for 25 minutes at 4° C. The pellets were washed once with Buffer A and centrifuged again under the same conditions. The pellets were suspended with Buffer B, which contains Tris.HCl (7.5), MgCl₂.6H₂O (12.5), EDTA (0.3), EGTA (1), sucrose (25) in mM with pH 7.5, and gently homogenized for several times with a glass homogenizer. The membrane preparation was aliquoted into Eppendorf tubes, 1 ml/tube and frozen at −70° C.

Membrane Protein Determination

The protein determination was done as described in the instruction provided with Pierce protein assay kit (Peirce Micro BCA Protein assay reagent kit, No 23235, Pierce, USA). Briefly, the Piece working reagent components A, B and C were mixed in the ratio 25:24:1. BSA (No. 23209, Pierce, USA) provided with the kits was used as standard, which the concentration in the curve is 1, 2, 4, 6, 8, 12, 16 and 24 μl/ml. The samples from membrane preparation were diluted for 50, 100, 200, 400 times. The standards or the samples 150 μl and the working reagent 150 μl were mixed in each well in a Costa 96 well microtiter plate and incubated at 37° C. for 2 hours. The plate was cooled down to room temperature and read at 595 nm with a Microplate reader from Molecular Devices, USA.

Receptor Binding by SPA

The WGA beads were re-constructed with reaction buffer, which contains Tris (50), MgCl₂ (5), EDTA (2.5) in mM with pH adjusted to 7.4, to 40 mg/ml as a stock suspension. To link the membrane with the bead, the beads and the membrane will be pre-incubated with for 30 minutes at room temperature with gentle shaking. The suspension of the beads was centrifuged at 3400 rpm for 2 minutes using centrifuge. The supernatant was discarded and the beads were re-suspended with binding buffer, HEPES (25 mM), MgCl₂ (5 mM), CaCl₂ (1 mM), BSA (0.5%) with peptidase inhibitors (1 μg/ml) Leupeptin, Aprotinin and pepstatin, pH 7.4.

Since appropriated beads and membrane construction is needed for SPA, the ratio of beads and membrane in link were tested and it will be indicated where the experiments are described.

The radio labeled [¹²⁵I]-MCH was diluted with cold MCH in ratio 1:3. In Kd determination, the concentrations of labeled peptide were 3 nM with 1:2 series dilution for 11 samples. The amount of the beads was 0.25 mg/well. The results were calculated using Excel program and the curves were drawn using a program GraphPad Prism.

For screening of the substances the amount of the beads used was 0.25 mg/well and the amount of the membrane protein was 4 μg/well 0.2 nM of labeled MCH was used. The total volume was 200 μl, which contained 50 μl [¹²⁵I]-MCH, 100 μl substances and 50 μl beads. The plate was gently shaken for 30 minute and incubated overnight. The samples were counted using Microbeta counter (Wallac Trilux 1450 Micro beta counter, Wallac, Finland) for 2 minutes and the results were calculated by using the computer program Activity Base.

Results

The equilibrium time of the binding was investigated at room temperature, 30 and 37° C. The equilibrium time was about 30 minutes at 37° C. but the binding was lower compared with that at room temperature and 30° C. The equilibrium time was about 2 hours at 30° C. while it took about 4 hours to reach stable binding at room temperature. Thus, room temperature was chosen since it is easy condition for experiments.

The [¹²⁵I]-MCH binding to hMCH R1 was further characterized by determination of Kd values. The Kd values are same, 0.19 nM, as reported by Chambers J, Ames R S, Bergsma D, Muir A, Fitzgerald L R, Hervieu G, Dytko G M, Foley J J, Martin J, Liu W S, Park J, Ellis C, Ganguly S, Konchar S, Cluderay J, Leslie R, Wilson S, Sarau H M. Melanin-concentrating hormone is the cognate ligand for the orphan G-protein-coupled receptor SLC-1. Nature 1999 Jul. 15; 400 (6741):261-5.

In all displacement experiments, 0.2 nM [¹²⁵I]-MCH was used for total binding and 300 nM MCH used as non-specific binding. The background is low and the signal is good. The Z′ factor was 0.83 which is considered very good for screening.

Kd values from present study were consistent with that from Macdonald D, Murgolo N, Zhang R, Durkin J P, Yao X, Strader C D, Graziano M P. Molecular characterization of the melanin-concentrating hormone/receptor complex: identification of critical residues involved in binding and activation. Mol Pharmacol 2000 July; 58 (1):217-25 but were slightly different from that 1.2 nM from Hervieu G J, Cluderay J E, Harrison D, Meakin J, Maycox P, Nasir S, Leslie R A, The distribution of the mRNA and protein products of the melanin-concentrating hormone (MCH) receptor gene, slc-1, in the central nervous system of the rat. Eur J Neurosci 2000 April; 12 (4): 1194-216. The reason for this is unknown but might be caused by different clones of the cells.

The calculation of the Kᵢ values for the inhibitors was performed by use of Activity Base. The Kᵢ value is calculated from IC₅₀ and the Kₘ value is calculated using the Cheng Prushoff equation (with reversible inhibition that follows the Michaelis-Menten equation): Kᵢ=IC₅₀(1+[S]/Kₘ) [Cheng, Y. C.; Prushoff, W. H. Biochem. Pharmacol. 1973, 22, 3099-3108]. The IC₅₀ is measured experimentally in an assay wherein the decrease of the turnover of cortisone to cortisol is dependent on the inhibition potential of each substance.

The compounds of formula (I) exhibit the IC₅₀ values for the MCH1R receptor in the range from 10 nM to 10 μM. Illustrative of the invention, the following Ki values have been determined in the assay (see Table 1):

TABLE 1
Ki values determined in the assay.

| Compound of Example | Ki (nM) |
|---|---|
| 70 | 52 |
| 159 | 75 |
| 208 | 258 |

1. A compound of the general formula (I) 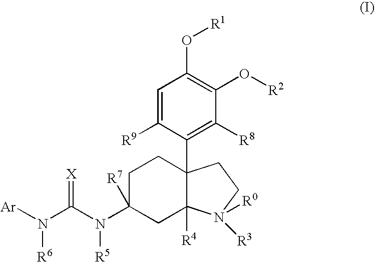
or a pharmaceutically acceptable salt, hydrates, geometrical isomers, racemates, tautomers, optical isomers, N-oxides and prodrug forms thereof, wherein:
R⁰ is C₁₋₆ alkyl or is absent;
each of R¹ and R² is independently hydrogen, C₁₋₆ alkyl, haloC₁₋₄ alkyl, aryl-C₁₋₆ alkyl; or
R¹ and R² are linked to form a C₁₋₃ alkylene;
R³ is H, carbamoyl, thiocarbamoyl, cyano, C₁₋₆ alkyl, C₂₋₆ alkenyl, C₂₋₆ alkenylsulfonyl, C₃₋₈ cycloalkyl optionally substituted by one or more of C₁₋₆ alkyl, C₃₋₈ cycloalkyl-C₁₋₆ alkyl, hydroxyC₁₋₆ alkyl, halo C₁₋₆ alkyl, halo C₁₋₆ alkylsulfonyl, halo C₁₋₆ acyl, mono- or di-C₁₋₆ alkylamino-C₁₋₆ alkyl, C₁₋₆ acyl, carboxyC₁₋₆ acyl, amino C₁₋₆ acyl, mono- or di-C₁₋₆ alkylamino C₁₋₆ acyl, hydroxyC₁₋₆ alkylaminoC₁₋₆ acyl, arylamino C₁₋₆ acyl, arylC₁₋₆ alkylaminoC₁₋₆ acyl, heteroarylC₁₋₆ alkylaminoC₁₋₆ acyl, heterocyclylC₁₋₆ acyl, azido-C₁₋₆ alkyl, C₁₋₆ alkoxycarbonylthiocarbamoyl, 3- to 7-membered heterocyclyl, heterocyclylC₁₋₆ alkyl, mono- or di-C₁₋₆ alkylaminocarbamoylC₁₋₆ alkyl, heterocyclyl, heterocyclylcarbonyl, aryl, aryl-C₁₋₄ alkyl, cyano C₁₋₆ alkyl, heteroaryl or heteroaryl-C₁₋₆ alkyl,
wherein any heterocyclyl, aryl or heteroaryl may be unsubstituted or independently substituted in one, two or three positions with oxo, C₁₋₄ alkyl, C₁₋₆ alkylamino, C₁₋₆ alkoxy, C₁₋₆ alkoxy-C₁₋₆ alkyl, C₁₋₆ alkylsilyl, halogen, aryl substituted by one or more of C₁₋₆ alkoxy, heteroaryl, arylC₁₋₆ alkyl, arylsulfonyl or a carbamimidoyl;
R⁴ is H, C₁₋₆ alkyl, or aryl;
each of R⁵ and R⁶ is independently H, C₁₋₆ alkyl, C₂₋₆ alkenyl, C₁₋₆ alkoxy-C₁₋₆ alkyl, C₃₋₈ cycloalkyl, 1-methyl-1,2,3,6-tetrahydro-pyridin-4-yl, arylC₁₋₆ alkyl, biphenylyl optionally substituted by cyano, heterocyclylC₁₋₆ alkyl, wherein the C₃₋₈ cycloalkyl, aryl or heterocyclyl may be unsubstituted or substituted by C₁₋₆ alkyl;
R⁷ is H or C₁₋₆ alkyl;
R⁸ is H or halogen;
R⁹ is H or forms CH₂ together with R³;
Ar is a 5- to 7-membered, aromatic carbon ring; a 5- to 7-membered, unsaturated heterocyclic ring containing 1 to 4 heteroatoms selected from oxygen, nitrogen and sulfur; a bicyclic ring system comprising at least one 5- to 7-membered, aromatic carbon ring; a bicyclic ring system comprising at least one 5- to 7-membered, unsaturated heterocyclic ring containing 1 to 4 heteroatoms selected from oxygen, nitrogen and sulfur; or a tricyclic ring system comprising at least two 5- to 7-membered, aromatic carbon rings;
wherein the group Ar may be unsubstituted or independently substituted in one, two, three, four or five positions with C₁₋₆ alkyl, halo-C₁₋₆ alkyl, C₁₋₆ alkoxy, halo C₁₋₆ alkoxy, C₁₋₆ acyl, C₁₋₆ alkoxycarbonyl, C₁₋₆ alkylthio, halo C₁₋₆ alkylthio, C₁₋₆ alkylsulfonyl, haloC₁₋₆ alkylsulfonyl, halogen, mono- or di-C₁₋₆ alkylamino, nitro, cyano, C₁₋₆ alkylaminosulfonyl, aryl optionally substituted by one or more halogen atoms, aryloxy, aryl-C₁₋₄ alkoxy, cyanoaryl, 3- to 7-membered saturated or partly unsaturated heterocyclyl, heterocyclylsulfonyl, heteroaryl, or a bicyclic ring system comprising at least one 5- to 7-membered, unsaturated heterocyclic ring containing 1 to 4 heteroatoms selected from oxygen, nitrogen and sulfur, which bicyclic ring system may be unsubstituted or substituted in one or two positions with C₁₋₆ alkyl; and
X is O, S, NH, CH—NO₂, or N—CN.

2. A compound according to claim 1 wherein R⁰ is methyl or is absent.

3. A compound according claim 1 wherein each of R¹ and R² independently is hydrogen, methyl, ethyl, n-propyl, isopropyl, benzyl, difluoromethyl, trifluoromethyl or together form a group —(CH₂)—, —(CH₂CH₂)— or —(CH₂CH₂CH₂)—.

4. The compound according to claim 1 wherein R³ is selected from hydrogen, methyl, ethyl, n-propyl, isopropyl, n-butyl, isobutyl, 3-methylbutyl, 2-ethylbutyl, 3,3-dimethylbutyl, cyclobutyl, cyclopentyl, cyclohexyl, cyclopropylmethyl, 2-hydroxyethyl, 2-fluoroethyl, 3,3,3-trifluoropropyl, 3,3,3-trifluoro-2-methylpropyl, dimethylaminoethyl, 3-dimethylamino-2,2-dimethyl-propyl, acetyl, dimethylaminoacetyl, propylaminoacetyl, 2-azidoethyl, 1-methylpiperidin-4-yl, benzyl, 4-methylbenzyl, 4-fluorobenzyl, tetrahydro-2H-pyran-4-yl, tetrahydro-3-thienyl, 3-furylmethyl, (5-methyl-2-furyl)methyl, (4,5-dimethyl-2-furyl)methyl, 3-thienylmethyl, (5-ethyl-2-thienyl)methyl, 1-methyl-1H-pyrrol-2-yl-methyl, pyridylmethyl, (3,5-dimethyl-1H-isooxazol-4-yl)methyl, 1,3-thiazol-2-yl-methyl, 1H-imidazol-2-yl-methyl, 1H-imidazol-4-yl-methyl, (1-methyl-1H-imidazol-2-yl)methyl, (2-methyl-1H-imidazol-4-yl)methyl, (2-ethyl-1H-imidazol-4-yl)methyl, (2-ethyl-4-methyl-1H-imidazol-5-yl)methyl, (4-methyl-1H-imidazol-5-yl)methyl, 1H-pyrazol-3-yl-methyl, (1,3-dimethyl-1H-pyrazol-5-yl)methyl, (5-chloro-1,3-dimethyl-1H-pyrazol-4-yl)methyl, 2-[4-(methoxymethyl)-1H-1,2,3-triazol-1-yl]ethyl, [5-(methoxymethyl)-1H-1,2,3-triazol-1-yl]ethyl, [4-(trimethylsilyl)-1H-1,2,3-triazol-1-yl]ethyl, {5-[(4-methylphenyl)sulfonyl]-1H-tetrazol-1-yl}ethyl, N-cyano-N′-ethyl-carbamimidoyl, isopropylaminoacetyl, phenylaminoacetyl, chloroacetyl, aminoacetyl, methylaminoacetyl, 3-(4-methoxyphenyl)isoxazol-5-ylmethyl, 1,2,3-triazol-5-ylmethyl, cyanomethyl, 2-furylmethylaminoacetyl, 4-pyridylmethylaminoacetyl, 4-chlorobenzylaminoacetyl, ethylaminoacetyl, 4-methyl-1-piperidinylacetyl, 1-pyrrolidinylacetyl, 2-hydroxyethylaminoacetyl, 1-benzyl-3-pyrrolidinyl, 3,3,5,5-tetramethyl-1-cyclohexyl, bicyclo[2.2.1]hept-2-yl, 4-methyl-1-cyclohexyl, 2-(5-methoxytetrazol-1-yl)ethyl, 2-(5-oxotetrazol-1-yl)ethyl, 2-(5-n-propylaminotetrazol-1-yl)ethyl, 2-[5-(1-imidazolyl)tetrazol-1-yl]ethyl, allyl, dimethylaminocarbamoylmethyl, tert-butyl, carbethoxythiocarbamoyl, 4-methyl-2-thiazolyl, 4-methyl-1-piperazinylcarbonyl, 2-carboxyethylcarbonyl, cyclopropyl, carbamoyl, thiocarbamoyl, cyano, 2-pyrimidinyl, vinylsulfonyl, 2-(4-methyl-1-piperidinyl)ethyl, 3-(1-piperidinyl)propyl, phenyl, chloromethylsulfonyl, formyl, and 2-(1-piperidinyl)ethyl.

5. The compound according to claim 1 wherein R⁴ is hydrogen.

6. The compound according claim 1 wherein R⁵ is selected from hydrogen, methyl, ethyl, n-propyl, isopropyl, n-butyl, allyl, methoxyethyl, cyclohexyl, benzyl, 3-cyano-(1,1′-biphenyl)-4-yl, 3-(4-methyl-1-piperazinyl)propyl, and tetrahydropyridyl substituted by methyl.

7. The compound according to claim 1 wherein R⁶ is hydrogen or methyl.

8. The compound according to claim 1 wherein R⁷ is hydrogen or n-propyl.

9. The compound according to claim 1 wherein R⁹ is hydrogen or fluoro.

10. The compound according to claim 1 wherein R³ and R⁹ form together CH₂.

11. The compound according to claim 1 or 2, wherein Ar is phenyl, furyl, thienyl, pyridinyl, isoxazolyl, isothiazolyl, thiazolyl, pyrazol, pyridazinyl, indenyl, dihydroindenyl, naphthyl, pyrimidinyl, fluorenyl, indolyl, quinolinyl, benzimidazolyl, benzofuranyl, 1,1-dioxobenzothienyl, dihydrobenzofuranyl, benzodioxolyl, benzodioxinyl, benzothiazolyl, benzothiadiazolyl, or benzotriazolyl, wherein the group Ar may be unsubstituted or independently substituted in one, two, three, four or five positions with C₁₋₆ alkyl, haloC₁-alkyl, C₁₋₆ alkoxy, haloC₁₋₆ alkoxy, C₁₋₆ acyl, C₁₋₆ alkoxycarbonyl, C₁₋₆ alkylthio, haloC₁₋₆ alkylthio, C₁₋₆ alkylsulfonyl, haloC₁₋₆ alkylsulfonyl, halogen, mono- or di-C₁₋₄ alkylamino, nitro, cyano, C₁₋₆ alkylaminosulfonyl, aryl optionally substituted by one or more halogen atoms, aryloxy, cyanoaryl, aryl-C₁₋₄ alkoxy, 3- to 7-membered saturated or partly unsaturated heterocyclyl, heterocyclylsulfonyl, heteroaryl, or C₁₋₄ alkyl substituted bicyclic ring system comprising at least one 5- to 7-membered, unsaturated heterocyclic ring containing 1 to 4 heteroatoms selected from oxygen, nitrogen and sulfur.

12. The compound according to claim 1, wherein X is oxygen.

13. The compound according to claim 1, wherein Ar is
phenyl, which may be unsubstituted or independently substituted in one, two, three, four or five positions with C₁₋₆ alkyl, haloC₁₋₄ alkyl, C₁₋₄ alkoxy, haloC₁₋₆ alkoxy, C₁₋₆ alkylthio, halo C₁₋₄ alkylthio, C₁₋₄ alkylsulfonyl, haloC₁₋₄ alkylsulfonyl, C₁₋₆ acyl, C₁₋₄ alkoxycarbonyl, halogen, mono- or di-C₁₋₄ alkylamino, nitro, cyano, C₁₋₄ alkylaminosulfonyl, phenyl, phenoxy, benzyloxy, cyanophenyl, tetrazolyl, oxazolyl, 4-bromo-1-methyl-1H-pyrazol-3-yl, heterocyclyl, C₁₋₆ alkylbenzothiazolyl, or thiadiazolyl;
furyl, which may be unsubstituted or independently substituted in one or two positions with C₁₋₄ alkyl or halo C₁₋₄ alkyl;
thienyl;
pyridinyl, which may be unsubstituted or independently substituted in one or two positions with C₁₋₄ alkyl, halo C₁₋₄ alkyl, C₁₋₄ alkoxy, halogen or nitro;
pyrazolyl, which may be unsubstituted or independently substituted in two or three positions with C₁₋₄ alkyl or halogen;
pyridazinyl;
isoxazolyl, which may be unsubstituted or independently substituted in one or two positions with C₁₋₆ alkyl;
isothiazolyl, which may be unsubstituted or substituted in one position with C₁₋₆ alkyl;
indenyl, which may be unsaturated or partly saturated;
naphthyl;
fluorenyl;
indolyl, which may be substituted in one position with C₁₋₆ alkyl;
quinolinyl; which may be unsubstituted or independently substituted in one position with C₁₋₆ alkyl, halogen, and haloC₁₋₆ alkyl;
benzofuranyl; which may be unsaturated or partly saturated;
benzodioxolyl;
benzodioxinyl, which may be unsubstituted or independently substituted in one or two positions with one or two halogen atoms in each position;
benzothiazolyl, which may be unsubstituted or independently substituted in one position with C₁₋₆ alkyl and halogen;
benzothiadiazolyl, which may be unsubstituted or substituted in one position with C₁₋₆ alkyl;
benzotriazolyl;
thiazolyl, which may be unsubstituted or independently substituted in one position with C₁₋₆ alkyl, halogen and phenyl substituted by one or more halogen atoms;
1,1-dioxobenzothienyl;
benzimidazolyl, which may be unsubstituted or independently substituted in one or more positions with haloC₁₋₆ alkyl; or
pyrimidinyl, which may be unsubstituted or independently substituted in one or more positions with halogen.

14. The compound according to claim 1 wherein Ar is
phenyl, which may be unsubstituted or independently substituted in one, two, three, four, or five positions with methyl, ethyl, isopropyl, n-butyl, tert-butyl, trifluoromethyl, methoxy, difluoromethoxy, trifluoromethoxy, 1,1,2,2-tetrafluoroethoxy, methylthio, trifluoromethylthio, acetyl, ethoxycarbonyl, methylsulfonyl, trifluoromethylsulfonyl, fluoro, chloro, bromo, iodo, dimethylamino, nitro, cyano, n-butylaminosulfonyl, morpholinyl, phenyl, cyanophenyl, thiadiazolyl, phenoxy, benzyloxy, tetrazolyl, oxazolyl, 4-bromo-1-methyl-1H-pyrazol-3-yl, 6-methyl-1,3-benzothiazol-2-yl;
difluoromethylenedioxyphenyl;
furyl, which may be unsubstituted or independently substituted in one or two positions with methyl or trifluoromethyl;
thienyl;
pyridinyl, which may be unsubstituted or independently substituted in one or two positions with methyl, ethyl, methoxy, ethoxy, trifluoromethyl, chloro, bromo or nitro;
pyrazolyl, which may be unsubstituted or independently substituted in one two or three positions with methyl or bromo;
pyridazinyl;
isoxazolyl, which may be unsubstituted or independently substituted in one or two positions with methyl;
isothiazolyl, substituted in one position with methyl;
dihydroindenyl;
naphthyl;
fluorenyl;
indolyl, substituted in one position with methyl;
quinolinyl, which may be unsubstituted or independently substituted in one position with methyl, halogen and trifluoromethyl;
dihydrobenzofuranyl;
benzodioxolyl;
benzodioxinyl, which may be unsubstituted or independently substituted in one or two positions with one or two fluoro atoms in each position;
benzothiazolyl, which may be unsubstituted or independently substituted in one position with methyl or halogen;
benzothiadiazolyl;
benzotriazolyl;
thiazolyl, which may be unsubstituted or independently substituted in one position with methyl, halogen and 2-chloro-6-fluorophenyl;
1,1-dioxobenzothienyl;
benzimidazolyl, which may be unsubstituted or independently substituted in one position with trifluoromethyl; or
pyrimidinyl, which may be unsubstituted or independently substituted in one or more positions with halogen.

15. The compound according to claim 1, wherein X is sulfur; R⁰ is absent; R¹ and R² are both methyl; R³ is methyl or benzyl; and R⁴-R⁷ are all hydrogen.

16. The compound according to claim 1, wherein Ar is
phenyl, which may be unsubstituted or independently substituted in one or two positions with methyl, isopropyl, methoxy, methylthio, trifluoromethyl, fluoro, chloro, dimethylamino, nitro, piperidinylsulfonyl, pyrazolyl or oxazolyl;
thienyl, which may be unsubstituted or substituted in one position with methoxycarbonyl;
pyridyl, which may be unsubstituted or substituted in one position with phenoxy or morpholinyl; or
dihydroindenyl.

17. The compound according to claim 1, wherein X is NH; R⁰ is absent; R¹ and R² are both methyl; R³ is methyl; R⁴-R⁷ are all hydrogen; and Ar is phenyl which is substituted in two positions with fluoro or which is substituted in two positions with chloro.

18. The compound according to claim 1, wherein X is CH—NO₂; R⁰ is absent; R¹, R² and R³ are all methyl; R⁴-R⁷ are all hydrogen; and Ar is phenyl which is substituted in two positions with chloro.

19. The compound according to claim 1, wherein X is N—CN; R⁰ is absent; R¹ and R² are both methyl; R³ is methyl; R⁴-R⁷ are all hydrogen; and Ar is phenyl that is independently substituted in one or two positions with trifluoromethyl, chloro or bromo; or Ar is pyridyl that is independently substituted in one or two positions with methoxy or chloro.

20. The compound according to claim 1, which is a compound selected from the group consisting of:
N-(3,4-difluorophenyl)-N′-[(3aS,6R,7aS)-3a-(3,4-dimethoxyphenyl)-1-methyloctahydro-1H-indol-6-yl]urea hydrochloride,
N-[(3aS*,6R*,7aS*)-1-benzyl-3a-(3,4-dimethoxyphenyl)octahydro-1H-indol-6-yl]-N′-pyridin-3-ylthiourea trifluoroacetate,
methyl 3-[({[(3aS*,6R*,7aS*)-1-benzyl-3a-(3,4-dimethoxyphenyl)octahydro-1H-indol-6-yl]amino}carbonothioyl)amino]thiophene-2-carboxylate trifluoroacetate,
N-[(3aS*,6R*,7aS*)-1-benzyl-3a-(3,4-dimethoxyphenyl)octahydro-1H-indol-6-yl]-N′-[4-(dimethylamino)phenyl]thiourea trifluoroacetate,
N-[(3aS*,6R*,7aS*)-1-benzyl-3a-(3,4-dimethoxyphenyl)octahydro-1H-indol-6-yl]-N′-[4-(dimethylamino)phenyl]urea trifluoroacetate,
N-[(3aS*,6R*,7aS*)-1-benzyl-3a-(3,4-dimethoxyphenyl)octahydro-1H-indol-6-yl]-N′-(3,5-dimethylisoxazol-4-yl)urea trifluoroacetate,
N-[(3aS*,6R*,7aS*)-1-benzyl-3a-(3,4-dimethoxyphenyl)octahydro-1H-indol-6-yl]-N′-(3,5-dimethoxyphenyl)urea trifluoroacetate,
N-[(3aS*,6R*,7aS*)-1-benzyl-3a-(3,4-dimethoxyphenyl)octahydro-1H-indol-6-yl]-N′-(2,6-dichloropyridin-4-yl)urea trifluoroacetate,
N-[(3aS*,6R*,7aS*)-1-benzyl-3a-(3,4-dimethoxyphenyl)octahydro-1H-indol-6-yl]-N′-pyridin-4-ylurea trifluoroacetate,
N-[(3aS*,6R*,7aS*)-1-benzyl-3a-(3,4-dimethoxyphenyl)octahydro-1H-indol-6-yl]-N′-(3,5-dichloropyridin-4-yl)urea trifluoroacetate,
N-[(3aS*,6R*,7aS*)-1-benzyl-3a-(3,4-dimethoxyphenyl)octahydro-1H-indol-6-yl]-N′-(1,3,5-trimethyl-1H-pyrazol-4-yl)urea trifluoroacetate,
N-[(3aS*,6S*,7aS*)-1-benzyl-3a-(3,4-dimethoxyphenyl)octahydro-1H-indol-6-yl]-N′-(2,6-dichloropyridin-4-yl)urea trifluoroacetate,
N-(2,3-dichlorophenyl)-N′-[(3aS*,7aS*)-3a-(3,4-dimethoxyphenyl)-1-methyloctahydro-1H-indol-6-yl]urea,
N-(2,6-dichloropyridin-4-yl)-N′-[(3aS*,6R*,7aS*)-3a-(3,4-dimethoxyphenyl)-1-methyloctahydro-1H-indol-6-yl]urea trifluoroacetate,
N-(3-chlorophenyl)-N′-[(3aS*,6R*,7aS*)-3a-(3,4-dimethoxyphenyl)-1-methyloctahydro-1H-indol-6-yl]urea trifluoroacetate,
N-(4-cyanophenyl)-N′-[(3aS*,6R*,7aS*)-3a-(3,4-dimethoxyphenyl)-1-methyloctahydro-1H-indol-6-yl]urea trifluoroacetate,
N-(2,4-difluorophenyl)-N′-[(3aS*,6R*,7aS*)-3a-(3,4-dimethoxyphenyl)-1-methyloctahydro-1H-indol-6-yl]urea trifluoroacetate,
N-(5-chloro-2-methoxyphenyl)-N′-[(3aS*,6R*,7aS*)-3a-(3,4-dimethoxyphenyl)-1-methyloctahydro-1H-indol-6-yl]urea trifluoroacetate,
N-[4-chloro-3-(trifluoromethyl)phenyl]-N′-[(3aS*,6R*,7aS*)-3a-(3,4-dimethoxyphenyl)-1-methyloctahydro-1H-indol-6-yl]urea trifluoroacetate,
N-(3-chloro-2-methylphenyl)-N′-[(3aS*,6R*,7aS*)-3a-(3,4-dimethoxyphenyl)-1-methyloctahydro-1H-indol-6-yl]urea trifluoroacetate,
N-1,3-benzodioxol-5-yl-N′-[(3aS*,6R*,7aS*)-3a-(3,4-dimethoxyphenyl)-1-methyloctahydro-1H-indol-6-yl]urea trifluoroacetate,
N-(2,3-dichlorophenyl)-N′-[(3aS*,6R*,7aS*)-3a-(3,4-dimethoxyphenyl)-1-methyloctahydro-1H-indol-6-yl]thiourea trifluoroacetate,
N-(2,4-difluorophenyl)-N′-[(3aS*,6R*,7aS*)-3a-(3,4-dimethoxyphenyl)-1-methyloctahydro-1H-indol-6-yl]thiourea trifluoroacetate,
N-[(3aS*,6R*,7aS*)-3a-(3,4-dimethoxyphenyl)-1-methyloctahydro-1H-indol-6-yl]-N′-[4-(methylthio)phenyl]thiourea trifluoroacetate,
N-(2,3-dihydro-1H-inden-5-yl)-N′-[(3aS*,6R*,7aS*)-3a-(3,4-dimethoxyphenyl)-1-methyloctahydro-1H-indol-6-yl]thiourea trifluoroacetate,
N-(3,5-dichlorophenyl)-N′-[(3aS*,6R*,7aS*)-3a-(3,4-dimethoxyphenyl)-1-methyloctahydro-1H-indol-6-yl]urea trifluoroacetate,
N-(4-bromophenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-phenylurea trifluoroacetate,
N-(3,5-difluorophenyl)-N′-[(3aS*,6R*,7aR*)-3a-(3,4-dimethoxyphenyl)-1-methyloctahydro-1H-indol-6-yl]urea trifluoroacetate,
N-(3-chloro-4-fluorophenyl)-N′-[(3aS*,6R*,7aS*)-3a-(3,4-dimethoxyphenyl)-1-methyloctahydro-1H-indol-6-yl]urea trifluoroacetate,
N-(2,3-dihydro-1H-inden-5-yl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-2-naphthylurea trifluoroacetate,
N-[(3aS*,6R*,7aS*)-3a-(3,4-dimethoxyphenyl)-1-methyloctahydro-1H-indol-6-yl]-N′-{3-[(trifluoromethyl)thio]phenyl}urea trifluoroacetate,
N-[(3aS*,6R*,7aS*)-3a-(3,4-dimethoxyphenyl)-1-methyloctahydro-1H-indol-6-yl]-N′-(3-methoxyphenyl)urea trifluoroacetate,
N-[(3aS*,6R*,7aS*)-3a-(3,4-dimethoxyphenyl)-1-methyloctahydro-1H-indol-6-yl]-N′-(2,6-dimethylphenyl)urea trifluoroacetate,
N-(2,6-dichlorophenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-[3-(trifluoromethyl)phenyl]urea trifluoroacetate,
N-[(3aS*,6R*,7aS*)-3a-(3,4-dimethoxyphenyl)-1-methyloctahydro-1H-indol-6-yl]-N′-[4-fluoro-3-(trifluoromethyl)phenyl]urea trifluoroacetate,
N-[(3aS*,6R*,7aS*)-3a-(3,4-dimethoxyphenyl)-1-methyloctahydro-1H-indol-6-yl]-N′-(pentafluorophenyl)urea trifluoroacetate,
N-[(3aS*,6R*,7aS*)-3a-(3,4-dimethoxyphenyl)-1-methyloctahydro-1H-indol-6-yl]-N′-(2,3,5,6-tetrachlorophenyl)urea trifluoroacetate,
N-(3-bromophenyl)-N′-[(3aS*,6R*,7aS*)-3a-(3,4-dimethoxyphenyl)-1-methyloctahydro-1H-indol-6-yl]urea hydrochloride,
N-(3-bromophenyl)-N′-[(3aS*,6R*,7aS*)-3a-(3,4-dimethoxyphenyl)-1-methyloctahydro-1H-indol-6-yl]urea trifluoroacetate,
N-(3-chloro-4-methoxyphenyl)-N′-[(3aS*,6R*,7aS*)-3a-(3,4-dimethoxyphenyl)-1-methyloctahydro-1H-indol-6-yl]urea trifluoroacetate,
N-(3,5-dichlorophenyl)-N′-[(3aS*,6R*,7aS*)-3a-(3,4-dimethoxyphenyl)-1-methyloctahydro-1H-indol-6-yl]guanidine trifluoroacetate,
N-(2,4-difluorophenyl)-N′-[(3aS*,6R*,7aS*)-3a-(3,4-dimethoxyphenyl)-1-methyloctahydro-1H-indol-6-yl]guanidine trifluoroacetate,
N-(3,5-difluorophenyl)-N′-[(3aS*,6R*,7aS*)-3a-(3,4-dimethoxyphenyl)-1-methyloctahydro-1H-indol-6-yl]guanidine trifluoroacetate,
N-[(3aS*,6R*,7aS*)-3a-(3,4-dimethoxyphenyl)-1-methyloctahydro-1H-indol-6-yl]-N′-pyridazin-4-ylurea trifluoroacetate,
N-[(3aS*,6R*,7aS*)-3a-(3,4-dimethoxyphenyl)-1-methyloctahydro-1H-indol-6-yl]-N′-(3,5-dimethylisoxazol-4-yl)urea trifluoroacetate,
N′-[4-chloro-3-(trifluoromethyl)phenyl]-N-[(3aS*,6S*,7aS*)-3a-(3,4-dimethoxyphenyl)-1-methyloctahydro-1H-indol-6-yl]-N-methylurea,
N′-(3-bromophenyl)-N-[(3aS*,6R*,7aS*)-3a-(3,4-dimethoxyphenyl)-1-methyloctahydro-1H-indol-6-yl]-N-methylurea trifluoroacetate,
N′-(3-bromophenyl)-N-[(3aS*,6S*,7aS*)-3a-(3,4-dimethoxyphenyl)-1-methyloctahydro-1H-indol-6-yl]-N-methylurea trifluoroacetate,
ethyl 4-[({[(3aS*,6R*,7aS*)-3a-(3,4-dimethoxyphenyl)-1-methyloctahydro-1H-indol-6-yl]amino}carbonyl)amino]benzoate trifluoroacetate,
ethyl 3-[({[(3aS*,6R*,7aS*)-3a-(3,4-dimethoxyphenyl)-1-methyloctahydro-1H-indol-6-yl]amino}carbonyl)amino]benzoate trifluoroacetate,
N-(3-cyanophenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(2-methoxy-5-nitrophenyl)urea trifluoroacetate,
N-(2-chlorophenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(4-methoxy-2-nitrophenyl)urea trifluoroacetate,
N-[(3aS*,6R*,7aS*)-3a-(3,4-dimethoxyphenyl)-1-methyloctahydro-1H-indol-6-yl]-N′-[4-(trifluoromethyl)phenyl]urea trifluoroacetate,
N-[(3aS*,6R*,7aS*)-3a-(3,4-dimethoxyphenyl)-1-methyloctahydro-1H-indol-6-yl]-N′-(2-methoxy-4-nitrophenyl)thiourea trifluoroacetate,
N-[(3aS*,6R*,7aS*)-3a-(3,4-dimethoxyphenyl)-1-methyloctahydro-1H-indol-6-yl]-N′-[4-(dimethylamino)phenyl]urea trifluoroacetate,
N-[(3aS*,6R*,7aS*)-3a-(3,4-dimethoxyphenyl)-1-methyloctahydro-1H-indol-6-yl]-N′-[4-(trifluoromethoxy)phenyl]urea trifluoroacetate,
N-[4-bromo-2-(trifluoromethyl)phenyl]-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(4-fluoro-3-nitrophenyl)urea trifluoroacetate,
N-[(3aS*,6R*,7aS*)-3a-(3,4-dimethoxyphenyl)-1-methyloctahydro-1H-indol-6-yl]-N′-(4-fluorophenyl)urea trifluoroacetate,
N-[(3aS*,6R*,7aS*)-3a-(3,4-dimethoxyphenyl)-1-methyloctahydro-1H-indol-6-yl]-N′-[3-(methylthio)phenyl]urea trifluoroacetate,
N-[(3aS*,6R*,7aS*)-3a-(3,4-dimethoxyphenyl)-1-methyloctahydro-1H-indol-6-yl]-N′-(4-methoxy-2-methylphenyl)urea trifluoroacetate,
methyl 3-[({[(3aS*,6R*,7aS*)-3a-(3,4-dimethoxyphenyl)-1-methyloctahydro-1H-indol-6-yl]amino}carbonothioyl)amino]thiophene-2-carboxylate trifluoroacetate,
N-(2-chloro-5-methylphenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(2-methylphenyl)urea trifluoroacetate,
N-[(3aS*,6R*,7aS*)-3a-(3,4-dimethoxyphenyl)-1-methyloctahydro-1H-indol-6-yl]-N′-(2-fluorophenyl)urea trifluoroacetate,
N-(2,4-dichlorophenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-[2-(trifluoromethyl)phenyl]urea trifluoroacetate,
N-[(3aS*,6R*,7aS*)-3a-(3,4-dimethoxyphenyl)-1-methyloctahydro-1H-indol-6-yl]-N′-(4-methyl-3-nitrophenyl)urea trifluoroacetate,
N-[(3aS*,6R*,7aS*)-3a-(3,4-dimethoxyphenyl)-1-methyloctahydro-1H-indol-6-yl]-N′-(2,4-dimethylphenyl)urea trifluoroacetate,
N-(4-tert-butylphenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-{4-[(trifluoromethyl)thio]phenyl urea trifluoroacetate,
N-[(3aS*,6R*,7aS*)-3a-(3,4-dimethoxyphenyl)-1-methyloctahydro-1H-indol-6-yl]-N′-(4-methoxyphenyl)urea trifluoroacetate,
N-(2-bromophenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-[3-(trifluoromethyl)phenyl]thiourea trifluoroacetate,
N-(4-chloro-2-nitrophenyl)-N′-[(3aS*,6R*,7aS*)-3a-(3,4-dimethoxyphenyl)-1-methyloctahydro-1H-indol-6-yl]urea trifluoroacetate,
N-(3-acetylphenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(3-fluoro-4-methylphenyl)urea trifluoroacetate,
N-[(3aS*,6R*,7aS*)-3a-(3,4-dimethoxyphenyl)-1-methyloctahydro-1H-indol-6-yl]-N′-(4-methylphenyl)urea trifluoroacetate,
N-[(3aS*,6R*,7aS*)-3a-(3,4-dimethoxyphenyl)-1-methyloctahydro-1H-indol-6-yl]-N′-(4,5-dimethyl-2-nitrophenyl)urea trifluoroacetate,
N-(5-chloro-2,4-dimethoxyphenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(2-methoxy-5-methylphenyl)urea trifluoroacetate,
N-(4-chloro-3-nitrophenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(3-nitrophenyl)urea trifluoroacetate,
N-[(3aS*,6R*,7aS*)-3a-(3,4-dimethoxyphenyl)-1-methyloctahydro-1H-indol-6-yl]-N′-(4-ethylphenyl)urea trifluoroacetate,
N-[2-chloro-5-(trifluoromethyl)phenyl]-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-[3-fluoro-5-(trifluoromethyl)phenyl]urea trifluoroacetate,
N-[(3aS*,6R*,7aS*)-3a-(3,4-dimethoxyphenyl)-1-methyloctahydro-1H-indol-6-yl]-N′-[2-fluoro-3-(trifluoromethyl)phenyl]urea hydrochloride,
N-[(3aS*,6R*,7aS*)-3a-(3,4-dimethoxyphenyl)-1-methyloctahydro-1H-indol-6-yl]-N′-[2-fluoro-3-(trifluoromethyl)phenyl]urea trifluoroacetate,
N-[(3aS*,6R*,7aS*)-3a-(3,4-dimethoxyphenyl)-1-methyloctahydro-1H-indol-6-yl]-N′-(2-fluoro-5-methylphenyl)urea trifluoroacetate,
N-[(3aS*,6R*,7aS*)-3a-(3,4-dimethoxyphenyl)-1-methyloctahydro-1H-indol-6-yl]-N′-(3,5-dinitrophenyl)urea trifluoroacetate,
N-(2,5-difluorophenyl)-N′-[(3aS*,6R*,7aS*)-3a-(3,4-dimethoxyphenyl)-1-methyloctahydro-1H-indol-6-yl]urea trifluoroacetate,
N-(3-chloro-4-methylphenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(2-methyl-5-nitrophenyl)urea trifluoroacetate,
N-[4-(difluoromethoxy)phenyl]-N′-[(3aS*,6R*,7aS*)-3a-(3,4-dimethoxyphenyl)-1-methyloctahydro-1H-indol-6-yl]urea trifluoroacetate,
N-(4-chloro-2-methylphenyl)-N′-[(3aS*,6R*,7aS*)-3a-(3,4-dimethoxyphenyl)-1-methyloctahydro-1H-indol-6-yl]urea trifluoroacetate,
N-(4-bromo-3-methylphenyl)-N′-[(3aS*,6R*,7aS*)-3a-(3,4-dimethoxyphenyl)-1-methyloctahydro-1H-indol-6-yl]urea trifluoroacetate,
N-(4-bromo-2-fluorophenyl)-N′-[(3aS*,6R*,7aS*)-3a-(3,4-dimethoxyphenyl)-1-methyloctahydro-1H-indol-6-yl]urea trifluoroacetate,
N-[3,5-bis(trifluoromethyl)phenyl]-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(4-fluoro-2-methylphenyl)urea trifluoroacetate,
N-(4-bromo-2-chlorophenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(3,5-dimethylphenyl)urea trifluoroacetate,
N-[(3aS*,6R*,7aS*)-3a-(3,4-dimethoxyphenyl)-1-methyloctahydro-1H-indol-6-yl]-N′-(2,5-dimethylphenyl)urea trifluoroacetate,
N-[(3aS*,6R*,7aS*)-3a-(3,4-dimethoxyphenyl)-1-methyloctahydro-1H-indol-6-yl]-N′-(3,4-dimethylphenyl)urea trifluoroacetate,
N-(3,4-difluorophenyl)-N′-[(3aS*,6R*,7aS*)-3a-(3,4-dimethoxyphenyl)-1-methyloctahydro-1H-indol-6-yl]urea,
N-(3,4-difluorophenyl)-N′-[(3aS*,6R*,7aS*)-3a-(3,4-dimethoxyphenyl)-1-methyloctahydro-1H-indol-6-yl]urea hydrochloride,
N-(3,4-difluorophenyl)-N′-[(3aS*,6R*,7aS*)-3a-(3,4-dimethoxyphenyl)-1-methyloctahydro-1H-indol-6-yl]urea trifluoroacetate,
N-[2-chloro-4-(trifluoromethyl)phenyl]-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(4-nitrophenyl)urea trifluoroacetate,
N-[(3aS*,6R*,7aS*)-3a-(3,4-dimethoxyphenyl)-1-methyloctahydro-1H-indol-6-yl]-N′-(2-fluoro-5-nitrophenyl)urea trifluoroacetate,
N-[(3aS*,6R*,7aS*)-3a-(3,4-dimethoxyphenyl)-1-methyloctahydro-1H-indol-6-yl]-N′-(3-ethylphenyl)urea trifluoroacetate,
N-(4-chlorophenyl)-N′-[(3aS*,6S*,7aS*)-3a-(3,4-dimethoxyphenyl)-1-methyloctahydro-1H-indol-6-yl]-N-methylurea trifluoroacetate,
N-biphenyl-4-yl-N′-[(3aS*,6R*,7aS*)-3a-(3,4-dimethoxyphenyl)-1-methyloctahydro-1H-indol-6-yl]urea trifluoroacetate,
N-(2,5-dichlorophenyl)-N′-[(3aS*,6R*,7aS*)-3a-(3,4-dimethoxyphenyl)-1-methyloctahydro-1H-indol-6-yl]urea trifluoroacetate,
N-(3,4-dichlorophenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(2,2,4,4-tetrafluoro-4H-1,3-benzodioxin-6-yl)urea trifluoroacetate,
N-(3,4-dichlorophenyl)-N′-[(3aS*,6S*,7aS*)-3a-(3,4-dimethoxyphenyl)-1-methyloctahydro-1H-indol-6-yl]-N-methylurea trifluoroacetate,
N-(5-chloro-2-methylphenyl)-N′-[(3aS*,6R*,7aS*)-3a-(3,4-dimethoxyphenyl)-1-methyloctahydro-1H-indol-6-yl]urea trifluoroacetate,
N-(3-bromophenyl)-N′-cyano-N″-[(3aS*,6R*,7aS*)-3a-(3,4-dimethoxyphenyl)-1-methyloctahydro-1H-indol-6-yl]guanidine trifluoroacetate,
N-[(3aS*,6R*,7aS*)-3a-(3,4-dimethoxyphenyl)-1-methyloctahydro-1H-indol-6-yl]-N′-(3-nitrophenyl)thiourea trifluoroacetate,
N-[(3aS*,6R*,7aS*)-3a-(3,4-dimethoxyphenyl)-1-methyloctahydro-1H-indol-6-yl]-N′-(4-fluorophenyl)thiourea,
N-(3-chlorophenyl)-N′-[(3aR,6S,7aR)-3a-(3,4-dimethoxyphenyl)-1-methyloctahydro-1H-indol-6-yl]urea,
N-[4-chloro-3-(trifluoromethyl)phenyl]-N′-[(3aR,6S,7aR)-3a-(3,4-dimethoxyphenyl)-1-methyloctahydro-1H-indol-6-yl]urea,
N-(3-chlorophenyl)-N′-[(3aS,6R,7aS)-3a-(3,4-dimethoxyphenyl)-1-methyloctahydro-1H-indol-6-yl]urea trifluoroacetate,
N-[4-chloro-3-(trifluoromethyl)phenyl]-N′-[(3aS,6R,7aS)-3a-(3,4-dimethoxyphenyl)-1-methyloctahydro-1H-indol-6-yl]urea hydrochloride,
N-[4-chloro-3-(trifluoromethyl)phenyl]-N′-[(3aS,6R,7aS)-3a-(3,4-dimethoxyphenyl)-methyloctahydro-1H-indol-6-yl]urea trifluoroacetate,
N-(3-bromophenyl)-N′-[(3aS,6R,7aS)-3a-(3,4-dimethoxyphenyl)-1-methyloctahydro-1H-indol-6-yl]urea hydrochloride,
N-(3-bromophenyl)-N′-[(3aS,6R,7aS)-3a-(3,4-dimethoxyphenyl)-1-methyloctahydro-1H-indol-6-yl]urea trifluoroacetate,
N-cyano-N′-(3,5-dichlorophenyl)-N″-[(3aS*,6R*,7aS*)-3a-(3,4-dimethoxyphenyl)-1-methyloctahydro-1H-indol-6-yl]guanidine,
N-[(3aS*,6R*,7aS*)-3a-(3,4-dimethoxyphenyl)-1-methyloctahydro-1H-indol-6-yl]-N′-[5-methyl-2-(trifluoromethyl)-3-furyl]urea trifluoroacetate,
N-(2,3-dihydro-1-benzofuran-5-yl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(6-fluoro-4H-1,3-benzodioxin-7-yl)urea trifluoroacetate,
N-[(3aS*,6R*,7aS*)-3a-(3,4-dimethoxyphenyl)-1-methyloctahydro-1H-indol-6-yl]-N′-3-thienylurea trifluoroacetate,
N-[(3aS*,6R*,7aS*)-3a-(3,4-dimethoxyphenyl)-1-methyloctahydro-1H-indol-6-yl]-N′-(6-morpholin-4-ylpyridin-3-yl)thiourea trifluoroacetate,
N-[(3aS*,6R*,7aS*)-3a-(3,4-dimethoxyphenyl)-1-methyloctahydro-1H-indol-6-yl]-N′-[4-(1H-pyrazol-1-yl)phenyl]thiourea trifluoroacetate,
(E)-N˜1˜-(3,5-dichlorophenyl)-N˜1˜-[(3aS*,6R*,7aS*)-3a-(3,4-dimethoxyphenyl)-1-methyloctahydro-1H-indol-6-yl]-2-nitroethylene-1,1-diamine trifluoroacetate,
N-[4-chloro-3-(trifluoromethyl)phenyl]-N′-cyano-N″-[(3aS*,6R*,7aS*)-3a-(3,4-dimethoxyphenyl)-1-methyloctahydro-1H-indol-6-yl]guanidine trifluoroacetate,
N′-[4-chloro-3-(trifluoromethyl)phenyl]-N-cyclohexyl-N-[(3aS*,6R*,7aS*)-3a-(3,4-dimethoxyphenyl)-1-methyloctahydro-1H-indol-6-yl]urea hydrochloride,
N′-[4-chloro-3-(trifluoromethyl)phenyl]-N-[(3aS*,6R*,7aS*)-3a-(3,4-dimethoxyphenyl)-1-methyloctahydro-1H-indol-6-yl]-N-(1-methylpiperidin-4-yl)urea,
N′-[4-chloro-3-(trifluoromethyl)phenyl]-N-[(3aS*,6R*,7aS*)-3a-(3,4-dimethoxyphenyl)-1-methyloctahydro-1H-indol-6-yl]-N-(1-methyl-1,2,3,6-tetrahydropyridin-4-yl)urea,
N-benzyl-N′-[4-chloro-3-(trifluoromethyl)phenyl]-N-[(3aS*,6R*,7aS*)-3a-(3,4-dimethoxyphenyl)-1-methyloctahydro-1H-indol-6-yl]urea,
N-benzyl-N′-[4-chloro-3-(trifluoromethyl)phenyl]-N-[(3aS*,6S*,7aS*)-3a-(3,4-dimethoxyphenyl)-1-methyloctahydro-1H-indol-6-yl]urea,
N-butyl-N′-[4-chloro-3-(trifluoromethyl)phenyl]-N-[(3aS*,6R*,7aS*)-3a-(3,4-dimethoxyphenyl)-1-methyloctahydro-1H-indol-6-yl]urea,
N-butyl-N′-[4-chloro-3-(trifluoromethyl)phenyl]-N-[(3aS*,6S*,7aS*)-3a-(3,4-dimethoxyphenyl)-1-methyloctahydro-1H-indol-6-yl]urea,
N′-[4-chloro-3-(trifluoromethyl)phenyl]-N-[(3aS*,6S*,7aS*)-3a-(3,4-dimethoxyphenyl)-1-methyloctahydro-1H-indol-6-yl]-N-isopropylurea,
N′-[4-chloro-3-(trifluoromethyl)phenyl]-N-[(3aS*,6R*,7aS*)-3a-(3,4-dimethoxyphenyl)-1-methyloctahydro-1H-indol-6-yl]-N-(2-methoxyethyl)urea,
N′-[4-chloro-3-(trifluoromethyl)phenyl]-N-[(3aS*,6S*,7aS*)-6-cyano-3a-(3,4-dimethoxyphenyl)-1-methyloctahydro-1H-indol-6-yl]-N-ethylurea,
N′-[4-chloro-3-(trifluoromethyl)phenyl]-N-[(3aS*,6R*,7aS*)-3a-(3,4-dimethoxyphenyl)-1-methyloctahydro-1H-indol-6-yl]-N-ethylurea,
N′-[4-chloro-3-(trifluoromethyl)phenyl]-N-[(3aS*,6S*,7aS*)-6-cyano-3a-(3,4-dimethoxyphenyl)-1-methyloctahydro-1H-indol-6-yl]-N-propylurea,
N′-[4-chloro-3-(trifluoromethyl)phenyl]-N-[(3aS*,6S*,7aS*)-3a-(3,4-dimethoxyphenyl)-1-methyloctahydro-1H-indol-6-yl]-N-propylurea,
N-ally-N′-[4-chloro-3-(trifluoromethyl)phenyl]-N-[(3aS*,6R*,7aS*)-3a-(3,4-dimethoxyphenyl)-1-methyloctahydro-1H-indol-6-yl]urea,
N-allyl-N′-[4-chloro-3-(trifluoromethyl)phenyl]-N-[(3aS*,6S*,7aS*)-3a-(3,4-dimethoxyphenyl)-1-methyloctahydro-1H-indol-6-yl]urea,
N-(3′-cyanobiphenyl-4-yl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-1-benzyl-3a-(3,4-dimethoxyphenyl)octahydro-1H-indol-6-yl]-N′-(3,4-difluorophenyl)urea,
N-[(3aS*,6R*,7aS*)-1-benzyl-3a-(3,4-dimethoxyphenyl)octahydro-1H-indol-6-yl]-N′-(3,4-difluorophenyl)urea trifluoroacetate,
N-[(3aS*,6R*,7aS*)-1-benzyl-3a-(3,4-dimethoxyphenyl)octahydro-1H-indol-6-yl]-N′-(3,4-difluorophenyl)urea,
N-[(3aS*,6R*,7aS*)-1-benzyl-3a-(3,4-dimethoxyphenyl)octahydro-1H-indol-6-yl]-N′-(3,4-difluorophenyl)urea trifluoroacetate,
N-[(3aS*,6R*,7aS*)-3a-(3,4-dimethoxyphenyl)-1-methyloctahydro-1H-indol-6-yl]-N′-(4-isopropylphenyl)thiourea trifluoroacetate,
N-[3,5-bis(trifluoromethyl)phenyl]-N′-[(3aS*,6R*,7aS*)-3a-(3,4-dimethoxyphenyl)-1-methyloctahydro-1H-indol-6-yl]thiourea trifluoroacetate,
N-(3,5-dichlorophenyl)-N′-[(3aS*,6R*,7aS*)-3a-(3,4-dimethoxyphenyl)-1-methyloctahydro-1H-indol-6-yl]thiourea trifluoroacetate,
N-[(3aS*,6R*,7aS*)-3a-(3,4-dimethoxyphenyl)-1-methyloctahydro-1H-indol-6-yl]-N′-(5-fluoro-2-methylphenyl)thiourea trifluoroacetate,
N-(3-chlorophenyl)-N′-[(3aS*,6R*,7aS*)-3a-(3,4-dimethoxyphenyl)-1-methyloctahydro-1H-indol-6-yl]thiourea trifluoroacetate,
N-(2,5-difluorophenyl)-N′-[(3aS*,6R*,7aS*)-3a-(3,4-dimethoxyphenyl)-1-methyloctahydro-1H-indol-6-yl]thiourea trifluoroacetate,
N-[(3aS*,6R*,7aS*)-3a-(3,4-dimethoxyphenyl)-1-methyloctahydro-1H-indol-6-yl]-N′-(3,5-dimethylphenyl)thiourea trifluoroacetate,
N-(3-chloro-4-fluorophenyl)-N′-[(3aS*,6R*,7aS*)-3a-(3,4-dimethoxyphenyl)-1-methyloctahydro-1H-indol-6-yl]thiourea trifluoroacetate,
N-(3,4-dichlorophenyl)-N′-[(3aS*,6R*,7aS*)-3a-(3,4-dimethoxyphenyl)-1-methyloctahydro-1H-indol-6-yl]thiourea trifluoroacetate,
N-[(3aS*,6R*,7aS*)-3a-(3,4-dimethoxyphenyl)-1-methyloctahydro-1H-indol-6-yl]-N′-(6-phenoxypyridin-3-yl)thiourea trifluoroacetate,
N-[(3aS*,6R*,7aS*)-3a-(3,4-dimethoxyphenyl)-1-methyloctahydro-1H-indol-6-yl]-N′-[4-(piperidin-1-ylsulfonyl)phenyl]thiourea trifluoroacetate,
N-[(3aS*,6R*,7aS*)-3a-(3,4-dimethoxyphenyl)-1-methyloctahydro-1H-indol-6-yl]-N′-[4-(1,3-oxazol-5-yl)phenyl]thiourea trifluoroacetate,
N-(3,4-difluorophenyl)-N′-[(3aS*,6R*,7aS*)-3a-(3,4-dimethoxyphenyl)octahydro-1H-indol-6-yl]urea,
N-(3,4-difluorophenyl)-N′-[(3aS*,6R*,7aS*)-3a-(3,4-dimethoxyphenyl)-1-propyloctahydro-1H-indol-6-yl]urea trifluoroacetate,
N-(3,4-difluorophenyl)-N′-[(3aS*,6R*,7aS*)-3a-(3,4-dimethoxyphenyl)-1-(2-hydroxyethyl)octahydro-1H-indol-6-yl]urea trifluoroacetate (salt),
N-(3,4-difluorophenyl)-N′-{(3aS*,6R*,7aS*)-3a-(3,4-dimethoxyphenyl)-1-[(1-methyl-1H-pyrrol-2-yl)methyl]octahydro-1H-indol-6-yl}urea trifluoroacetate,
N′-[4-chloro-3-(trifluoromethyl)phenyl]-N-[(3aS*,6R*,7aS*)-3a-(3,4-dimethoxyphenyl) 1-methyloctahydro-1H-indol-6-yl]-N-isopropylurea hydrochloride,
N-(3-chlorophenyl)-N′-[(3aS*,6R*,7aS*)-3a-(3,4-dimethoxyphenyl)-1-methyloctahydro-1H-indol-6-yl]-N-methylurea,
N-[(3aS*,6R*,7aS*)-3a-(3,4-dimethoxyphenyl)-1-methyloctahydro-1H-indol-6-yl]-N′-pyridin-2-ylurea,
N-[(3aS*,6R*,7aS*)-3a-(3,4-dimethoxyphenyl)-1-methyloctahydro-1H-indol-6-yl]-N′-pyridin-3-ylurea,
N-[(3aS*,6R*,7aS*)-3a-(3,4-dimethoxyphenyl)-1-methyloctahydro-1H-indol-6-yl]-N′-pyridin-4-ylurea,
N-(3,4-difluorophenyl)-N′-[(3aS*,6R*,7aS*)-3a-(3,4-dimethoxyphenyl)-1-(1H-pyrazol-3-ylmethyl)octahydro-1H-indol-6-yl]urea trifluoroacetate,
N-(3,4-difluorophenyl)-N′-[(3aS*,6R*,7aS*)-3a-(3,4-dimethoxyphenyl)-1-(3,3,3-trifluoro-2-methylpropyl)octahydro-1H-indol-6-yl]urea trifluoroacetate,
N-[(3aS*,6R*,7aS*)-1-butyl-3a-(3,4-dimethoxyphenyl)octahydro-1H-indol-6-yl]-N′-(3,4-difluorophenyl)urea trifluoroacetate,
N-(3,4-difluorophenyl)-N′-[(3aS*,6R*,7aS*)-3a-(3,4-dimethoxyphenyl)-1-(1H-imidazol-4-ylmethyl)octahydro-1H-indol-6-yl]urea trifluoroacetate,
N-(3,4-difluorophenyl)-N′-[(3aS*,6R*,7aS*)-3a-(3,4-dimethoxyphenyl)-1-(1H-imidazol-2-ylmethyl)octahydro-1H-indol-6-yl]urea trifluoroacetate,
N-(3,4-difluorophenyl)-N′-[(3aS*,6R*,7aS*)-3a-(3,4-dimethoxyphenyl)-1-ethyloctahydro-1H-indol-6-yl]urea trifluoroacetate,
N-[(3aS*,6R*,7aS*)-1-acetyl-3a-(3,4-dimethoxyphenyl)octahydro-1H-indol-6-yl]-N′-(3,4-difluorophenyl)urea trifluoroacetate,
N-[(3aS*,6R*,7aS*)-3a-(3,4-dimethoxyphenyl)-1-methyloctahydro-1H-indol-6-yl]-N′-quinolin-3-ylurea,
N′-[(3aS*,6R*,7aS*)-3a-(3,4-dimethoxyphenyl)-1-methyloctahydro-1H-indol-6-yl]-N-(4-fluorophenyl)-N-methylurea trifluoroacetate,
N′-[(3aS*,6R*,7aS*)-3a-(3,4-dimethoxyphenyl)-1-methyloctahydro-1H-indol-6-yl]-N-(4-methoxyphenyl)-N-methylurea trifluoroacetate,
N-(4-bromophenyl)-N′-[(3aS*,6R*,7aS*)-3a-(3,4-dimethoxyphenyl)-1-methyloctahydro-1H-indol-6-yl]-N-methylurea trifluoroacetate,
N-[(3aS*,6R*,7aS*)-3a-(3,4-dimethoxyphenyl)-1-methyloctahydro-1H-indol-6-yl]-N′-(5-ethylpyridin-2-yl)urea,
N-(5-bromopyridin-3-yl)-N′-[(3aS*,6R*,7aS*)-3a-(3,4-dimethoxyphenyl)-1-methyloctahydro-1H-indol-6-yl]urea,
N′-[(3aS*,6R*,7aS*)-3a-(3,4-dimethoxyphenyl)-1-methyloctahydro-1H-indol-6-yl]-N-methyl-N-(4-methylphenyl)urea trifluoroacetate,
N-(4-chlorophenyl)-N′-[(3aS*,6R*,7aS*)-3a-(3,4-dimethoxyphenyl)-1-methyloctahydro-1H-indol-6-yl]-N-methylurea trifluoroacetate,
N-(2-chloro-6-methoxypyridin-4-yl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(2,6-dimethoxypyridin-3-yl)urea trifluoroacetate,
N-(6-chloropyridin-3-yl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-quinolin-4-ylurea,
N-(4-chloropyridin-2-yl)-N′-[(3aS*,6R*,7aS*)-3a-(3,4-dimethoxyphenyl)-1-methyloctahydro-1H-indol-6-yl]urea,
N-(6-bromopyridin-2-yl)-N′-[(3aS*,6R*,7aS*)-3a-(3,4-dimethoxyphenyl)-1-methyloctahydro-1H-indol-6-yl]urea,
N-[(3aS*,6R*,7aS*)-3a-(3,4-dimethoxyphenyl)-1-methyloctahydro-1H-indol-6-yl]-N′-(3-nitropyridin-2-yl)urea,
N-[(3aS*,6R*,7aS*)-3a-(3,4-dimethoxyphenyl)-1-methyloctahydro-1H-indol-6-yl]-N′-[6-(trifluoromethyl)pyridin-3-yl]urea,
N-(6-chloropyridin-3-yl)-N′-[(3aS*,6R*,7aS*)-3a-(3,4-dimethoxyphenyl)-1-methyloctahydro-1H-indol-6-yl]urea,
N-[(3aS*,6R*,7aS*)-3a-(3,4-diethoxyphenyl)-1-methyloctahydro-1H-indol-6-yl]-N′-(3,4-difluorophenyl)urea trifluoroacetate,
N-[(3aS*,6R*,7aS*)-3a-(3,4-dimethoxyphenyl)-1-methyloctahydro-1H-indol-6-yl]-N′-(2-methoxypyridin-3-yl)urea,
N-[(3aS*,6R*,7aS*)-3a-(3,4-dimethoxyphenyl)-1-methyloctahydro-1H-indol-6-yl]-N′-(4-ethoxypyridin-3-yl)urea,
N-(3,4-difluorophenyl)-N′-[(3aS*,6R*,7aS*)-3a-(3,4-dipropoxyphenyl)-1-methyloctahydro-1H-indol-6-yl]urea trifluoroacetate,
N-(3,4-difluorophenyl)-N′-[(3aS*,6R*,7aS*)-3a-(3,4-dihydro-2H-1,5-benzodioxepin-7-yl)-1-methyloctahydro-1H-indol-6-yl]urea trifluoroacetate,
N-(3,4-difluorophenyl)-N′-[(3aS*,6R*,7aS*)-3a-(3,4-diisopropoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(2,6-dimethylpyridin-4-yl)urea,
N-(3,4-difluorophenyl)-N′-[(3aS*,6R*,7aS*)-3a-(2,3-dihydro-1,4-benzodioxin-6-yl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(4,6-dimethylpyridin-2-yl)urea,
N-cyano-N′-[(3aS*,6R*,7aS*)-3a-(3,4-dimethoxyphenyl)-1-methyloctahydro-1H-indol-6-yl]-N″-(2,6-dimethoxypyridin-3-yl)guanidine,
N-(6-chloropyridin-3-yl)-N′-cyano-N″-[(3aS,6R,7aS)-3a-(3,4-dimethoxyphenyl)-1-methyloctahydro-1H-indol-6-yl]guanidine,
N-cyano-N′-[(3aS*,6R*,7aS*)-3a-(3,4-dimethoxyphenyl)-1-methyloctahydro-1H-indol-6-yl]-N″-(2-methoxypyridin-3-yl)guanidine,
N-biphenyl-2-yl-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(3-methylphenyl)urea trifluoroacetate,
ethyl 2-[({[(3aS*,6R*,7aS*)-3a-(3,4-dimethoxyphenyl)-1-methyloctahydro-1H-indol-6-yl]amino}carbonyl)amino]benzoate trifluoroacetate,
N-(2-tert-butylphenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-[2-(methylthio)phenyl]urea trifluoroacetate,
N-[(3aS*,6R*,7aS*)-3a-(3,4-dimethoxyphenyl)-1-methyloctahydro-1H-indol-6-yl]-N′-(2-methoxyphenyl)urea trifluoroacetate,
N-[(3aS*,6R*,7aS*)-3a-(3,4-dimethoxyphenyl)-1-methyloctahydro-1H-indol-6-yl]-N′-(2-nitrophenyl)urea trifluoroacetate,
N-[(3aS*,6R*,7aS*)-3a-(3,4-dimethoxyphenyl)-1-methyloctahydro-1H-indol-6-yl]-N′-(3-phenoxyphenyl)urea trifluoroacetate,
N-[(3aS,6R,7aS)-3a-(3,4-dimethoxyphenyl)-1-methyloctahydro-1H-indol-6-yl]-N′-[2-fluoro-3-(trifluoromethyl)phenyl]urea,
N-[(3aS*,6R*,7aS*)-3a-(3,4-dimethoxyphenyl)-1-methyloctahydro-1H-indol-6-yl]-N′-[2-fluoro-3-(trifluoromethyl)phenyl]urea hydrochloride,
N-[(3aS*,6R*,7aS*)-3a-(3,4-dimethoxyphenyl)-1-methyloctahydro-1H-indol-6-yl]-N′-(2-iodophenyl)urea trifluoroacetate,
N-[(3aS*,6R*,7aS*)-3a-(3,4-dimethoxyphenyl)-1-methyloctahydro-1H-indol-6-yl]-N′-(3-iodophenyl)urea trifluoroacetate,
N-[(3aS*,6R*,7aS*)-3a-(3,4-dimethoxyphenyl)-1-methyloctahydro-1H-indol-6-yl]-N′-(4-iodophenyl)urea trifluoroacetate,
N-[(3aS*,6R*,7aS*)-3a-(3,4-dimethoxyphenyl)-1-methyloctahydro-1H-indol-6-yl]-N′-[4-(methylthio)phenyl]urea trifluoroacetate,
N-[(3aS*,6R*,7aS*)-3a-(3,4-dimethoxyphenyl)-1-methyloctahydro-1H-indol-6-yl]-N′-(3-fluorophenyl)urea trifluoroacetate,
N-[(3aS*,6R*,7aS*)-3a-(3,4-dimethoxyphenyl)-1-methyloctahydro-1H-indol-6-yl]-N′-(2-ethylphenyl)urea trifluoroacetate,
N-(2-cyanophenyl)-N′-[(3aS*,6R*,7aS*)-3a-(3,4-dimethoxyphenyl)-1-methyloctahydro-1H-indol-6-yl]urea trifluoroacetate,
N-(4-butyl-2-methylphenyl)-N′-[(3aS*,6R*,7aS*)-3a-(3,4-dimethoxyphenyl)-1-methyloctahydro-1H-indol-6-yl]urea trifluoroacetate,
N-[(3aS,6R,7aS)-3a-(3,4-dimethoxyphenyl)-1-methyloctahydro-1H-indol-6-yl]-N′-[4-fluoro-2-(trifluoromethyl)phenyl]urea trifluoroacetate,
N-(2-bromo-4,6-difluorophenyl)-N′-[(3aS*,6R*,7aS*)-3a-(3,4-dimethoxyphenyl)-1-methyloctahydro-1H-indol-6-yl]urea trifluoroacetate,
N-(2,4-dibromo-6-fluorophenyl)-N′-[(3aS*,6R*,7aS*)-3a-(3,4-dimethoxyphenyl)-1-methyloctahydro-1H-indol-6-yl]urea trifluoroacetate,
N-(4-bromo-2,6-difluorophenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(2,3,4-trifluorophenyl)urea trifluoroacetate,
N-[2-(difluoromethoxy)phenyl]-N′-[(3aS*,7aS*)-3a-(3,4-dimethoxyphenyl)-1-methyloctahydro-1H-indol-6-yl]urea trifluoroacetate,
N-[4-chloro-2-(trifluoromethyl)phenyl]-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-[4-(6-methyl-1,3-benzothiazol-2-yl)phenyl]urea trifluoroacetate,
N-[(3aS*,6R*,7aS*)-3a-(3,4-dimethoxyphenyl)-1-methyloctahydro-1H-indol-6-yl]-N′-9H-fluoren-2-ylurea trifluoroacetate,
N-[4-(benzyloxy)phenyl]-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-[2-fluoro-5-(trifluoromethyl)phenyl]urea trifluoroacetate,
N-(4-butylphenyl)-N′-[(3aS*,6R*,7aS*)-3a-(3,4-dimethoxyphenyl)-1-methyloctahydro-1H-indol-6-yl]urea trifluoroacetate,
N-(3,5-dimethoxyphenyl)-N′-[(3aS*,6R*,7aS*)-3a-(3,4-dimethoxyphenyl)-1-methyloctahydro-1H-indol-6-yl]urea trifluoroacetate,
N-(2,5-difluorophenyl)-N′-[(3aS*,6R*,7aS*)-3a-(3,4-dimethoxyphenyl)-1-methyloctahydro-1H-indol-6-yl]urea hydrochloride,
N-{(3aS*,6R*,7aS*)-3a-[4-(benzyloxy)-3-methoxyphenyl]-1-methyloctahydro-1H-indol-6-yl}-N′-(3,4-difluorophenyl)urea,
N-(3,4-difluorophenyl)-N′-{(3aS*,6R*,7aS*)-3a-(3,4-dimethoxyphenyl)-1-[(dimethylamino)acetyl]octahydro-1H-indol-6-yl}urea,
N-(3,4-difluorophenyl)-N′-{(3aS*,6R*,7aS*)-3a-(3,4-dimethoxyphenyl)-1-[(1,3-dimethyl-1H-pyrazol-5-yl)methyl]octahydro-1H-indol-6-yl}urea trifluoroacetate,
N-(3,4-difluorophenyl)-N′-{(3aS*,6R*,7aS*)-3a-(3,4-dimethoxyphenyl)-1-[(3,5-dimethylisoxazol-4-yl)methyl]octahydro-1H-indol-6-yl}urea trifluoroacetate,
N-(3,4-difluorophenyl)-N′-[(3aS*,6R*,7aS*)-3a-(3,4-dimethoxyphenyl)-1-(1,3-thiazol-2-ylmethyl)octahydro-1H-indol-6-yl]urea trifluoroacetate,
N-(3,4-difluorophenyl)-N′-{(3aS*,6R*,7aS*)-3a-(3,4-dimethoxyphenyl)-1-[(1-methyl-1H-imidazol-2-yl)methyl]octahydro-1H-indol-6-yl}urea trifluoroacetate,
N-{(3aS*,6S*,7aS*)-3a-[4-(benzyloxy)-3-methoxyphenyl]-1-methyloctahydro-1H-indol-6-yl}-N′-(3,4-difluorophenyl)urea,
N-[(3aR*,6S*,7aR*)-3a-(3,4-dimethoxyphenyl)octahydro-1H-indol-6-yl]-N′-[2-fluoro-3-(trifluoromethyl)phenyl]urea trifluoroacetate,
N-(3-bromophenyl)-N′-[(3aR*,6S*,7aR*)-3a-(3,4-dimethoxyphenyl)octahydro-1H-indol-6-yl]urea trifluoroacetate,
N-(3,4-difluorophenyl)-N′-{(3aS*,6R*,7aS*)-3a-(3,4-dimethoxyphenyl)-1-[(2-methyl-1H-imidazol-4-yl)methyl]octahydro-1H-indol-6-yl}urea trifluoroacetate,
N-(3,4-difluorophenyl)-N′-[(3aS*,6R*,7aS*)-3a-(4-ethoxy-3-methoxyphenyl)-1-methyloctahydro-1H-indol-6-yl]urea trifluoroacetate,
N-[(3aS*,6R*,7aS*)-1-(2-azidoethyl)-3a-(3,4-dimethoxyphenyl)octahydro-1H-indol-6-yl]-N′-(3,4-difluorophenyl)urea,
N-(3,4-difluorophenyl)-N′-{(3aS*,6R*,7aS*)-3a-(3,4-dimethoxyphenyl)-1-[(4,5-dimethyl-2-furyl)methyl]octahydro-1H-indol-6-yl}urea trifluoroacetate,
N-(3,4-difluorophenyl)-N′-[(3aS*,6R*,7aS*)-3a-(3,4-dimethoxyphenyl)-1-(3-furylmethyl)octahydro-1H-indol-6-yl]urea trifluoroacetate,
N-(3,4-difluorophenyl)-N′-[(3aS*,6R*,7aS*)-3a-(3,4-dimethoxyphenyl)-1-isopropyloctahydro-1H-indol-6-yl]urea trifluoroacetate,
N-(3,4-difluorophenyl)-N′-[(3aS*,6R*,7aS*)-3a-(3,4-dimethoxyphenyl)-1-(pyridin-4-ylmethyl)octahydro-1H-indol-6-yl]urea bis(trifluoroacetate),
N-(3,4-difluorophenyl)-N′-[(3aS*,6R*,7aS*)-3a-(3,4-dimethoxyphenyl)-1-(pyridin-3-ylmethyl)octahydro-1H-indol-6-yl]urea bis(trifluoroacetate),
N-(3,4-difluorophenyl)-N′-{(3aS*,6R*,7aS*)-3a-(3,4-dimethoxyphenyl)-1-[(5-methyl-2-furyl)methyl]octahydro-1H-indol-6-yl}urea trifluoroacetate,
N-(3,4-difluorophenyl)-N′-[(3aS*,6R*,7aS*)-3a-(3,4-dimethoxyphenyl)-1-(3-thienylmethyl)octahydro-1H-indol-6-yl]urea trifluoroacetate,
N-(3,4-difluorophenyl)-N′-[(3aS*,6R*,7aS*)-3a-(3,4-dimethoxyphenyl)-1-(4-methylbenzyl)octahydro-1H-indol-6-yl]urea trifluoroacetate,
N-(3,4-difluorophenyl)-N′-[(3aS*,6R*,7aS*)-3a-(3,4-dimethoxyphenyl)-1-(4-fluorobenzyl)octahydro-1H-indol-6-yl]urea trifluoroacetate,
N-(3,4-difluorophenyl)-N′-{(3aS*,6R*,7aS*)-3a-(3,4-dimethoxyphenyl)-1-[(5-methyl-2-thienyl)methyl]octahydro-1H-indol-6-yl}urea trifluoroacetate, N-(3,4-difluorophenyl)-N′-{(3aS*,6R*,7aS*)-3a-(3,4-dimethoxyphenyl)-1-[(2-ethyl-4-methyl-1H-imidazol-5-yl)methyl]octahydro-1H-indol-6-yl}urea trifluoroacetate,
N-[(3aS*,6R*,7aS*)-1-[(5-chloro-1,3-dimethyl-1H-pyrazol-4-yl)methyl]-3a-(3,4-dimethoxyphenyl)octahydro-1H-indol-6-yl]-N′-(3,4-difluorophenyl)urea trifluoroacetate,
N-(3,4-difluorophenyl)-N′-{(3aS*,6R*,7aS*)-3a-(3,4-dimethoxyphenyl)-1-[(2-ethyl-1H-imidazol-4-yl)methyl]octahydro-1H-indol-6-yl}urea trifluoroacetate,
N-(3,4-difluorophenyl)-N′-{(3aS*,6R*,7aS*)-3a-(3,4-dimethoxyphenyl)-1-[2-(dimethylamino)ethyl]octahydro-1H-indol-6-yl}urea bis(trifluoroacetate),
N-(3,4-difluorophenyl)-N′-[(3aS*,6R*,7aS*)-3a-(3-methoxy-4-propoxyphenyl)-1-methyloctahydro-1H-indol-6-yl]urea trifluoroacetate,
N-(3,4-difluorophenyl)-N′-[(3aS*,6R*,7aS*)-3a-(4-isopropoxy-3-methoxyphenyl)-1-methyloctahydro-1H-indol-6-yl]urea trifluoroacetate,
N-(3,4-difluorophenyl)-N′-{(3aS*,6R*,7aS*)-3a-(3,4-dimethoxyphenyl)-1-[(4-methyl-1H-imidazol-5-yl)methyl]octahydro-1H-indol-6-yl}urea,
N-(3,4-difluorophenyl)-N′-[(3aS*,6R*,7aS*)-3a-(3,4-dimethoxyphenyl)-1-methyl-6-propyloctahydro-1H-indol-6-yl]urea,
N-(3,4-difluorophenyl)-N′-[(3aS*,6S*,7aS*)-3a-(3,4-dimethoxyphenyl)-1-methyl-6-propyloctahydro-1H-indol-6-yl]urea,
(3aS*,6R*,7aS*)-6-({[(3,4-difluorophenyl)amino]carbonyl}amino)-3a-(3,4-dimethoxyphenyl)-1,1-dimethyloctahydro-1H-indolium trifluoroacetate,
N-(3,4-difluorophenyl)-N′-[(3aS*,6R*,7aS*)-3a-(3,4-dimethoxyphenyl)-1-isobutyloctahydro-1H-indol-6-yl]urea trifluoroacetate,
N-[(3aS*,6R*,7aS*)-1-cyclopentyl-3a-(3,4-dimethoxyphenyl)octahydro-1H-indol-6-yl]-N′-(3,4-difluorophenyl)urea trifluoroacetate,
N-(3,4-difluorophenyl)-N′-[(3aS*,6R*,7aS*)-3a-(3,4-dimethoxyphenyl)-1-(3-methylbutyl)octahydro-1H-indol-6-yl]urea trifluoroacetate,
N-[(3aS*,6R*,7aS*)-1-(cyclopropylmethyl)-3a-(3,4-dimethoxyphenyl)octahydro-1H-indol-6-yl]-N′-(3,4-difluorophenyl)urea trifluoroacetate,
N-(3,4-difluorophenyl)-N′-[(3aS*,6R*,7aS*)-3a-(3,4-dimethoxyphenyl)-1-(2-ethylbutyl)octahydro-1H-indol-6-yl]urea trifluoroacetate,
N-(3,4-difluorophenyl)-N′-[(3aS*,6R*,7aS*)-3a-(3,4-dimethoxyphenyl)-1-(3,3-dimethylbutyl)octahydro-1H-indol-6-yl]urea trifluoroacetate,
N-(3,4-difluorophenyl)-N′-[(3aS*,6R*,7aS*)-3a-(3,4-dimethoxyphenyl)-1-(3,3,3-trifluoropropyl)octahydro-1H-indol-6-yl]urea trifluoroacetate,
N-(3,4-difluorophenyl)-N′-[(3aS*,6R*,7aS*)-3a-(3,4-dimethoxyphenyl)-1-(pyridin-2-ylmethyl)octahydro-1H-indol-6-yl]urea bis(trifluoroacetate),
N-(3,4-difluorophenyl)-N′-{(3aS*,6R*,7aS*)-3a-(3,4-dimethoxyphenyl)-1-[3-(dimethylamino)-2,2-dimethylpropyl]octahydro-1H-indol-6-yl}urea bis(trifluoroacetate),
N-(3,4-difluorophenyl)-N′-[(3aS*,6R*,7aS*)-3a-(3,4-dimethoxyphenyl)-1-(tetrahydro-2H-pyran-4-yl)octahydro-1H-indol-6-yl]urea trifluoroacetate,
N-(3,4-difluorophenyl)-N′-[(3aS*,6R*,7aS*)-3a-(3,4-dimethoxyphenyl)-1-(2-fluoroethyl)octahydro-1H-indol-6-yl]urea trifluoroacetate,
N-(3,4-difluorophenyl)-N′-((3aS*,6R*,7aS*)-3a-(3,4-dimethoxyphenyl)-1-{2-[4-(trimethylsilyl)-1H-1,2,3-triazol-1-yl]ethyl}octahydro-1H-indol-6-yl)urea,
N-(3,4-difluorophenyl)-N′-[(3aS*,6R*,7aS*)-3a-(3,4-dimethoxyphenyl)-1-(2-{5-[(4-methylphenyl)sulfonyl]-1H-tetrazol-1-yl}ethyl)octahydro-1H-indol-6-yl]urea,
N-(3,4-difluorophenyl)-N′-((3aS*,6R*,7aS*)-3a-(3,4-dimethoxyphenyl)-1-{2-[4-(methoxymethyl)-1H-1,2,3-triazol-1-yl]ethyl}octahydro-1H-indol-6-yl)urea,
N-[(3aS*,6R*,7aS*)-3a-(3,4-dimethoxyphenyl)-1-methyloctahydro-1H-indol-6-yl]-N′-(3-methylisothiazol-5-yl)urea trifluoroacetate,
N-[(3aS*,6R*,7aS*)-3a-(3,4-dimethoxyphenyl)-1-methyloctahydro-1H-indol-6-yl]-N′-isoxazol-3-ylurea trifluoroacetate,
N-(3,4-difluorophenyl)-N′-((3aS*,6R*,7aS*)-3a-(3,4-dimethoxyphenyl)-{2-[4-(methoxymethyl)-1H-1,2,3-triazol-1-yl]ethyl}octahydro-1H-indol-6-yl)urea trifluoroacetate,
N-(3,4-difluorophenyl)-N′-((3aS*,6R*,7aS*)-3a-(3,4-dimethoxyphenyl)-1-{2-[5-(methoxymethyl)-1H-1,2,3-triazol-1-yl]ethyl}octahydro-1H-indol-6-yl)urea trifluoroacetate,
N-(4-bromo-3-chlorophenyl)-N′-[(3aS*,6R*,7aS*)-3a-(3,4-dimethoxyphenyl)-1-methyloctahydro-1H-indol-6-yl]urea trifluoroacetate,
N-(3-chloro-4-cyanophenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-quinolin-6-ylurea trifluoroacetate,
N-[(3aS*,6R*,7aS*)-3a-(3,4-dimethoxyphenyl)-1-methyloctahydro-1H-indol-6-yl]-N′-(2-methylquinolin-6-yl)urea trifluoroacetate,
N-[(3aS*,6R*,7aS*)-3a-(3,4-dimethoxyphenyl)-1-methyloctahydro-1H-indol-6-yl]-N′-(2-methyl-1H-indol-5-yl)urea trifluoroacetate,
N-[(3aS*,6R*,7aS*)-3a-(3,4-dimethoxyphenyl)-1-methyloctahydro-1H-indol-6-yl]-N′-(2-methyl-1,3-benzothiazol-5-yl)urea trifluoroacetate,
N-(3,5-dibromo-4-methylphenyl)-N′-[(3aS*,6R*,7aS*)-3a-(3,4-dimethoxyphenyl)-1-methyloctahydro-1H-indol-6-yl]urea trifluoroacetate,
N-1H-1,2,3-benzotriazol-5-yl-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-[4-(1,2,3-thiadiazol-4-yl)phenyl]urea trifluoroacetate,
N-[4-(4-bromo-1-methyl-1H-pyrazol-3-yl)phenyl]-N′-[(3aS*,6R*,7aS*)-3a-(3,4-dimethoxyphenyl)-1-methyloctahydro-1H-indol-6-yl]urea trifluoroacetate,
N-(3,4-dicyanophenyl)-N′-[(3aS*,6R*,7aS*)-3a-(3,4-dimethoxyphenyl)-1-methyloctahydro-1H-indol-6-yl]urea trifluoroacetate,
N-(3,4-difluorophenyl)-N′-{(3aS*,6R*,7aS*)-3a-(3,4-dimethoxyphenyl)-1-[(propylamino)acetyl]octahydro-1H-indol-6-yl}urea trifluoroacetate,
N-[(3aS*,6R*,7aS*)-1-cyclohexyl-3a-(3,4-dimethoxyphenyl)octahydro-1H-indol-6-yl]-N′-(3,4-difluorophenyl)urea trifluoroacetate,
(3aS*,6R*,7aS*)-N-cyano-6-({[(3,4-difluorophenyl)amino]carbonyl}amino)-3a-(3,4-dimethoxyphenyl)-N′-ethyloctahydro-1H-indole-1-carboximidamide,
N-(3,4-difluorophenyl)-N′-[(3aS*,6R*,7aS*)-3a-(3,4-dimethoxyphenyl)-1-(1-methylpiperidin-4-yl)octahydro-1H-indol-6-yl]urea bis(trifluoroacetate),
N-(3,4-difluorophenyl)-N′-[(3aS*,6R*,7aS*)-3a-(3,4-dimethoxyphenyl)-1-(tetrahydro-3-thienyl)octahydro-1H-indol-6-yl]urea,
N-[(3aS*,6R*,7aS*)-1-cyclobutyl-3a-(3,4-dimethoxyphenyl)octahydro-1H-indol-6-yl]-N′-(3,4-difluorophenyl)urea trifluoroacetate,
N-[(3aS*,6R*,7aS*)-3a-(3,4-dimethoxyphenyl)-1-methyloctahydro-1H-indol-6-yl]-N′-[4-methoxy-3-(trifluoromethyl)phenyl]urea trifluoroacetate,
N-(3-chloro-4-morpholin-4-ylphenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-[3-methoxy-5-(trifluoromethyl)phenyl]urea trifluoroacetate,
N-[4-cyano-3-(trifluoromethyl)phenyl]-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(3,4,5-trifluorophenyl)urea trifluoroacetate,
N-[(3aS*,6R*,7aS*)-3a-(3,4-dimethoxyphenyl)-1-methyloctahydro-1H-indol-6-yl]-N′-[3-(1H-tetrazol-5-yl)phenyl]urea trifluoroacetate,
N-[(3aS*,6R*,7aS*)-3a-(3,4-dimethoxyphenyl)-1-methyloctahydro-1H-indol-6-yl]-N′-{3-[(trifluoromethyl)sulfonyl]phenyl}urea trifluoroacetate,
N-1,3-benzothiazol-6-yl-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-[4-(methylsulfonyl)phenyl]urea trifluoroacetate,
N-(3-chloro-4-fluorophenyl)-N′-[(3aS*,6R*,7aS*)-3a-(3,4-dimethoxyphenyl)-1-methyloctahydro-1H-indol-6-yl]urea hydrochloride,
N-(3,4-difluorophenyl)-N′-[(3aS*,6R*,7aS*)-3a-(3,4-dihydroxyphenyl)-1-methyloctahydro-1H-indol-6-yl]urea hydrobromide,
N-(3,4-difluorophenyl)-N′-[(3aS*,6R*,7aS*)-3a-(4-hydroxy-3-methoxyphenyl)-1-methyloctahydro-1H-indol-6-yl]urea hydrochloride,
N-2,1,3-benzothiadiazol-4-yl-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-[3-(trifluoromethyl)phenyl]thiourea trifluoroacetate,
N-[(3aS*,6S*,7aS*)-3a-(1,3-benzodioxol-5-yl)-1-benzyloctahydro-1H-indol-6-yl]-N′-(3-chlorophenyl)urea,
N-[(3aS*,6R*,7aS*)-3a-(1,3-benzodioxol-5-yl)-1-benzyloctahydro-1H-indol-6-yl]-N′-(3-chlorophenyl)urea,
N-[(3aS*,6R*,7aS*)-3a-(1,3-benzodioxol-5-yl)-1-methyloctahydro-1H-indol-6-yl]-N′-[3-(trifluoromethyl)phenyl]urea,
N-(3-bromophenyl)-N′-[(3aS*,6R*,7aS*)-3a-(3,4-dimethoxyphenyl)-1-isopropyloctahydro-1H-indol-6-yl]urea trifluoroacetate,
N-[(3aS*,6R*,7aS*)-3a-(3,4-dimethoxyphenyl)-1-isopropyloctahydro-1H-indol-6-yl]-N′-(2,3,4-trifluorophenyl)urea trifluoroacetate,
N-(3,5-difluorophenyl)-N′-[(3aS*,6R*,7aS*)-3a-(3,4-dimethoxyphenyl)-1-isopropyloctahydro-1H-indol-6-yl]urea trifluoroacetate,
N-[3-chloro-4-(trifluoromethyl)phenyl]-N′-[(3aS*,6R*,7aS*)-3a-(3,4-dimethoxyphenyl)-1-isopropyloctahydro-1H-indol-6-yl]urea trifluoroacetate,
N-[(3aS*,6R*,7aS*)-3a-(3,4-dimethoxyphenyl)-1-isopropyloctahydro-1H-indol-6-yl]-N′-(3,5-dimethylphenyl)urea trifluoroacetate,
N-[(3aS*,6R*,7aS*)-3a-(3,4-dimethoxyphenyl)-1-isopropyloctahydro-1H-indol-6-yl]-N′-[4-(trifluoromethyl)phenyl]urea trifluoroacetate,
N-[(3aS*,6R*,7aS*)-3a-(3,4-dimethoxyphenyl)-1-isopropyloctahydro-1H-indol-6-yl]-N′-[3-(trifluoromethyl)phenyl]urea trifluoroacetate,
N-[(3aS*,6R*,7aS*)-3a-(3,4-dimethoxyphenyl)-1-isopropyloctahydro-1H-indol-6-yl]-N′-[4-fluoro-3-(trifluoromethyl)phenyl]urea trifluoroacetate,
N-[(3aS*,6R*,7aS*)-3a-(3,4-dimethoxyphenyl)-1-isopropyloctahydro-1H-indol-6-yl]-N′-[3-fluoro-5-(trifluoromethyl)phenyl]urea trifluoroacetate,
N-(5-chloro-2-methylphenyl)-N′-[(3aS*,6R*,7aS*)-3a-(3,4-dimethoxyphenyl)-1-isopropyloctahydro-1H-indol-6-yl]urea trifluoroacetate,
N-[4-chloro-3-(trifluoromethyl)phenyl]-N′-[(3aS*,6R*,7aS*)-3a-(3,4-dimethoxyphenyl)-1-isopropyloctahydro-1H-indol-6-yl]urea trifluoroacetate,
N-(2,4-difluorophenyl)-N′-[(3aS*,6R*,7aS*)-3a-(3,4-dimethoxyphenyl)-1-isopropyloctahydro-1H-indol-6-yl]urea trifluoroacetate,
N-[(3aS*,6R*,7aS*)-3a-(3,4-dimethoxyphenyl)-1-isopropyloctahydro-1H-indol-6-yl]-N′-(4-fluorophenyl)urea trifluoroacetate,
N-[(3aS*,6R*,7aS*)-3a-(3,4-dimethoxyphenyl)-1-isopropyloctahydro-1H-indol-6-yl]-N′-[2-fluoro-5-(trifluoromethyl)phenyl]urea trifluoroacetate,
N-[(3aS*,6R*,7aS*)-3a-(3,4-dimethoxyphenyl)-1-isopropyloctahydro-1H-indol-6-yl]-N′-(3-fluoro-4-methylphenyl)urea trifluoroacetate,
N-[(3aS*,6R*,7aS*)-1-cyclobutyl-3a-(3,4-dimethoxyphenyl)octahydro-1H-indol-6-yl]-N′-[2-fluoro-3-(trifluoromethyl)phenyl]urea trifluoroacetate,
N-[(3aS*,6R*,7aS*)-1-cyclobutyl-3a-(3,4-dimethoxyphenyl)octahydro-1H-indol-6-yl]-N′-[4-(trifluoromethyl)phenyl]urea trifluoroacetate,
N-[(3aS*,6R*,7aS*)-1-cyclobutyl-3a-(3,4-dimethoxyphenyl)octahydro-1H-indol-6-yl]-N′-(3-fluorophenyl)urea trifluoroacetate,
N-[(3aS*,6R*,7aS*)-1-cyclobutyl-3a-(3,4-dimethoxyphenyl)octahydro-1H-indol-6-yl]-N′-(2,5-difluorophenyl)urea trifluoroacetate,
N-[(3aS*,6R*,7aS*)-1-cyclobutyl-3a-(3,4-dimethoxyphenyl)octahydro-1H-indol-6-yl]-N′-[3-(trifluoromethyl)phenyl]urea trifluoroacetate,
N-(3-chloro-4-fluorophenyl)-N′-[(3aS*,6R*,7aS*)-1-cyclobutyl-3a-(3,4-dimethoxyphenyl)octahydro-1H-indol-6-yl]urea trifluoroacetate,
N-[(3aS*,6R*,7aS*)-1-cyclobutyl-3a-(3,4-dimethoxyphenyl)octahydro-1H-indol-6-yl]-N′-(2,3,4-trifluorophenyl)urea trifluoroacetate,
N-[(3aS*,6R*,7aS*)-1-cyclobutyl-3a-(3,4-dimethoxyphenyl)octahydro-1H-indol-6-yl]-N′-[4-fluoro-3-(trifluoromethyl)phenyl]urea trifluoroacetate,
N-(3-bromophenyl)-N′-[(3aS*,6R*,7aS*)-1-cyclobutyl-3a-(3,4-dimethoxyphenyl)octahydro-1H-indol-6-yl]urea trifluoroacetate,
N-[(3aS*,6R*,7aS*)-1-cyclobutyl-3a-(3,4-dimethoxyphenyl)octahydro-1H-indol-6-yl]-N′-[3-fluoro-5-(trifluoromethyl)phenyl]urea trifluoroacetate,
N-(5-chloro-2-methylphenyl)-N′-[(3aS*,6R*,7aS*)-1-cyclobutyl-3a-(3,4-dimethoxyphenyl)octahydro-1H-indol-6-yl]urea trifluoroacetate,
N-(3-chlorophenyl)-N′-[(3aS*,6R*,7aS*)-1-cyclobutyl-3a-(3,4-dimethoxyphenyl)octahydro-1H-indol-6-yl]urea trifluoroacetate,
N-[(3aS*,6R*,7aS*)-3a-(3,4-dimethoxyphenyl)-1-(tetrahydro-2H-pyran-4-yl)octahydro-1H-indol-6-yl]-N′-[2-fluoro-3-(trifluoromethyl)phenyl]urea trifluoroacetate,
N-[(3aS*,6R*,7aS*)-3a-(3,4-dimethoxyphenyl)-1-(tetrahydro-2H-pyran-4-yl)octahydro-1H-indol-6-yl]-N′-[4-(trifluoromethyl)phenyl]urea trifluoroacetate,
N-[(3aS*,6R*,7aS*)-3a-(3,4-dimethoxyphenyl)-1-(tetrahydro-2H-pyran-4-yl)octahydro-1H-indol-6-yl]-N′-(3-fluorophenyl)urea trifluoroacetate,
N-(2,5-difluorophenyl)-N′-[(3aS*,6R*,7aS*)-3a-(3,4-dimethoxyphenyl)-1-(tetrahydro-2H-pyran-4-yl)octahydro-1H-indol-6-yl]urea trifluoroacetate,
N-[(3aS*,6R*,7aS*)-3a-(3,4-dimethoxyphenyl)-1-(tetrahydro-2H-pyran-4-yl)octahydro-1H-indol-6-yl]-N′-[3-(trifluoromethyl)phenyl]urea trifluoroacetate,
N-(3-chloro-4-fluorophenyl)-N′-[(3aS*,6R*,7aS*)-3a-(3,4-dimethoxyphenyl)-1-(tetrahydro-2H-pyran-4-yl)octahydro-1H-indol-6-yl]urea trifluoroacetate,
N-[(3aS*,6R*,7aS*)-3a-(3,4-dimethoxyphenyl)-1-(tetrahydro-2H-pyran-4-yl)octahydro-1H-indol-6-yl]-N′-(2,3,4-trifluorophenyl)urea trifluoroacetate,
N-[(3aS*,6R*,7aS*)-3a-(3,4-dimethoxyphenyl)-1-(tetrahydro-2H-pyran-4-yl)octahydro-1H-indol-6-yl]-N′-[4-fluoro-3-(trifluoromethyl)phenyl]urea trifluoroacetate,
N-(3-bromophenyl)-N′-[(3aS*,6R*,7aS*)-3a-(3,4-dimethoxyphenyl)-1-(tetrahydro-2H-pyran-4-yl)octahydro-1H-indol-6-yl]urea trifluoroacetate,
N-[(3aS*,6R*,7aS*)-3a-(3,4-dimethoxyphenyl)-1-(tetrahydro-2H-pyran-4-yl)octahydro-1H-indol-6-yl]-N′-[3-fluoro-5-(trifluoromethyl)phenyl]urea trifluoroacetate,
N-(5-chloro-2-methylphenyl)-N′-[(3aS*,6R*,7aS*)-3a-(3,4-dimethoxyphenyl)-1-(tetrahydro-2H-pyran-4-yl)octahydro-1H-indol-6-yl]urea trifluoroacetate,
N-(3-chlorophenyl)-N′-[(3aS*,6R*,7aS*)-3a-(3,4-dimethoxyphenyl)-1-(tetrahydro-2H-pyran-4-yl)octahydro-1H-indol-6-yl]urea trifluoroacetate,
N-[(3aS*,6R*,7aS*)-1-cyclohexyl-3a-(3,4-dimethoxyphenyl)octahydro-1H-indol-6-yl]-N′-[2-fluoro-3-(trifluoromethyl)phenyl]urea trifluoroacetate,
N-[(3aS*,6R*,7aS*)-1-cyclohexyl-3a-(3,4-dimethoxyphenyl)octahydro-1H-indol-6-yl]-N′-[4-(trifluoromethyl)phenyl]urea trifluoroacetate,
N-[(3aS*,6R*,7aS*)-1-cyclohexyl-3a-(3,4-dimethoxyphenyl)octahydro-1H-indol-6-yl]-N′-(3-fluorophenyl)urea trifluoroacetate,
N-[(3aS*,6R*,7aS*)-1-cyclohexyl-3a-(3,4-dimethoxyphenyl)octahydro-1H-indol-6-yl]-N′-(2,5-difluorophenyl)urea trifluoroacetate,
N-[(3aS*,6R*,7aS*)-1-cyclohexyl-3a-(3,4-dimethoxyphenyl)octahydro-1H-indol-6-yl]-N′-[3-(trifluoromethyl)phenyl]urea trifluoroacetate,
N-(3-chloro-4-fluorophenyl)-N′-[(3aS*,6R*,7aS*)-1-cyclohexyl-3a-(3,4-dimethoxyphenyl)octahydro-1H-indol-6-yl]urea trifluoroacetate,
N-[(3aS*,6R*,7aS*)-1-cyclohexyl-3a-(3,4-dimethoxyphenyl)octahydro-1H-indol-6-yl]-N′-(2,3,4-trifluorophenyl)urea trifluoroacetate,
N-[(3aS*,6R*,7aS*)-1-cyclohexyl-3a-(3,4-dimethoxyphenyl)octahydro-1H-indol-6-yl]-N′-[4-fluoro-3-(trifluoromethyl)phenyl]urea trifluoroacetate,
N-(3-bromophenyl)-N′-[(3aS*,6R*,7aS*)-1-cyclohexyl-3a-(3,4-dimethoxyphenyl)octahydro-1H-indol-6-yl]urea trifluoroacetate,
N-[(3aS*,6R*,7aS*)-1-cyclohexyl-3a-(3,4-dimethoxyphenyl)octahydro-1H-indol-6-yl]-N′-[3-fluoro-5-(trifluoromethyl)phenyl]urea trifluoroacetate,
N-(5-chloro-2-methylphenyl)-N′-[(3aS*,6R*,7aS*)-1-cyclohexyl-3a-(3,4-dimethoxyphenyl)octahydro-1H-indol-6-yl]urea trifluoroacetate,
N-(3-chlorophenyl)-N′-[(3aS*,6R*,7aS*)-1-cyclohexyl-3a-(3,4-dimethoxyphenyl)octahydro-1H-indol-6-yl]urea trifluoroacetate,
N-[(3aS*,6R*,7aS*)-3a-(3,4-dimethoxyphenyl)-1-(1-methylpiperidin-4-yl)octahydro-1H-indol-6-yl]-N′-[2-fluoro-3-(trifluoromethyl)phenyl]urea trifluoroacetate,
N-[(3aS*,6R*,7aS*)-3a-(3,4-dimethoxyphenyl)-1-(1-methylpiperidin-4-yl)octahydro-1H-indol-6-yl]-N′-[4-(trifluoromethyl)phenyl]urea trifluoroacetate,
N-[(3aS*,6R*,7aS*)-3a-(3,4-dimethoxyphenyl)-1-(1-methylpiperidin-4-yl)octahydro-1H-indol-6-yl]-N′-(3-fluorophenyl)urea trifluoroacetate,
N-(2,5-difluorophenyl)-N′-[(3aS*,6R*,7aS*)-3a-(3,4-dimethoxyphenyl)-1-(1-methylpiperidin-4-yl)octahydro-1H-indol-6-yl]urea trifluoroacetate,
N-[(3aS*,6R*,7aS*)-3a-(3,4-dimethoxyphenyl)-1-(1-methylpiperidin-4-yl)octahydro-1H-indol-6-yl]-N′-[3-(trifluoromethyl)phenyl]urea trifluoroacetate,
N-(3-chloro-4-fluorophenyl)-N′-[(3aS*,6R*,7aS*)-3a-(3,4-dimethoxyphenyl)-1-(1-methylpiperidin-4-yl)octahydro-1H-indol-6-yl]urea trifluoroacetate,
N-[(3aS*,6R*,7aS*)-3a-(3,4-dimethoxyphenyl)-1-(1-methylpiperidin-4-yl)octahydro-1H-indol-6-yl]-N′-(2,3,4-trifluorophenyl)urea trifluoroacetate,
N-[(3aS*,6R*,7aS*)-3a-(3,4-dimethoxyphenyl)-1-(1-methylpiperidin-4-yl)octahydro-1H-indol-6-yl]-N′-[4-fluoro-3-(trifluoromethyl)phenyl]urea trifluoroacetate,
N-(3-bromophenyl)-N′-[(3aS*,6R*,7aS*)-3a-(3,4-dimethoxyphenyl)-1-(1-methylpiperidin-4-yl)octahydro-1H-indol-6-yl]urea trifluoroacetate,
N-[(3aS*,6R*,7aS*)-3a-(3,4-dimethoxyphenyl)-1-(1-methylpiperidin-4-yl)octahydro-1H-indol-6-yl]-N′-[3-fluoro-5-(trifluoromethyl)phenyl]urea trifluoroacetate,
N-(5-chloro-2-methylphenyl)-N′-[(3aS*,6R*,7aS*)-3a-(3,4-dimethoxyphenyl)-1-(1-methylpiperidin-4-yl)octahydro-1H-indol-6-yl]urea trifluoroacetate,
N-(3-chlorophenyl)-N′-[(3aS*,6R*,7aS*)-3a-(3,4-dimethoxyphenyl)-1-(1-methylpiperidin-4-yl)octahydro-1H-indol-6-yl]urea trifluoroacetate,
N-[(3aS*,6R*,7aS*)-1-(cyclopropylmethyl)-3a-(3,4-dimethoxyphenyl)octahydro-1H-indol-6-yl]-N′-[2-fluoro-3-(trifluoromethyl)phenyl]urea trifluoroacetate,
N-[(3aS*,6R*,7aS*)-1-(cyclopropylmethyl)-3a-(3,4-dimethoxyphenyl)octahydro-1H-indol-6-yl]-N′-[4-(trifluoromethyl)phenyl]urea trifluoroacetate,
N-[(3aS*,6R*,7aS*)-1-(cyclopropylmethyl)-3a-(3,4-dimethoxyphenyl)octahydro-1H-indol-6-yl]-N′-(3-fluorophenyl)urea trifluoroacetate,
N-[(3aS*,6R*,7aS*)-1-(cyclopropylmethyl)-3a-(3,4-dimethoxyphenyl)octahydro-1H-indol-6-yl]-N′-(2,5-difluorophenyl)urea trifluoroacetate,
N-[(3aS*,6R*,7aS*)-1-(cyclopropylmethyl)-3a-(3,4-dimethoxyphenyl)octahydro-1H-indol-6-yl]-N′-[3-(trifluoromethyl)phenyl]urea trifluoroacetate,
N-(3-chloro-4-fluorophenyl)-N′-[(3aS*,6R*,7aS*)-1-(cyclopropylmethyl)-3a-(3,4-dimethoxyphenyl)octahydro-1H-indol-6-yl]urea trifluoroacetate,
N-[(3aS*,6R*,7aS*)-3a-(3,4-dimethoxyphenyl)-1-(1-methylpiperidin-4-yl)octahydro-1H-indol-6-yl]-N′-(2,3,4-trifluorophenyl)urea trifluoroacetate,
N-[(3aS*,6R*,7aS*)-1-(cyclopropylmethyl)-3a-(3,4-dimethoxyphenyl)octahydro-1H-indol-6-yl]-N′-[4-fluoro-3-(trifluoromethyl)phenyl]urea trifluoroacetate,
N-(3-bromophenyl)-N′-[(3aS*,6R*,7aS*)-1-(cyclopropylmethyl)-3a-(3,4-dimethoxyphenyl)octahydro-1H-indol-6-yl]urea trifluoroacetate,
N-[(3aS*,6R*,7aS*)-1-(cyclopropylmethyl)-3a-(3,4-dimethoxyphenyl)octahydro-1H-indol-6-yl]-N′-[3-fluoro-5-(trifluoromethyl)phenyl]urea trifluoroacetate,
N-(5-chloro-2-methylphenyl)-N′-[(3aS*,6R*,7aS*)-1-(cyclopropylmethyl)-3a-(3,4-dimethoxyphenyl)octahydro-1H-indol-6-yl]urea trifluoroacetate,
N-(3-chlorophenyl)-N′-[(3aS*,6R*,7aS*)-1-(cyclopropylmethyl)-3a-(3,4-dimethoxyphenyl)octahydro-1H-indol-6-yl]urea trifluoroacetate,
N-[(3aS*,6R*,7aS*)-3a-(3,4-dimethoxyphenyl)-1-propyloctahydro-1H-indol-6-yl]-N′-[2-fluoro-3-(trifluoromethyl)phenyl]urea trifluoroacetate,
N-[(3aS*,6R*,7aS*)-3a-(3,4-dimethoxyphenyl)-1-propyloctahydro-1H-indol-6-yl]-N′-[4-(trifluoromethyl)phenyl]urea trifluoroacetate,
N-[(3aS*,6R*,7aS*)-3a-(3,4-dimethoxyphenyl)-1-propyloctahydro-1H-indol-6-yl]-N′-(3-fluorophenyl)urea trifluoroacetate,
N-(2,5-difluorophenyl)-N′-[(3aS*,6R*,7aS*)-3a-(3,4-dimethoxyphenyl)-1-propyloctahydro-1H-indol-6-yl]urea trifluoroacetate,
N-[(3aS*,6R*,7aS*)-3a-(3,4-dimethoxyphenyl)-1-propyloctahydro-1H-indol-6-yl]-N′-[3-(trifluoromethyl)phenyl]urea trifluoroacetate,
N-(3-chloro-4-fluorophenyl)-N′-[(3aS*,6R*,7aS*)-3a-(3,4-dimethoxyphenyl)-1-propyloctahydro-1H-indol-6-yl]urea trifluoroacetate,
N-[(3aS*,6R*,7aS*)-3a-(3,4-dimethoxyphenyl)-1-propyloctahydro-1H-indol-6-yl]-N′-(2,3,4-trifluorophenyl)urea trifluoroacetate,
N-[(3aS*,6R*,7aS*)-3a-(3,4-dimethoxyphenyl)-1-propyloctahydro-1H-indol-6-yl]-N′-[4-fluoro-3-(trifluoromethyl)phenyl]urea trifluoroacetate,
N-(3-bromophenyl)-N′-[(3aS*,6R*,7aS*)-3a-(3,4-dimethoxyphenyl)-1-propyloctahydro-1H-indol-6-yl]urea trifluoroacetate,
N-[(3aS*,6R*,7aS*)-3a-(3,4-dimethoxyphenyl)-1-propyloctahydro-1H-indol-6-yl]-N′-[3-fluoro-5-(trifluoromethyl)phenyl]urea trifluoroacetate,
N-(5-chloro-2-methylphenyl)-N′-[(3aS*,6R*,7aS*)-3a-(3,4-dimethoxyphenyl)-1-propyloctahydro-1H-indol-6-yl]urea trifluoroacetate,
N-(3-chlorophenyl)-N′-[(3aS*,6R*,7aS*)-3a-(3,4-dimethoxyphenyl)-1-propyloctahydro-1H-indol-6-yl]urea trifluoroacetate,
N-2,1,3-benzothiadiazol-4-yl-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-[3-(1,1,2,2-tetrafluoroethoxy)phenyl]urea trifluoroacetate,
N-[(3aS*,6R*,7aS*)-3a-(3,4-dimethoxyphenyl)-1-methyloctahydro-1H-indol-6-yl]-N′-[4-nitro-3-(trifluoromethyl)phenyl]urea trifluoroacetate,
N-butyl-3-[([(3aS*,6R*,7aS*)-3a-(3,4-dimethoxyphenyl)-1-methyloctahydro-1H-indol-6-yl]amino}carbonyl)amino]benzenesulfonamide trifluoroacetate,
N-(2,2-difluoro-1,3-benzodioxol-5-yl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(1,1-dioxido-1-benzothien-6-yl)urea trifluoroacetate,
N-(2,5-difluorophenyl)-N′-[(3aS,6R,7aS)-3a-(3,4-dimethoxyphenyl)-1-methyloctahydro-1H-indol-6-yl]urea hydrochloride,
N-(3-chloro-4-fluorophenyl)-N′-[(3aS,6R,7aS)-3a-(3,4-dimethoxyphenyl) 1-methyloctahydro-1H-indol-6-yl]urea hydrochloride,
N-[(3aS,6R,7aS)-3a-(3,4-dimethoxyphenyl)-1-methyloctahydro-1H-indol-6-yl]-N′-[3-fluoro-5-(trifluoromethyl)phenyl]urea hydrochloride,
N-[(3aS,6R,7aS)-3a-(3,4-dimethoxyphenyl)-1-methyloctahydro-1H-indol-6-yl]-N′-(3-fluorophenyl)urea hydrochloride,
N-[(3aS,6R,7aS)-1-(cyclopropylmethyl)-3a-(3,4-dimethoxyphenyl)octahydro-1H-indol-6-yl]-N′-(2,3,4-trifluorophenyl)urea hydrochloride,
N-(3,4-difluorophenyl)-N′-[(1S,10S,12R)-4,5-dimethoxy-9-azatetracyclo[7.5.2.0˜1,10˜.0˜2,7˜]hexadeca-2,4,6-trien-12-yl]urea trifluoroacetate,
N-(3,4-difluorophenyl)-N′-[(3aS,6R,7aS)-3a-(3,4-dimethoxyphenyl)-1-butyloctahydro-1H-indol-6-yl]urea hydrochloride,
N-(3,4-difluorophenyl)-N′-[(3aS,6R,7aS)-3a-(3,4-dimethoxyphenyl)-1-isopropyloctahydro-1H-indol-6-yl]urea hydrochloride,
N-(3,4-difluorophenyl)-N′-[(3aS,6R,7aS)-3a-(3,4-dimethoxyphenyl)-1-propyloctahydro-1H-indol-6-yl]urea hydrochloride,
N-[(3aS,6R,7aS)-1-(cyclopropylmethyl)-3a-(3,4-dimethoxyphenyl)octahydro-1H-indol-6-yl]-N′-(3,4-difluorophenyl)urea hydrochloride,
N-[4-cyano-3-(trifluoromethyl)phenyl]-N′-[(3aS,6R,7aS)-3a-(3,4-dimethoxyphenyl)-1-methyloctahydro-1H-indol-6-yl]urea hydrochloride,
N-[(3aS,6R,7aS)-3a-(3,4-dimethoxyphenyl)-1-methyloctahydro-1H-indol-6-yl]-N′-(3,4,5-trifluorophenyl)urea hydrochloride,
N-[(3aS,6R,7aS)-3a-(3,4-dimethoxyphenyl)-1-methyloctahydro-1H-indol-6-yl]-N′-[4-methoxy-3-(trifluoromethyl)phenyl]urea hydrochloride,
(3aS,6R,7aS)-6-({[(3,4-difluorophenyl)amino]carbonyl}amino)-3a-(3,4-dimethoxyphenyl)-1, 1-dimethyloctahydro-1H-indolium chloride,
N-(3-cyano-4-fluorophenyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-[3-(1,3-oxazol-5-yl)phenyl]urea trifluoroacetate,
N-[(3aS*,6R*,7aS*)-3a-(3,4-dimethoxyphenyl)-1-methyloctahydro-1H-indol-6-yl]-N′-[8-fluoro-2-(trifluoromethyl)quinolin-4-yl]urea trifluoroacetate,
N-[(3aR,6S,7aR)-3a-(3,4-dimethoxyphenyl)-1-methyloctahydro-1H-indol-6-yl]-N′-[3-fluoro-5-(trifluoromethyl)phenyl]urea trifluoroacetate,
N-(4-bromophenyl)-N′-[(3aR,6S,7aR)-3a-(3,4-dimethoxyphenyl)-1-methyloctahydro-1H-indol-6-yl]urea trifluoroacetate,
N-(2,4-difluorophenyl)-N′-[(3aR,6S,7aR)-3a-(3,4-dimethoxyphenyl)-1-methyloctahydro-1H-indol-6-yl]urea trifluoroacetate,
N-(2,5-difluorophenyl)-N′-[(3aR,6S,7aR)-3a-(3,4-dimethoxyphenyl)-1-methyloctahydro-1H-indol-6-yl]urea trifluoroacetate,
N-[(3aR,6S,7aR)-3a-(3,4-dimethoxyphenyl)-1-methyloctahydro-1H-indol-6-yl]-N′-[4-methoxy-3-(trifluoromethyl)phenyl]urea trifluoroacetate,
N-[4-cyano-3-(trifluoromethyl)phenyl]-N′-[(3aR,6S,7aR)-3a-(3,4-dimethoxyphenyl)-1-methyloctahydro-1H-indol-6-yl]urea trifluoroacetate,
N-[(3aR,6S,7aR)-3a-(3,4-dimethoxyphenyl)-1-methyloctahydro-1H-indol-6-yl]-N′-(3,4,5-trifluorophenyl)urea trifluoroacetate,
N-(3-chloro-4-cyanophenyl)-N′-[(3aR,6S,7aR)-3a-(3,4-dimethoxyphenyl)-1-methyloctahydro-1H-indol-6-yl]urea trifluoroacetate,
N-[(3aS*,6R*,7aS*)-1-(chloroacetyl)-3a-(3,4-dimethoxyphenyl)octahydro-1H-indol-6-yl]-N′-(3,4-difluorophenyl)urea,
N-(3,4-difluorophenyl)-N′-{(3aS*,6R*,7aS*)-3a-(3,4-dimethoxyphenyl)-1-[(isopropylamino)acetyl]octahydro-1H-indol-6-yl}urea trifluoroacetate,
N-[(3aS*,6R*,7aS*)-1-(anilinoacetyl)-3a-(3,4-dimethoxyphenyl)octahydro-1H-indol-6-yl]-N′-(3,4-difluorophenyl)urea,
N-(3,4-difluorophenyl)-N′-{(3aS*,6R*,7aS*)-3a-(3,4-dimethoxyphenyl)-1-[(methylamino)acetyl]octahydro-1H-indol-6-yl}urea trifluoroacetate,
N-[(3aS*,6R*,7aS*)-1-(aminoacetyl)-3a-(3,4-dimethoxyphenyl)octahydro-1H-indol-6-yl]-N′-(3,4-difluorophenyl)urea,
N-(3,4-difluorophenyl)-N′-((3aS*,6R*,7aS*)-3a-(3,4-dimethoxyphenyl)-1-{[(2-furylmethyl)amino]acetyl}octahydro-1H-indol-6-yl)urea trifluoroacetate,
N-(3,4-difluorophenyl)-N′-((3aS*,6R*,7aS*)-3a-(3,4-dimethoxyphenyl)-1-{[(pyridin-4-ylmethyl)amino]acetyl}octahydro-1H-indol-6-yl)urea bis(trifluoroacetate),
N-[(3aS*,6R*,7aS*)-1-{[(4-chlorobenzyl)amino]acetyl}-3a-(3,4-dimethoxyphenyl)octahydro-1H-indol-6-yl]-N′-(3,4-difluorophenyl)urea trifluoroacetate,
N-(3,4-difluorophenyl)-N′-{(3aS*,6R*,7aS*)-3a-(3,4-dimethoxyphenyl)-1-[(ethylamino)acetyl]octahydro-1H-indol-6-yl}urea trifluoroacetate,
N-(3,4-difluorophenyl)-N′-{(3aS*,6R*,7aS*)-3a-(3,4-dimethoxyphenyl)-1-[(4-methylpiperidin-1-yl)acetyl]octahydro-1H-indol-6-yl}urea trifluoroacetate,
N-(3,4-difluorophenyl)-N′-[(3aS*,6R*,7aS*)-3a-(3,4-dimethoxyphenyl)-1-(pyrrolidin-1-ylacetyl)octahydro-1H-indol-6-yl]urea trifluoroacetate,
N-(3,4-difluorophenyl)-N′-((3aS*,6R*,7aS*)-3a-(3,4-dimethoxyphenyl)-1-{[(2-hydroxyethyl)amino]acetyl}octahydro-1H-indol-6-yl)urea trifluoroacetate,
N-(3,4-difluorophenyl)-N′-[(3aS*,6R*,7aS*)-3a-(3,4-dimethoxyphenyl)-1-(3,3,5,5-tetramethylcyclohexyl)octahydro-1H-indol-6-yl]urea trifluoroacetate,
N-[(3aS*,6R*,7aS*)-1-bicyclo[2.2.1]hept-2-yl-3a-(3,4-dimethoxyphenyl)octahydro-1H-indol-6-yl]-N′-(3,4-difluorophenyl)urea trifluoroacetate,
N-(3,4-difluorophenyl)-N′-[(3aS*,6R*,7aS*)-3a-(3,4-dimethoxyphenyl)-1-(4-methylcyclohexyl)octahydro-1H-indol-6-yl]urea trifluoroacetate,
N-[(3aS*,6R*,7aS*)-1-allyl-3a-(3,4-dimethoxyphenyl)octahydro-1H-indol-6-yl]-N′-(3,4-difluorophenyl)urea trifluoroacetate,
2-[(3aS*,6R*,7aS*)-6-({[(3,4-difluorophenyl)amino]carbonyl}amino)-3a-(3,4-dimethoxyphenyl)octahydro-1H-indol-1-yl]-N,N-dimethylacetamide trifluoroacetate,
Ethyl {[(3aS*,6R*,7aS*)-6-({[(3,4-difluorophenyl)amino]carbonyl}amino)-3a-(3,4-dimethoxyphenyl)octahydro-1H-indol-1-yl]carbonothioyl}carbamate,
(3aS*,6R*,7aS*)-6-({[(3,4-difluorophenyl)amino]carbonyl}amino)-3a-(3,4-dimethoxyphenyl)octahydro-1H-indole-1-carbothioamide,
N-(3,4-difluorophenyl)-N′-[(3aS*,6R*,7aS*)-3a-(3,4-dimethoxyphenyl)-1-(4-methyl-1,3-thiazol-2-yl)octahydro-1H-indol-6-yl]urea,
N-(3,4-difluorophenyl)-N′-{(3aS*,6R*,7aS*)-3a-(3,4-dimethoxyphenyl)-1-[(4-methylpiperazin-1-yl)carbonyl]octahydro-1H-indol-6-yl}urea trifluoroacetate,
4-[(3aS*,6R*,7aS*)-6-({[(3,4-difluorophenyl)amino]carbonyl}amino)-3a-(3,4-dimethoxyphenyl)octahydro-1H-indol-1-yl]-4-oxobutanoic acid,
(3aS*,6R*,7aS*)-6-({[(3,4-difluorophenyl)amino]carbonyl}amino)-3a-(3,4-dimethoxyphenyl)octahydro-1H-indole-1-carboxamide,
N-(3,4-difluorophenyl)-N′-[(3aS*,6R*,7aS*)-3a-(3,4-dimethoxyphenyl)-1-pyrimidin-2-yloctahydro-1H-indol-6-yl]urea,
N-(3,4-difluorophenyl)-N′-((3aS*,6R*,7aS*)-3a-(3,4-dimethoxyphenyl)-1-{[2-(4-methylpiperidin-1-yl)ethyl]sulfonyl}octahydro-1H-indol-6-yl)urea trifluoroacetate,
N-(3,4-difluorophenyl)-N′-[(3aS*,6R*,7aS*)-3a-(3,4-dimethoxyphenyl)-1-(3-piperidin-1-ylpropyl)octahydro-1H-indol-6-yl]urea bis(trifluoroacetate),
N-(3,4-difluorophenyl)-N′-[(3aS*,6R*,7aS*)-3a-(3,4-dimethoxyphenyl)-1-(2-piperidin-1-ylethyl)octahydro-1H-indol-6-yl]urea trifluoroacetate,
N-[(3aS*,6R*,7aS*)-1-[(chloromethyl)sulfonyl]-3a-(3,4-dimethoxyphenyl)octahydro-1H-indol-6-yl]-N′-(3,4-difluorophenyl)urea,
N-(3,4-difluorophenyl)-N′-[(3aS*,6R*,7aS*)-3a-(3,4-dimethoxyphenyl)-1-phenyloctahydro-1H-indol-6-yl]urea,
N-(3,4-difluorophenyl)-N′-[(3aS*,6R*,7aS*)-3a-(3,4-dimethoxyphenyl)-1-formyloctahydro-1H-indol-6-yl]urea,
N-[(3aS*,6R*,7aS*)-1-cyclopropyl-3a-(3,4-dimethoxyphenyl)octahydro-1H-indol-6-yl]-N′-(3,4-difluorophenyl)urea trifluoroacetate,
N-(2,5-difluorophenyl)-N′-[(3aS*,6R*,7aS*)-3a-(2-fluoro-3,4-dimethoxyphenyl)-1-methyloctahydro-1H-indol-6-yl]urea trifluoroacetate,
N-[(3aS*,6R*,7aS*)-3a-(2-fluoro-3,4-dimethoxyphenyl)-1-methyloctahydro-1H-indol-6-yl]-N′-[2-fluoro-3-(trifluoromethyl)phenyl]urea trifluoroacetate,
N-[(3aS*,6R*,7aS*)-3a-(2-fluoro-3,4-dimethyloctahydro-1H-indol-6-yl]-N′-[3-fluoro-5-(trifluoromethyl)phenyl]urea trifluoroacetate,
N-(3,4-difluorophenyl)-N′-[(3aS*,6R*,7aS*)-3a-(2-fluoro-3,4-dimethoxyphenyl)-1-methyloctahydro-1H-indol-6-yl]urea,
N-[(3aS*,6R*,7aS*)-3a-(2-fluoro-3,4-dimethoxyphenyl)-1-methyloctahydro-1H-indol-6-yl]-N′-(3,4,5-trifluorophenyl)urea trifluoroacetate,
N-[(3aS*,6R*,7aS*)-1-tert-butyl-3a-(3,4-dimethoxyphenyl)octahydro-1H-indol-6-yl]-N′-[2-fluoro-3-(trifluoromethyl)phenyl]urea trifluoroacetate,
N-[(3aS*,6R*,7aS*)-1-tert-butyl-3a-(3,4-dimethoxyphenyl)octahydro-1H-indol-6-yl]-N′-(2,5-difluorophenyl)urea trifluoroacetate,
N-[(3aS*,6R*,7aS*)-1-tert-butyl-3a-(3,4-dimethoxyphenyl)octahydro-1H-indol-6-yl]-N′-[3-fluoro-5-(trifluoromethyl)phenyl]urea trifluoroacetate,
N-[(3aS*,6R*,7aS*)-1-tert-butyl-3a-(3,4-dimethoxyphenyl)octahydro-1H-indol-6-yl]-N′-(3,4,5-trifluorophenyl)urea trifluoroacetate,
N-{(3aS*,6R*,7aS*)-3a-[3-(benzyloxy)-4-methoxyphenyl]-1-methyloctahydro-1H-indol-6-yl}-N′-(3,4-difluorophenyl)urea,
N-{(3aS*,6S*,7aS*)-3a-[3-(benzyloxy)-4-methoxyphenyl]-1-methyloctahydro-1H-indol-6-yl}-N′-(3,4-difluorophenyl)urea,
N-(3,4-difluorophenyl)-N′-[(3aS*,6R*,7aS*)-3a-(3-hydroxy-4-methoxyphenyl)-1-methyloctahydro-1H-indol-6-yl]urea,
N-(3,4-difluorophenyl)-N′-[(3aS*,6R*,7aS*)-3a-(3-isopropoxy-4-methoxyphenyl)-1-methyloctahydro-1H-indol-6-yl]urea trifluoroacetate,
N-(3,4-difluorophenyl)-N′-[(3aS*,6R*,7aS*)-3a-(3-ethoxy-4-methoxyphenyl)-1-methyloctahydro-1H-indol-6-yl]urea trifluoroacetate,
N-(3,4-difluorophenyl)-N′-[(3aS*,6R*,7aS*)-3a-(4-methoxy-3-propoxyphenyl)-1-methyloctahydro-1H-indol-6-yl]urea trifluoroacetate,
N-{(3aS*,6S*,7aS*)-3a-[4-(difluoromethoxy)-3-methoxyphenyl]-1-methyloctahydro-1H-indol-6-yl}-N′-[2-fluoro-3-(trifluoromethyl)phenyl]urea,
N-{(3aS*,6R*,7aS*)-3a-[4-(difluoromethoxy)-3-methoxyphenyl]-1-methyloctahydro-1H-indol-6-yl}-N′-[2-fluoro-3-(trifluoromethyl)phenyl]urea,
N-{(3aS*,6R*,7aS*)-3a-[4-(difluoromethoxy)-3-methoxyphenyl]-1-methyloctahydro-1H-indol-6-yl}-N′-(3,4-difluorophenyl)urea,
N-{(3aS*,6R*,7aS*)-3a-[4-(difluoromethoxy)-3-methoxyphenyl]-1-methyloctahydro-1H-indol-6-yl}-N′-[3-fluoro-5-(trifluoromethyl)phenyl]urea,
N-{(3aS*,6S*,7aS*)-3a-[4-(difluoromethoxy)-3-methoxyphenyl]-1-methyloctahydro-1H-indol-6-yl}-N′-[3-fluoro-5-(trifluoromethyl)phenyl]urea,
N-{(3aS*,6R*,7aS*)-3a-[4-(difluoromethoxy)-3-methoxyphenyl]-1-methyloctahydro-1H-indol-6-yl}-N′-(2,5-difluorophenyl)urea,
N-{(3aS*,6R*,7aS*)-3a-[4-(difluoromethoxy)-3-methoxyphenyl]-1-methyloctahydro-1H-indol-6-yl}-N′-(3,4,5-trifluorophenyl)urea,
N-[3-fluoro-5-(trifluoromethyl)phenyl]-N′-{(3aS*,6R*,7aS*)-3a-[4-methoxy-3-(trifluoromethoxy)phenyl]-1-methyloctahydro-1H-indol-6-yl}urea,
N-[3-fluoro-5-(trifluoromethyl)phenyl]-N′-{(3aS*,6S*,7aS*)-3a-[4-methoxy-3-(trifluoromethoxy)phenyl]-1-methyloctahydro-1H-indol-6-yl}urea,
N-[(3aS*,6R*,7aS*)-3a-(3,4-dimethoxyphenyl)-1-methyloctahydro-1H-indol-6-yl]-N′-(6-fluoro-1,3-benzothiazol-2-yl)urea trifluoroacetate,
N-[(3aS*,6R*,7aS*)-3a-(3,4-dimethoxyphenyl)-1-methyloctahydro-1H-indol-6-yl]-N′-(4-methyl-1,3-thiazol-2-yl)urea trifluoroacetate,
N-(4-chloro-1,3-benzothiazol-2-yl)-N′-[(3aS*,6R*,7aS*)-3a-(3,4-dimethoxyphenyl)-1-methyloctahydro-1H-indol-6-yl]urea trifluoroacetate,
N-[4-(2-chloro-6-fluorophenyl)-1,3-thiazol-2-yl]-N′-[(3aS*,6R*,7aS*)-3a-(3,4-dimethoxyphenyl)-1-methyloctahydro-1H-indol-6-yl]urea trifluoroacetate,
N-(6-chloro-1,3-benzothiazol-2-yl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-isopropyloctahydro-1H-indol-6-yl]-N′-(3-fluorophenyl)urea trifluoroacetate,
N-(3-chloro-4-fluorophenyl)-N′-[(3aS*,6R*,7aS*)-3a-(3,4-dimethoxyphenyl)-1-isopropyloctahydro-1H-indol-6-yl]urea trifluoroacetate,
N-(3′-cyanobiphenyl-4-yl)-N′-(3,4-difluorophenyl)-N-[(3aS*,6R*,7aS*)-3a-(3,4-dimethoxyphenyl)-1-methyloctahydro-1H-indol-6-yl]urea trifluoroacetate,
N-(3′-cyanobiphenyl-4-yl)-N′-(3,4-difluorophenyl)-N-[(3aS*,6S*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-isopropyloctahydro-1H-indol-6-yl]-N′-[2-fluoro-3-(trifluoromethyl)phenyl]urea trifluoroacetate,
N-(2,5-difluorophenyl)-N′-[(3aS*,6R*,7aS*)-3a-(3,4-dimethoxyphenyl)-1-isopropyloctahydro-1H-indol-6-yl]urea trifluoroacetate,
N-(2-chloro-6-methoxypyridin-4-yl)-N′-[(3aS,6R,7aS)-3a-(3,4-dimethoxyphenyl)-1-methyloctahydro-1H-indol-6-yl]urea hydrochloride,
N-(2,3-difluoro-4-methylphenyl)-N′-[(3aS,6R,7aS)-3a-(3,4-dimethoxyphenyl)-1-methyloctahydro-1H-indol-6-yl]urea trifluoroacetate,
N-(3-chloro-5-fluorophenyl)-N′-[(3aS,6R,7aS)-3a-(3,4-dimethoxyphenyl)-1-methyloctahydro-1H-indol-6-yl]urea trifluoroacetate,
N-[(3aS,6R,7aS)-3a-(3,4-dimethoxyphenyl)-1-methyloctahydro-1H-indol-6-yl]-N′-(4-fluoro-3-methoxyphenyl)urea trifluoroacetate,
N-[3,5-difluoro-4-(trifluoromethyl)phenyl]-N′-[(3aS,6R,7aS)-3a-(3,4-dimethoxyphenyl)-1-methyloctahydro-1H-indol-6-yl]urea trifluoroacetate,
N-[2,4-difluoro-5-(trifluoromethyl)phenyl]-N′-[(3aS,6R,7aS)-3a-(3,4-dimethoxyphenyl)-1-methyloctahydro-1H-indol-6-yl]urea trifluoroacetate,
N-[2,5-difluoro-4-(trifluoromethyl)phenyl]-N′-[(3aS,6R,7aS)-3a-(3,4-dimethoxyphenyl)-1-methyloctahydro-1H-indol-6-yl]urea trifluoroacetate,
N-(2-chloro-4-fluoro-5-methylphenyl)-N′-[(3aS,6R,7aS)-3a-(3,4-dimethoxyphenyl)-1-methyloctahydro-1H-indol-6-yl]urea trifluoroacetate,
N-[(3aS,6R,7aS)-3a-(3,4-dimethoxyphenyl)-1-methyloctahydro-1H-indol-6-yl]-N′-(2,3,5-trifluorophenyl)urea trifluoroacetate,
N-(2-chloro-5-fluorophenyl)-N′-[(3aS,6R,7aS)-3a-(3,4-dimethoxyphenyl)-1-methyloctahydro-1H-indol-6-yl]urea trifluoroacetate,
N-biphenyl-3-yl-N′-[(3aS,6R,7aS)-3a-(3,4-dimethoxyphenyl)-1-methyloctahydro-1H-indol-6-yl]urea trifluoroacetate,
N-(2-chloro-4,6-difluorophenyl)-N′-[(3aS,6R,7aS)-3a-(3,4-dimethoxyphenyl)-1-methyloctahydro-1H-indol-6-yl]urea trifluoroacetate,
N-[(3aS,6R,7aS)-3a-(3,4-dimethoxyphenyl)-1-methyloctahydro-1H-indol-6-yl]-N′-[2-methyl-5-(trifluoromethyl)phenyl]urea trifluoroacetate,
N-(2,2-difluoro-1,3-benzodioxol-5-yl)-N′-[(3aS,6R,7aS)-3a-(3,4-dimethoxyphenyl)-1-methyloctahydro-1H-indol-6-yl]urea trifluoroacetate,
N-[(3aS,6R,7aS)-3a-(3,4-dimethoxyphenyl)-1-methyloctahydro-1H-indol-6-yl]-N′-[3-methyl-5-(trifluoromethyl)phenyl]urea trifluoroacetate,
N-[(3aS,6R,7aS)-3a-(3,4-dimethoxyphenyl)-1-methyloctahydro-1H-indol-6-yl]-N′-[3-fluoro-4-(trifluoromethyl)phenyl]urea trifluoroacetate,
N-[(3aS,6R,7aS)-3a-(3,4-dimethoxyphenyl)-1-methyloctahydro-1H-indol-6-yl]-N′-{2-[(trifluoromethyl)thio]phenyl}urea trifluoroacetate),
N-(2-chloro-3,6-difluorophenyl)-N′-[(3aS,6R,7aS)-3a-(3,4-dimethoxyphenyl)-1-methyloctahydro-1H-indol-6-yl]urea trifluoroacetate,
N-(3-chloro-2,6-difluorophenyl)-N′-[(3aS,6R,7aS)-3a-(3,4-dimethoxyphenyl)-1-methyloctahydro-1H-indol-6-yl]urea trifluoroacetate,
N-[2,3-difluoro-4-(trifluoromethyl)phenyl]-N′-[(3aS,6R,7aS)-3a-(3,4-dimethoxyphenyl)-1-methyloctahydro-1H-indol-6-yl]urea trifluoroacetate,
N-(4-cyano-2,5-difluorophenyl)-N′-[(3aS,6R,7aS)-3a-(3,4-dimethoxyphenyl)-1-methyloctahydro-1H-indol-6-yl]urea trifluoroacetate, and
N-(2-chloro-6-methoxypyridin-4-yl)-N′-[(3aS,6R,7aS)-3a-(3,4-dimethoxyphenyl)-1-methyloctahydro-1H-indol-6-yl]urea trifluoroacetate.

21. A process for the preparation of a compound according to claim 1 comprising at least one of the following reaction sequences:
(a) treatment of an amine with an isocyanate or isothiocyanate,
(b) treatment of a first amine with para-nitrophenyl chloroformate and then with Hunigs base to give a PNP-carbamate, and finally treatment of a second amine with the PNP-carbamate,
(c) treatment of an amine with a carboximidamide hydrochloride in the presence of a base,
(d) treatment of mesembrine with an amine, reduction of the imine formed and then treatment of the resultant amine with an isocyanate,
(e) treatment of an amine with a cyanoimidocarbamate,
(f) treatment of an amine with a nitrovinyl compound,
(g) alkylation of an amine with a ketone via reductive amination and then treatment with an isocyanate,
(h) N-debenzylation of an amine by treatment with hydrogen,
(i) alkylation of an amine with an aldehyde via reductive amination,
(j) treatment of a first amine with triphosgene and then with a second amine,
(k) acylation of an exocyclic amino nitrogen of an amine with a carboxylic acid in the presence of a base and diphenylphosphorylazide,
(l) O-alkylation of an amine with an alkyl halide,
(m) acylation of an endocyclic amino nitrogen of an amine with a carboxylic acid in the presence of a base and a coupling agent,
(n) alkylation of an amine with a halo substituted aldehyde via reductive amination, then nucleophilic displacement of the halogen atom with an azide, and optionally ring closure to give either a triazole or a tetrazole,
(o) alkylation of an amine with a ketone via reductive amination,
(p) alkylation of an amine with a halo substituted aldehyde via reductive amination and then nucleophilic displacement of the halogen atom with an amine,
(q) treatment of a ketone with benzylamine, then a Grignard reagent, debenzylation and finally with an isocyanate,
(r) N-alkylation of an amine with an alkyl halide,
(s) O-dealkylation of an amine by treatment with boron tribromide, and
(t) O-debenzylation of an amine by treatment with hydrogen,
(u) alkylation of a nitrile by treatment with 1-bromo-2-chloroethane,
(v) reduction of a nitrile to an aldehyde,
(w) reaction of an aldehyde with benzylamine to give an imine,
(x) reaction of an imine with but-3-en-2-one to give a ketone,
(y) reaction of a ketone with a reducing agent and an ammonium salt to give an amine,
(z) acylation of an amine with a haloacyl halide to give a haloamide,
(aa) amidation of a haloamide with ammonia or an amine,
(bb) treatment of an amine with an isothiocyanate and the subsequent basic hydrolysis to give a thiourea derivative,
(cc) ring closure of a thiourea derivative with a haloketone to give a thiazole derivative,
(dd) acylation of an amine with an acyl halide to give an amide,
(ee) acylation of an amine with a carboxylic acid anhydride to give a carboxylic acid,
(ff) treatment of an amine with cyanogen bromide,
(gg) treatment of an amine with an aryl halide,
(hh) treatment of an amine with a sulfonyl halide,
(ii) reaction of an aldehyde with an aromatic amine to give an imine,
(jj) acylation of an amine with a carboxylic ester,
(kk) alkylation of an amine with a silane, and
(ll) ring closure of an amine with formaldehyde.

22. A pharmaceutical composition comprising the compound of claim 1.

23. A method for treating or preventing of obesity, diabetes mellitus, hyperlipidemia, hyperglycemia, depression, anxiety, or urinary incontinence, the method comprising administering the compound of claim 1.

24. A method for modulating appetite comprising administering the compound of claim 1.

25. A method for modulating the acitivty of the MCH1R receptor comprising administering the compound of claim 1.

26. The metod of any of claims 23-25 further comprising administering a compound selected from MC-4 agonists, 5HT_{2c} agonists, and 5HT₆ antagonists.

27. The pharmaceutical composition of claim 22 further comprising an anti-obesity medicament.
